(19) United States
(12) Patent Application Publication
(10) Pub. No.: US 2026/0008871 A1
(43) Pub. Date: Jan. 8, 2026

(54) ANTI-MERTK ANTIBODIES AND THEIR METHODS OF USE

(71) Applicant: Genentech, Inc., South San Francisco, CA (US)

(72) Inventors: Wei-Ching LIANG, Foster City, CA (US); WeiYu LIN, Millbrae, CA (US); Yan WU, Foster City, CA (US); Minhong YAN, Foster City, CA (US)

(73) Assignee: Genentech, Inc., South San Francisco, CA (US)

(21) Appl. No.: 19/183,564

(22) Filed: Apr. 18, 2025

Related U.S. Application Data
(63) Continuation of application No. 17/500,741, filed on Oct. 13, 2021, now abandoned.
Application No. 17/500,741 is a continuation of application No. PCT/US2020/028828, filed on Apr. 17, 2020.
(60) Provisional application No. 62/836,580, filed on Apr. 19, 2019.
(60) Provisional application No. 62/890,858, filed on Aug. 23, 2019.

(51) Int. Cl. C07K 16/40; A61K 39/00; A61P 35/00

(52) U.S. Cl. CPC C07K 16/40; A61P 35/00; A61K 2039/505; C07K 2317/24; C07K 2317/92

(57) ABSTRACT

The present disclosure provides anti-MerTK antibodies and methods of use thereof. The methods comprise administering an anti-MerTK antibody or an immunoconjugate thereof.

### CROSS REFERENCE TO RELATED APPLICATIONS

This application is a continuation of U.S. application Ser. No. 17/500,741, filed Oct. 13, 2021, which is a continuation of International Application No. PCT/US2020/028828, filed Apr. 17, 2020, which claims the priority benefit of U.S. Provisional Application Nos. 62/836,580, filed Apr. 19, 2019; and 62/890,858, filed Aug. 23, 2019; each of which is hereby incorporated by reference in its entirety.

### REFERENCE TO AN ELECTRONIC SEQUENCE LISTING

The content of the electronic sequence listing (146392047902seglist.xml; Size: 184,978 bytes; and Date of Creation: Apr. 16, 2025) is herein incorporated by reference in its entirety.

### FIELD

The present disclosure relates to anti-MerTK antibodies and methods of use thereof.

### BACKGROUND

Currently most cancer immuno-oncology (IO) therapies focus on modulating the activity of T cells, the adaptive arm of immune system, by blocking inhibitory pathways that serve as immunological checkpoints. However, long-lasting responses triggered by these therapies are limited to subpopulations of cancer patients. The relatively low response rate is caused by various immunosuppressive mechanisms in the tumor microenvironment. The innate immune system is an integral part of an effective immune response. Innate immune cells play a crucial part in initiating and subsequent direction of the adaptive immune response. Targeting the innate immune system may complement the adaptive immuno-oncology therapies (Mullard, A., Nat. Rev. Drug Discov., 17: 3-5 (2018)).

Macrophages of the innate immune system are abundant in various types of solid tumors and may contribute to the relatively low response rate to T-cell based therapy. They are versatile cells capable of carrying out various functions, including phagocytosis. Macrophages are professional phagocytes highly specialized in removal of dying or dead cells, and cellular debris. It is estimated that billions of cells die every day in the human body. But it is rare to find apoptotic cells in tissues under normal physiological conditions thanks to the rapid and efficient clearance by phagocytes. In homeostasis, apoptotic cells are removed at the early stage of cell death before loss of plasma membrane integrity. Therefore, in general apoptosis is immunologically silent. In solid tumors, uncontrolled tumor growth is often accompanied by increased cell death due to hypoxia and metabolic stress. To evade immune surveillance, tumors take advantage of the non-immunogenic nature of apoptosis. Tumor associated macrophages (TAMs) actively remove the dying tumor cells to avoid alerting the immune system.

MerTK has been shown to play a role in clearance of apoptotic cells. Therefore, reduction of MerTK-mediated clearance of apoptotic cells using MerTK inhibitors is an attractive therapeutic approach in treating cancer. Existing anti-MerTK antibodies have been described but may be unsuitable for therapeutic development. For example, White et al. (“MERTK-Specific Antibodies That Have Therapeutic Antitumor Activity in Mice Disrupt the Integrity of the Retinal Pigmented Epithelium in Cynomolgus Monkeys,” presented at the American Association for Cancer Research Annual Meeting; Mar. 31, 2019; Atlanta, GA) describe two anti-MerTK antibodies: one that binds to human MerTK with higher affinity (8.7×10⁻¹¹M; SRF1), and one that binds to human MerTK with lower affinity (4.4×10⁹) but cross-reacts with murine MerTK (SRF2). These antibodies were shown to inhibit various MerTK functions and inhibit tumor growth in combination with anti-PD-L1 antibody in a mouse model. However, both antibodies were found to promote retinal toxicity in cynomolgus monkey. As such, neither antibody would be acceptable as a therapeutic candidate. These findings underscore the importance of examining multiple factors, not simply antibody affinity, in developing an efficacious therapeutic candidate with an acceptable safety profile.

Thus, there remains a need for an optimal therapy for treating, stabilizing, preventing, and/or delaying development of various cancers. In particular, anti-MerTK antibodies having optimal binding characteristics (e.g., on and off rates) as well as desired biological effects are needed.

All references cited herein, including patent applications, patent publications, and UniProtKB/Swiss-Prot Accession numbers are herein incorporated by reference in their entirety, as if each individual reference were specifically and individually indicated to be incorporated by reference.

### SUMMARY

Described herein are anti-MerTK antibodies and methods of use thereof that meet the need for optimized therapy for treating, stabilizing, preventing, and/or delaying development of various cancers.

In one aspect, the present disclosure provides an isolated antibody that binds to MerTK where the antibody reduces MerTK-mediated clearance of apoptotic cells. In some embodiments, the antibody reduces MerTK mediated clearance of apoptotic cells by phagocytes. In some embodiments, the phagocytes are macrophages. In an exemplary embodiment, the macrophages are tumor-associated macrophages. In some embodiments, the clearance of apoptotic cells is reduced as measured in an apoptotic cell clearance assay at room temperature.

In some embodiments, anti-MerTK antibodies of the present disclosure reduce ligand-mediated MerTK signaling. In some embodiments, the antibodies induce a pro-inflammatory response, including but not limited to a type I IFN response.

In some embodiments, anti-MerTK antibodies of the present disclosure are monoclonal antibodies. In some embodiments, the antibodies are human, humanized, or chimeric antibodies. In some embodiments, the antibodies are antibody fragments that bind to MerTK. In some embodiments, the antibody binds to a fibronectin-like domain or an immunoglobulin-like domain of MerTK.

In an exemplary embodiment, an anti-MerTK antibody of the present disclosure binds to a fibronectin-like domain of MerTK.

In one aspect, the present disclosure provides an anti-MerTK antibody binding to a fibronectin-like domain of MerTK comprises (a) an HVR-H1 comprising the amino acid sequence of SEQ ID NO: 4, (b) an HVR-H2 comprising the amino acid sequence of SEQ ID NO: 5, and (c) an HVR-H3 comprising the amino acid sequence of SEQ ID NO: 6. In some embodiments, the antibody further comprises (a) an HVR-L1 comprising the amino acid sequence of SEQ ID NO: 1; (b) an HVR-L2 comprising the amino acid sequence of SEQ ID NO: 2; and (c) an HVR-L3 comprising the amino acid sequence of SEQ ID NO: 3. In some embodiments, the antibody comprises (a) a heavy chain variable domain (VH) comprising a sequence having at least 95% sequence identity to the amino acid sequence of SEQ ID NO: 83; (b) a light chain variable domain (VL) comprising a sequence having at least 95% sequence identity to the amino acid sequence of SEQ ID NO: 65; or (c) a VH as in (a) and a VL as in (b). In some embodiments, the antibody comprises a VH comprising the amino acid sequence of SEQ ID NO: 83. In some embodiments, the antibody comprises a VL comprising the amino acid sequence of SEQ ID NO: 65. In some embodiments, the antibody comprises a VH comprising the amino acid sequence of SEQ ID NO: 83 and a VL comprising the amino acid sequence of SEQ ID NO: 65.

In one aspect, the present disclosure provides an anti-MerTK antibody binding to a fibronectin-like domain of MerTK comprises the antibody comprises (a) an HVR-H1 comprising the amino acid sequence of SEQ ID NO: 10, (b) an HVR-H2 comprising the amino acid sequence of SEQ ID NO: 11 and (c) an HVR-H3 comprising the amino acid sequence of SEQ ID NO: 12. In some embodiments, the antibody further comprises (a) an HVR-L1 comprising the amino acid sequence of SEQ ID NO: 7; (b) an HVR-L2 comprising the amino acid sequence of SEQ ID NO: 8; and (c) an HVR-L3 comprising the amino acid sequence of SEQ ID NO: 9. In some embodiments, the antibody comprises (a) a heavy chain variable domain (VH) comprising a sequence having at least 95% sequence identity to the amino acid sequence of SEQ ID NO: 84; (b) a light chain variable domain (VL) comprising a sequence having at least 95% sequence identity to the amino acid sequence of SEQ ID NO: 66; or (c) a VH as in (a) and a VL as in (b). In some embodiments, the antibody comprises a VH comprising the amino acid sequence of SEQ ID NO: 84. In some embodiments, the antibody comprises a VL comprising the amino acid sequence of SEQ ID NO: 66. In some embodiments, the antibody comprises a VH comprising the amino acid sequence of SEQ ID NO: 84 and a VL comprising the amino acid sequence of SEQ ID NO: 66.

In some embodiments, the antibody comprises (a) a heavy chain variable domain (VH) comprising a sequence having at least 95% sequence identity to the amino acid sequence of SEQ ID NO: 85; (b) a light chain variable domain (VL) comprising a sequence having at least 95% sequence identity to the amino acid sequence of SEQ ID NO: 67; or (c) a VH as in (a) and a VL as in (b). In some embodiments, the antibody comprises a VH comprising the amino acid sequence of SEQ ID NO: 85. In some embodiments, the antibody comprises a VL comprising the amino acid sequence of SEQ ID NO: 67. In some embodiments, the antibody comprises a VH comprising the amino acid sequence of SEQ ID NO: 85 and a VL comprising the amino acid sequence of SEQ ID NO: 67.

In some embodiments, the antibody comprises a heavy chain comprising the amino acid sequence of SEQ ID NO: 102. In some embodiments, the antibody comprises a light chain comprising the amino acid sequence of SEQ ID NO: 110.

In some embodiments, the antibody comprises (a) a heavy chain variable domain (VH) comprising a sequence having at least 95% sequence identity to the amino acid sequence of SEQ ID NO: 86; (b) a light chain variable domain (VL) comprising a sequence having at least 95% sequence identity to the amino acid sequence of SEQ ID NO: 68; or (c) a VH as in (a) and a VL as in (b). In some embodiments, the antibody comprises a VH comprising the amino acid sequence of SEQ ID NO: 86. In some embodiments, the antibody comprises a VL comprising the amino acid sequence of SEQ ID NO: 68. In some embodiments, the antibody comprises a VH comprising the amino acid sequence of SEQ ID NO: 86 and a VL comprising the amino acid sequence of SEQ ID NO: 68.

In some embodiments, the antibody comprises a heavy chain comprising the amino acid sequence of SEQ ID NO: 103. In some embodiments, the antibody comprises a light chain comprising the amino acid sequence of SEQ ID NO: 111.

In one aspect, the present disclosure provides an anti-MerTK antibody binding to a fibronectin-like domain of MerTK comprises the antibody comprises (a) an HVR-H1 comprising the amino acid sequence of SEQ ID NO: 16, (b) an HVR-H2 comprising the amino acid sequence of SEQ ID NO: 17 and (c) an HVR-H3 comprising the amino acid sequence of SEQ ID NO: 18. In some embodiments, the antibody further comprises (a) an HVR-L1 comprising the amino acid sequence of SEQ ID NO: 13; (b) an HVR-L2 comprising the amino acid sequence of SEQ ID NO: 14; and (c) an HVR-L3 comprising the amino acid sequence of SEQ ID NO: 15. In some embodiments, the antibody comprises (a) a heavy chain variable domain (VH) comprising a sequence having at least 95% sequence identity to the amino acid sequence of SEQ ID NO: 87; (b) a light chain variable domain (VL) comprising a sequence having at least 95% sequence identity to the amino acid sequence of SEQ ID NO: 69; or (c) a VH as in (a) and a VL as in (b). In some embodiments, the antibody comprises a VH comprising the amino acid sequence of SEQ ID NO: 87. In some embodiments, the antibody comprises a VL comprising the amino acid sequence of SEQ ID NO: 69. In some embodiments, the antibody comprises a VH comprising the amino acid sequence of SEQ ID NO: 87 and a VL comprising the amino acid sequence of SEQ ID NO: 69.

In some embodiments, the antibody comprises (a) a heavy chain variable domain (VH) comprising a sequence having at least 95% sequence identity to the amino acid sequence of SEQ ID NO: 88; (b) a light chain variable domain (VL) comprising a sequence having at least 95% sequence identity to the amino acid sequence of SEQ ID NO: 70; or (c) a VH as in (a) and a VL as in (b). In some embodiments, the antibody comprises a VH comprising the amino acid sequence of SEQ ID NO: 88. In some embodiments, the antibody comprises a VL comprising the amino acid sequence of SEQ ID NO: 70. In some embodiments, the antibody comprises a VH comprising the amino acid sequence of SEQ ID NO: 88 and a VL comprising the amino acid sequence of SEQ ID NO: 70.

In some embodiments, the antibody comprises a heavy chain comprising the amino acid sequence of SEQ ID NO: 104. In some embodiments, the antibody comprises a light chain comprising the amino acid sequence of SEQ ID NO: 112.

In some embodiments, the antibody comprises (a) a heavy chain variable domain (VH) comprising a sequence having at least 95% sequence identity to the amino acid sequence of SEQ ID NO: 89; (b) a light chain variable domain (VL) comprising a sequence having at least 95% sequence identity to the amino acid sequence of SEQ ID NO: 70; or (c) a VH as in (a) and a VL as in (b). In some embodiments, the antibody comprises a VH comprising the amino acid sequence of SEQ ID NO: 89. In some embodiments, the antibody comprises a VL comprising the amino acid sequence of SEQ ID NO: 70. In some embodiments, the antibody comprises a VH comprising the amino acid sequence of SEQ ID NO: 89 and a VL comprising the amino acid sequence of SEQ ID NO: 70.

In some embodiments, the antibody comprises a heavy chain comprising the amino acid sequence of SEQ ID NO: 105. In some embodiments, the antibody comprises a light chain comprising the amino acid sequence of SEQ ID NO: 113.

In one aspect, the present disclosure provides an anti-MerTK antibody binding to a fibronectin-like domain of MerTK comprises (a) an HVR-H1 comprising the amino acid sequence of SEQ ID NO: 22, (b) an HVR-H2 comprising the amino acid sequence of SEQ ID NO: 23 and (c) an HVR-H3 comprising the amino acid sequence of SEQ ID NO: 24. In some embodiments, the antibody further comprises (a) an HVR-L1 comprising the amino acid sequence of SEQ ID NO: 19; (b) an HVR-L2 comprising the amino acid sequence of SEQ ID NO: 20; and (c) an HVR-L3 comprising the amino acid sequence of SEQ ID NO: 21. In some embodiments, the antibody comprises (a) a heavy chain variable domain (VH) comprising a sequence having at least 95% sequence identity to the amino acid sequence of SEQ ID NO: 90; (b) a light chain variable domain (VL) comprising a sequence having at least 95% sequence identity to the amino acid sequence of SEQ ID NO: 71; or (c) a VH as in (a) and a VL as in (b). In some embodiments, the antibody comprises a VH comprising the amino acid sequence of SEQ ID NO: 90. In some embodiments, the antibody comprises a VL comprising the amino acid sequence of SEQ ID NO: 71. In some embodiments, the antibody comprises the amino acid sequence of SEQ ID NO: 90 and a VL comprising the amino acid sequence of SEQ ID NO: 71.

In some embodiments, the antibody comprises (a) a heavy chain variable domain (VH) comprising a sequence having at least 95% sequence identity to the amino acid sequence of SEQ ID NO: 91; (b) a light chain variable domain (VL) comprising a sequence having at least 95% sequence identity to the amino acid sequence of SEQ ID NO: 72; or (c) a VH as in (a) and a VL as in (b). In some embodiments, the antibody comprises a VH comprising the amino acid sequence of SEQ ID NO: 91. In some embodiments, the antibody comprises a VL comprising the amino acid sequence of SEQ ID NO: 72. In some embodiments, the antibody comprises a VH comprising the amino acid sequence of SEQ ID NO: 91 and a VL comprising the amino acid sequence of SEQ ID NO: 72.

In some embodiments, the antibody comprises a heavy chain comprising the amino acid sequence of SEQ ID NO: 106. In some embodiments, the antibody comprises a light chain comprising the amino acid sequence of SEQ ID NO: 114.

In some embodiments, the antibody comprises (a) a heavy chain variable domain (VH) comprising a sequence having at least 95% sequence identity to the amino acid sequence of SEQ ID NO: 92; (b) a light chain variable domain (VL) comprising a sequence having at least 95% sequence identity to the amino acid sequence of SEQ ID NO: 73; or (c) a VH as in (a) and a VL as in (b). In some embodiments, the antibody comprises a VH comprising the amino acid sequence of SEQ ID NO: 92. In some embodiments, the antibody comprises a VL comprising the amino acid sequence of SEQ ID NO: 73. In some embodiments, the antibody comprises a VH comprising the amino acid sequence of SEQ ID NO: 92 and a VL comprising the amino acid sequence of SEQ ID NO: 73.

In some embodiments, the antibody comprises a heavy chain comprising the amino acid sequence of SEQ ID NO: 107. In some embodiments, the antibody comprises the antibody comprises a light chain comprising the amino acid sequence of SEQ ID NO: 115.

In one aspect, the present disclosure provides an anti-MerTK antibody binding to a fibronectin-like domain of MerTK comprises (a) an HVR-H1 comprising the amino acid sequence of SEQ ID NO: 27, (b) an HVR-H2 comprising the amino acid sequence of SEQ ID NO: 28 and (c) an HVR-H3 comprising the amino acid sequence of SEQ ID NO: 29. In some embodiments, the antibody further comprises (a) an HVR-L1 comprising the amino acid sequence of SEQ ID NO: 25; (b) an HVR-L2 comprising the amino acid sequence of SEQ ID NO: 14; and (c) an HVR-L3 comprising the amino acid sequence of SEQ ID NO: 26. In some embodiments, the antibody comprises (a) a heavy chain variable domain (VH) comprising a sequence having at least 95% sequence identity to the amino acid sequence of SEQ ID NO: 93; (b) a light chain variable domain (VL) comprising a sequence having at least 95% sequence identity to the amino acid sequence of SEQ ID NO: 74; or (c) a VH as in (a) and a VL as in (b). In some embodiments, the antibody comprises a VH comprising the amino acid sequence of SEQ ID NO: 93. In some embodiments, the antibody comprises a VL comprising the amino acid sequence of SEQ ID NO: 74. In some embodiments, the antibody comprises a VH comprising the amino acid sequence of SEQ ID NO: 93 and a VL comprising the amino acid sequence of SEQ ID NO: 74.

In one aspect, the present disclosure provides an anti-MerTK antibody binding to a fibronectin-like domain of MerTK comprises (a) an HVR-H1 comprising the amino acid sequence of SEQ ID NO: 33, (b) an HVR-H2 comprising the amino acid sequence of SEQ ID NO: 34 and (c) an HVR-H3 comprising the amino acid sequence of SEQ ID NO: 35. In some embodiments, the antibody further comprises (a) an HVR-L1 comprising the amino acid sequence of SEQ ID NO: 30; (b) an HVR-L2 comprising the amino acid sequence of SEQ ID NO: 31; and (c) an HVR-L3 comprising the amino acid sequence of SEQ ID NO: 32. In some embodiments, the antibody comprises (a) a heavy chain variable domain (VH) comprising a sequence having at least 95% sequence identity to the amino acid sequence of SEQ ID NO: 94; (b) a light chain variable domain (VL) comprising a sequence having at least 95% sequence identity to the amino acid sequence of SEQ ID NO: 75; or (c) a VH as in (a) and a VL as in (b). In some embodiments, the antibody comprises a VH comprising the amino acid sequence of SEQ ID NO: 94. In some embodiments, the antibody comprises a VL comprising the amino acid sequence of SEQ ID NO: 75. In some embodiments, the antibody comprises a VH comprising the amino acid sequence of SEQ ID NO: 94 and a VL comprising the amino acid sequence of SEQ ID NO: 75.

In an exemplary embodiment, an anti-MerTK antibody of the present disclosure binds to an immunoglobulin-like domain of MerTK.

In one aspect, the present disclosure provides an anti-MerTK antibody binding to an immunoglobulin-like domain of MerTK comprises (a) an HVR-H1 comprising the amino acid sequence of SEQ ID NO: 38, (b) an HVR-H2 comprising the amino acid sequence of SEQ ID NO: 39, and (c) an HVR-H3 comprising the amino acid sequence of SEQ ID NO: 40. In some embodiments, the antibody further comprises (a) an HVR-L1 comprising the amino acid sequence of SEQ ID NO: 36; (b) an HVR-L2 comprising the amino acid sequence of SEQ ID NO: 14; and (c) an HVR-L3 comprising the amino acid sequence of SEQ ID NO: 37. In some embodiments, the antibody comprises (a) a heavy chain variable domain (VH) comprising a sequence having at least 95% sequence identity to the amino acid sequence of SEQ ID NO: 95; (b) a light chain variable domain (VL) comprising a sequence having at least 95% sequence identity to the amino acid sequence of SEQ ID NO: 76; or (c) a VH as in (a) and a VL as in (b). In some embodiments, the antibody comprises a VH comprising the amino acid sequence of SEQ ID NO: 95. In some embodiments, the antibody comprises a VL comprising the amino acid sequence of SEQ ID NO: 76. In some embodiments, the antibody comprises a VH comprising the amino acid sequence of SEQ ID NO: 95 and a VL comprising the amino acid sequence of SEQ ID NO: 76.

In one aspect, the present disclosure provides an anti-MerTK antibody binding to an immunoglobulin-like domain of MerTK comprises (a) an HVR-H1 comprising the amino acid sequence of SEQ ID NO: 44, (b) an HVR-H2 comprising the amino acid sequence of SEQ ID NO: 45, and (c) an HVR-H3 comprising the amino acid sequence of SEQ ID NO: 46. In some embodiments, the antibody further comprises (a) an HVR-L1 comprising the amino acid sequence of SEQ ID NO: 41; (b) an HVR-L2 comprising the amino acid sequence of SEQ ID NO: 42; and (c) an HVR-L3 comprising the amino acid sequence of SEQ ID NO: 43. In some embodiments, the antibody comprises (a) a heavy chain variable domain (VH) comprising a sequence having at least 95% sequence identity to the amino acid sequence of SEQ ID NO: 96; (b) a light chain variable domain (VL) comprising a sequence having at least 95% sequence identity to the amino acid sequence of SEQ ID NO: 77; or (c) a VH as in (a) and a VL as in (b). In some embodiments, the antibody comprises a VH comprising the amino acid sequence of SEQ ID NO: 96. In some embodiments, the antibody comprises a VL comprising the amino acid sequence of SEQ ID NO: 77. In some embodiments, the antibody comprises a VH comprising the amino acid sequence of SEQ ID NO: 96 and a VL comprising the amino acid sequence of SEQ ID NO: 77.

In one aspect, the present disclosure provides an anti-MerTK antibody binding to an immunoglobulin-like domain of MerTK comprises (a) an HVR-H1 comprising the amino acid sequence of SEQ ID NO: 50, (b) an HVR-H2 comprising the amino acid sequence of SEQ ID NO: 51, and (c) an HVR-H3 comprising the amino acid sequence of SEQ ID NO: 52. In some embodiments, the antibody further comprises (a) an HVR-L1 comprising the amino acid sequence of SEQ ID NO: 47; (b) an HVR-L2 comprising the amino acid sequence of SEQ ID NO: 48; and (c) an HVR-L3 comprising the amino acid sequence of SEQ ID NO: 49. In some embodiments, the antibody comprises (a) a heavy chain variable domain (VH) comprising a sequence having at least 95% sequence identity to the amino acid sequence of SEQ ID NO: 97; (b) a light chain variable domain (VL) comprising a sequence having at least 95% sequence identity to the amino acid sequence of SEQ ID NO: 78; or (c) a VH as in (a) and a VL as in (b). In some embodiments, the antibody comprises a VH comprising the amino acid sequence of SEQ ID NO: 97. In some embodiments, the antibody comprises a VL comprising the amino acid sequence of SEQ ID NO: 78. In some embodiments, the antibody comprises a VH comprising the amino acid sequence of SEQ ID NO: 97 and a VL comprising the amino acid sequence of SEQ ID NO: 78.

In some embodiments, the antibody comprises (a) a heavy chain variable domain (VH) comprising a sequence having at least 95% sequence identity to the amino acid sequence of SEQ ID NO: 98; (b) a light chain variable domain (VL) comprising a sequence having at least 95% sequence identity to the amino acid sequence of SEQ ID NO: 79; or (c) a VH as in (a) and a VL as in (b). In some embodiments, the antibody comprises a VH comprising the amino acid sequence of SEQ ID NO: 98. In some embodiments, the antibody comprises a VL comprising the amino acid sequence of SEQ ID NO: 79. In some embodiments, the antibody comprises a VH comprising the amino acid sequence of SEQ ID NO: 98 and a VL comprising the amino acid sequence of SEQ ID NO: 79.

In some embodiments, the antibody comprises a heavy chain comprising the amino acid sequence of SEQ ID NO: 108. In some embodiments, the antibody comprises a light chain comprising the amino acid sequence of SEQ ID NO: 116.

In some embodiments, the antibody comprises (a) a heavy chain variable domain (VH) comprising a sequence having at least 95% sequence identity to the amino acid sequence of SEQ ID NO: 99; (b) a light chain variable domain (VL) comprising a sequence having at least 95% sequence identity to the amino acid sequence of SEQ ID NO: 80; or (c) a VH as in (a) and a VL as in (b). In some embodiments, the antibody comprises a VH comprising the amino acid sequence of SEQ ID NO: 99. In some embodiments, the antibody comprises a VL comprising the amino acid sequence of SEQ ID NO: 80. In some embodiments, the antibody comprises a VH comprising the amino acid sequence of SEQ ID NO: 99 and a VL comprising the amino acid sequence of SEQ ID NO: 80.

In some embodiments, the antibody comprises a heavy chain comprising the amino acid sequence of SEQ ID NO: 109. In some embodiments, the antibody comprises a light chain comprising the amino acid sequence of SEQ ID NO: 117.

In one aspect, the present disclosure provides an anti-MerTK antibody binding to an immunoglobulin-like domain of MerTK comprises (a) an HVR-H1 comprising the amino acid sequence of SEQ ID NO: 56, (b) an HVR-H2 comprising the amino acid sequence of SEQ ID NO: 57, and (c) an HVR-H3 comprising the amino acid sequence of SEQ ID NO: 58. In some embodiments, the antibody further comprises (a) an HVR-L1 comprising the amino acid sequence of SEQ ID NO: 53; (b) an HVR-L2 comprising the amino acid sequence of SEQ ID NO: 54; and (c) an HVR-L3 comprising the amino acid sequence of SEQ ID NO: 55. In some embodiments, the antibody comprises (a) a heavy chain variable domain (VH) comprising a sequence having at least 95% sequence identity to the amino acid sequence of SEQ ID NO: 100; (b) a light chain variable domain (VL) comprising a sequence having at least 95% sequence identity to the amino acid sequence of SEQ ID NO: 81; or (c) a VH as in (a) and a VL as in (b). In some embodiments, the antibody comprises a VH comprising the amino acid sequence of SEQ ID NO: 100. In some embodiments, the antibody comprises a VL comprising the amino acid sequence of SEQ ID NO: 81. In some embodiments, the antibody comprises a VH comprising the amino acid sequence of SEQ ID NO: 100 and a VL comprising the amino acid sequence of SEQ ID NO: 81.

In one aspect, the present disclosure provides an anti-MerTK antibody binding to an immunoglobulin-like domain of MerTK comprises (a) an HVR-H1 comprising the amino acid sequence of SEQ ID NO: 62, (b) an HVR-H2 comprising the amino acid sequence of SEQ ID NO: 63, and (c) an HVR-H3 comprising the amino acid sequence of SEQ ID NO: 64. In some embodiments, the antibody further comprises (a) an HVR-L1 comprising the amino acid sequence of SEQ ID NO: 59; (b) an HVR-L2 comprising the amino acid sequence of SEQ ID NO: 60; and (c) an HVR-L3 comprising the amino acid sequence of SEQ ID NO: 61. In some embodiments, the antibody comprises (a) a heavy chain variable domain (VH) comprising a sequence having at least 95% sequence identity to the amino acid sequence of SEQ ID NO: 101; (b) a light chain variable domain (VL) comprising a sequence having at least 95% sequence identity to the amino acid sequence of SEQ ID NO: 82; or (c) a VH as in (a) and a VL as in (b). In some embodiments, the antibody comprises a VH comprising the amino acid sequence of SEQ ID NO: 101. In some embodiments, the antibody comprises a VL comprising the amino acid sequence of SEQ ID NO: 82. In some embodiments, the antibody comprises a VH comprising the amino acid sequence of SEQ ID NO: 101 and a VL comprising the amino acid sequence of SEQ ID NO: 82.

In some embodiments, an anti-MerTK antibody of the present disclosure is a full length IgG1, IgG2, IgG3, or IgG4 antibody. In certain embodiments, the antibody is a full length IgG1 antibody. In certain embodiments, the antibody comprises a LALAPG mutation. In some embodiments, the antibody comprises Q2 and L4 residues in the light chain variable region and 148, G49, and K71 residues in the heavy chain variable region. In some embodiments, the antibody comprises L4 and F87 in the light chain variable region and V24, 148, G49, and K71 in the heavy chain variable region. In some embodiments, the antibody comprises L4 and P43 in the light chain variable region and K71 in the heavy chain variable region. In some embodiments, the antibody comprises G49 and V78 residues in the heavy chain variable region.

In certain embodiments, the anti-MerTK antibodies provided herein bind to human MerTK with a dissociation constant (Kd) of ≤100 nM at 25° C. In certain embodiments, the anti-MerTK antibodies provided herein bind to cyno MerTK with a dissociation constant (Kd) of ≤100 nM at 25° C. In certain embodiments, the anti-MerTK antibodies provided herein bind to mouse MerTK with a dissociation constant (Kd) of ≤10 nM at 25° C. In certain embodiments, the anti-MerTK antibodies provided herein bind to rat MerTK with a dissociation constant (Kd) of ≤10 nM at 25° C. In certain embodiments, the anti-MerTK antibodies provided herein bind to human MerTK with a dissociation constant (Kd) of ≤10 nM, ≤5 nM, or ≤2 nM at 25° C.

In one aspect, the present disclosure provides isolated antibodies that compete for binding to MerTK with a reference antibody. Such reference antibodies include an antibody comprising a VH comprising the amino acid sequence of SEQ ID NO: 83 and a VL comprising the amino acid sequence of SEQ ID NO: 65; an antibody comprising a VH comprising the amino acid sequence of SEQ ID NO: 84 and a VL comprising the amino acid sequence of SEQ ID NO: 66; an antibody comprising a VH comprising the amino acid sequence of SEQ ID NO: 85 and a VL comprising the amino acid sequence of SEQ ID NO: 67; an antibody comprising a VH comprising the amino acid sequence of SEQ ID NO: 86 and a VL comprising the amino acid sequence of SEQ ID NO: 68; an antibody comprising a VH comprising the amino acid sequence of SEQ ID NO: 87 and a VL comprising the amino acid sequence of SEQ ID NO: 69; an antibody comprising a VH comprising the amino acid sequence of SEQ ID NO: 88 and a VL comprising the amino acid sequence of SEQ ID NO: 70; an antibody comprising a VH comprising the amino acid sequence of SEQ ID NO: 89 and a VL comprising the amino acid sequence of SEQ ID NO: 70; an antibody comprising a VH comprising the amino acid sequence of SEQ ID NO: 90 and a VL comprising the amino acid sequence of SEQ ID NO: 71; an antibody comprising a VH comprising the amino acid sequence of SEQ ID NO: 91 and a VL comprising the amino acid sequence of SEQ ID NO: 72; an antibody comprising a VH comprising the amino acid sequence of SEQ ID NO: 92 and a VL comprising the amino acid sequence of SEQ ID NO: 73; an antibody comprising a VH comprising the amino acid sequence of SEQ ID NO: 93 and a VL comprising the amino acid sequence of SEQ ID NO: 74; an antibody comprising a VH comprising the amino acid sequence of SEQ ID NO: 94 and a VL comprising the amino acid sequence of SEQ ID NO: 75; an antibody comprising a VH comprising the amino acid sequence of SEQ ID NO: 95 and a VL comprising the amino acid sequence of SEQ ID NO: 76; an antibody comprising a VH comprising the amino acid sequence of SEQ ID NO: 96 and a VL comprising the amino acid sequence of SEQ ID NO: 77; an antibody comprising a VH comprising the amino acid sequence of SEQ ID NO: 97 and a VL comprising the amino acid sequence of SEQ ID NO: 78; an antibody comprising a VH comprising the amino acid sequence of SEQ ID NO: 98 and a VL comprising the amino acid sequence of SEQ ID NO: 79; an antibody comprising a VH comprising the amino acid sequence of SEQ ID NO: 99 and a VL comprising the amino acid sequence of SEQ ID NO: 80; an antibody comprising a VH comprising the amino acid sequence of SEQ ID NO: 100 and a VL comprising the amino acid sequence of SEQ ID NO: 81; and an antibody comprising a VH comprising the amino acid sequence of SEQ ID NO: 101 and a VL comprising the amino acid sequence of SEQ ID NO: 82. In some embodiments, the isolated antibody binds to human MerTK. In some embodiments, the reference antibody is Y323.

In one aspect, the present disclosure provides isolated antibodies that compete for binding to the same epitope on MerTK as a reference antibody. Such reference antibodies include an antibody comprising a VH comprising the amino acid sequence of SEQ ID NO: 83 and a VL comprising the amino acid sequence of SEQ ID NO: 65; an antibody comprising a VH comprising the amino acid sequence of SEQ ID NO: 84 and a VL comprising the amino acid sequence of SEQ ID NO: 66; an antibody comprising a VH comprising the amino acid sequence of SEQ ID NO: 85 and a VL comprising the amino acid sequence of SEQ ID NO: 67; an antibody comprising a VH comprising the amino acid sequence of SEQ ID NO: 86 and a VL comprising the amino acid sequence of SEQ ID NO: 68; an antibody comprising a VH comprising the amino acid sequence of SEQ ID NO: 87 and a VL comprising the amino acid sequence of SEQ ID NO: 69; an antibody comprising a VH comprising the amino acid sequence of SEQ ID NO: 88 and a VL comprising the amino acid sequence of SEQ ID NO: 70; an antibody comprising a VH comprising the amino acid sequence of SEQ ID NO: 89 and a VL comprising the amino acid sequence of SEQ ID NO: 70; an antibody comprising a VH comprising the amino acid sequence of SEQ ID NO: 90 and a VL comprising the amino acid sequence of SEQ ID NO: 71; an antibody comprising a VH comprising the amino acid sequence of SEQ ID NO: 91 and a VL comprising the amino acid sequence of SEQ ID NO: 72; an antibody comprising a VH comprising the amino acid sequence of SEQ ID NO: 92 and a VL comprising the amino acid sequence of SEQ ID NO: 73; an antibody comprising a VH comprising the amino acid sequence of SEQ ID NO: 93 and a VL comprising the amino acid sequence of SEQ ID NO: 74; an antibody comprising a VH comprising the amino acid sequence of SEQ ID NO: 94 and a VL comprising the amino acid sequence of SEQ ID NO: 75; an antibody comprising a VH comprising the amino acid sequence of SEQ ID NO: 95 and a VL comprising the amino acid sequence of SEQ ID NO: 76; an antibody comprising a VH comprising the amino acid sequence of SEQ ID NO: 96 and a VL comprising the amino acid sequence of SEQ ID NO: 77; an antibody comprising a VH comprising the amino acid sequence of SEQ ID NO: 97 and a VL comprising the amino acid sequence of SEQ ID NO: 78; an antibody comprising a VH comprising the amino acid sequence of SEQ ID NO: 98 and a VL comprising the amino acid sequence of SEQ ID NO: 79; an antibody comprising a VH comprising the amino acid sequence of SEQ ID NO: 99 and a VL comprising the amino acid sequence of SEQ ID NO: 80; an antibody comprising a VH comprising the amino acid sequence of SEQ ID NO: 100 and a VL comprising the amino acid sequence of SEQ ID NO: 81; and an antibody comprising a VH comprising the amino acid sequence of SEQ ID NO: 101 and a VL comprising the amino acid sequence of SEQ ID NO: 82. In some embodiments, the isolated antibody binds to human MerTK. In some embodiments, the reference antibody is Y323.

In one aspect, the present disclosure provides an isolated antibody that binds to MerTK, wherein the antibody comprises a heavy chain comprising the amino acid sequence of SEQ ID NO: 102 and a light chain comprising the amino acid sequence of SEQ ID NO: 110. In one aspect, the present disclosure provides an isolated antibody that binds to MerTK, wherein the antibody comprises a heavy chain comprising the amino acid sequence of SEQ ID NO: 103 and a light chain comprising the amino acid sequence of SEQ ID NO: 111. In one aspect, the present disclosure provides an isolated antibody that binds to MerTK, wherein the antibody comprises a heavy chain comprising the amino acid sequence of SEQ ID NO: 104 and a light chain comprising the amino acid sequence of SEQ ID NO: 112. In another aspect, the present disclosure provides an isolated antibody that binds to MerTK, wherein the antibody comprises a heavy chain comprising the amino acid sequence of SEQ ID NO: 105 and a light chain comprising the amino acid sequence of SEQ ID NO: 113. In one aspect, the present disclosure provides an isolated antibody that binds to MerTK, wherein the antibody comprises a heavy chain comprising the amino acid sequence of SEQ ID NO: 106 and a light chain comprising the amino acid sequence of SEQ ID NO: 114. In one aspect, the present disclosure provides an isolated antibody that binds to MerTK, wherein the antibody comprises a heavy chain comprising the amino acid sequence of SEQ ID NO: 107 and a light chain comprising the amino acid sequence of SEQ ID NO: 115. In another aspect, the present disclosure provides an isolated antibody that binds to MerTK, wherein the antibody comprises a heavy chain comprising the amino acid sequence of SEQ ID NO: 108 and a light chain comprising the amino acid sequence of SEQ ID NO: 116. In still another aspect, the present disclosure provides an isolated antibody that binds to MerTK, wherein the antibody comprises a heavy chain comprising the amino acid sequence of SEQ ID NO: 109 and a light chain comprising the amino acid sequence of SEQ ID NO: 117.

In some embodiments, an anti-MerTK of the present disclosure reduces MerTK-mediated clearance of apoptotic cells. In a specific embodiment, the anti-MerTK antibody reduces MerTK-mediated clearance of apoptotic cells by phagocytes. In certain embodiments, the phagocytes are macrophages. In a specific embodiment, the macrophages are tumor-associated macrophages. In some embodiments, the clearance of apoptotic cells is reduced as measured in an apoptotic cell clearance assay at room temperature. In some embodiments, an anti-MerTK antibody of the present disclosure increases circulating tumor DNA (ctDNA) in blood or plasma. In some embodiments, an anti-MerTK antibody of the present disclosure increases cell-free DNA (cfDNA) in blood or plasma.

In some embodiments, an anti-MerTK of the present disclosure is a monoclonal antibody. In certain embodiments, the anti-MerTK antibody is a humanized or chimeric antibody. In certain embodiments, the anti-MerTK antibody is a human, humanized, or chimeric antibody. In certain embodiments, the anti-MerTK antibody is an antibody fragment that binds MerTK. In certain embodiments, the anti-MerTK antibody binds to a fibronectin-like domain or an immunoglobulin-like domain of MerTK. In certain embodiments, the anti-MerTK antibody binds to the fibronectin-like domain of MerTK. In certain embodiments, the anti-MerTK antibody binds to an immunoglobulin-like domain of MerTK.

In one aspect, the present disclosure provides an isolated nucleic acid that encodes any of the anti-MerTK antibodies described herein. In another aspect, the present disclosure provides a vector including the nucleic acid encoding any of the anti-MerTK antibodies described herein. In a still further aspect, the present disclosure provides a host cell containing the vector suitable for expression of the nucleic acid encoding any of the anti-MerTK antibodies described herein.

Further provided herein is a method of producing an anti-MerTK antibody of the present disclosure including culturing a host cell containing a nucleic acid that encodes an anti-MerTK antibody under conditions suitable for expression of the antibody. In some embodiments, the method further includes recovering the anti-MerTK antibody from the cell culture.

In one aspect, the present disclosure pertains to an immunoconjugate including an anti-MerTK antibody provided herein conjugated to a cytotoxic agent. In another aspect, the present disclosure pertains to a pharmaceutical formulation including any of the above described anti-MerTK antibodies and a pharmaceutically-acceptable carrier. In another aspect, the present disclosure pertains to a pharmaceutical formulation including any of the above described anti-MerTK immunoconjugates and a pharmaceutically-acceptable carrier.

In one aspect, the present disclosure provides the anti-MerTK antibodies or immunoconjugates as described above for use as a medicament. In some embodiments, the use is in treating cancer. In some embodiments, the use is in reducing MerTK-mediated clearance of apoptotic cells.

Further provided herein are uses of the anti-MerTK antibodies or immunoconjugates as described above in the manufacture of a medicament. In some embodiments, the medicament is for treatment of cancer. In some embodiments, the cancer expresses functional STING, functional Cx43, and functional cGAS polypeptides. In some embodiments, the cancer comprises tumor-associated macrophages that express functional STING polypeptides. In some embodiments, the cancer comprises tumor cells that express functional cGAS polypeptides. In some embodiments, the cancer comprises tumor cells that express functional Cx43 polypeptides. In certain embodiments, the cancer is colon cancer. In some embodiments, the medicament is for reducing MerTK-mediated clearance of apoptotic cells.

In some embodiments, the uses may further include an additional therapy or administration of an effective amount of an additional therapeutic agent. In some embodiments, the additional therapy is selected from one or more of tamoxifen, letrozole, exemestane, anastrozole, irinotecan, cetuximab, fulvestrant, vinorelbine, erlotinib, bevacizumab, vincristine, imatinib mesylate, sorafenib, lapatinib, trastuzumab, cisplatin, gemcitabine, methotrexate, vinblastine, carboplatin, paclitaxel, 5-fluorouracil, doxorubicin, bortezomib, melphalan, prednisone, and docetaxel. In some embodiments, the additional therapeutic agent is an immune checkpoint inhibitor. In some embodiments, the immune checkpoint inhibitor is selected from one or more of a cytotoxic T-lymphocyte-associated protein 4 (CTLA4) inhibitor, a programmed cell death protein 1 (PD-1) binding antagonist, or a programmed death-ligand 1 (PDL1) binding antagonist. In some embodiments, the immune checkpoint inhibitor is a PDL1 binding antagonist. In an exemplary embodiment, the PDL1 binding antagonist is an anti-PDL1 antibody. In some such embodiments, the anti-PDL1 antibody is atezolizumab. In some embodiments, the medicament is further used in combination with an effective amount of a chemotherapeutic agent.

In another aspect, provided herein are methods for treating or delaying progression of cancer in an individual including administering to the individual an effective amount of an anti-MerTK antibody or an immunoconjugate thereof as described in the present disclosure. In some embodiments, the cancer expresses functional STING, functional Cx43, and functional cGAS polypeptides. In some embodiments, the cancer comprises tumor-associated macrophages that express functional STING polypeptides. In some embodiments, the cancer comprises tumor cells that express functional cGAS polypeptides. In some embodiments, the cancer comprises tumor cells that express functional Cx43 polypeptides. In certain embodiments, the cancer is colon cancer.

In some embodiments, the methods may further include an additional therapy or administration of an effective amount of an additional therapeutic agent. In some embodiments, the additional therapy is selected from one or more of tamoxifen, letrozole, exemestane, anastrozole, irinotecan, cetuximab, fulvestrant, vinorelbine, erlotinib, bevacizumab, vincristine, imatinib mesylate, sorafenib, lapatinib, trastuzumab, cisplatin, gemcitabine, methotrexate, vinblastine, carboplatin, paclitaxel, 5-fluorouracil, doxorubicin, bortezomib, melphalan, prednisone, and docetaxel.

In some embodiments, the additional therapeutic agent is an immune checkpoint inhibitor. In some embodiments, the immune checkpoint inhibitor is selected from one or more of a cytotoxic T-lymphocyte-associated protein 4 (CTLA4) inhibitor, a programmed cell death protein 1 (PD-1) binding antagonist, or a programmed death-ligand 1 (PDL1) binding antagonist. In some embodiments, the immune checkpoint inhibitor is a PDL1 binding antagonist. In an exemplary embodiment, the PDL1 binding antagonist is an anti-PDL1 antibody. In some such embodiments, the anti-PDL1 antibody is atezolizumab. In some embodiments, the methods may further comprise administering an effective amount of an additional chemotherapeutic agent to the individual.

In another aspect, provided herein are methods of reducing MerTK-mediated clearance of apoptotic cells in an individual including administering to the individual an effective amount of an anti-MerTK antibody or an immunoconjugate thereof as described in the present disclosure to reduce MerTK-mediated clearance of apoptotic cells. In some embodiments, the clearance of apoptotic cells is reduced by about 1-10, 1-8, 1-5, 1-4, 1-3, 1-2, 2-10, 2-8, 2-5, 2-4, 2-3, 3-10, 3-8, 3-5, or 3-4 fold or by about 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0 fold.

It is to be understood that one, some, or all of the properties of the various embodiments described herein may be combined to form other embodiments of the present disclosure. These and other aspects of the disclosure will become apparent to one of skill in the art. These and other embodiments of the disclosure are further described by the detailed description that follows.

### BRIEF DESCRIPTION OF THE DRAWINGS

FIGS. 1A and 1B: The light chain and heavy chain variable regions of each MerTK specific antibody generated in rabbits were amplified by PCR and cloned into expression vectors for purification and sequencing. The amino acid sequences for the light chain variable region (FIG. 1A) and heavy chain variable region (FIG. 1B) were aligned. Residue numbers referenced are matched to the sequence defined in Kabat et al. and the CDR sequences are underlined. SEQ ID NOs are as follows: Rbt8F4 (SEQ ID NO: 65), Rbt9E3.FN (SEQ ID NO: 66), Rbt10C3 (SEQ ID NO: 69), Rbt10F7 (SEQ ID NO: 71), Rbt11G11 (SEQ ID NO: 76), Rbt12H4 (SEQ ID NO: 77), Rbt13B4 (SEQ ID NO: 78), Rbt13D8 (SEQ ID NO: 74), Rbt14C9 (SEQ ID NO: 81), Rbt18G7 (SEQ ID NO: 82), and Rbt22C4 (SEQ ID NO: 75). SEQ ID NO in FIG. 1B are as follows: Rbt8F4 (SEQ ID NO: 83), Rbt9E3.FN (SEQ ID NO: 84), Rbt10C3 (SEQ ID NO: 87), Rbt10F7 (SEQ ID NO: 90), Rbt11G11 (SEQ ID NO: 95), Rbt12H4 (SEQ ID NO: 96), Rbt13B4 (SEQ ID NO: 97), Rbt13D8 (SEQ ID NO: 93), Rbt14C9 (SEQ ID NO: 100), Rbt18G7 (SEQ ID NO: 101), and Rbt22C4 (SEQ ID NO: 94).

FIGS. 2A, 2B, 2C & 2D: Antibodies 10F7, 9E3, 13B4, and 10C3 were selected for humanization. The amino acid sequences of the light chain and heavy chain variable regions for antibody 10F7 before humanization, following phase 1 of humanization (.v1), and following phase 2 of humanization (.v16) were aligned (FIG. 2A). The amino acid sequences of light chain and heavy chain variable regions for antibody 9E3 before humanization, following phase 1 of humanization (.v1), and following phase 2 of humanization (.v16) were aligned (FIG. 2B). The amino acid sequences of light chain and heavy chain variable regions for antibody 13B4 before humanization, following phase 1 of humanization (.v1), and following phase 2 of humanization (.v16) were aligned (FIG. 2C). The amino acid sequences of light chain and heavy chain variable regions for antibody 10C3 before humanization, following phase 1 of humanization (.v1), and following phase 2 of humanization (.v14) were aligned (FIG. 2D). Residue numbers referenced are matched to the sequence defined in Kabat et al. and the CDR sequences are underlined. SEQ ID NOs for light chain sequences are as follows: Rbt10F7 (SEQ ID NO: 71), h10F7.v1 (SEQ ID NO: 72), h10F7.v16 (SEQ ID NO: 73), Rbt9E3.FN (SEQ ID NO: 66), h9E3.FN.v1 (SEQ ID NO: 67), h9E3.FN.v16 (SEQ ID NO: 68), Rbt13B4 (SEQ ID NO: 78), h13B4.v1 (SEQ ID NO: 79), h13B4.v16 (SEQ ID NO: 80), Rbt10C3 (SEQ ID NO: 69), h10C3.v1 and h10C3.v14 (SEQ ID NO: 70). SEQ ID NO for heavy chain sequences in FIG. 2A-2D are as follows: Rbt10F7 (SEQ ID NO: 90), h10F7.v1 (SEQ ID NO: 91), h10F7.v16 (SEQ ID NO: 92), Rbt9E3.FN (SEQ ID NO: 84), h9E3.FN.v1 (SEQ ID NO: 85), h9E3.FN.v16 (SEQ ID NO: 86), Rbt13B4 (SEQ ID NO: 97), h13B4.v1 (SEQ ID NO: 98), h13B4.v16 (SEQ ID NO: 99), Rbt10C3 (SEQ ID NO: 87), h10C3.v1 (SEQ ID NO: 88), and h10C3.v14 (SEQ ID NO: 89).

FIG. 3: Epitope binning was used to determine epitope domain specificity for each anti-MerTK antibody. Antibodies 8F4, 22C4, and 13D8, raised against mouse MerTK, and antibodies 10C3, 9E3.FN, 10F7, 22C4, 8F4, and 13D8, raised against human MerTK, competed for binding with each other. Antibodies 12H4, 18G7, 14C9, and 11G11, raised against mouse MerTK, and antibodies 13B4, 12H4, 18G7, and 11G11, raised against human MerTK, competed with each other. As described further in the Examples below, antibodies 10C3, 9E3.FN, 10F7, 22C4, 8F4, and 13D8 bind to MerTK's fibronectin-like domain, and antibodies 13B4, 12H4, 18G7, and 11G11 bind to MerTK's Ig-like domain.

FIGS. 4A, 4B, 4C, 4D & 4E: Efferocytosis assays were carried out to evaluate the in vitro phagocytosis inhibiting activity of anti-MerTK antibodies. Anti-MerTK antibodies inhibited the phagocytic activity of human macrophages isolated from three different donors (FIGS. 4A-4C). Anti-MerTK antibody h13B4.v16 (13B4 Fully Humanized), an Ig-domain binding antibody, was 5.2× more potent at inhibiting human macrophage phagocytosis compared to anti-MerTK antibody h10F7.v16 (10F7 Fully Humanized), a fibronectin-domain binding antibody (FIG. 4D). Anti-MerTK antibody 14C9 mIgG2a LALAPG, was 4.8× more potent at inhibiting mouse macrophage phagocytosis compared to anti-MerTK antibody h10F7.v16 (10F7 Fully Humanized) (FIG. 4E).

FIGS. 5A, 5B & 5C: An apoptotic cell clearance assay was carried out to evaluate the in vivo activity of anti-MerTK antibodies. Apoptotic cells accumulated 8 hours after Dex treatment and were mostly cleared by 24 hours (FIG. 5A). Anti-MerTK (clone 14C9, mIgG2a, LALAPG) but not the control antibody anti-gp120 (mIgG2a, LALAPG) blocked the clearance of apoptotic cells in the thymus 24 hours after Dex treatment (FIG. 5B). Anti-MerTK antibodies blocked the clearance of apoptotic cells relative to the anti-gp120 control (FIG. 5C).

FIGS. 6A, 6B, 6C & 6D: Tumor efficacy studies were carried out in the MC-38 syngeneic tumor model to determine whether anti-MerTK antibodies affect tumor growth. Changes in individual tumor size (FIGS. 6A & 6B; each line represents a single tumor) and mean tumor size (FIGS. 6C & 6D), were measured over time for each treatment group. In the tumor volume tracking plots, gray lines represent the tumor size of animals that were still in the study as of the date of data collection (FIGS. 6A & 6B). Red lines represent animals with ulcerated or progressed tumors that were euthanized and removed from study (FIGS. 6A & 6B). Red horizontal dashed lines indicate a doubling in tumor volume from the start of treatment while green horizontal dashed lines represent the smallest measureable tumor volume (FIGS. 6A & 6B). Animals with tumors in the area below the green dashed line are considered to have had a complete response. The treatment combination of anti-gp120 and anti-PDL1 antibodies did not inhibit tumor growth to a large degree. However, treatments combining anti-PDL1 and anti-MerTK antibodies exhibited enhanced anti-tumor activity (FIGS. 6A-6D).

FIGS. 7A, 7B & 7C: A schematic depiction of blocking MerTK-dependent efferocytosis by anti-MerTK antibody (FIG. 7A). An in vitro efferocytosis assay was carried out to evaluate the phagocytosis inhibiting effect of anti-MerTK 14C9 (mIgG2a LALAPG) antibody treatment. Peritoneal macrophages (green) treated with anti-MerTK 14C9 (mIgG2a LALAPG) antibody exhibited approximately 8× less phagocytic clearance of apoptotic thymocytes (red) as compared to macrophages treated with control antibody anti-gp120 (mIgG2a LALAPG) (black) (FIG. 7B). An in vivo apoptotic cell clearance assay was carried out to determine the effect of anti-MerTK treatment on the clearance of apoptotic cells in the thymus. At 24 hours following induction of thymocyte apoptosis with dexamethasone (Dex), mice treated with anti-MerTK 14C9 (mIgG2a LALAPG) antibody accumulated approximately 6× more apoptotic thymus cells (red) as compared to mice treated with control antibody anti-gp120 (mIgG2a LALAPG) (black) (FIG. 7C).

FIGS. 8A, 8B, 8C, 8D & 8E: An in vitro assay to quantify the effect of anti-MerTK 14C9 (mIgG2a LALAPG) antibody treatment on ligand-mediated MerTK signaling was performed by measuring pAKT levels in macrophages incubated with the ligand hGAS6-Fc (EC50=˜84 pM). Treatment of J774A.1 macrophages with increasing concentrations of anti-MerTK 14C9 (mIgG2a LALAPG) antibody blocked ligand-mediated MerTK signaling, as evidenced by lower levels of pAKT in macrophages treated with anti-MerTK 14C9 (mIgG2a LALAPG) as compared to macrophages treated with the isotype control antibody (FIG. 8A). An apoptotic cell clearance assay was carried out to evaluate the in vivo effect of Dex on thymocytes. Apoptotic thymocytes accumulated 8 hours after Dex treatment and were mostly cleared by 24 hours (FIG. 8B). The distribution of the MerTK protein within MC38 tumor sections was imaged using fluorescence microscopy. MerTK protein co-localized with CD68, a marker of tumor-associated macrophages (TAMs), indicating that MerTK is specifically expressed in TAMs (FIG. 8C). No background signal in Mertk^{-/-} tissue sections stained with anti-MerTK 14C9 (mIgG2a LALAPG) antibody was observed. (FIG. 8C). The distribution of MerTK expression was determined using expression data from The Cancer Genome Atlas (TCGA). MerTK expression exhibited greater correlation with the abundance of TAMs compared to other immune cell types (FIG. 8D). An efferocytosis assay was carried out to evaluate the inhibiting effect of anti-MerTK 14C9 (mIgG2a LALAPG) antibody on in vitro phagocytosis of apoptotic thymocytes (AC, red) by TAMs (TAM, green). Anti-MerTK 14C9 (mIgG2a LALAPG) antibody inhibited the phagocytic activity of TAMs isolated from MC38 tumors (FIG. 8E).

FIGS. 9A, 9B, 9C, 9D & 9E: An RNA-sequencing experiment to evaluate the effect of anti-MerTK 14C9 (mIgG2a LALAPG) antibody treatment on the gene expression pattern of MC38 TAMs. Anti-MerTK 14C9 (mIgG2a LALAPG) antibody treatment caused significant changes to gene expression in TAMs (FIG. 9A). A Gene Set Enrichment Analysis (GSEA) was carried out to uncover gene groups that were differentially regulated in response to treatment with anti-MerTK 14C9 (mIgG2a LALAPG) antibody. The IFN-alpha response gene group was enriched following anti-MerTK 14C9 (mIgG2a LALAPG) antibody treatment (FIG. 9B). The effect of anti-MerTK 14C9 (mIgG2a LALAPG) antibody treatment on the expression of Ifnb1 and multiple interferon stimulated genes (ISGs) in TAMs was evaluated by qPCR. The indicated genes were more highly expressed following anti-MerTK 14C9 (mIgG2a LALAPG) antibody treatment relative to control antibody treatment (FIG. 9C). A quantitative ELISA was carried out to determine the effect of anti-MerTK 14C9 (mIgG2a LALAPG) antibody treatment on IFN-beta protein levels. Anti-MerTK 14C9 (mIgG2a LALAPG) antibody treatment led to a significant accumulation of IFN-beta protein in MC38 tumors (FIG. 9D). The effect of anti-MerTK 14C9 (mIgG2a LALAPG) antibody treatment on IFN-beta expression in the indicated MC38 tumor-derived cell types was evaluated by qPCR. IFN-beta was more highly expressed in CD45+ cells and TAMs treated with anti-MerTK 14C9 (mIgG2a LALAPG) relative to cells treated with the control antibody. No significant changes in IFN-beta expression were observed in CD45− cells or dendritic cells (DC) (FIG. 9E).

FIGS. 10A, 10B, 10C, 10D & 10E: A method to isolate TAMs from tumor-derived single cell suspensions is depicted (FIG. 10A). The purity of isolated TAMs was evaluated by FACS (FIG. 10B). Statistical analysis, depicted as a Volcano plot, identified genes whose expression was increased, decreased, or unchanged by anti-MerTK 14C9 (mIgG2a LALAPG) antibody treatment (FIG. 10C). A Gene Set Enrichment Analysis (GSEA) was carried out to uncover gene groups that were differentially regulated in response to treatment with anti-MerTK 14C9 (mIgG2a LALAPG) antibody. The indicated gene groups are ranked according to their degree of enrichment following anti-MerTK 14C9 (mIgG2a LALAPG) antibody treatment (FIG. 10D). qPCR analysis was undertaken to quantify the effect of anti-MerTK 14C9 (mIgG2a LALAPG) antibody treatment on the expression of the indicated ISGs in total MC38 tumors. The indicated genes were more highly expressed following anti-MerTK 14C9 (mIgG2a LALAPG) antibody treatment relative to a control antibody (FIG. 10E).

FIGS. 11A & 11B: qPCR analysis was carried out to quantify the effect of anti-MerTK 14C9 (mIgG2a LALAPG) antibody treatment on the expression of the indicated genes in MC38 tumor-derived TAMS (FIG. 11A) or total MC38 tumor homogenate (FIG. 11B). Actb, Gapdh, Rp113a, Rp119, Hprt, and Rp14 were used as housekeeping genes.

FIGS. 12A, 12B & 12C: An in vivo antigen presentation assay was employed to evaluate the effect of anti-MerTK 14C9 (mIgG2a LALAPG) antibody treatment on antigen presentation by TAMs and dendritic cells (DCs). Anti-MerTK 14C9 (mIgG2a LALAPG) antibody treatment significantly increased the presentation of the MC38.OVA tumor-derived SIINFEKL antigen bound to the MHC Class I molecule, H-2K^{b} by TAMs but not DCs (FIG. 12A). The expression of CD86, a protein that promotes T cell activation, was quantified to evaluate the effect of the anti-MerTK 14C9 (mIgG2a LALAPG) antibody on T cell activation. Anti-MerTK 14C9 (mIgG2a LALAPG) antibody treatment induced higher levels of CD86 on TAMs but not on DCs (FIG. 12A). The effect of anti-MerTK 14C9 (mIgG2a LALAPG) treatment on productive rearrangements and clonality of T cell receptors (TCR) was measured by genomic DNA sequencing of MC38 tumor-derived T cells. Anti-MerTK 14C9 (mIgG2a LALAPG) antibody treatment led to significantly more TCR clonality and productive rearrangements relative to a control antibody (FIG. 12B). The relative abundance of CD8+, CD4+ and p15e tetramer-reactive T cells in MC38 tumors was quantified to determine the effect of anti-MerTK 14C9 (mIgG2a LALAPG) treatment on antitumor immune response. Anti-MerTK 14C9 (mIgG2a LALAPG) treatment significantly enhanced the antitumor response relative to a control antibody, as evidenced by significant increases in the relative abundance of CD8+ and p15e tetramer-reactive T cells following anti-MerTK antibody treatment (FIG. 12C).

FIGS. 13A, 13B & 13C: The protein levels of CCL3, CCL4, CCL5, CCL7 and CCL12 were quantified in tumor homogenates to evaluate the effect of anti-MerTK 14C9 (mIgG2a LALAPG) treatment on autocrine and paracrine cytokines and chemokines. Anti-MerTK 14C9 (mIgG2a LALAPG) antibody treatment caused a significant enrichment of all tested proteins relative to treatment with a control antibody (FIG. 13A). The expression of ISGs was determined by qPCR analysis in peripheral blood mononuclear cells (PBMCs) to determine the effect of anti-MerTK 14C9 (mIgG2a LALAPG) treatment. No significant difference in the expression of the indicated genes was observed following anti-MerTK 14C9 (mIgG2a LALAPG) antibody treatment relative to a control antibody (FIG. 13B). Quantification of the expression of the indicated cytokines and chemokines in tumors (n=10) revealed no significant effect of anti-MerTK 14C9 (mIgG2a LALAPG) treatment.

FIGS. 14A, 14B & 14C: Gating strategies as depicted in the representative FACS plots in FIG. 14A were employed to isolate specific cell types from single cell suspensions of MC38 tumors (FIG. 14A). The frequency of TAMs and DCs over time in MC38 tumors (n=8, day 8; n=10, days 13 and 15) was quantified. TAMs were considerably more abundant than DCs in MC38 tumors and the frequency of CD45+ TAMs increased in tumors over time while DCs remain constant (FIG. 14B). To evaluate the effects of anti-MerTK 14C9 (mIgG2a LALAPG) treatment on CD206 expression in TAMs, flow cytometric analysis was carried out and the MFI, median fluorescence intensity (n=10) was reported. Anti-MerTK 14C9 (mIgG2a LALAPG) antibody treatment caused a decrease of CD206 expression on TAMs (FIG. 14C).

FIGS. 15A, 15B & 15C: MC38 tumors were treated either with single agent anti-MerTK 14C9 (mIgG2a LALAPG) treatment started at early progression stage (FIG. 15A) or combination treatment with anti-MerTK 14C9 (mIgG2a LALAPG) and anti-PD-L1 at established stage (n=10) (FIG. 15B). Single agent anti-MerTK 14C9 (mIgG2a LALAPG) treatment inhibited the growth of early progression stage tumors (FIG. 15A). Combination treatment with anti-PD-L1 and anti-MerTK 14C9 (mIgG2a LALAPG) antibody at established stage inhibited the growth of MC38 tumors, while single agent anti-MerTK 14C9 (mIgG2a LALAPG) or anti-PD-L1 treatment had marginal or modest effects, respectively (FIG. 15B). Established MC38 tumors were treated with anti-MerTK 14C9 (mIgG2a LALAPG) in combination with gemcitabine (Gem) and anti-PD-1 (n=15, control Ab group; n=8, anti-PD-1+anti-MerTK 14C9 (mIgG2a LALAPG); n=10, other groups). Anti-MerTK 14C9 (mIgG2a LALAPG) treatment in combination with gemcitabine (Gem) and anti-PD-1 inhibited tumor growth. Single agent anti-PD-1 or Gem therapy inhibited tumor growth to a lesser extent than anti-PD-1 and/or Gem combination treatments with anti-MerTK 14C9 (mIgG2a LALAPG) (FIG. 15C). Both individual tumor growth curves and LME-fitted tumor growth curves of each group are presented (FIGS. 15A, 15B & 15C).

FIGS. 16A & 16B: The expression of representative ISGs in tumors treated with anti-MerTK 14C9 (mIgG2a LALAPG) in the presence or absence of anti-IFNAR1 (n=5) was quantified to evaluate the effect of Type 1 IFN signaling on anti-MerTK 14C9 (mIgG2a LALAPG) treatment. Anti-IFNAR1 treatment abolished the enhanced expression of the indicated ISGs caused by anti-MerTK 14C9 (mIgG2a LALAPG) (FIG. 16A). The growth of MC38 tumors treated with a combination of anti-MerTK 14C9 (mIgG2a LALAPG) and anti-PD-L1 along with anti-IFNAR1 was evaluated to determine the effect of Type 1 IFN signaling on combination anti-MerTK 14C9 (mIgG2a LALAPG) and anti-PD-L1 treatment (n=10). Anti-IFNAR1 antibody treatment reduced the tumor-inhibiting effect of combination anti-MerTK 14C9 (mIgG2a LALAPG) and anti-PD-L1 therapy. Both individual tumor growth curves and LME-fitted tumor growth curves of each group are presented (FIG. 16B).

FIGS. 17A, 17B & 17C: The growth of MC38 tumors treated with single agent anti-MerTK 14C9 (mIgG2a LALAPG) therapy along with anti-IFNAR1 antibody was evaluated (n=10) to determine the effect of Type 1 IFN signaling on anti-MerTK 14C9 (mIgG2a LALAPG) treatment. Anti-IFNAR1 antibody treatment negated the tumor-inhibiting effect of anti-MerTK 14C9 (mIgG2a LALAPG) antibody therapy (FIG. 17A). The expression of representative ISGs in MC38 tumors growing in WT or Sting^{gt/gt} mice was quantified to evaluate the effect of STING on anti-MerTK 14C9 (mIgG2a LALAPG) antibody treatment (n=9, WT host; n=10, Sting^{gt/gt} host). STING disruption abolished the enhanced expression of the indicated ISGs caused by anti-MerTK 14C9 (mIgG2a LALAPG) (FIG. 17B). The growth of MC38 tumors in WT or Sting^{gt/gt} host mice was quantified to evaluate the effect of STING on anti-MerTK 14C9 (mIgG2a LALAPG) antibody treatment (n=10). STING disruption abolished the tumor-inhibiting effect of anti-MerTK 14C9 (mIgG2a LALAPG) treatment (FIG. 17C). Both individual tumor growth curves and LME-fitted tumor growth curves of each group are presented (FIGS. 17A & 17C).

FIGS. 18A, 18B, 18C, 18D, 18E & 18F: Cytoplasmic DNA transfection experiments were carried out to evaluate the functions of STING and cGAS in the response to cytoplasmic DNA in macrophages. Accumulation of IFN-beta required both functional STING (FIG. 18A) and cGAS (FIG. 18B) expression in macrophages in response to DNA transfection. Western blot analysis of cGAS and STING expression in MC38 tumor cells and J774A.1 macrophages determined that J774A.1 macrophages express cGAS and STING, while MC38 tumor cells only express cGAS (FIG. 18C). The expression of representative ISGs in WT and cGAS^{-/-} AB22 tumors was quantified to evaluate the role of cGAS expression in tumor cells during anti-MerTK treatment. Disruption of cGAS expression in tumor cells abolished the accumulation of the indicated ISGs in response to anti-MerTK treatment (FIG. 18D). The growth of WT or cGAS^{-/-} MC38 tumors was quantified to evaluate the effect of cGAS expression in tumor cells on anti-MerTK as a single agent or in combination with anti-PD-L1 (n=9, WT MC38 with combination treatment; n=10, other groups). Tumor growth inhibition by anti-MerTK and anti-PD-L1 combination therapy was reduced in cGAS^{-/-} MC38 tumors. Both individual tumor growth curves and LME-fitted tumor growth curves of each group are presented (FIG. 18E). Protein quantification by LC-MS/MS was used to measure cGAMP production in MC38 tumor cells, which increased in WT tumor cells following transfection with HT-DNA, but not in cGAS^{-/-} tumor cells (FIG. 18F).

FIGS. 19A, 19B, 19C, 19D, & 19E: The production of IFN-beta protein from WT and Sting^{gt/gt} BMDMs (FIG. 19A) or WT and cGAS^{-/-} J774A.1 macrophages (FIG. 19B) cocultured with UV-irradiated WT or cGAS^{-/-} MC38 cells was quantified. IFN-beta protein accumulation was dependent on cGAS expression in tumor cells and STING expression in macrophages (FIGS. 19A & 19B). cGAS expression in macrophages was dispensable for IFN-beta protein accumulation (FIG. 19B). The expression of representative ISGs in WT or cGAS^{-/-} MC38 tumors growing in WT host mice (n=10) was quantified to evaluate the effect of cGAS expression in tumor cells on anti-MerTK single agent therapy. cGAS disruption in tumor cells abolished the enhanced expression of the indicated ISGs in response to anti-MerTK treatment (FIG. 19C). The growth of WT or cGAS/early stage MC38 tumors grown in WT host mice was measured following single agent anti-MerTK or anti-PD-L1 treatments (n=10). cGAS deficient tumor cells were resistant to single agent anti-MerTK or anti-PD-L1 treatments, as measured by tumor growth inhibition. Both individual tumor growth curves and LME-fitted tumor growth curves of each group are presented (FIGS. 19D & 19E).

FIGS. 20A, 20B, 20C, 20D & 20E: Western blot analysis was carried out to confirm loss of Cx43 protein in Cx43′ MC38 tumor cells (FIG. 20A). A schematic diagram of a dye transfer assay measuring calcein movement between cells through Cx43 is depicted (FIG. 20B). The dye transfer assay of FIG. 20B was carried out to quantify the role of Cx43 in calcein transfer between MC38 tumor cells (FIG. 20C) or from macrophages to tumor cells (FIG. 20D). Loss of Cx43 compromised the transfer of fluorescent dye calcein between MC38 cells (FIG. 20C) and from J774A.1 macrophages to MC38 tumor cells (FIG. 20D). The growth of WT or Cx43′ MC38 tumors in WT host mice was measured following treatment with anti-MerTK and anti-PD-L1 combination therapy (n=10, WT MC38; n=8, Cx43′ MC38). Cx43 deficient tumor cells were resistant to anti-MerTK and anti-PD-L1 combination therapy, as measured by tumor growth inhibition. Both individual tumor growth curves and LME-fitted tumor growth curves of each group are presented (FIG. 20E).

FIGS. 21A, 21B, 21C & 21D: Schematic diagram of gap junction-dependent transfer of cGAMP from MC38 cells, and production of IFN-beta by macrophages (FIG. 21A). The production of IFN-beta protein from J774A.1 macrophages in coculture with HT-DNA transfected (+ DNA) WT or Cx43^{-/-} MC38 tumor cells was quantified. Disruption of Cx43 in tumor cells abolished the increased production of IFN-beta by macrophages caused by DNA transfection of tumor cells (FIG. 21B). The mRNA expression of representative ISGs in Cx43^{-/-} MC38 tumors was quantified to determine the effect of Cx43 disruption in tumor cells on anti-MerTK treatment (n=10, control Ab; n=9, anti-MerTK). Treatment of Cx43^{-/-} MC38 tumors with anti-MerTK led to no significant changes in the expression of ISGs (FIG. 21C). A model depicting blockade of MerTK-dependent innate immune checkpoint. MerTK signaling in TAMs mediates rapid clearance of stressed or dying tumor cells, resulting in quiescent disposal of tumor-derived materials without alerting the immune system. Treatment with anti-MerTK prevents efferocytosis, allowing prolonged production of cGAMP by cGAS-expressing tumor cells and increased transfer of cGAMP via gap junctions to host macrophages. IFN-beta produced by TAMs acts in an autocrine/paracrine manner to increase antigen presentation and expression of co-stimulatory molecules by antigen presenting cells, ultimately leading to enhanced T cell response (FIG. 21D).

FIGS. 22A & 22B: Quantification of circulating tumor DNA (ctDNA) and cell-free DNA (cfDNA) in a mouse MC38 tumor model upon treatment with anti-MerTK or control antibody. MC38 tumor cells were inoculated into C57BL/6J mice. Anti-MerTK or control antibody was administered after tumors were established. Three days after anti-MerTK treatment, a significant increase of ctDNA in the plasma of tumor-bearing mice was detected (FIG. 22A). Anti-MerTK also increased the level of host-derived cfDNA in blood circulation (FIG. 22B). Indicated p-values are based on unpaired, two-tailed Student's t-test. These results demonstrate that in tumor microenvironment anti-MerTK treatment was able to block the ongoing clearance of apoptotic cells by TAMs.

FIG. 23 shows the analysis of anti-MerTK antibody binding affinity to human MerTK using surface plasmon resonance (SPR). Binding affinity of 10 commercial antibodies and h13B4.v16 to human MerTK was determined. Binding affinities were observed as follows: 0.4 nM for Y323, 6.8 nM for A3KCAT, 7.6 nM for 590H11G1E3, 17.3 nM for MAB8912-100 and 1.6 nM for h13B4.v16. The remaining six antibodies (10g86_D2F11, 2D2, 7E5G1, 7N-20, MAB891, and MAB8911) showed no binding to human MerTK.

FIGS. 24A-24C show the results of competitive binding experiments examining anti-MerTK antibodies. Anti-MerTK antibodies Y323, A3KCAT, 590H11G1E3 and MAB8912-100 were tested for competition with antibody h13B4.v16 for binding to human MerTK using the classic sandwich format (FIG. 24A). Antibody Y323 was found to compete with h13B4.v16 for binding to human MerTK (FIG. 24B), whereas antibodies A3KCAT, 590H11G1E3 and MAB8912-100 did not compete with h13B4.v16 for binding to human MerTK (FIG. 24C).

### DETAILED DESCRIPTION

### I. Definitions

It is to be understood that this disclosure is not limited to particular compositions or biological systems, which can, of course, vary. It is also to be understood that the terminology used herein is for the purpose of describing particular embodiments only, and is not intended to be limiting.

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the present disclosure include “comprising,” “consisting,” and “consisting essentially of” aspects and embodiments.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less, or 1 or less. In some embodiments, the VL acceptor human framework is identical in sequence to the VL human immunoglobulin framework sequence or human consensus framework sequence. In some embodiments, the VH acceptor human framework is identical in sequence to the VH human immunoglobulin framework sequence or human consensus framework sequence. In some embodiments, the VL and VH acceptor human frameworks are identical in sequence to a VH and VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The terms “anti-MerTK antibody” and “an antibody that binds to MerTK” refer to an antibody that is capable of binding MerTK with sufficient affinity such that the antibody is useful as a diagnostic and/or therapeutic agent in targeting MerTK. In one embodiment, the extent of binding of an anti-MerTK antibody to an unrelated, non-MerTK protein is less than about 10% of the binding of the antibody to MerTK as measured, e.g., by a radioimmunoassay (RIA). In certain embodiments, an antibody that binds to MerTK has a dissociation constant (Kd) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M). In certain embodiments, an anti-MerTK antibody binds to an epitope of MerTK that is conserved among MerTK from different species.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

An “effective amount” of an agent, e.g., a pharmaceutical formulation, refers to an amount effective, at dosages and for periods of time necessary, to achieve the desired therapeutic or prophylactic result.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

In one embodiment, HVR residues comprise those identified in TABLE 6 of the present disclosure.

Unless otherwise indicated, HVR residues and other residues in the variable domain (e.g., FR residues) are numbered herein according to Kabat et al., supra.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-MerTK antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “LALAPG mutation” as used herein refers to a mutation in the Fc region of an antibody comprising the following three mutations: leucine 234 to alanine (L234A), leucine 235 to alanine (L235A), and proline 239 to glycine (P329G), which has previously been shown to reduce binding to Fc receptors and complement (see e.g., US Publication No. 2012/0251531 and U.S. Pat. No. 8,969,526). The numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “PD-1 axis binding antagonist” refers to a molecule that inhibits the interaction of a PD-1 axis binding partner with either one or more of its binding partners, so as to remove T-cell dysfunction resulting from signaling on the PD-1 signaling axis—with a result being to restore or enhance T-cell function (e.g., proliferation, cytokine production, target cell killing). As used herein, a PD-1 axis binding antagonist includes a PD-1 binding antagonist, a PD-L1 binding antagonist and a PD-L2 binding antagonist.

The term “PD-1 binding antagonist” refers to a molecule that decreases, blocks, inhibits, abrogates or interferes with signal transduction resulting from the interaction of PD-1 with one or more of its binding partners, such as PD-L1 and/or PD-L2. In some embodiments, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PD-L1 and/or PD-L2. In one embodiment, a PD-1 binding antagonist reduces the negative co-stimulatory signal mediated by or through cell surface proteins expressed on T lymphocytes that mediate signaling through PD-1 so as to render a dysfunctional T-cell less dysfunctional (e.g., enhancing effector responses to antigen recognition). In some embodiments, the PD-1 binding antagonist is an anti-PD-1 antibody. Specific examples of PD-1 binding antagonists are provided infra.

The term “PD-L1 binding antagonist” refers to a molecule that decreases, blocks, inhibits, abrogates or interferes with signal transduction resulting from the interaction of PD-L1 with either one or more of its binding partners, such as PD-1 and/or B7-1. In some embodiments, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some embodiments, the PD-L1 binding antagonists include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1 and/or B7-1. In one embodiment, a PD-L1 binding antagonist reduces the negative co-stimulatory signal mediated by or through cell surface proteins expressed on T lymphocytes that mediate signaling through PD-L1 so as to render a dysfunctional T-cell less dysfunctional (e.g., enhancing effector responses to antigen recognition). In some embodiments, a PD-L1 binding antagonist is an anti-PD-L1 antibody. Specific examples of PD-L1 binding antagonists are provided infra.

The term “PD-L2 binding antagonist” refers to a molecule that decreases, blocks, inhibits, abrogates or interferes with signal transduction resulting from the interaction of PD-L2 with either one or more of its binding partners, such as PD-1. In some embodiments, a PD-L2 binding antagonist is a molecule that inhibits the binding of PD-L2 to one or more of its binding partners. In a specific aspect, the PD-L2 binding antagonist inhibits binding of PD-L2 to PD-1. In some embodiments, the PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with one or more of its binding partners, such as PD-1. In one embodiment, a PD-L2 binding antagonist reduces the negative co-stimulatory signal mediated by or through cell surface proteins expressed on T lymphocytes that mediate signaling through PD-L2 so as to render a dysfunctional T-cell less dysfunctional (e.g., enhancing effector responses to antigen recognition). In some embodiments, a PD-L2 binding antagonist is an immunoadhesin.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “MerTK,” as used herein, refers to any native MerTK from any vertebrate source, including mammals such as primates (e.g. humans) and rodents (e.g., mice and rats), unless otherwise indicated. The term encompasses “full-length,” unprocessed MerTK as well as any form of MerTK that results from processing in the cell. The term also encompasses naturally occurring variants of MerTK, e.g., splice variants or allelic variants. The amino acid sequence of an exemplary human MerTK is described in US 2006/0121562.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The term “variable region” or “variable domain” refers to the domain of an antibody heavy or light chain that is involved in binding of the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### II. Anti-MerTK Antibodies

The present disclosure is based on the discovery of novel anti-MerTK antibodies. Such novel anti-MerTK antibodies find use in the treatment of cancer. In particular, the present disclosure is based on the discovery that the anti-MerTK antibodies described herein enhance the effectiveness of immune checkpoint inhibitor-based therapy.

C-Mer proto-oncogene tyrosine kinase (MerTK) is a receptor tyrosine kinase which transduces extracellular signals upon binding to various ligands, such as galectin-3, Protein S, and Gas6, thus activating expression of effector genes. The MerTK pathway regulates essential cellular processes, including cell survival, cytokine production, migration, differentiation, and phagocytosis (Cabernoy N., et al. J Cell Physio. 227 (2012): 401-407; Wu, G., et al. Cell Death & Disease 8 (2017): e2700). Expression of MerTK is found in a variety of hematopoeitic cell types, such as macrophages, dendritic cells, natural killer (NK) cells. Importantly, the MerTK receptor pathway is active in several solid and hematological cancers, including colon cancer (Wu, G., et al. Cell Death & Disease 8 (2017): e2700).

The MerTK receptor is composed of an extracellular component, a transmembrane (TM) domain, and an intracellular component. As shown in the diagram below, the extracellular or ligand-binding region of MerTK contains two immunoglobulin (Ig)-like domains and two fibronectin (FN) type III-like domains.

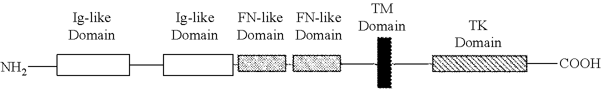

In human MerTK, for example, the two Ig-like domains are defined by amino acid residues 76-195 and amino acid residues 199-283, respectively. Additionally, the two fibronectin-like domains of human MerTK are defined by amino acid residues 286-384 and amino acid residues 388-480, respectively. The intracellular region of MerTK contains a tyrosine kinase (TK) domain, which autophosphorylates specific tyrosine residues following ligand binding to the extracellular region and facilitates MerTK receptor dimerization, thus activating downstream effector gene expression (Toledo, R. A, et al. Clin Can. Res. 22 (2016): 2301-2312). Human MerTK comprises the amino acid sequence:

(SEQ ID NO: 137)
MGPAPLPLLLGLFLPALWRRAITEAREEAKPYPLFPGPFPGSLQTDHTPL
LSLPHASGYQPALMFSPTQPGRPHTGNVAIPQVTSVESKPLPPLAFKHTV
GHIILSEHKGVKFNCSISVPNIYQDTTISWWKDGKELLGAHHAITQFYPD
DEVTAIIASFSITSVQRSDNGSYICKMKINNEEIVSDPIYIEVQGLPHFT
KQPESMNVTRNTAFNLTCQAVGPPEPVNIFWVQNSSRVNEQPEKSPSVLT
VPGLTEMAVFSCEAHNDKGLTVSKGVQINIKAIPSPPTEVSIRNSTAHSI
LISWVPGFDGYSPFRNCSIQVKEADPLSNGSVMIFNTSALPHLYQIKQLQ
ALANYSIGVSCMNEIGWSAVSPWILASTTEGAPSVAPLNVTVFLNESSDN
VDIRWMKPPTKQQDGELVGYRISHVWQSAGISKELLEEVGQNGSRARISV
QVHNATCTVRIAAVTRGGVGPFSDPVKIFIPAHGWVDYAPSSTPAPGNAD
PVLIIFGCFCGFILIGLILYISLAIRKRVQETKFGNAFTEEDSELVVNYI
AKKSFCRRAIELTLHSLGVSEELQNKLEDVVIDRNLLILGKILGEGEFGS
VMEGNLKQEDGTSLKVAVKTMKLDNSSQREIEEFLSEAACMKDFSHPNVI
RLLGVCIEMSSQGIPKPMVILPFMKYGDLHTYLLYSRLETGPKHIPLQTL
LKFMVDIALGMEYLSNRNFLHRDLAARNCMLRDDMTVCVADFGLSKKIYS
GDYYRQGRIAKMPVKWIAIESLADRVYTSKSDVWAFGVTMWEIATRGMTP
YPGVQNHEMYDYLLHGHRLKQPEDCLDELYEIMYSCWRTDPLDRPTFSVL
RLQLEKLLESLPDVRNQADVIYVNTQLLESSEGLAQGSTLAPLDLNIDPD
SIIASCTPRAAISVVTAEVHDSKPHEGRYILNGGSEEWEDLTSAPSAAVT
AEKNSVLPGERLVRNGVSWSHSSMLPLGSSLPDELLFADDSSEGSEVLM.

Provided herein are isolated antibodies that bind to MerTK, wherein the antibodies have one or more of the following properties: (i) antagonizes one or more biological activities of MerTK, (ii) reduces MerTK-mediated clearance of apoptotic cells, (iii) reduces MerTK-mediated phagocytic activity, (iv) enhances tumor immunogenicity of a checkpoint inhibitor, (v) binds to a fibronectin-like domain of MerTK, (vi) binds to an Ig-like domain on MerTK, (vii) binds specifically to human MerTK, (viii) binds to one or more of human, mouse and/or cyno MerTK, and/or (ix) binds to MerTK with a K_{D} of less than 20 nM (e.g., less than 10 nM, less than 5 nM, or less than 2 nM).

### A. Exemplary Anti-MerTK Antibodies

In one aspect, the invention provides an anti-MerTK antibody comprising at least one, at least two, or all three VH HVR sequences selected from (a) HVR-H1 comprising the amino acid sequence of SEQ ID NO: 4; (b) HVR-H2 comprising the amino acid sequence of SEQ ID NO: 5; and (c) HVR-H3 comprising the amino acid sequence of SEQ ID NO: 6. In one embodiment, the antibody comprises (a) HVR-H1 comprising the amino acid sequence of SEQ ID NO: 4; (b) HVR-H2 comprising the amino acid sequence of SEQ ID NO: 5; and (c) HVR-H3 comprising the amino acid sequence of SEQ ID NO: 6. In an exemplary embodiment, the anti-MerTK antibody binds to a fibronectin-like domain of MerTK.

In another aspect, the invention provides an anti-MerTK antibody comprising at least one, at least two, or all three VL HVR sequences selected from a) HVR-L1 comprising the amino acid sequence of SEQ ID NO: 1; (b) HVR-L2 comprising the amino acid sequence of SEQ ID NO: 2; and (c) HVR-L3 comprising the amino acid sequence of SEQ ID NO: 3. In one embodiment, the antibody comprises (a) HVR-L1 comprising the amino acid sequence of SEQ ID NO: 1; (b) HVR-L2 comprising the amino acid sequence of SEQ ID NO: 2; and (c) HVR-L3 comprising the amino acid sequence of SEQ ID NO: 3. In an exemplary embodiment, the anti-MerTK antibody binds to a fibronectin-like domain of MerTK.

In another aspect, an anti-MerTK antibody of the invention comprises (a) a VH domain comprising at least one, at least two, or all three VH HVR sequences selected from (i) HVR-H1 comprising the amino acid sequence of SEQ ID NO: 4, (ii) HVR-H2 comprising the amino acid sequence of SEQ ID NO: 5, and (iii) HVR-H3 comprising an amino acid sequence selected from SEQ ID NO: 6; and (b) a VL domain comprising at least one, at least two, or all three VL HVR sequences selected from (i) HVR-L1 comprising the amino acid sequence of SEQ ID NO: 1, (ii) HVR-L2 comprising the amino acid sequence of SEQ ID NO: 2, and (c) HVR-L3 comprising the amino acid sequence of SEQ ID NO: 3. In an exemplary embodiment, the anti-MerTK antibody binds to a fibronectin-like domain of MerTK.

In another aspect, the invention provides an antibody comprising (a) HVR-H1 comprising the amino acid sequence of SEQ ID NO: 4; (b) HVR-H2 comprising the amino acid sequence of SEQ ID NO: 5; (c) HVR-H3 comprising the amino acid sequence of SEQ ID NO: 6; (d) HVR-L1 comprising the amino acid sequence of SEQ ID NO: 1; (e) HVR-L2 comprising the amino acid sequence of SEQ ID NO: 2; and (f) HVR-L3 comprising an amino acid sequence selected from SEQ ID NO: 3. In an exemplary embodiment, the anti-MerTK antibody binds to a fibronectin-like domain of MerTK.

In any of the above embodiments, an anti-MerTK antibody is humanized. In one embodiment, an anti-MerTK antibody comprises HVRs as in any of the above embodiments, and further comprises an acceptor human framework, e.g. a human immunoglobulin framework or a human consensus framework, optionally with up to 10 amino acid substitutions (e.g. from 1-2, 1-3, 1-4, 1-5, 1-6, 1-7, 1-8, 1-9 or 1-10 amino acid substitutions). In exemplary embodiments, such amino acid substitutions correspond to the amino acid residues from a rabbit framework region sequence, such as, for example, one or more of the following residues: Q2, L4, P43, and/or F87 in the light chain variable region framework sequences and/or one or more of the following residues: V24, 148, G49, K71, and/or V78 in the heavy chain variable region framework sequences. The numbering of amino acid residues is according to the EU numbering system, also called the EU index, as described in Kabat et al., Sequences of Proteins of Immunological Interest, 5th Ed. Public Health Service, National Institutes of Health, Bethesda, MD, 1991. In an exemplary embodiment, the anti-MerTK antibody binds to a fibronectin-like domain of MerTK.

In another aspect, an anti-MerTK antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 83. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 83. In certain embodiments, substitutions, insertions, or deletions occur in regions outside the HVRs (i.e., in the FRs). Optionally, the anti-MerTK antibody comprises the VH sequence in SEQ ID NO: 83, including post-translational modifications of that sequence. In a particular embodiment, the VH comprises one, two or three HVRs selected from: (a) HVR-H1 comprising the amino acid sequence of SEQ ID NO: 4, (b) HVR-H2 comprising the amino acid sequence of SEQ ID NO: 5, and (c) HVR-H3 comprising the amino acid sequence of SEQ ID NO: 6. In an exemplary embodiment, the anti-MerTK antibody binds to a fibronectin-like domain of MerTK.

In another aspect, an anti-MerTK antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 65. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 65. In certain embodiments, the substitutions, insertions, or deletions occur in regions outside the HVRs (i.e., in the FRs). Optionally, the anti-MerTK antibody comprises the VL sequence in SEQ ID NO: 65, including post-translational modifications of that sequence. In a particular embodiment, the VL comprises one, two or three HVRs selected from (a) HVR-L1 comprising the amino acid sequence of SEQ ID NO: 1; (b) HVR-L2 comprising the amino acid sequence of SEQ ID NO: 2; and (c) HVR-L3 comprising the amino acid sequence of SEQ ID NO: 3. In an exemplary embodiment, the anti-MerTK antibody binds to a fibronectin-like domain of MerTK.

In another aspect, an anti-MerTK antibody is provided, wherein the antibody comprises a VH as in any of the embodiments provided above, and a VL as in any of the embodiments provided above. In one embodiment, the antibody comprises the VH and VL sequences in SEQ ID NO: 83 and SEQ ID NO: 65, respectively, including post-translational modifications of those sequences. In an exemplary embodiment, the anti-MerTK antibody binds to a fibronectin-like domain of MerTK.

In one aspect, the invention provides an anti-MerTK antibody comprising at least one, at least two, or all three VH HVR sequences selected from (a) HVR-H1 comprising the amino acid sequence of SEQ ID NO: 10; (b) HVR-H2 comprising the amino acid sequence of SEQ ID NO: 11; and (c) HVR-H3 comprising the amino acid sequence of SEQ ID NO: 12. In one embodiment, the antibody comprises (a) HVR-H1 comprising the amino acid sequence of SEQ ID NO: 10; (b) HVR-H2 comprising the amino acid sequence of SEQ ID NO: 11; and (c) HVR-H3 comprising the amino acid sequence of SEQ ID NO: 12. In an exemplary embodiment, the anti-MerTK antibody binds to a fibronectin-like domain of MerTK.

In another aspect, the invention provides an anti-MerTK antibody comprising at least one, at least two, or all three VL HVR sequences selected from (a) HVR-L1 comprising the amino acid sequence of SEQ ID NO:7; (b) HVR-L2 comprising the amino acid sequence of SEQ ID NO: 8; and (c) HVR-L3 comprising the amino acid sequence of SEQ ID NO: 9. In one embodiment, the antibody comprises (a) HVR-L1 comprising the amino acid sequence of SEQ ID NO: 7; (b) HVR-L2 comprising the amino acid sequence of SEQ ID NO: 8; and (c) HVR-L3 comprising the amino acid sequence of SEQ ID NO: 9. In an exemplary embodiment, the anti-MerTK antibody binds to a fibronectin-like domain of MerTK.

In another aspect, an anti-MerTK antibody of the invention comprises (a) a VH domain comprising at least one, at least two, or all three VH HVR sequences selected from (i) HVR-H1 comprising the amino acid sequence of SEQ ID NO: 10, (ii) HVR-H2 comprising the amino acid sequence of SEQ ID NO: 11, and (iii) HVR-H3 comprising an amino acid sequence selected from SEQ ID NO: 12; and (b) a VL domain comprising at least one, at least two, or all three VL HVR sequences selected from (i) HVR-L1 comprising the amino acid sequence of SEQ ID NO: 7, (ii) HVR-L2 comprising the amino acid sequence of SEQ ID NO: 8, and (c) HVR-L3 comprising the amino acid sequence of SEQ ID NO: 9. In an exemplary embodiment, the anti-MerTK antibody binds to a fibronectin-like domain of MerTK.

In another aspect, the invention provides an anti-MerTK antibody comprising (a) HVR-H1 comprising the amino acid sequence of SEQ ID NO: 10; (b) HVR-H2 comprising the amino acid sequence of SEQ ID NO: 11; (c) HVR-H3 comprising the amino acid sequence of SEQ ID NO: 12; (d) HVR-L1 comprising the amino acid sequence of SEQ ID NO: 7; (e) HVR-L2 comprising the amino acid sequence of SEQ ID NO: 8; and (f) HVR-L3 comprising an amino acid sequence selected from SEQ ID NO: 9. In an exemplary embodiment, the anti-MerTK antibody binds to a fibronectin-like domain of MerTK.

In any of the above embodiments, an anti-MerTK antibody is humanized. In one embodiment, an anti-MerTK antibody comprises HVRs as in any of the above embodiments, and further comprises an acceptor human framework, e.g. a human immunoglobulin framework or a human consensus framework, optionally with up to 10 amino acid substitutions (e.g. from 1-2, 1-3, 1-4, 1-5, 1-6, 1-7, 1-8, 1-9 or 1-10 amino acid substitutions). In exemplary embodiments, such amino acid substitutions correspond to the amino acid residues from a rabbit framework region sequence, such as, for example, one or more of the following residues: Q2, L4, P43, and/or F87 in the light chain variable region framework sequences and/or one or more of the following residues: V24, 148, G49, K71, and/or V78 in the heavy chain variable region framework sequences. The numbering of amino acid residues is according to the EU numbering system, also called the EU index, as described in Kabat et al., Sequences of Proteins of Immunological Interest, 5th Ed. Public Health Service, National Institutes of Health, Bethesda, MD, 1991. In an exemplary embodiment, the anti-MerTK antibody binds to a fibronectin-like domain of MerTK.

In another aspect, an anti-MerTK antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 84. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 84. In certain embodiments, substitutions, insertions, or deletions occur in regions outside the HVRs (i.e., in the FRs). Optionally, the anti-MerTK antibody comprises the VH sequence in SEQ ID NO: 84, including post-translational modifications of that sequence. In a particular embodiment, the VH comprises one, two or three HVRs selected from: (a) HVR-H1 comprising the amino acid sequence of SEQ ID NO: 10, (b) HVR-H2 comprising the amino acid sequence of SEQ ID NO: 11, and (c) HVR-H3 comprising the amino acid sequence of SEQ ID NO: 12. In an exemplary embodiment, the anti-MerTK antibody binds to a fibronectin-like domain of MerTK.

In another aspect, an anti-MerTK antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 66. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 66. In certain embodiments, the substitutions, insertions, or deletions occur in regions outside the HVRs (i.e., in the FRs). Optionally, the anti-MerTK antibody comprises the VL sequence in SEQ ID NO: 66, including post-translational modifications of that sequence. In a particular embodiment, the VL comprises one, two or three HVRs selected from (a) HVR-L1 comprising the amino acid sequence of SEQ ID NO: 7; (b) HVR-L2 comprising the amino acid sequence of SEQ ID NO: 8; and (c) HVR-L3 comprising the amino acid sequence of SEQ ID NO: 9. In an exemplary embodiment, the anti-MerTK antibody binds to a fibronectin-like domain of MerTK.

In another aspect, an anti-MerTK antibody is provided, wherein the antibody comprises a VH as in any of the embodiments provided above, and a VL as in any of the embodiments provided above. In one embodiment, the antibody comprises the VH and VL sequences in SEQ ID NO: 84 and SEQ ID NO: 66, respectively, including post-translational modifications of those sequences. In an exemplary embodiment, the anti-MerTK antibody binds to a fibronectin-like domain of MerTK.

In another aspect, an anti-MerTK antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 85. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 85. In certain embodiments, substitutions, insertions, or deletions occur in regions outside the HVRs (i.e., in the FRs). Optionally, the anti-MerTK antibody comprises the VH sequence in SEQ ID NO: 85, including post-translational modifications of that sequence. In a particular embodiment, the VH comprises one, two or three HVRs selected from: (a) HVR-H1 comprising the amino acid sequence of SEQ ID NO: 10, (b) HVR-H2 comprising the amino acid sequence of SEQ ID NO: 11, and (c) HVR-H3 comprising the amino acid sequence of SEQ ID NO: 12. In an exemplary embodiment, the anti-MerTK antibody binds to a fibronectin-like domain of MerTK.

In another aspect, an anti-MerTK antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 67. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 67. In certain embodiments, the substitutions, insertions, or deletions occur in regions outside the HVRs (i.e., in the FRs). Optionally, the anti-MerTK antibody comprises the VL sequence in SEQ ID NO: 67, including post-translational modifications of that sequence. In a particular embodiment, the VL comprises one, two or three HVRs selected from (a) HVR-L1 comprising the amino acid sequence of SEQ ID NO: 7; (b) HVR-L2 comprising the amino acid sequence of SEQ ID NO: 8; and (c) HVR-L3 comprising the amino acid sequence of SEQ ID NO: 9.

In another aspect, an anti-MerTK antibody is provided, wherein the antibody comprises a VH as in any of the embodiments provided above, and a VL as in any of the embodiments provided above. In one embodiment, the antibody comprises the VH and VL sequences in SEQ ID NO: 85 and SEQ ID NO: 67, respectively, including post-translational modifications of those sequences. In an exemplary embodiment, the anti-MerTK antibody binds to a fibronectin-like domain of MerTK.

In another aspect, an anti-MerTK antibody comprises a heavy chain sequence having at least 90%, 91%, 92%, 93%, 94%, 95%, 96%, 97%, 98%, 99%, or 100% sequence identity to the amino acid sequence of SEQ ID NO: 102. In certain embodiments, a heavy chain sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 102. In certain embodiments, substitutions, insertions, or deletions occur in regions outside the HVRs (i.e., in the FRs). Optionally, the anti-MerTK antibody comprises the heavy chain sequence in SEQ ID NO: 102, including post-translational modifications of that sequence. In a particular embodiment, the heavy chain comprises one, two or three HVRs selected from: (a) HVR-H1 comprising the amino acid sequence of SEQ ID NO: 10, (b) HVR-H2 comprising the amino acid sequence of SEQ ID NO: 11, and (c) HVR-H3 comprising the amino acid sequence of SEQ ID NO: 12. In an exemplary embodiment, the anti-MerTK antibody binds to a fibronectin-like domain of MerTK.

In another aspect, an anti-MerTK antibody is provided, wherein the antibody comprises a light chain having at least 90%, 91%, 92%, 93%, 94%, 95%, 96%, 97%, 98%, 99%, or 100% sequence identity to the amino acid sequence of SEQ ID NO: 110. In certain embodiments, a light chain sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 110. In certain embodiments, the substitutions, insertions, or deletions occur in regions outside the HVRs (i.e., in the FRs). Optionally, the anti-MerTK antibody comprises the light chain sequence in SEQ ID NO: 110, including post-translational modifications of that sequence. In a particular embodiment, the light chain comprises one, two or three HVRs selected from (a) HVR-L1 comprising the amino acid sequence of SEQ ID NO: 7; (b) HVR-L2 comprising the amino acid sequence of SEQ ID NO: 8; and (c) HVR-L3 comprising the amino acid sequence of SEQ ID NO: 9. In an exemplary embodiment, the anti-MerTK antibody binds to a fibronectin-like domain of MerTK.

In another aspect, an anti-MerTK antibody is provided, wherein the antibody comprises a heavy chain as in any of the embodiments provided above, and a light chain as in any of the embodiments provided above. In one embodiment, the antibody comprises the heavy chain and light chain sequences in SEQ ID NO: 102 and SEQ ID NO: 110, respectively, including post-translational modifications of those sequences. In an exemplary embodiment, the anti-MerTK antibody binds to a fibronectin-like domain of MerTK.

In another aspect, an anti-MerTK antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 86. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 86. In certain embodiments, substitutions, insertions, or deletions occur in regions outside the HVRs (i.e., in the FRs). Optionally, the anti-MerTK antibody comprises the VH sequence in SEQ ID NO: 86, including post-translational modifications of that sequence. In a particular embodiment, the VH comprises one, two or three HVRs selected from: (a) HVR-H1 comprising the amino acid sequence of SEQ ID NO: 10, (b) HVR-H2 comprising the amino acid sequence of SEQ ID NO: 11, and (c) HVR-H3 comprising the amino acid sequence of SEQ ID NO: 12. In an exemplary embodiment, the anti-MerTK antibody binds to a fibronectin-like domain of MerTK.

In another aspect, an anti-MerTK antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 68. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 68. In certain embodiments, the substitutions, insertions, or deletions occur in regions outside the HVRs (i.e., in the FRs). Optionally, the anti-MerTK antibody comprises the VL sequence in SEQ ID NO: 68, including post-translational modifications of that sequence. In a particular embodiment, the VL comprises one, two or three HVRs selected from (a) HVR-L1 comprising the amino acid sequence of SEQ ID NO: 7; (b) HVR-L2 comprising the amino acid sequence of SEQ ID NO: 8; and (c) HVR-L3 comprising the amino acid sequence of SEQ ID NO: 9. In an exemplary embodiment, the anti-MerTK antibody binds to a fibronectin-like domain of MerTK.

In another aspect, an anti-MerTK antibody is provided, wherein the antibody comprises a VH as in any of the embodiments provided above, and a VL as in any of the embodiments provided above. In one embodiment, the antibody comprises the VH and VL sequences in SEQ ID NO: 86 and SEQ ID NO: 68, respectively, including post-translational modifications of those sequences. In an exemplary embodiment, the anti-MerTK antibody binds to a fibronectin-like domain of MerTK.

In another aspect, an anti-MerTK antibody comprises a heavy chain sequence having at least 90%, 91%, 92%, 93%, 94%, 95%, 96%, 97%, 98%, 99%, or 100% sequence identity to the amino acid sequence of SEQ ID NO: 103. In certain embodiments, a heavy chain sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 103. In certain embodiments, substitutions, insertions, or deletions occur in regions outside the HVRs (i.e., in the FRs). Optionally, the anti-MerTK antibody comprises the heavy chain sequence in SEQ ID NO: 103, including post-translational modifications of that sequence. In a particular embodiment, the heavy chain comprises one, two or three HVRs selected from: (a) HVR-H1 comprising the amino acid sequence of SEQ ID NO: 10, (b) HVR-H2 comprising the amino acid sequence of SEQ ID NO: 11, and (c) HVR-H3 comprising the amino acid sequence of SEQ ID NO: 12. In an exemplary embodiment, the anti-MerTK antibody binds to a fibronectin-like domain of MerTK.

In another aspect, an anti-MerTK antibody is provided, wherein the antibody comprises a light chain having at least 90%, 91%, 92%, 93%, 94%, 95%, 96%, 97%, 98%, 99%, or 100% sequence identity to the amino acid sequence of SEQ ID NO: 111. In certain embodiments, a light chain sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 111. In certain embodiments, the substitutions, insertions, or deletions occur in regions outside the HVRs (i.e., in the FRs). Optionally, the anti-MerTK antibody comprises the light chain sequence in SEQ ID NO: 111, including post-translational modifications of that sequence. In a particular embodiment, the light chain comprises one, two or three HVRs selected from (a) HVR-L1 comprising the amino acid sequence of SEQ ID NO: 7; (b) HVR-L2 comprising the amino acid sequence of SEQ ID NO: 8; and (c) HVR-L3 comprising the amino acid sequence of SEQ ID NO: 9. In an exemplary embodiment, the anti-MerTK antibody binds to a fibronectin-like domain of MerTK.

In another aspect, an anti-MerTK antibody is provided, wherein the antibody comprises a heavy chain as in any of the embodiments provided above, and a light chain as in any of the embodiments provided above. In one embodiment, the antibody comprises the heavy chain and light chain sequences in SEQ ID NO: 103 and SEQ ID NO: 111, respectively, including post-translational modifications of those sequences. In an exemplary embodiment, the anti-MerTK antibody binds to a fibronectin-like domain of MerTK.

In one aspect, the invention provides an anti-MerTK antibody comprising at least one, at least two, or all three VH HVR sequences selected from (a) HVR-H1 comprising the amino acid sequence of SEQ ID NO: 16; (b) HVR-H2 comprising the amino acid sequence of SEQ ID NO: 17; and (c) HVR-H3 comprising the amino acid sequence of SEQ ID NO: 18. In one embodiment, the antibody comprises (a) HVR-H1 comprising the amino acid sequence of SEQ ID NO: 16; (b) HVR-H2 comprising the amino acid sequence of SEQ ID NO: 17; and (c) HVR-H3 comprising the amino acid sequence of SEQ ID NO: 18. In an exemplary embodiment, the anti-MerTK antibody binds to a fibronectin-like domain of MerTK.

In another aspect, the invention provides an anti-MerTK antibody comprising at least one, at least two, or all three VL HVR sequences selected from (a) HVR-L1 comprising the amino acid sequence of SEQ ID NO: 13; (b) HVR-L2 comprising the amino acid sequence of SEQ ID NO: 14; and (c) HVR-L3 comprising the amino acid sequence of SEQ ID NO: 15. In one embodiment, the antibody comprises (a) HVR-L1 comprising the amino acid sequence of SEQ ID NO: 13; (b) HVR-L2 comprising the amino acid sequence of SEQ ID NO: 14; and (c) HVR-L3 comprising the amino acid sequence of SEQ ID NO: 15. In an exemplary embodiment, the anti-MerTK antibody binds to a fibronectin-like domain of MerTK.

In another aspect, an anti-MerTK antibody of the invention comprises (a) a VH domain comprising at least one, at least two, or all three VH HVR sequences selected from (i) HVR-H1 comprising the amino acid sequence of SEQ ID NO: 16, (ii) HVR-H2 comprising the amino acid sequence of SEQ ID NO: 17, and (iii) HVR-H3 comprising an amino acid sequence selected from SEQ ID NO: 18; and (b) a VL domain comprising at least one, at least two, or all three VL HVR sequences selected from (i) HVR-L1 comprising the amino acid sequence of SEQ ID NO: 13, (ii) HVR-L2 comprising the amino acid sequence of SEQ ID NO: 14, and (c) HVR-L3 comprising the amino acid sequence of SEQ ID NO: 15. In an exemplary embodiment, the anti-MerTK antibody binds to a fibronectin-like domain of MerTK.

In another aspect, the invention provides an anti-MerTK antibody comprising (a) HVR-H1 comprising the amino acid sequence of SEQ ID NO: 16; (b) HVR-H2 comprising the amino acid sequence of SEQ ID NO: 17; (c) HVR-H3 comprising the amino acid sequence of SEQ ID NO: 18; (d) HVR-L1 comprising the amino acid sequence of SEQ ID NO: 13; (e) HVR-L2 comprising the amino acid sequence of SEQ ID NO: 14; and (f) HVR-L3 comprising an amino acid sequence selected from SEQ ID NO: 15. In an exemplary embodiment, the anti-MerTK antibody binds to a fibronectin-like domain of MerTK.

In any of the above embodiments, an anti-MerTK antibody is humanized. In one embodiment, an anti-MerTK antibody comprises HVRs as in any of the above embodiments, and further comprises an acceptor human framework, e.g. a human immunoglobulin framework or a human consensus framework, optionally with up to 10 amino acid substitutions (e.g. from 1-2, 1-3, 1-4, 1-5, 1-6, 1-7, 1-8, 1-9 or 1-10 amino acid substitutions). In exemplary embodiments, such amino acid substitutions correspond to the amino acid residues from a rabbit framework region sequence, such as, for example, one or more of the following residues: Q2, L4, P43, and/or F87 in the light chain variable region framework sequences and/or one or more of the following residues: V24, 148, G49, K71, and/or V78 in the heavy chain variable region framework sequences. The numbering of amino acid residues is according to the EU numbering system, also called the EU index, as described in Kabat et al., Sequences of Proteins of Immunological Interest, 5th Ed. Public Health Service, National Institutes of Health, Bethesda, MD, 1991. In an exemplary embodiment, the anti-MerTK antibody binds to a fibronectin-like domain of MerTK.

In another aspect, an anti-MerTK antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 87. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 87. In certain embodiments, substitutions, insertions, or deletions occur in regions outside the HVRs (i.e., in the FRs). Optionally, the anti-MerTK antibody comprises the VH sequence in SEQ ID NO: 87, including post-translational modifications of that sequence. In a particular embodiment, the VH comprises one, two or three HVRs selected from: (a) HVR-H1 comprising the amino acid sequence of SEQ ID NO: 16, (b) HVR-H2 comprising the amino acid sequence of SEQ ID NO: 17, and (c) HVR-H3 comprising the amino acid sequence of SEQ ID NO: 18. In an exemplary embodiment, the anti-MerTK antibody binds to a fibronectin-like domain of MerTK.

In another aspect, an anti-MerTK antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 69. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 69. In certain embodiments, the substitutions, insertions, or deletions occur in regions outside the HVRs (i.e., in the FRs). Optionally, the anti-MerTK antibody comprises the VL sequence in SEQ ID NO: 69, including post-translational modifications of that sequence. In a particular embodiment, the VL comprises one, two or three HVRs selected from (a) HVR-L1 comprising the amino acid sequence of SEQ ID NO: 13; (b) HVR-L2 comprising the amino acid sequence of SEQ ID NO: 14; and (c) HVR-L3 comprising the amino acid sequence of SEQ ID NO: 15. In an exemplary embodiment, the anti-MerTK antibody binds to a fibronectin-like domain of MerTK.

In another aspect, an anti-MerTK antibody is provided, wherein the antibody comprises a VH as in any of the embodiments provided above, and a VL as in any of the embodiments provided above. In one embodiment, the antibody comprises the VH and VL sequences in SEQ ID NO: 87 and SEQ ID NO: 69, respectively, including post-translational modifications of those sequences. In an exemplary embodiment, the anti-MerTK antibody binds to a fibronectin-like domain of MerTK.

In another aspect, an anti-MerTK antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 88. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 88. In certain embodiments, substitutions, insertions, or deletions occur in regions outside the HVRs (i.e., in the FRs). Optionally, the anti-MerTK antibody comprises the VH sequence in SEQ ID NO: 88, including post-translational modifications of that sequence. In a particular embodiment, the VH comprises one, two or three HVRs selected from: (a) HVR-H1 comprising the amino acid sequence of SEQ ID NO: 16, (b) HVR-H2 comprising the amino acid sequence of SEQ ID NO: 17, and (c) HVR-H3 comprising the amino acid sequence of SEQ ID NO: 18. In an exemplary embodiment, the anti-MerTK antibody binds to a fibronectin-like domain of MerTK.

In another aspect, an anti-MerTK antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 70. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 70. In certain embodiments, the substitutions, insertions, or deletions occur in regions outside the HVRs (i.e., in the FRs). Optionally, the anti-MerTK antibody comprises the VL sequence in SEQ ID NO: 70, including post-translational modifications of that sequence. In a particular embodiment, the VL comprises one, two or three HVRs selected from (a) HVR-L1 comprising the amino acid sequence of SEQ ID NO: 13; (b) HVR-L2 comprising the amino acid sequence of SEQ ID NO: 14; and (c) HVR-L3 comprising the amino acid sequence of SEQ ID NO: 15. In an exemplary embodiment, the anti-MerTK antibody binds to a fibronectin-like domain of MerTK.

In another aspect, an anti-MerTK antibody is provided, wherein the antibody comprises a VH as in any of the embodiments provided above, and a VL as in any of the embodiments provided above. In one embodiment, the antibody comprises the VH and VL sequences in SEQ ID NO: 88 and SEQ ID NO: 70, respectively, including post-translational modifications of those sequences. In an exemplary embodiment, the anti-MerTK antibody binds to a fibronectin-like domain of MerTK.

In another aspect, an anti-MerTK antibody comprises a heavy chain sequence having at least 90%, 91%, 92%, 93%, 94%, 95%, 96%, 97%, 98%, 99%, or 100% sequence identity to the amino acid sequence of SEQ ID NO: 104. In certain embodiments, a heavy chain sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 104. In certain embodiments, substitutions, insertions, or deletions occur in regions outside the HVRs (i.e., in the FRs). Optionally, the anti-MerTK antibody comprises the heavy chain sequence in SEQ ID NO: 104, including post-translational modifications of that sequence. In a particular embodiment, the heavy chain comprises one, two or three HVRs selected from: (a) HVR-H1 comprising the amino acid sequence of SEQ ID NO: 16, (b) HVR-H2 comprising the amino acid sequence of SEQ ID NO: 17, and (c) HVR-H3 comprising the amino acid sequence of SEQ ID NO: 18. In an exemplary embodiment, the anti-MerTK antibody binds to a fibronectin-like domain of MerTK.

In another aspect, an anti-MerTK antibody is provided, wherein the antibody comprises a light chain having at least 90%, 91%, 92%, 93%, 94%, 95%, 96%, 97%, 98%, 99%, or 100% sequence identity to the amino acid sequence of SEQ ID NO: 112. In certain embodiments, a light chain sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 112. In certain embodiments, the substitutions, insertions, or deletions occur in regions outside the HVRs (i.e., in the FRs). Optionally, the anti-MerTK antibody comprises the light chain sequence in SEQ ID NO: 112, including post-translational modifications of that sequence. In a particular embodiment, the light chain comprises one, two or three HVRs selected from (a) HVR-L1 comprising the amino acid sequence of SEQ ID NO: 13; (b) HVR-L2 comprising the amino acid sequence of SEQ ID NO: 14; and (c) HVR-L3 comprising the amino acid sequence of SEQ ID NO: 15. In an exemplary embodiment, the anti-MerTK antibody binds to a fibronectin-like domain of MerTK.

In another aspect, an anti-MerTK antibody is provided, wherein the antibody comprises a heavy chain as in any of the embodiments provided above, and a light chain as in any of the embodiments provided above. In one embodiment, the antibody comprises the heavy chain and light chain sequences in SEQ ID NO: 104 and SEQ ID NO: 112, respectively, including post-translational modifications of those sequences. In an exemplary embodiment, the anti-MerTK antibody binds to a fibronectin-like domain of MerTK.

In another aspect, an anti-MerTK antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 89. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 89. In certain embodiments, substitutions, insertions, or deletions occur in regions outside the HVRs (i.e., in the FRs). Optionally, the anti-MerTK antibody comprises the VH sequence in SEQ ID NO: 89, including post-translational modifications of that sequence. In a particular embodiment, the VH comprises one, two or three HVRs selected from: (a) HVR-H1 comprising the amino acid sequence of SEQ ID NO: 16, (b) HVR-H2 comprising the amino acid sequence of SEQ ID NO: 17, and (c) HVR-H3 comprising the amino acid sequence of SEQ ID NO: 18. In an exemplary embodiment, the anti-MerTK antibody binds to a fibronectin-like domain of MerTK.

In another aspect, an anti-MerTK antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 70. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 70. In certain embodiments, the substitutions, insertions, or deletions occur in regions outside the HVRs (i.e., in the FRs). Optionally, the anti-MerTK antibody comprises the VL sequence in SEQ ID NO: 70, including post-translational modifications of that sequence. In a particular embodiment, the VL comprises one, two or three HVRs selected from (a) HVR-L1 comprising the amino acid sequence of SEQ ID NO: 13; (b) HVR-L2 comprising the amino acid sequence of SEQ ID NO: 14; and (c) HVR-L3 comprising the amino acid sequence of SEQ ID NO: 15. In an exemplary embodiment, the anti-MerTK antibody binds to a fibronectin-like domain of MerTK.

In another aspect, an anti-MerTK antibody is provided, wherein the antibody comprises a VH as in any of the embodiments provided above, and a VL as in any of the embodiments provided above. In one embodiment, the antibody comprises the VH and VL sequences in SEQ ID NO: 89 and SEQ ID NO: 70, respectively, including post-translational modifications of those sequences. In an exemplary embodiment, the anti-MerTK antibody binds to a fibronectin-like domain of MerTK.

In another aspect, an anti-MerTK antibody comprises a heavy chain sequence having at least 90%, 91%, 92%, 93%, 94%, 95%, 96%, 97%, 98%, 99%, or 100% sequence identity to the amino acid sequence of SEQ ID NO: 105. In certain embodiments, a heavy chain sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 105. In certain embodiments, substitutions, insertions, or deletions occur in regions outside the HVRs (i.e., in the FRs). Optionally, the anti-MerTK antibody comprises the heavy chain sequence in SEQ ID NO: 105, including post-translational modifications of that sequence. In a particular embodiment, the heavy chain comprises one, two or three HVRs selected from: (a) HVR-H1 comprising the amino acid sequence of SEQ ID NO: 16, (b) HVR-H2 comprising the amino acid sequence of SEQ ID NO: 17, and (c) HVR-H3 comprising the amino acid sequence of SEQ ID NO: 18. In an exemplary embodiment, the anti-MerTK antibody binds to a fibronectin-like domain of MerTK.

In another aspect, an anti-MerTK antibody is provided, wherein the antibody comprises a light chain having at least 90%, 91%, 92%, 93%, 94%, 95%, 96%, 97%, 98%, 99%, or 100% sequence identity to the amino acid sequence of SEQ ID NO: 113. In certain embodiments, a light chain sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 113. In certain embodiments, the substitutions, insertions, or deletions occur in regions outside the HVRs (i.e., in the FRs). Optionally, the anti-MerTK antibody comprises the light chain sequence in SEQ ID NO: 113, including post-translational modifications of that sequence. In a particular embodiment, the light chain comprises one, two or three HVRs selected from (a) HVR-L1 comprising the amino acid sequence of SEQ ID NO: 13; (b) HVR-L2 comprising the amino acid sequence of SEQ ID NO: 14; and (c) HVR-L3 comprising the amino acid sequence of SEQ ID NO: 15. In an exemplary embodiment, the anti-MerTK antibody binds to a fibronectin-like domain of MerTK.

In another aspect, an anti-MerTK antibody is provided, wherein the antibody comprises a heavy chain as in any of the embodiments provided above, and a light chain as in any of the embodiments provided above. In one embodiment, the antibody comprises the heavy chain and light chain sequences in SEQ ID NO: 105 and SEQ ID NO: 113, respectively, including post-translational modifications of those sequences. In an exemplary embodiment, the anti-MerTK antibody binds to a fibronectin-like domain of MerTK.

In one aspect, the invention provides an anti-MerTK antibody comprising at least one, at least two, or all three VH HVR sequences selected from (a) HVR-H1 comprising the amino acid sequence of SEQ ID NO: 22; (b) HVR-H2 comprising the amino acid sequence of SEQ ID NO: 23; and (c) HVR-H3 comprising the amino acid sequence of SEQ ID NO: 24. In one embodiment, the antibody comprises (a) HVR-H1 comprising the amino acid sequence of SEQ ID NO: 22; (b) HVR-H2 comprising the amino acid sequence of SEQ ID NO: 23; and (c) HVR-H3 comprising the amino acid sequence of SEQ ID NO: 24. In an exemplary embodiment, the anti-MerTK antibody binds to a fibronectin-like domain of MerTK.

In another aspect, the invention provides an anti-MerTK antibody comprising at least one, at least two, or all three VL HVR sequences selected from (a) HVR-L1 comprising the amino acid sequence of SEQ ID NO: 19; (b) HVR-L2 comprising the amino acid sequence of SEQ ID NO: 20; and (c) HVR-L3 comprising the amino acid sequence of SEQ ID NO: 21. In one embodiment, the antibody comprises (a) HVR-L1 comprising the amino acid sequence of SEQ ID NO: 19; (b) HVR-L2 comprising the amino acid sequence of SEQ ID NO: 20; and (c) HVR-L3 comprising the amino acid sequence of SEQ ID NO: 21. In an exemplary embodiment, the anti-MerTK antibody binds to a fibronectin-like domain of MerTK.

In another aspect, an anti-MerTK antibody of the invention comprises (a) a VH domain comprising at least one, at least two, or all three VH HVR sequences selected from (i) HVR-H1 comprising the amino acid sequence of SEQ ID NO: 22, (ii) HVR-H2 comprising the amino acid sequence of SEQ ID NO: 23, and (iii) HVR-H3 comprising an amino acid sequence selected from SEQ ID NO: 24; and (b) a VL domain comprising at least one, at least two, or all three VL HVR sequences selected from (i) HVR-L1 comprising the amino acid sequence of SEQ ID NO: 19, (ii) HVR-L2 comprising the amino acid sequence of SEQ ID NO: 20, and (c) HVR-L3 comprising the amino acid sequence of SEQ ID NO: 21. In an exemplary embodiment, the anti-MerTK antibody binds to a fibronectin-like domain of MerTK.

In another aspect, the invention provides an anti-MerTK antibody comprising (a) HVR-H1 comprising the amino acid sequence of SEQ ID NO: 22; (b) HVR-H2 comprising the amino acid sequence of SEQ ID NO: 23; (c) HVR-H3 comprising the amino acid sequence of SEQ ID NO: 24; (d) HVR-L1 comprising the amino acid sequence of SEQ ID NO: 19; (e) HVR-L2 comprising the amino acid sequence of SEQ ID NO: 20; and (f) HVR-L3 comprising an amino acid sequence selected from SEQ ID NO: 21. In an exemplary embodiment, the anti-MerTK antibody binds to a fibronectin-like domain of MerTK.

In any of the above embodiments, an anti-MerTK antibody is humanized. In one embodiment, an anti-MerTK antibody comprises HVRs as in any of the above embodiments, and further comprises an acceptor human framework, e.g. a human immunoglobulin framework or a human consensus framework, optionally with up to 10 amino acid substitutions (e.g. from 1-2, 1-3, 1-4, 1-5, 1-6, 1-7, 1-8, 1-9 or 1-10 amino acid substitutions). In exemplary embodiments, such amino acid substitutions correspond to the amino acid residues from a rabbit framework region sequence, such as, for example, one or more of the following residues: Q2, L4, P43, and/or F87 in the light chain variable region framework sequences and/or one or more of the following residues: V24, 148, G49, K71, and/or V78 in the heavy chain variable region framework sequences. The numbering of amino acid residues is according to the EU numbering system, also called the EU index, as described in Kabat et al., Sequences of Proteins of Immunological Interest, 5th Ed. Public Health Service, National Institutes of Health, Bethesda, MD, 1991. In an exemplary embodiment, the anti-MerTK antibody binds to a fibronectin-like domain of MerTK.

In another aspect, an anti-MerTK antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 90. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 90. In certain embodiments, substitutions, insertions, or deletions occur in regions outside the HVRs (i.e., in the FRs). Optionally, the anti-MerTK antibody comprises the VH sequence in SEQ ID NO: 90, including post-translational modifications of that sequence. In a particular embodiment, the VH comprises one, two or three HVRs selected from: (a) HVR-H1 comprising the amino acid sequence of SEQ ID NO: 22, (b) HVR-H2 comprising the amino acid sequence of SEQ ID NO: 23, and (c) HVR-H3 comprising the amino acid sequence of SEQ ID NO: 24. In an exemplary embodiment, the anti-MerTK antibody binds to a fibronectin-like domain of MerTK.

In another aspect, an anti-MerTK antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 71. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 71. In certain embodiments, the substitutions, insertions, or deletions occur in regions outside the HVRs (i.e., in the FRs). Optionally, the anti-MerTK antibody comprises the VL sequence in SEQ ID NO: 71, including post-translational modifications of that sequence. In a particular embodiment, the VL comprises one, two or three HVRs selected from (a) HVR-L1 comprising the amino acid sequence of SEQ ID NO: 19; (b) HVR-L2 comprising the amino acid sequence of SEQ ID NO: 20; and (c) HVR-L3 comprising the amino acid sequence of SEQ ID NO: 21. In an exemplary embodiment, the anti-MerTK antibody binds to a fibronectin-like domain of MerTK.

In another aspect, an anti-MerTK antibody is provided, wherein the antibody comprises a VH as in any of the embodiments provided above, and a VL as in any of the embodiments provided above. In one embodiment, the antibody comprises the VH and VL sequences in SEQ ID NO: 90 and SEQ ID NO: 71, respectively, including post-translational modifications of those sequences. In an exemplary embodiment, the anti-MerTK antibody binds to a fibronectin-like domain of MerTK.

In another aspect, an anti-MerTK antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 91. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 91. In certain embodiments, substitutions, insertions, or deletions occur in regions outside the HVRs (i.e., in the FRs). Optionally, the anti-MerTK antibody comprises the VH sequence in SEQ ID NO: 91, including post-translational modifications of that sequence. In a particular embodiment, the VH comprises one, two or three HVRs selected from: (a) HVR-H1 comprising the amino acid sequence of SEQ ID NO: 22, (b) HVR-H2 comprising the amino acid sequence of SEQ ID NO: 23, and (c) HVR-H3 comprising the amino acid sequence of SEQ ID NO: 24. In an exemplary embodiment, the anti-MerTK antibody binds to a fibronectin-like domain of MerTK.

In another aspect, an anti-MerTK antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 72. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 72. In certain embodiments, the substitutions, insertions, or deletions occur in regions outside the HVRs (i.e., in the FRs). Optionally, the anti-MerTK antibody comprises the VL sequence in SEQ ID NO: 72, including post-translational modifications of that sequence. In a particular embodiment, the VL comprises one, two or three HVRs selected from (a) HVR-L1 comprising the amino acid sequence of SEQ ID NO: 19; (b) HVR-L2 comprising the amino acid sequence of SEQ ID NO: 20; and (c) HVR-L3 comprising the amino acid sequence of SEQ ID NO: 21. In an exemplary embodiment, the anti-MerTK antibody binds to a fibronectin-like domain of MerTK.

In another aspect, an anti-MerTK antibody is provided, wherein the antibody comprises a VH as in any of the embodiments provided above, and a VL as in any of the embodiments provided above. In one embodiment, the antibody comprises the VH and VL sequences in SEQ ID NO: 91 and SEQ ID NO: 72, respectively, including post-translational modifications of those sequences. In an exemplary embodiment, the anti-MerTK antibody binds to a fibronectin-like domain of MerTK.

In another aspect, an anti-MerTK antibody comprises a heavy chain sequence having at least 90%, 91%, 92%, 93%, 94%, 95%, 96%, 97%, 98%, 99%, or 100% sequence identity to the amino acid sequence of SEQ ID NO: 106. In certain embodiments, a heavy chain sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 106. In certain embodiments, substitutions, insertions, or deletions occur in regions outside the HVRs (i.e., in the FRs). Optionally, the anti-MerTK antibody comprises the heavy chain sequence in SEQ ID NO: 106, including post-translational modifications of that sequence. In a particular embodiment, the heavy chain comprises one, two or three HVRs selected from: (a) HVR-H1 comprising the amino acid sequence of SEQ ID NO: 22, (b) HVR-H2 comprising the amino acid sequence of SEQ ID NO: 23, and (c) HVR-H3 comprising the amino acid sequence of SEQ ID NO: 24. In an exemplary embodiment, the anti-MerTK antibody binds to a fibronectin-like domain of MerTK.

In another aspect, an anti-MerTK antibody is provided, wherein the antibody comprises a light chain having at least 90%, 91%, 92%, 93%, 94%, 95%, 96%, 97%, 98%, 99%, or 100% sequence identity to the amino acid sequence of SEQ ID NO: 114. In certain embodiments, a light chain sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 114. In certain embodiments, the substitutions, insertions, or deletions occur in regions outside the HVRs (i.e., in the FRs). Optionally, the anti-MerTK antibody comprises the light chain sequence in SEQ ID NO: 114, including post-translational modifications of that sequence. In a particular embodiment, the light chain comprises one, two or three HVRs selected from (a) HVR-L1 comprising the amino acid sequence of SEQ ID NO: 19; (b) HVR-L2 comprising the amino acid sequence of SEQ ID NO: 20; and (c) HVR-L3 comprising the amino acid sequence of SEQ ID NO: 21. In an exemplary embodiment, the anti-MerTK antibody binds to a fibronectin-like domain of MerTK.

In another aspect, an anti-MerTK antibody is provided, wherein the antibody comprises a heavy chain as in any of the embodiments provided above, and a light chain as in any of the embodiments provided above. In one embodiment, the antibody comprises the heavy chain and light chain sequences in SEQ ID NO: 106 and SEQ ID NO: 114, respectively, including post-translational modifications of those sequences. In an exemplary embodiment, the anti-MerTK antibody binds to a fibronectin-like domain of MerTK.

In another aspect, an anti-MerTK antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 92. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 92. In certain embodiments, substitutions, insertions, or deletions occur in regions outside the HVRs (i.e., in the FRs). Optionally, the anti-MerTK antibody comprises the VH sequence in SEQ ID NO: 92, including post-translational modifications of that sequence. In a particular embodiment, the VH comprises one, two or three HVRs selected from: (a) HVR-H1 comprising the amino acid sequence of SEQ ID NO: 22, (b) HVR-H2 comprising the amino acid sequence of SEQ ID NO: 23, and (c) HVR-H3 comprising the amino acid sequence of SEQ ID NO: 24. In an exemplary embodiment, the anti-MerTK antibody binds to a fibronectin-like domain of MerTK.

In another aspect, an anti-MerTK antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 73. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 73. In certain embodiments, the substitutions, insertions, or deletions occur in regions outside the HVRs (i.e., in the FRs). Optionally, the anti-MerTK antibody comprises the VL sequence in SEQ ID NO: 73, including post-translational modifications of that sequence. In a particular embodiment, the VL comprises one, two or three HVRs selected from (a) HVR-L1 comprising the amino acid sequence of SEQ ID NO: 19; (b) HVR-L2 comprising the amino acid sequence of SEQ ID NO: 20; and (c) HVR-L3 comprising the amino acid sequence of SEQ ID NO: 21. In an exemplary embodiment, the anti-MerTK antibody binds to a fibronectin-like domain of MerTK.

In another aspect, an anti-MerTK antibody is provided, wherein the antibody comprises a VH as in any of the embodiments provided above, and a VL as in any of the embodiments provided above. In one embodiment, the antibody comprises the VH and VL sequences in SEQ ID NO: 92 and SEQ ID NO: 73, respectively, including post-translational modifications of those sequences. In an exemplary embodiment, the anti-MerTK antibody binds to a fibronectin-like domain of MerTK.

In another aspect, an anti-MerTK antibody comprises a heavy chain sequence having at least 90%, 91%, 92%, 93%, 94%, 95%, 96%, 97%, 98%, 99%, or 100% sequence identity to the amino acid sequence of SEQ ID NO: 107. In certain embodiments, a heavy chain sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 107. In certain embodiments, substitutions, insertions, or deletions occur in regions outside the HVRs (i.e., in the FRs). Optionally, the anti-MerTK antibody comprises the heavy chain sequence in SEQ ID NO: 107, including post-translational modifications of that sequence. In a particular embodiment, the heavy chain comprises one, two or three HVRs selected from: (a) HVR-H1 comprising the amino acid sequence of SEQ ID NO: 22, (b) HVR-H2 comprising the amino acid sequence of SEQ ID NO: 23, and (c) HVR-H3 comprising the amino acid sequence of SEQ ID NO:24. In an exemplary embodiment, the anti-MerTK antibody binds to a fibronectin-like domain of MerTK.

In another aspect, an anti-MerTK antibody is provided, wherein the antibody comprises a light chain having at least 90%, 91%, 92%, 93%, 94%, 95%, 96%, 97%, 98%, 99%, or 100% sequence identity to the amino acid sequence of SEQ ID NO: 115. In certain embodiments, a light chain sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 115. In certain embodiments, the substitutions, insertions, or deletions occur in regions outside the HVRs (i.e., in the FRs). Optionally, the anti-MerTK antibody comprises the light chain sequence in SEQ ID NO: 115, including post-translational modifications of that sequence. In a particular embodiment, the light chain comprises one, two or three HVRs selected from (a) HVR-L1 comprising the amino acid sequence of SEQ ID NO: 19; (b) HVR-L2 comprising the amino acid sequence of SEQ ID NO: 20; and (c) HVR-L3 comprising the amino acid sequence of SEQ ID NO: 20. In an exemplary embodiment, the anti-MerTK antibody binds to a fibronectin-like domain of MerTK.

In another aspect, an anti-MerTK antibody is provided, wherein the antibody comprises a heavy chain as in any of the embodiments provided above, and a light chain as in any of the embodiments provided above. In one embodiment, the antibody comprises the heavy chain and light chain sequences in SEQ ID NO: 107 and SEQ ID NO: 115, respectively, including post-translational modifications of those sequences. In an exemplary embodiment, the anti-MerTK antibody binds to a fibronectin-like domain of MerTK.

In one aspect, the invention provides an anti-MerTK antibody comprising at least one, at least two, or all three VH HVR sequences selected from (a) HVR-H1 comprising the amino acid sequence of SEQ ID NO: 27; (b) HVR-H2 comprising the amino acid sequence of SEQ ID NO: 28; and (c) HVR-H3 comprising the amino acid sequence of SEQ ID NO: 29. In one embodiment, the antibody comprises (a) HVR-H1 comprising the amino acid sequence of SEQ ID NO: 27; (b) HVR-H2 comprising the amino acid sequence of SEQ ID NO: 28; and (c) HVR-H3 comprising the amino acid sequence of SEQ ID NO: 29. In an exemplary embodiment, the anti-MerTK antibody binds to a fibronectin-like domain of MerTK.

In another aspect, the invention provides an anti-MerTK antibody comprising at least one, at least two, or all three VL HVR sequences selected from (a) HVR-L1 comprising the amino acid sequence of SEQ ID NO: 25; (b) HVR-L2 comprising the amino acid sequence of SEQ ID NO: 14; and (c) HVR-L3 comprising the amino acid sequence of SEQ ID NO: 26. In one embodiment, the antibody comprises (a) HVR-L1 comprising the amino acid sequence of SEQ ID NO: 25; (b) HVR-L2 comprising the amino acid sequence of SEQ ID NO: 14; and (c) HVR-L3 comprising the amino acid sequence of SEQ ID NO: 26. In an exemplary embodiment, the anti-MerTK antibody binds to a fibronectin-like domain of MerTK.

In another aspect, an anti-MerTK antibody of the invention comprises (a) a VH domain comprising at least one, at least two, or all three VH HVR sequences selected from (i) HVR-H1 comprising the amino acid sequence of SEQ ID NO: 27, (ii) HVR-H2 comprising the amino acid sequence of SEQ ID NO: 28, and (iii) HVR-H3 comprising an amino acid sequence selected from SEQ ID NO: 29; and (b) a VL domain comprising at least one, at least two, or all three VL HVR sequences selected from (i) HVR-L1 comprising the amino acid sequence of SEQ ID NO: 25, (ii) HVR-L2 comprising the amino acid sequence of SEQ ID NO: 14, and (c) HVR-L3 comprising the amino acid sequence of SEQ ID NO: 26. In an exemplary embodiment, the anti-MerTK antibody binds to a fibronectin-like domain of MerTK.

In another aspect, the invention provides an anti-MerTK antibody comprising (a) HVR-H1 comprising the amino acid sequence of SEQ ID NO: 27; (b) HVR-H2 comprising the amino acid sequence of SEQ ID NO: 28; (c) HVR-H3 comprising the amino acid sequence of SEQ ID NO: 29; (d) HVR-L1 comprising the amino acid sequence of SEQ ID NO: 25; (e) HVR-L2 comprising the amino acid sequence of SEQ ID NO: 14; and (f) HVR-L3 comprising an amino acid sequence selected from SEQ ID NO: 26. In an exemplary embodiment, the anti-MerTK antibody binds to a fibronectin-like domain of MerTK.

In any of the above embodiments, an anti-MerTK antibody is humanized. In one embodiment, an anti-MerTK antibody comprises HVRs as in any of the above embodiments, and further comprises an acceptor human framework, e.g. a human immunoglobulin framework or a human consensus framework, optionally with up to 10 amino acid substitutions (e.g. from 1-2, 1-3, 1-4, 1-5, 1-6, 1-7, 1-8, 1-9 or 1-10 amino acid substitutions). In exemplary embodiments, such amino acid substitutions correspond to the amino acid residues from a rabbit framework region sequence, such as, for example, one or more of the following residues: Q2, L4, P43, and/or F87 in the light chain variable region framework sequences and/or one or more of the following residues: V24, 148, G49, K71, and/or V78 in the heavy chain variable region framework sequences. The numbering of amino acid residues is according to the EU numbering system, also called the EU index, as described in Kabat et al., Sequences of Proteins of Immunological Interest, 5th Ed. Public Health Service, National Institutes of Health, Bethesda, MD, 1991. In an exemplary embodiment, the anti-MerTK antibody binds to a fibronectin-like domain of MerTK.

In another aspect, an anti-MerTK antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 93. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 93. In certain embodiments, substitutions, insertions, or deletions occur in regions outside the HVRs (i.e., in the FRs). Optionally, the anti-MerTK antibody comprises the VH sequence in SEQ ID NO: 93, including post-translational modifications of that sequence. In a particular embodiment, the VH comprises one, two or three HVRs selected from: (a) HVR-H1 comprising the amino acid sequence of SEQ ID NO: 27, (b) HVR-H2 comprising the amino acid sequence of SEQ ID NO: 28, and (c) HVR-H3 comprising the amino acid sequence of SEQ ID NO: 29. In an exemplary embodiment, the anti-MerTK antibody binds to a fibronectin-like domain of MerTK.

In another aspect, an anti-MerTK antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 74. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 74. In certain embodiments, the substitutions, insertions, or deletions occur in regions outside the HVRs (i.e., in the FRs). Optionally, the anti-MerTK antibody comprises the VL sequence in SEQ ID NO: 74, including post-translational modifications of that sequence. In a particular embodiment, the VL comprises one, two or three HVRs selected from (a) HVR-L1 comprising the amino acid sequence of SEQ ID NO: 25; (b) HVR-L2 comprising the amino acid sequence of SEQ ID NO: 14; and (c) HVR-L3 comprising the amino acid sequence of SEQ ID NO: 26. In an exemplary embodiment, the anti-MerTK antibody binds to a fibronectin-like domain of MerTK.

In another aspect, an anti-MerTK antibody is provided, wherein the antibody comprises a VH as in any of the embodiments provided above, and a VL as in any of the embodiments provided above. In one embodiment, the antibody comprises the VH and VL sequences in SEQ ID NO: 93 and SEQ ID NO:74, respectively, including post-translational modifications of those sequences. In an exemplary embodiment, the anti-MerTK antibody binds to a fibronectin-like domain of MerTK.

In one aspect, the invention provides an anti-MerTK antibody comprising at least one, at least two, or all three VH HVR sequences selected from (a) HVR-H1 comprising the amino acid sequence of SEQ ID NO:33; (b) HVR-H2 comprising the amino acid sequence of SEQ ID NO:34; and (c) HVR-H3 comprising the amino acid sequence of SEQ ID NO:35. In one embodiment, the antibody comprises (a) HVR-H1 comprising the amino acid sequence of SEQ ID NO:33; (b) HVR-H2 comprising the amino acid sequence of SEQ ID NO:34; and (c) HVR-H3 comprising the amino acid sequence of SEQ ID NO:35. In an exemplary embodiment, the anti-MerTK antibody binds to a fibronectin-like domain of MerTK.

In another aspect, the invention provides an anti-MerTK antibody comprising at least one, at least two, or all three VL HVR sequences selected from (a) HVR-L1 comprising the amino acid sequence of SEQ ID NO: 30; (b) HVR-L2 comprising the amino acid sequence of SEQ ID NO: 31; and (c) HVR-L3 comprising the amino acid sequence of SEQ ID NO: 32. In one embodiment, the antibody comprises (a) HVR-L1 comprising the amino acid sequence of SEQ ID NO: 30; (b) HVR-L2 comprising the amino acid sequence of SEQ ID NO: 31; and (c) HVR-L3 comprising the amino acid sequence of SEQ ID NO: 32. In an exemplary embodiment, the anti-MerTK antibody binds to a fibronectin-like domain of MerTK.

In another aspect, an anti-MerTK antibody of the invention comprises (a) a VH domain comprising at least one, at least two, or all three VH HVR sequences selected from (i) HVR-H1 comprising the amino acid sequence of SEQ ID NO: 33, (ii) HVR-H2 comprising the amino acid sequence of SEQ ID NO: 34, and (iii) HVR-H3 comprising an amino acid sequence selected from SEQ ID NO: 35; and (b) a VL domain comprising at least one, at least two, or all three VL HVR sequences selected from (i) HVR-L1 comprising the amino acid sequence of SEQ ID NO: 30, (ii) HVR-L2 comprising the amino acid sequence of SEQ ID NO: 31, and (c) HVR-L3 comprising the amino acid sequence of SEQ ID NO: 32. In an exemplary embodiment, the anti-MerTK antibody binds to a fibronectin-like domain of MerTK.

In another aspect, the invention provides an anti-MerTK antibody comprising (a) HVR-H1 comprising the amino acid sequence of SEQ ID NO: 33; (b) HVR-H2 comprising the amino acid sequence of SEQ ID NO: 34; (c) HVR-H3 comprising the amino acid sequence of SEQ ID NO: 35; (d) HVR-L1 comprising the amino acid sequence of SEQ ID NO: 30; (e) HVR-L2 comprising the amino acid sequence of SEQ ID NO: 31; and (f) HVR-L3 comprising an amino acid sequence selected from SEQ ID NO: 32. In an exemplary embodiment, the anti-MerTK antibody binds to a fibronectin-like domain of MerTK.

In any of the above embodiments, an anti-MerTK antibody is humanized. In one embodiment, an anti-MerTK antibody comprises HVRs as in any of the above embodiments, and further comprises an acceptor human framework, e.g. a human immunoglobulin framework or a human consensus framework, optionally with up to 10 amino acid substitutions (e.g. from 1-2, 1-3, 1-4, 1-5, 1-6, 1-7, 1-8, 1-9 or 1-10 amino acid substitutions). In exemplary embodiments, such amino acid substitutions correspond to the amino acid residues from a rabbit framework region sequence, such as, for example, one or more of the following residues: Q2, L4, P43, and/or F87 in the light chain variable region framework sequences and/or one or more of the following residues: V24, 148, G49, K71, and/or V78 in the heavy chain variable region framework sequences. The numbering of amino acid residues is according to the EU numbering system, also called the EU index, as described in Kabat et al., Sequences of Proteins of Immunological Interest, 5th Ed. Public Health Service, National Institutes of Health, Bethesda, MD, 1991. In an exemplary embodiment, the anti-MerTK antibody binds to a fibronectin-like domain of MerTK.

In another aspect, an anti-MerTK antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 94. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 94. In certain embodiments, substitutions, insertions, or deletions occur in regions outside the HVRs (i.e., in the FRs). Optionally, the anti-MerTK antibody comprises the VH sequence in SEQ ID NO: 94, including post-translational modifications of that sequence. In a particular embodiment, the VH comprises one, two or three HVRs selected from: (a) HVR-H1 comprising the amino acid sequence of SEQ ID NO: 33, (b) HVR-H2 comprising the amino acid sequence of SEQ ID NO: 34, and (c) HVR-H3 comprising the amino acid sequence of SEQ ID NO: 35. In an exemplary embodiment, the anti-MerTK antibody binds to a fibronectin-like domain of MerTK.

In another aspect, an anti-MerTK antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 75. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 75. In certain embodiments, the substitutions, insertions, or deletions occur in regions outside the HVRs (i.e., in the FRs). Optionally, the anti-MerTK antibody comprises the VL sequence in SEQ ID NO: 75, including post-translational modifications of that sequence. In a particular embodiment, the VL comprises one, two or three HVRs selected from (a) HVR-L1 comprising the amino acid sequence of SEQ ID NO: 30; (b) HVR-L2 comprising the amino acid sequence of SEQ ID NO: 31; and (c) HVR-L3 comprising the amino acid sequence of SEQ ID NO: 32. In an exemplary embodiment, the anti-MerTK antibody binds to a fibronectin-like domain of MerTK.

In another aspect, an anti-MerTK antibody is provided, wherein the antibody comprises a VH as in any of the embodiments provided above, and a VL as in any of the embodiments provided above. In one embodiment, the antibody comprises the VH and VL sequences in SEQ ID NO: 94 and SEQ ID NO: 75, respectively, including post-translational modifications of those sequences. In an exemplary embodiment, the anti-MerTK antibody binds to a fibronectin-like domain of MerTK.

In one aspect, the invention provides an anti-MerTK antibody comprising at least one, at least two, or all three VH HVR sequences selected from (a) HVR-H1 comprising the amino acid sequence of SEQ ID NO: 38; (b) HVR-H2 comprising the amino acid sequence of SEQ ID NO: 39; and (c) HVR-H3 comprising the amino acid sequence of SEQ ID NO: 40. In one embodiment, the antibody comprises (a) HVR-H1 comprising the amino acid sequence of SEQ ID NO: 38; (b) HVR-H2 comprising the amino acid sequence of SEQ ID NO: 39; and (c) HVR-H3 comprising the amino acid sequence of SEQ ID NO: 40. In an exemplary embodiment, the anti-MerTK antibody binds to a Ig-like domain of MerTK.

In another aspect, the invention provides an anti-MerTK antibody comprising at least one, at least two, or all three VL HVR sequences selected from (a) HVR-L1 comprising the amino acid sequence of SEQ ID NO: 36; (b) HVR-L2 comprising the amino acid sequence of SEQ ID NO: 14; and (c) HVR-L3 comprising the amino acid sequence of SEQ ID NO: 37. In one embodiment, the antibody comprises (a) HVR-L1 comprising the amino acid sequence of SEQ ID NO: 36; (b) HVR-L2 comprising the amino acid sequence of SEQ ID NO: 14; and (c) HVR-L3 comprising the amino acid sequence of SEQ ID NO: 37. In an exemplary embodiment, the anti-MerTK antibody binds to a Ig-like domain of MerTK.

In another aspect, an anti-MerTK antibody of the invention comprises (a) a VH domain comprising at least one, at least two, or all three VH HVR sequences selected from (i) HVR-H1 comprising the amino acid sequence of SEQ ID NO: 38, (ii) HVR-H2 comprising the amino acid sequence of SEQ ID NO: 39, and (iii) HVR-H3 comprising an amino acid sequence selected from SEQ ID NO: 40; and (b) a VL domain comprising at least one, at least two, or all three VL HVR sequences selected from (i) HVR-L1 comprising the amino acid sequence of SEQ ID NO: 36, (ii) HVR-L2 comprising the amino acid sequence of SEQ ID NO: 14, and (c) HVR-L3 comprising the amino acid sequence of SEQ ID NO: 37. In an exemplary embodiment, the anti-MerTK antibody binds to a Ig-like domain of MerTK.

In another aspect, the invention provides an anti-MerTK antibody comprising (a) HVR-H1 comprising the amino acid sequence of SEQ ID NO: 38; (b) HVR-H2 comprising the amino acid sequence of SEQ ID NO: 39; (c) HVR-H3 comprising the amino acid sequence of SEQ ID NO: 40; (d) HVR-L1 comprising the amino acid sequence of SEQ ID NO: 36; (e) HVR-L2 comprising the amino acid sequence of SEQ ID NO: 14; and (f) HVR-L3 comprising an amino acid sequence selected from SEQ ID NO: 37. In an exemplary embodiment, the anti-MerTK antibody binds to a Ig-like domain of MerTK.

In any of the above embodiments, an anti-MerTK antibody is humanized. In one embodiment, an anti-MerTK antibody comprises HVRs as in any of the above embodiments, and further comprises an acceptor human framework, e.g. a human immunoglobulin framework or a human consensus framework, optionally with up to 10 amino acid substitutions (e.g. from 1-2, 1-3, 1-4, 1-5, 1-6, 1-7, 1-8, 1-9 or 1-10 amino acid substitutions). In exemplary embodiments, such amino acid substitutions correspond to the amino acid residues from a rabbit framework region sequence, such as, for example, one or more of the following residues: Q2, L4, P43, and/or F87 in the light chain variable region framework sequences and/or one or more of the following residues: V24, 148, G49, K71, and/or V78 in the heavy chain variable region framework sequences. The numbering of amino acid residues is according to the EU numbering system, also called the EU index, as described in Kabat et al., Sequences of Proteins of Immunological Interest, 5th Ed. Public Health Service, National Institutes of Health, Bethesda, MD, 1991. In an exemplary embodiment, the anti-MerTK antibody binds to a fibronectin-like domain of MerTK.

In another aspect, an anti-MerTK antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 95. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 95. In certain embodiments, substitutions, insertions, or deletions occur in regions outside the HVRs (i.e., in the FRs). Optionally, the anti-MerTK antibody comprises the VH sequence in SEQ ID NO: 95, including post-translational modifications of that sequence. In a particular embodiment, the VH comprises one, two or three HVRs selected from: (a) HVR-H1 comprising the amino acid sequence of SEQ ID NO: 38, (b) HVR-H2 comprising the amino acid sequence of SEQ ID NO: 39, and (c) HVR-H3 comprising the amino acid sequence of SEQ ID NO: 40. In an exemplary embodiment, the anti-MerTK antibody binds to a Ig-like domain of MerTK.

In another aspect, an anti-MerTK antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 76. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 76. In certain embodiments, the substitutions, insertions, or deletions occur in regions outside the HVRs (i.e., in the FRs). Optionally, the anti-MerTK antibody comprises the VL sequence in SEQ ID NO: 76, including post-translational modifications of that sequence. In a particular embodiment, the VL comprises one, two or three HVRs selected from (a) HVR-L1 comprising the amino acid sequence of SEQ ID NO: 36; (b) HVR-L2 comprising the amino acid sequence of SEQ ID NO: 14; and (c) HVR-L3 comprising the amino acid sequence of SEQ ID NO: 37. In an exemplary embodiment, the anti-MerTK antibody binds to a Ig-like domain of MerTK.

In another aspect, an anti-MerTK antibody is provided, wherein the antibody comprises a VH as in any of the embodiments provided above, and a VL as in any of the embodiments provided above. In one embodiment, the antibody comprises the VH and VL sequences in SEQ ID NO: 95 and SEQ ID NO: 76, respectively, including post-translational modifications of those sequences. In an exemplary embodiment, the anti-MerTK antibody binds to a Ig-like domain of MerTK.

In one aspect, the invention provides an anti-MerTK antibody comprising at least one, at least two, or all three VH HVR sequences selected from (a) HVR-H1 comprising the amino acid sequence of SEQ ID NO: 44; (b) HVR-H2 comprising the amino acid sequence of SEQ ID NO: 45; and (c) HVR-H3 comprising the amino acid sequence of SEQ ID NO: 46. In one embodiment, the antibody comprises (a) HVR-H1 comprising the amino acid sequence of SEQ ID NO: 44; (b) HVR-H2 comprising the amino acid sequence of SEQ ID NO: 45; and (c) HVR-H3 comprising the amino acid sequence of SEQ ID NO: 46. In an exemplary embodiment, the anti-MerTK antibody binds to a Ig-like domain of MerTK.

In another aspect, the invention provides an anti-MerTK antibody comprising at least one, at least two, or all three VL HVR sequences selected from (a) HVR-L1 comprising the amino acid sequence of SEQ ID NO: 41; (b) HVR-L2 comprising the amino acid sequence of SEQ ID NO: 42; and (c) HVR-L3 comprising the amino acid sequence of SEQ ID NO: 43. In one embodiment, the antibody comprises (a) HVR-L1 comprising the amino acid sequence of SEQ ID NO: 41; (b) HVR-L2 comprising the amino acid sequence of SEQ ID NO: 42; and (c) HVR-L3 comprising the amino acid sequence of SEQ ID NO: 43. In an exemplary embodiment, the anti-MerTK antibody binds to a Ig-like domain of MerTK.

In another aspect, an anti-MerTK antibody of the invention comprises (a) a VH domain comprising at least one, at least two, or all three VH HVR sequences selected from (i) HVR-H1 comprising the amino acid sequence of SEQ ID NO: 44, (ii) HVR-H2 comprising the amino acid sequence of SEQ ID NO: 45, and (iii) HVR-H3 comprising an amino acid sequence selected from SEQ ID NO: 46; and (b) a VL domain comprising at least one, at least two, or all three VL HVR sequences selected from (i) HVR-L1 comprising the amino acid sequence of SEQ ID NO: 41, (ii) HVR-L2 comprising the amino acid sequence of SEQ ID NO: 42, and (c) HVR-L3 comprising the amino acid sequence of SEQ ID NO: 43. In an exemplary embodiment, the anti-MerTK antibody binds to a Ig-like domain of MerTK.

In another aspect, the invention provides an anti-MerTK antibody comprising (a) HVR-H1 comprising the amino acid sequence of SEQ ID NO: 44; (b) HVR-H2 comprising the amino acid sequence of SEQ ID NO: 45; (c) HVR-H3 comprising the amino acid sequence of SEQ ID NO: 46; (d) HVR-L1 comprising the amino acid sequence of SEQ ID NO: 41; (e) HVR-L2 comprising the amino acid sequence of SEQ ID NO: 42; and (f) HVR-L3 comprising an amino acid sequence selected from SEQ ID NO:43. In an exemplary embodiment, the anti-MerTK antibody binds to a Ig-like domain of MerTK.

In any of the above embodiments, an anti-MerTK antibody is humanized. In one embodiment, an anti-MerTK antibody comprises HVRs as in any of the above embodiments, and further comprises an acceptor human framework, e.g. a human immunoglobulin framework or a human consensus framework, optionally with up to 10 amino acid substitutions (e.g. from 1-2, 1-3, 1-4, 1-5, 1-6, 1-7, 1-8, 1-9 or 1-10 amino acid substitutions). In exemplary embodiments, such amino acid substitutions correspond to the amino acid residues from a rabbit framework region sequence, such as, for example, one or more of the following residues: Q2, L4, P43, and/or F87 in the light chain variable region framework sequences and/or one or more of the following residues: V24, 148, G49, K71, and/or V78 in the heavy chain variable region framework sequences. The numbering of amino acid residues is according to the EU numbering system, also called the EU index, as described in Kabat et al., Sequences of Proteins of Immunological Interest, 5th Ed. Public Health Service, National Institutes of Health, Bethesda, MD, 1991. In an exemplary embodiment, the anti-MerTK antibody binds to a fibronectin-like domain of MerTK.

In another aspect, an anti-MerTK antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 96. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 96. In certain embodiments, substitutions, insertions, or deletions occur in regions outside the HVRs (i.e., in the FRs). Optionally, the anti-MerTK antibody comprises the VH sequence in SEQ ID NO: 96, including post-translational modifications of that sequence. In a particular embodiment, the VH comprises one, two or three HVRs selected from: (a) HVR-H1 comprising the amino acid sequence of SEQ ID NO: 44, (b) HVR-H2 comprising the amino acid sequence of SEQ ID NO:45, and (c) HVR-H3 comprising the amino acid sequence of SEQ ID NO: 46. In an exemplary embodiment, the anti-MerTK antibody binds to a Ig-like domain of MerTK.

In another aspect, an anti-MerTK antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 77. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 77. In certain embodiments, the substitutions, insertions, or deletions occur in regions outside the HVRs (i.e., in the FRs). Optionally, the anti-MerTK antibody comprises the VL sequence in SEQ ID NO: 77, including post-translational modifications of that sequence. In a particular embodiment, the VL comprises one, two or three HVRs selected from (a) HVR-L1 comprising the amino acid sequence of SEQ ID NO: 41; (b) HVR-L2 comprising the amino acid sequence of SEQ ID NO: 42; and (c) HVR-L3 comprising the amino acid sequence of SEQ ID NO: 43. In an exemplary embodiment, the anti-MerTK antibody binds to a Ig-like domain of MerTK.

In another aspect, an anti-MerTK antibody is provided, wherein the antibody comprises a VH as in any of the embodiments provided above, and a VL as in any of the embodiments provided above. In one embodiment, the antibody comprises the VH and VL sequences in SEQ ID NO: 96 and SEQ ID NO: 77, respectively, including post-translational modifications of those sequences. In an exemplary embodiment, the anti-MerTK antibody binds to a Ig-like domain of MerTK.

In one aspect, the invention provides an anti-MerTK antibody comprising at least one, at least two, or all three VH HVR sequences selected from (a) HVR-H1 comprising the amino acid sequence of SEQ ID NO: 50; (b) HVR-H2 comprising the amino acid sequence of SEQ ID NO: 51; and (c) HVR-H3 comprising the amino acid sequence of SEQ ID NO: 52. In one embodiment, the antibody comprises (a) HVR-H1 comprising the amino acid sequence of SEQ ID NO: 50; (b) HVR-H2 comprising the amino acid sequence of SEQ ID NO: 51; and (c) HVR-H3 comprising the amino acid sequence of SEQ ID NO: 52. In an exemplary embodiment, the anti-MerTK antibody binds to a Ig-like domain of MerTK.

In another aspect, the invention provides an anti-MerTK antibody comprising at least one, at least two, or all three VL HVR sequences selected from (a) HVR-L1 comprising the amino acid sequence of SEQ ID NO: 47; (b) HVR-L2 comprising the amino acid sequence of SEQ ID NO: 48; and (c) HVR-L3 comprising the amino acid sequence of SEQ ID NO: 49. In one embodiment, the antibody comprises (a) HVR-L1 comprising the amino acid sequence of SEQ ID NO: 47; (b) HVR-L2 comprising the amino acid sequence of SEQ ID NO: 48; and (c) HVR-L3 comprising the amino acid sequence of SEQ ID NO: 49. In an exemplary embodiment, the anti-MerTK antibody binds to a Ig-like domain of MerTK.

In another aspect, an anti-MerTK antibody of the invention comprises (a) a VH domain comprising at least one, at least two, or all three VH HVR sequences selected from (i) HVR-H1 comprising the amino acid sequence of SEQ ID NO: 50, (ii) HVR-H2 comprising the amino acid sequence of SEQ ID NO: 51, and (iii) HVR-H3 comprising an amino acid sequence selected from SEQ ID NO: 52; and (b) a VL domain comprising at least one, at least two, or all three VL HVR sequences selected from (i) HVR-L1 comprising the amino acid sequence of SEQ ID NO: 47, (ii) HVR-L2 comprising the amino acid sequence of SEQ ID NO: 48, and (c) HVR-L3 comprising the amino acid sequence of SEQ ID NO: 49. In an exemplary embodiment, the anti-MerTK antibody binds to a Ig-like domain of MerTK.

In another aspect, the invention provides an anti-MerTK antibody comprising (a) HVR-H1 comprising the amino acid sequence of SEQ ID NO: 50; (b) HVR-H2 comprising the amino acid sequence of SEQ ID NO: 51; (c) HVR-H3 comprising the amino acid sequence of SEQ ID NO: 52; (d) HVR-L1 comprising the amino acid sequence of SEQ ID NO: 47; (e) HVR-L2 comprising the amino acid sequence of SEQ ID NO: 48; and (f) HVR-L3 comprising an amino acid sequence selected from SEQ ID NO: 49. In an exemplary embodiment, the anti-MerTK antibody binds to a Ig-like domain of MerTK.

In any of the above embodiments, an anti-MerTK antibody is humanized. In one embodiment, an anti-MerTK antibody comprises HVRs as in any of the above embodiments, and further comprises an acceptor human framework, e.g. a human immunoglobulin framework or a human consensus framework, optionally with up to 10 amino acid substitutions (e.g. from 1-2, 1-3, 1-4, 1-5, 1-6, 1-7, 1-8, 1-9 or 1-10 amino acid substitutions). In exemplary embodiments, such amino acid substitutions correspond to the amino acid residues from a rabbit framework region sequence, such as, for example, one or more of the following residues: Q2, L4, P43, and/or F87 in the light chain variable region framework sequences and/or one or more of the following residues: V24, 148, G49, K71, and/or V78 in the heavy chain variable region framework sequences. The numbering of amino acid residues is according to the EU numbering system, also called the EU index, as described in Kabat et al., Sequences of Proteins of Immunological Interest, 5th Ed. Public Health Service, National Institutes of Health, Bethesda, MD, 1991. In an exemplary embodiment, the anti-MerTK antibody binds to a fibronectin-like domain of MerTK.

In another aspect, an anti-MerTK antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 97. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 97. In certain embodiments, substitutions, insertions, or deletions occur in regions outside the HVRs (i.e., in the FRs). Optionally, the anti-MerTK antibody comprises the VH sequence in SEQ ID NO: 97, including post-translational modifications of that sequence. In a particular embodiment, the VH comprises one, two or three HVRs selected from: (a) HVR-H1 comprising the amino acid sequence of SEQ ID NO: 50, (b) HVR-H2 comprising the amino acid sequence of SEQ ID NO: 51, and (c) HVR-H3 comprising the amino acid sequence of SEQ ID NO: 52. In an exemplary embodiment, the anti-MerTK antibody binds to a Ig-like domain of MerTK.

In another aspect, an anti-MerTK antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 78. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 78. In certain embodiments, the substitutions, insertions, or deletions occur in regions outside the HVRs (i.e., in the FRs). Optionally, the anti-MerTK antibody comprises the VL sequence in SEQ ID NO: 78, including post-translational modifications of that sequence. In a particular embodiment, the VL comprises one, two or three HVRs selected from (a) HVR-L1 comprising the amino acid sequence of SEQ ID NO: 47; (b) HVR-L2 comprising the amino acid sequence of SEQ ID NO: 48; and (c) HVR-L3 comprising the amino acid sequence of SEQ ID NO: 49. In an exemplary embodiment, the anti-MerTK antibody binds to a Ig-like domain of MerTK.

In another aspect, an anti-MerTK antibody is provided, wherein the antibody comprises a VH as in any of the embodiments provided above, and a VL as in any of the embodiments provided above. In one embodiment, the antibody comprises the VH and VL sequences in SEQ ID NO: 97 and SEQ ID NO: 78, respectively, including post-translational modifications of those sequences. In an exemplary embodiment, the anti-MerTK antibody binds to a Ig-like domain of MerTK.

In another aspect, an anti-MerTK antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 98. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 98. In certain embodiments, substitutions, insertions, or deletions occur in regions outside the HVRs (i.e., in the FRs). Optionally, the anti-MerTK antibody comprises the VH sequence in SEQ ID NO: 98, including post-translational modifications of that sequence. In a particular embodiment, the VH comprises one, two or three HVRs selected from: (a) HVR-H1 comprising the amino acid sequence of SEQ ID NO: 50, (b) HVR-H2 comprising the amino acid sequence of SEQ ID NO: 51, and (c) HVR-H3 comprising the amino acid sequence of SEQ ID NO: 52. In an exemplary embodiment, the anti-MerTK antibody binds to a Ig-like domain of MerTK.

In another aspect, an anti-MerTK antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 79. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 79. In certain embodiments, the substitutions, insertions, or deletions occur in regions outside the HVRs (i.e., in the FRs). Optionally, the anti-MerTK antibody comprises the VL sequence in SEQ ID NO: 79, including post-translational modifications of that sequence. In a particular embodiment, the VL comprises one, two or three HVRs selected from (a) HVR-L1 comprising the amino acid sequence of SEQ ID NO: 47; (b) HVR-L2 comprising the amino acid sequence of SEQ ID NO: 48; and (c) HVR-L3 comprising the amino acid sequence of SEQ ID NO: 49. In an exemplary embodiment, the anti-MerTK antibody binds to a Ig-like domain of MerTK.

In another aspect, an anti-MerTK antibody is provided, wherein the antibody comprises a VH as in any of the embodiments provided above, and a VL as in any of the embodiments provided above. In one embodiment, the antibody comprises the VH and VL sequences in SEQ ID NO: 98 and SEQ ID NO: 79, respectively, including post-translational modifications of those sequences. In an exemplary embodiment, the anti-MerTK antibody binds to a Ig-like domain of MerTK.

In another aspect, an anti-MerTK antibody comprises a heavy chain sequence having at least 90%, 91%, 92%, 93%, 94%, 95%, 96%, 97%, 98%, 99%, or 100% sequence identity to the amino acid sequence of SEQ ID NO: 108. In certain embodiments, a heavy chain sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 108. In certain embodiments, substitutions, insertions, or deletions occur in regions outside the HVRs (i.e., in the FRs). Optionally, the anti-MerTK antibody comprises the heavy chain sequence in SEQ ID NO: 108, including post-translational modifications of that sequence. In a particular embodiment, the heavy chain comprises one, two or three HVRs selected from: (a) HVR-H1 comprising the amino acid sequence of SEQ ID NO: 50, (b) HVR-H2 comprising the amino acid sequence of SEQ ID NO:51, and (c) HVR-H3 comprising the amino acid sequence of SEQ ID NO:52. In an exemplary embodiment, the anti-MerTK antibody binds to a Ig-like domain of MerTK.

In another aspect, an anti-MerTK antibody is provided, wherein the antibody comprises a light chain having at least 90%, 91%, 92%, 93%, 94%, 95%, 96%, 97%, 98%, 99%, or 100% sequence identity to the amino acid sequence of SEQ ID NO: 116. In certain embodiments, a light chain sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 116. In certain embodiments, the substitutions, insertions, or deletions occur in regions outside the HVRs (i.e., in the FRs). Optionally, the anti-MerTK antibody comprises the light chain sequence in SEQ ID NO: 116, including post-translational modifications of that sequence. In a particular embodiment, the light chain comprises one, two or three HVRs selected from (a) HVR-L1 comprising the amino acid sequence of SEQ ID NO: 47; (b) HVR-L2 comprising the amino acid sequence of SEQ ID NO: 48; and (c) HVR-L3 comprising the amino acid sequence of SEQ ID NO: 49. In an exemplary embodiment, the anti-MerTK antibody binds to a Ig-like domain of MerTK.

In another aspect, an anti-MerTK antibody is provided, wherein the antibody comprises a heavy chain as in any of the embodiments provided above, and a light chain as in any of the embodiments provided above. In one embodiment, the antibody comprises the heavy chain and light chain sequences in SEQ ID NO: 108 and SEQ ID NO: 116, respectively, including post-translational modifications of those sequences. In an exemplary embodiment, the anti-MerTK antibody binds to a Ig-like domain of MerTK.

In another aspect, an anti-MerTK antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 99. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 99. In certain embodiments, substitutions, insertions, or deletions occur in regions outside the HVRs (i.e., in the FRs). Optionally, the anti-MerTK antibody comprises the VH sequence in SEQ ID NO: 99, including post-translational modifications of that sequence. In a particular embodiment, the VH comprises one, two or three HVRs selected from: (a) HVR-H1 comprising the amino acid sequence of SEQ ID NO: 50, (b) HVR-H2 comprising the amino acid sequence of SEQ ID NO: 51, and (c) HVR-H3 comprising the amino acid sequence of SEQ ID NO: 52. In an exemplary embodiment, the anti-MerTK antibody binds to a Ig-like domain of MerTK.

In another aspect, an anti-MerTK antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 80. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 80. In certain embodiments, the substitutions, insertions, or deletions occur in regions outside the HVRs (i.e., in the FRs). Optionally, the anti-MerTK antibody comprises the VL sequence in SEQ ID NO: 80, including post-translational modifications of that sequence. In a particular embodiment, the VL comprises one, two or three HVRs selected from (a) HVR-L1 comprising the amino acid sequence of SEQ ID NO: 47; (b) HVR-L2 comprising the amino acid sequence of SEQ ID NO: 48; and (c) HVR-L3 comprising the amino acid sequence of SEQ ID NO: 49. In an exemplary embodiment, the anti-MerTK antibody binds to a Ig-like domain of MerTK.

In another aspect, an anti-MerTK antibody is provided, wherein the antibody comprises a VH as in any of the embodiments provided above, and a VL as in any of the embodiments provided above. In one embodiment, the antibody comprises the VH and VL sequences in SEQ ID NO: 99 and SEQ ID NO: 80, respectively, including post-translational modifications of those sequences. In an exemplary embodiment, the anti-MerTK antibody binds to a Ig-like domain of MerTK.

In another aspect, an anti-MerTK antibody comprises a heavy chain sequence having at least 90%, 91%, 92%, 93%, 94%, 95%, 96%, 97%, 98%, 99%, or 100% sequence identity to the amino acid sequence of SEQ ID NO: 109. In certain embodiments, a heavy chain sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 109. In certain embodiments, substitutions, insertions, or deletions occur in regions outside the HVRs (i.e., in the FRs). Optionally, the anti-MerTK antibody comprises the heavy chain sequence in SEQ ID NO: 109, including post-translational modifications of that sequence. In a particular embodiment, the heavy chain comprises one, two or three HVRs selected from: (a) HVR-H1 comprising the amino acid sequence of SEQ ID NO: 50, (b) HVR-H2 comprising the amino acid sequence of SEQ ID NO: 51, and (c) HVR-H3 comprising the amino acid sequence of SEQ ID NO: 52. In an exemplary embodiment, the anti-MerTK antibody binds to a Ig-like domain of MerTK.

In another aspect, an anti-MerTK antibody is provided, wherein the antibody comprises a light chain having at least 90%, 91%, 92%, 93%, 94%, 95%, 96%, 97%, 98%, 99%, or 100% sequence identity to the amino acid sequence of SEQ ID NO: 117. In certain embodiments, a light chain sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 117. In certain embodiments, the substitutions, insertions, or deletions occur in regions outside the HVRs (i.e., in the FRs). Optionally, the anti-MerTK antibody comprises the light chain sequence in SEQ ID NO: 117, including post-translational modifications of that sequence. In a particular embodiment, the light chain comprises one, two or three HVRs selected from (a) HVR-L1 comprising the amino acid sequence of SEQ ID NO: 47; (b) HVR-L2 comprising the amino acid sequence of SEQ ID NO: 48; and (c) HVR-L3 comprising the amino acid sequence of SEQ ID NO: 49. In an exemplary embodiment, the anti-MerTK antibody binds to a Ig-like domain of MerTK.

In another aspect, an anti-MerTK antibody is provided, wherein the antibody comprises a heavy chain as in any of the embodiments provided above, and a light chain as in any of the embodiments provided above. In one embodiment, the antibody comprises the heavy chain and light chain sequences in SEQ ID NO: 109 and SEQ ID NO: 117, respectively, including post-translational modifications of those sequences. In an exemplary embodiment, the anti-MerTK antibody binds to a Ig-like domain of MerTK.

In one aspect, the invention provides an anti-MerTK antibody comprising at least one, at least two, or all three VH HVR sequences selected from (a) HVR-H1 comprising the amino acid sequence of SEQ ID NO: 56; (b) HVR-H2 comprising the amino acid sequence of SEQ ID NO: 57; and (c) HVR-H3 comprising the amino acid sequence of SEQ ID NO: 58. In one embodiment, the antibody comprises (a) HVR-H1 comprising the amino acid sequence of SEQ ID NO: 56; (b) HVR-H2 comprising the amino acid sequence of SEQ ID NO: 57; and (c) HVR-H3 comprising the amino acid sequence of SEQ ID NO: 58. In an exemplary embodiment, the anti-MerTK antibody binds to a Ig-like domain of MerTK.

In another aspect, the invention provides an anti-MerTK antibody comprising at least one, at least two, or all three VL HVR sequences selected from (a) HVR-L1 comprising the amino acid sequence of SEQ ID NO: 53; (b) HVR-L2 comprising the amino acid sequence of SEQ ID NO: 54; and (c) HVR-L3 comprising the amino acid sequence of SEQ ID NO: 55. In one embodiment, the antibody comprises (a) HVR-L1 comprising the amino acid sequence of SEQ ID NO: 53; (b) HVR-L2 comprising the amino acid sequence of SEQ ID NO: 54; and (c) HVR-L3 comprising the amino acid sequence of SEQ ID NO: 55. In an exemplary embodiment, the anti-MerTK antibody binds to a Ig-like domain of MerTK.

In another aspect, an anti-MerTK antibody of the invention comprises (a) a VH domain comprising at least one, at least two, or all three VH HVR sequences selected from (i) HVR-H1 comprising the amino acid sequence of SEQ ID NO: 56, (ii) HVR-H2 comprising the amino acid sequence of SEQ ID NO: 57, and (iii) HVR-H3 comprising an amino acid sequence selected from SEQ ID NO: 58; and (b) a VL domain comprising at least one, at least two, or all three VL HVR sequences selected from (i) HVR-L1 comprising the amino acid sequence of SEQ ID NO: 53, (ii) HVR-L2 comprising the amino acid sequence of SEQ ID NO: 54, and (c) HVR-L3 comprising the amino acid sequence of SEQ ID NO: 55. In an exemplary embodiment, the anti-MerTK antibody binds to a Ig-like domain of MerTK.

In another aspect, the invention provides an anti-MerTK antibody comprising (a) HVR-H1 comprising the amino acid sequence of SEQ ID NO: 56; (b) HVR-H2 comprising the amino acid sequence of SEQ ID NO: 57; (c) HVR-H3 comprising the amino acid sequence of SEQ ID NO: 58; (d) HVR-L1 comprising the amino acid sequence of SEQ ID NO: 53; (e) HVR-L2 comprising the amino acid sequence of SEQ ID NO: 54; and (f) HVR-L3 comprising an amino acid sequence selected from SEQ ID NO: 55. In an exemplary embodiment, the anti-MerTK antibody binds to a Ig-like domain of MerTK.

In any of the above embodiments, an anti-MerTK antibody is humanized. In one embodiment, an anti-MerTK antibody comprises HVRs as in any of the above embodiments, and further comprises an acceptor human framework, e.g. a human immunoglobulin framework or a human consensus framework, optionally with up to 10 amino acid substitutions (e.g. from 1-2, 1-3, 1-4, 1-5, 1-6, 1-7, 1-8, 1-9 or 1-10 amino acid substitutions). In exemplary embodiments, such amino acid substitutions correspond to the amino acid residues from a rabbit framework region sequence, such as, for example, one or more of the following residues: Q2, L4, P43, and/or F87 in the light chain variable region framework sequences and/or one or more of the following residues: V24, 148, G49, K71, and/or V78 in the heavy chain variable region framework sequences. The numbering of amino acid residues is according to the EU numbering system, also called the EU index, as described in Kabat et al., Sequences of Proteins of Immunological Interest, 5th Ed. Public Health Service, National Institutes of Health, Bethesda, MD, 1991. In an exemplary embodiment, the anti-MerTK antibody binds to a fibronectin-like domain of MerTK.

In another aspect, an anti-MerTK antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 100. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 100. In certain embodiments, substitutions, insertions, or deletions occur in regions outside the HVRs (i.e., in the FRs). Optionally, the anti-MerTK antibody comprises the VH sequence in SEQ ID NO: 100, including post-translational modifications of that sequence. In a particular embodiment, the VH comprises one, two or three HVRs selected from: (a) HVR-H1 comprising the amino acid sequence of SEQ ID NO: 56, (b) HVR-H2 comprising the amino acid sequence of SEQ ID NO: 57, and (c) HVR-H3 comprising the amino acid sequence of SEQ ID NO: 58. In an exemplary embodiment, the anti-MerTK antibody binds to a Ig-like domain of MerTK.

In another aspect, an anti-MerTK antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 81. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 81. In certain embodiments, the substitutions, insertions, or deletions occur in regions outside the HVRs (i.e., in the FRs). Optionally, the anti-MerTK antibody comprises the VL sequence in SEQ ID NO: 81, including post-translational modifications of that sequence. In a particular embodiment, the VL comprises one, two or three HVRs selected from (a) HVR-L1 comprising the amino acid sequence of SEQ ID NO: 53; (b) HVR-L2 comprising the amino acid sequence of SEQ ID NO: 54; and (c) HVR-L3 comprising the amino acid sequence of SEQ ID NO: 55. In an exemplary embodiment, the anti-MerTK antibody binds to a Ig-like domain of MerTK.

In another aspect, an anti-MerTK antibody is provided, wherein the antibody comprises a VH as in any of the embodiments provided above, and a VL as in any of the embodiments provided above. In one embodiment, the antibody comprises the VH and VL sequences in SEQ ID NO: 100 and SEQ ID NO: 81, respectively, including post-translational modifications of those sequences. In an exemplary embodiment, the anti-MerTK antibody binds to a Ig-like domain of MerTK.

In one aspect, the invention provides an anti-MerTK antibody comprising at least one, at least two, or all three VH HVR sequences selected from (a) HVR-H1 comprising the amino acid sequence of SEQ ID NO: 62; (b) HVR-H2 comprising the amino acid sequence of SEQ ID NO: 63; and (c) HVR-H3 comprising the amino acid sequence of SEQ ID NO: 64. In one embodiment, the antibody comprises (a) HVR-H1 comprising the amino acid sequence of SEQ ID NO: 62; (b) HVR-H2 comprising the amino acid sequence of SEQ ID NO: 63; and (c) HVR-H3 comprising the amino acid sequence of SEQ ID NO: 64. In an exemplary embodiment, the anti-MerTK antibody binds to a Ig-like domain of MerTK.

In another aspect, the invention provides an anti-MerTK antibody comprising at least one, at least two, or all three VL HVR sequences selected from (a) HVR-L1 comprising the amino acid sequence of SEQ ID NO: 59; (b) HVR-L2 comprising the amino acid sequence of SEQ ID NO: 60; and (c) HVR-L3 comprising the amino acid sequence of SEQ ID NO: 61. In one embodiment, the antibody comprises (a) HVR-L1 comprising the amino acid sequence of SEQ ID NO: 59; (b) HVR-L2 comprising the amino acid sequence of SEQ ID NO: 60; and (c) HVR-L3 comprising the amino acid sequence of SEQ ID NO: 61. In an exemplary embodiment, the anti-MerTK antibody binds to a Ig-like domain of MerTK.

In another aspect, an anti-MerTK antibody of the invention comprises (a) a VH domain comprising at least one, at least two, or all three VH HVR sequences selected from (i) HVR-H1 comprising the amino acid sequence of SEQ ID NO: 62, (ii) HVR-H2 comprising the amino acid sequence of SEQ ID NO: 63, and (iii) HVR-H3 comprising an amino acid sequence selected from SEQ ID NO: 64; and (b) a VL domain comprising at least one, at least two, or all three VL HVR sequences selected from (i) HVR-L1 comprising the amino acid sequence of SEQ ID NO: 59, (ii) HVR-L2 comprising the amino acid sequence of SEQ ID NO: 60, and (c) HVR-L3 comprising the amino acid sequence of SEQ ID NO: 61. In an exemplary embodiment, the anti-MerTK antibody binds to a Ig-like domain of MerTK.

In another aspect, the invention provides an anti-MerTK antibody comprising (a) HVR-H1 comprising the amino acid sequence of SEQ ID NO: 62; (b) HVR-H2 comprising the amino acid sequence of SEQ ID NO: 63; (c) HVR-H3 comprising the amino acid sequence of SEQ ID NO: 64; (d) HVR-L1 comprising the amino acid sequence of SEQ ID NO: 59; (e) HVR-L2 comprising the amino acid sequence of SEQ ID NO: 60; and (f) HVR-L3 comprising an amino acid sequence selected from SEQ ID NO: 61. In an exemplary embodiment, the anti-MerTK antibody binds to a Ig-like domain of MerTK.

In any of the above embodiments, an anti-MerTK antibody is humanized. In one embodiment, an anti-MerTK antibody comprises HVRs as in any of the above embodiments, and further comprises an acceptor human framework, e.g. a human immunoglobulin framework or a human consensus framework, optionally with up to 10 amino acid substitutions (e.g. from 1-2, 1-3, 1-4, 1-5, 1-6, 1-7, 1-8, 1-9 or 1-10 amino acid substitutions). In exemplary embodiments, such amino acid substitutions correspond to the amino acid residues from a rabbit framework region sequence, such as, for example, one or more of the following residues: Q2, L4, P43, and/or F87 in the light chain variable region framework sequences and/or one or more of the following residues: V24, 148, G49, K71, and/or V78 in the heavy chain variable region framework sequences. The numbering of amino acid residues is according to the EU numbering system, also called the EU index, as described in Kabat et al., Sequences of Proteins of Immunological Interest, 5th Ed. Public Health Service, National Institutes of Health, Bethesda, MD, 1991. In an exemplary embodiment, the anti-MerTK antibody binds to a fibronectin-like domain of MerTK.

In another aspect, an anti-MerTK antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 101. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 101. In certain embodiments, substitutions, insertions, or deletions occur in regions outside the HVRs (i.e., in the FRs). Optionally, the anti-MerTK antibody comprises the VH sequence in SEQ ID NO: 101, including post-translational modifications of that sequence. In a particular embodiment, the VH comprises one, two or three HVRs selected from: (a) HVR-H1 comprising the amino acid sequence of SEQ ID NO: 62, (b) HVR-H2 comprising the amino acid sequence of SEQ ID NO: 63, and (c) HVR-H3 comprising the amino acid sequence of SEQ ID NO: 64. In an exemplary embodiment, the anti-MerTK antibody binds to a Ig-like domain of MerTK.

In another aspect, an anti-MerTK antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 82. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-MerTK antibody comprising that sequence retains the ability to bind to MerTK. In certain embodiments, a total of 1 to 10 amino acids have been substituted, inserted and/or deleted in SEQ ID NO: 82. In certain embodiments, the substitutions, insertions, or deletions occur in regions outside the HVRs (i.e., in the FRs). Optionally, the anti-MerTK antibody comprises the VL sequence in SEQ ID NO: 82, including post-translational modifications of that sequence. In a particular embodiment, the VL comprises one, two or three HVRs selected from (a) HVR-L1 comprising the amino acid sequence of SEQ ID NO: 59; (b) HVR-L2 comprising the amino acid sequence of SEQ ID NO: 60; and (c) HVR-L3 comprising the amino acid sequence of SEQ ID NO: 61. In an exemplary embodiment, the anti-MerTK antibody binds to a Ig-like domain of MerTK.

In another aspect, an anti-MerTK antibody is provided, wherein the antibody comprises a VH as in any of the embodiments provided above, and a VL as in any of the embodiments provided above. In one embodiment, the antibody comprises the VH and VL sequences in SEQ ID NO: 101 and SEQ ID NO: 82, respectively, including post-translational modifications of those sequences. In an exemplary embodiment, the anti-MerTK antibody binds to a Ig-like domain of MerTK.

In a further aspect, the invention provides an antibody that competes for binding to MerTK with an anti-MerTK reference antibody provided herein. For example, in certain embodiments, an antibody is provided that competes for binding to MerTK with one or more of the following anti-MerTK reference antibodies: an antibody comprising a VH comprising the amino acid sequence of SEQ ID NO: 83 and a VL comprising the amino acid sequence of SEQ ID NO: 65; an antibody comprising a VH comprising the amino acid sequence of SEQ ID NO: 84 and a VL comprising the amino acid sequence of SEQ ID NO: 66; an antibody comprising a VH comprising the amino acid sequence of SEQ ID NO: 85 and a VL comprising the amino acid sequence of SEQ ID NO: 67; an antibody comprising a heavy chain comprising the amino acid sequence of SEQ ID NO: 102 and a light chain comprising the amino acid sequence of SEQ ID NO: 110; an antibody comprising a VH comprising the amino acid sequence of SEQ ID NO: 86 and a VL comprising the amino acid sequence of SEQ ID NO: 68; an antibody comprising a heavy chain comprising the amino acid sequence of SEQ ID NO: 103 and a light chain comprising the amino acid sequence of SEQ ID NO: 111; an antibody comprising a VH comprising the amino acid sequence of SEQ ID NO: 87 and a VL comprising the amino acid sequence of SEQ ID NO: 69; an antibody comprising a VH comprising the amino acid sequence of SEQ ID NO: 88 and a VL comprising the amino acid sequence of SEQ ID NO: 70; an antibody comprising a heavy chain comprising the amino acid sequence of SEQ ID NO: 104 and a light chain comprising the amino acid sequence of SEQ ID NO: 112; an antibody comprising a VH comprising the amino acid sequence of SEQ ID NO: 89 and a VL comprising the amino acid sequence of SEQ ID NO: 70; an antibody comprising a heavy chain comprising the amino acid sequence of SEQ ID NO: 105 and a light chain comprising the amino acid sequence of SEQ ID NO: 113; an antibody comprising a VH comprising the amino acid sequence of SEQ ID NO: 90 and a VL comprising the amino acid sequence of SEQ ID NO: 71; an antibody comprising a VH comprising the amino acid sequence of SEQ ID NO: 91 and a VL comprising the amino acid sequence of SEQ ID NO: 72; an antibody comprising a heavy chain comprising the amino acid sequence of SEQ ID NO: 106 and a light chain comprising the amino acid sequence of SEQ ID NO: 114; an antibody comprising a VH comprising the amino acid sequence of SEQ ID NO: 92 and a VL comprising the amino acid sequence of SEQ ID NO: 73; an antibody comprising a heavy chain comprising the amino acid sequence of SEQ ID NO: 107 and a light chain comprising the amino acid sequence of SEQ ID NO: 115; an antibody comprising a VH comprising the amino acid sequence of SEQ ID NO: 93 and a VL comprising the amino acid sequence of SEQ ID NO: 74; an antibody comprising a VH comprising the amino acid sequence of SEQ ID NO: 94 and a VL comprising the amino acid sequence of SEQ ID NO: 75; an antibody comprising a VH comprising the amino acid sequence of SEQ ID NO: 95 and a VL comprising the amino acid sequence of SEQ ID NO: 76; an antibody comprising a VH comprising the amino acid sequence of SEQ ID NO: 96 and a VL comprising the amino acid sequence of SEQ ID NO: 77; an antibody comprising a VH comprising the amino acid sequence of SEQ ID NO: 97 and a VL comprising the amino acid sequence of SEQ ID NO: 78; an antibody comprising a VH comprising the amino acid sequence of SEQ ID NO: 98 and a VL comprising the amino acid sequence of SEQ ID NO: 79; an antibody comprising a heavy chain comprising the amino acid sequence of SEQ ID NO: 108 and a light chain comprising the amino acid sequence of SEQ ID NO: 116; an antibody comprising a VH comprising the amino acid sequence of SEQ ID NO: 99 and a VL comprising the amino acid sequence of SEQ ID NO: 80; an antibody comprising a heavy chain comprising the amino acid sequence of SEQ ID NO: 109 and a light chain comprising the amino acid sequence of SEQ ID NO: 117; an antibody comprising a VH comprising the amino acid sequence of SEQ ID NO: 100 and a VL comprising the amino acid sequence of SEQ ID NO: 81; and an antibody comprising a VH comprising the amino acid sequence of SEQ ID NO: 101 and a VL comprising the amino acid sequence of SEQ ID NO: 82. In some embodiments, the isolated antibody binds to human MerTK. In some embodiments, the reference antibody is Y323, which is commercially available (abeam catalog no. ab52968).

In a further aspect, the invention provides an antibody that binds to the same epitope as an anti-MerTK antibody provided herein. For example, in certain embodiments, an antibody is provided that binds to the same epitope as any one of the following anti-MerTK antibodies: an antibody comprising a VH comprising the amino acid sequence of SEQ ID NO: 83 and a VL comprising the amino acid sequence of SEQ ID NO: 65; an antibody comprising a VH comprising the amino acid sequence of SEQ ID NO: 84 and a VL comprising the amino acid sequence of SEQ ID NO: 66; an antibody comprising a VH comprising the amino acid sequence of SEQ ID NO: 85 and a VL comprising the amino acid sequence of SEQ ID NO: 67; an antibody comprising a heavy chain comprising the amino acid sequence of SEQ ID NO: 102 and a light chain comprising the amino acid sequence of SEQ ID NO: 110; an antibody comprising a VH comprising the amino acid sequence of SEQ ID NO: 86 and a VL comprising the amino acid sequence of SEQ ID NO: 68; an antibody comprising a heavy chain comprising the amino acid sequence of SEQ ID NO: 103 and a light chain comprising the amino acid sequence of SEQ ID NO: 111; an antibody comprising a VH comprising the amino acid sequence of SEQ ID NO: 87 and a VL comprising the amino acid sequence of SEQ ID NO: 69; an antibody comprising a VH comprising the amino acid sequence of SEQ ID NO: 88 and a VL comprising the amino acid sequence of SEQ ID NO: 70; an antibody comprising a heavy chain comprising the amino acid sequence of SEQ ID NO: 104 and a light chain comprising the amino acid sequence of SEQ ID NO: 112; an antibody comprising a VH comprising the amino acid sequence of SEQ ID NO: 89 and a VL comprising the amino acid sequence of SEQ ID NO: 70; an antibody comprising a heavy chain comprising the amino acid sequence of SEQ ID NO: 105 and a light chain comprising the amino acid sequence of SEQ ID NO: 113; an antibody comprising a VH comprising the amino acid sequence of SEQ ID NO: 90 and a VL comprising the amino acid sequence of SEQ ID NO: 71; an antibody comprising a VH comprising the amino acid sequence of SEQ ID NO: 91 and a VL comprising the amino acid sequence of SEQ ID NO: 72; an antibody comprising a heavy chain comprising the amino acid sequence of SEQ ID NO: 106 and a light chain comprising the amino acid sequence of SEQ ID NO: 114; an antibody comprising a VH comprising the amino acid sequence of SEQ ID NO: 92 and a VL comprising the amino acid sequence of SEQ ID NO: 73; an antibody comprising a heavy chain comprising the amino acid sequence of SEQ ID NO: 107 and a light chain comprising the amino acid sequence of SEQ ID NO: 115; an antibody comprising a VH comprising the amino acid sequence of SEQ ID NO: 93 and a VL comprising the amino acid sequence of SEQ ID NO: 74; an antibody comprising a VH comprising the amino acid sequence of SEQ ID NO: 94 and a VL comprising the amino acid sequence of SEQ ID NO: 75. In certain embodiments, an antibody is provided that binds to an epitope within an Fibronectin-like domain of MerTK consisting of amino acid residues 286-384 or 388-480 of MerTK SEQ ID NO: 129. In some embodiments, the antibody binds to the same epitope as antibody is Y323, which is commercially available (abeam catalog no. ab52968).

In a further aspect, the invention provides an antibody that binds to the same epitope as an anti-MerTK antibody provided herein. For example, in certain embodiments, an antibody is provided that binds to the same epitope as any one of the following anti-MerTK antibodies: an antibody comprising a VH comprising the amino acid sequence of SEQ ID NO: 95 and a VL comprising the amino acid sequence of SEQ ID NO: 76; an antibody comprising a VH comprising the amino acid sequence of SEQ ID NO: 96 and a VL comprising the amino acid sequence of SEQ ID NO: 77; an antibody comprising a VH comprising the amino acid sequence of SEQ ID NO: 97 and a VL comprising the amino acid sequence of SEQ ID NO: 78; an antibody comprising a VH comprising the amino acid sequence of SEQ ID NO: 98 and a VL comprising the amino acid sequence of SEQ ID NO: 79; an antibody comprising a heavy chain comprising the amino acid sequence of SEQ ID NO: 108 and a light chain comprising the amino acid sequence of SEQ ID NO: 116; an antibody comprising a VH comprising the amino acid sequence of SEQ ID NO: 99 and a VL comprising the amino acid sequence of SEQ ID NO: 80; an antibody comprising a heavy chain comprising the amino acid sequence of SEQ ID NO: 109 and a light chain comprising the amino acid sequence of SEQ ID NO: 117; an antibody comprising a VH comprising the amino acid sequence of SEQ ID NO: 100 and a VL comprising the amino acid sequence of SEQ ID NO: 81; and an antibody comprising a VH comprising the amino acid sequence of SEQ ID NO: 101 and a VL comprising the amino acid sequence of SEQ ID NO: 82. In certain embodiments, an antibody is provided that binds to an epitope within an Ig-like domain of MerTK consisting of amino acid residues 76-195 or 199-283 of MerTK SEQ ID NO: 129.

In a further aspect of the invention, an anti-MerTK antibody according to any of the above embodiments is a monoclonal antibody, including a chimeric, humanized or human antibody. In one embodiment, an anti-MerTK antibody is an antibody fragment, e.g., a Fv, Fab, Fab′, scFv, diabody, or F(ab′)₂ fragment. In another embodiment, the antibody is a full length antibody, e.g., an intact IgG1 antibody or other antibody class or isotype as defined herein. In certain embodiments, the antibody comprises a mutation in the Fc region that reduces binding to Fc receptors and/or complement. In one embodiment, the antibody comprises a LALAPG mutation in the Fc region.

In a further aspect, an anti-MerTK antibody according to any of the above embodiments may incorporate any of the features, singly or in combination, as described in Sections 1-8 below:

### 1. MerTK Biological Activity

In some embodiments, the antibodies reduce MerTK mediated clearance of apoptotic cells by phagocytes, e.g., the clearance of apoptotic cells is reduced by 1-10 fold, 1-8 fold, 1-5 fold, 1-4 fold, 1-3 fold, 1-2 fold, 2-10 fold, 2-8 fold, 2-5 fold, 2-4 fold, 2-3 fold, 3-10 fold, 3-8 fold, 3-5 fold, 3-4 fold, or by about 1.1 fold, 1.2 fold, 1.3 fold, 1.4 fold, 1.5 fold, 1.6 fold, 1.7 fold, 1.8 fold, 1.9 fold, 2.0 fold, 2.1 fold, 2.2 fold, 2.3 fold, 2.4 fold, 2.5 fold, 2.6 fold, 2.7 fold, 2.8 fold, 2.9 fold, 3.0 fold, 3.1 fold, 3.2 fold, 3.3 fold, 3.4 fold, 3.5 fold, 3.6 fold, 3.7 fold, 3.8 fold, 3.9 fold, 4.0 fold, 4.1 fold, 4.2 fold, 4.3 fold, 4.4 fold, 4.5 fold, 4.6 fold, 4.7 fold, 4.8 fold, 4.9 fold, 5.0 fold, 5.1 fold, 5.2 fold, 5.3 fold, 5.4 fold, 5.5 fold, 5.6 fold, 5.7 fold, 5.8 fold, 5.9 fold, 6.0 fold, 6.1 fold, 6.2 fold, 6.3 fold, 6.4 fold, 6.5 fold, 6.6 fold, 6.7 fold, 6.8 fold, 6.9 fold, 7.0 fold, 7.1 fold, 7.2 fold, 7.3 fold, 7.4 fold, 7.5 fold, 7.6 fold, 7.7 fold, 7.8 fold, 7.9 fold, or 8.0 fold. In some embodiments, the phagocytes are macrophages. In some such embodiments, the macrophages are tumor-associated macrophages (TAMs). In humans, TAMs may be identified based on expression of various cell-surface markers, including CD14, HLA-DR (MHC class II), CD312, CD115, CD16, CD163, CD204, CD206, and CD301. Furthermore, the production of specific functional biomarkers, such as matrix metalloproteinases, IL-10, inducible nitric oxide synthase (iNOS), TNF-alpha, or IL-12 may be combined with cell-surface biomarkers to accurately identify TAM populations (Quatromoni, J., et al., Am J Transl Res. 4 (2012): 376-389.) The clearance of apoptotic cells may be measured by any assay known to one of skill in the art for such purpose. For example, for in vitro apoptotic cell clearance assays, phagocytes such as mouse peritoneal macrophages or human monocyte derived macrophages are used. Apoptotic cells are generated by treatment with dexamethasone and labeled with a detection probe. Phagocytosis can be analyzed by microscopy or flow cytometry after incubation apoptotic cells with phagocytes. In some embodiments, the clearance of apoptotic cells is reduced as measured in such an apoptotic cell clearance assay at room temperature. For example, for in vivo apoptotic clearance assays, mice are injected with dexamethasone to induce thymocyte death. Resident macrophages in the thymus recognize and engulf the dying/dead cells (Seitz, H. M. J Immunol. 178(9) 5635-5642 (2007). In some embodiments, the clearance of apoptotic cells is reduced as measured in such an apoptotic cell clearance assay in vivo. In some embodiments, the antibodies reduce ligand-mediated MerTK signaling. In some embodiments, the ligand is hGAS6-Fc (EC50=˜84 pM). In some embodiments, the antibodies induce a pro-inflammatory response. In some embodiments, the antibodies induce a type I IFN response.

In some embodiments, an anti-MerTK antibody of the present disclosure reduces phagocytic activity of apoptotic cells by about 10-100%, 20-100%, 30-100%, 40-100%, 50-100%, 60-100%, 70-100%, 75-100%, 80-100%, 85-100%, 90-100%, 95-100%, 10-95%, 20-95%, 30-95%, 40-95%, 50-95%, 60-95%, 70-95%, 75-95%, 80-95%, 85-95%, 90-95%, 10-90%, 20-90%, 30-90%, 40-90%, 50-90%, 60- 90%, 70-90%, 75-90%, 80-90%, 85-90%, 10-85%, 20-85%, 30-85%, 40-85%, 50-85%, 60-85%, 70-85%, 75-85%, 80-85%, 10-80%, 20-80%, 30-80%, 40-80%, 50-80%, 60-80%, 70-80%, 75-80%, 10-75%, 20- 75%, 30-75%, 40-75%, 50-75%, 60-75%, 70-75%, 10-70%, 20-70%, 30-70%, 40-70%, 50-70%, 60-70%, 10-65%, 20-65%, 30-65%, 40-65%, 50-65%, 60-65%, 10-60%, 20-60%, 30-60%, 40-60%, 50-60%, 10- 55%, 20-55%, 30-55%, 40-55%, 50-55%, 10-40%, 20-40%, or 30-40%, or by at least about 10%, 20%, 30%, 40%, 50%, 60%, 65%, 70%, 75%, 80%, 85%, 90%, 95%, 96%, 97%, 98% or 99%. In some embodiments, the anti-MerTK antibody has a half maximal inhibitory concentration (IC50) for reducing phagocytic activity of apoptotic cells of about 1 pM-50 pM, 1 pM-100 pM, 1 pM-500 pM, 1 pM-1 nM, 1 pM-1.5 nM, 5 pM-50 pM, 5 pM-100 pM, 5 pM-500 pM, 5 pM-1 nM, 5 pM-1.5 nM, 10 pM-50 pM, 10 pM-100 pM, 10 pM-500 pM, 10 pM-1 nM, 10 pM-1.5 nM, 50 pM-100 pM, 50 pM-500 pM, 50 pM-1 nM, 50 pM-1.5 nM, 100 pM-500 pM, 100 pM-1 nM, or 100 pM-1.5 nM. Exemplary methods for determining phagocytic activity and IC50 are described in the Examples herein below.

In some embodiments, an anti-MerTK antibody of the present disclosure enhances the activity of a checkpoint inhibitor by about 1-2 fold, 1-5 fold, 1-10 fold, 1-15 fold, 1-20 fold, 1-25 fold, 1-30 fold, 1-50 fold, 1-75 fold, 1-100 fold, 1-150 fold, 1-200 fold, 1-250 fold, 1.5-2 fold, 1.5-5 fold, 1.5-10 fold, 1.5-15 fold, 1.5-20 fold, 1.5-25 fold, 1.5-30 fold, 1.5-50 fold, 1.5-75 fold, 1.5-100 fold, 1.5-150 fold, 1.5-200 fold, 1.5-250 fold, 2-5 fold, 2-10 fold, 2-15 fold, 2-20 fold, 2-25 fold, 2-30 fold, 2-50 fold, 2-75 fold, 2-100 fold, 2-150 fold, 2-200 fold, 2-250 fold, 2.5-5 fold, 2.5-10 fold, 2.5-15 fold, 2.5-20 fold, 2.5-25 fold, 2.5-30 fold, 2.5-50 fold, 2.5-75 fold, 2.5-100 fold, 2.5-150 fold, 2.5-200 fold, 2.5-250 fold, 5-10 fold, 5-15 fold, 5-20 fold, 5-25 fold, 5-30 fold, 5-50 fold, 5-75 fold, 5-100 fold, 5-150 fold, 5-200 fold, 5-250 fold, 10-15 fold, 10-20 fold, 10-25 fold, 10-30 fold, 10-50 fold, 10-75 fold, 10-100 fold, 10-150 fold, 10-200 fold, 10-250 fold, 20-25 fold, 20-30 fold, 20-50 fold, 20-75 fold, 20-100 fold, 20-150 fold, 20-200 fold, 20-250 fold, 25-30 fold, 25-50 fold, 25-75 fold, 25-100 fold, 25-150 fold, 25-200 fold, or 25-250 fold or by at least about 1 fold, 2 fold, 5 fold, 10 fold, 15 fold 20 fold 25 fold, 30 fold, 40 fold, 50 fold 60 fold, 70 fold, 75 fold, 80 fold, 90 fold, 100 fold, 125 fold, 150 fold, 200 fold, 225 fold or 250 fold. In certain embodiments, an anti-MerTK antibody of the present disclosure enhances the activity of a checkpoint inhibitor as determined using an assay as described in the Examples herein below, such as, for example, by determining a reduction in tumor volume in a mouse tumor model using a combination of an anti-MerTK antibody plus a checkpoint inhibitor as compared to the reduction in tumor volume using the checkpoint inhibitor alone. In certain embodiments, the reduction in tumor volume is determined after at least 10 days, 14 days, 20 days, 21 days or 30 days after treatment with the therapeutic agents. In certain embodiments, the checkpoint inhibitor is a anti-PD1 axis antagonist. In one exemplary embodiment, the checkpoint inhibitor is an anti-PD-L1 antibody. In another embodiment, the checkpoint inhibitor is an anti-PD1 antibody

In some embodiments, an anti-MerTK antibody of the present disclosure increases cell-free DNA (cfDNA) and/or circulating tumor DNA (ctDNA), e.g., in a blood or plasma sample, by about 1-2 fold, 1-3 fold, 1-4 fold, 1-5 fold, 1-10 fold, 1.5-2 fold, 1.5-3 fold, 1.5-4 fold, 1.5-5 fold, 1.5-10 fold, 2-3 fold, 2-4 fold, 2-5 fold, 2-10 fold, 3-5 fold, 3-10 fold, 4-5 fold, 4-10 fold, 5-10 fold, or by at least about 1 fold, 2 fold, 3 fold, 4 fold, 5 fold, or 10 fold. In certain embodiments, an anti-MerTK antibody of the present disclosure increases cell-free DNA (cfDNA) and/or circulating tumor DNA (ctDNA) as determined using an assay as described in the Examples herein below, such as, for example, by isolating cfDNA and/or ctDNA from a blood or plasma sample and detecting levels of cfDNA and/or ctDNA using PCR and quantitative DNA electrophoresis.

### 2. Antibody Affinity & Specificity

In certain embodiments, an anti-MerTK antibody provided herein has a dissociation constant (Kd) of ≤1 M, ≤100 nM, ≤10 nM, ≤1 nM, ≤0.1 nM, ≤0.01 nM, or ≤0.001 nM, or about 1 pM-0.1 nM, 1 pM-0.2 nM, 1 pM-0.5 nM, 1 pM-1 nM, 1 pM-2 nM, 1 pM-5 nM, 1 pM-10 nM, 1 pM-15 nM, 5 pM-0.1 nM, 5 pM-0.2 nM, 5 pM-0.5 nM, 5 pM-1 nM, 5 pM-2 nM, 5 pM-5 nM, 5 pM-10 nM, 5 pM-15 nM, 10 pM-0.1 nM, 10 pM-0.2 nM, 10 pM-0.5 nM, 10 pM-1 nM, 10 pM-2 nM, 10 pM-5 nM, 10 pM-10 nM, 10 pM-15 nM, 20 pM-0.1 nM, 20 pM-0.2 nM, 20 pM-0.5 nM, 20 pM-1 nM, 20 pM-2 nM, 20 pM-5 nM, 20 pM-10 nM, 20 pM-15 nM, 25 pM-0.1 nM, 25 pM-0.2 nM, 25 pM-0.5 nM, 25 pM-1 nM, 25 pM-2 nM, 25 pM-5 nM, 25 pM-10 nM, 25 pM-15 nM, 50 pM-0.1 nM, 50 pM-0.2 nM, 50 pM-0.5 nM, 50 pM-1 nM, 50 pM-2 nM, 50 pM-5 nM, 50 pM-10 nM, 50 pM-15 nM, 100 pM-0.2 nM, 100 pM-0.5 nM, 100 pM-1 nM, 100 pM-2 nM, 100 pM-5 nM, 100 pM-10 nM, or 100 pM-15 nM. In certain embodiments, the Kd of the anti-MerTK antibody as disclosed herein is measured at 25° C. In certain embodiments, the Kd of the anti-MerTK antibody as disclosed herein is measured at 37° C.

In one embodiment, Kd is measured by a radiolabeled antigen binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using a BIACORE® surface plasmon resonance assay. For example, an assay using a BIACORE®-2000 or a BIACORE®-3000 (BIAcore, Inc., Piscataway, NJ) is performed at 25° C. with immobilized antigen CM5 chips at ˜10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 106 M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

In certain embodiments, an anti-MerTK antibody as disclosed herein binds to one or more of human MerTK, cyno MerTK, mouse MerTK and/or rat MerTK. In one embodiment, an anti-MerTK antibody as disclosed herein binds specifically to human MerTK. In one embodiment, an anti-MerTK antibody as disclosed herein binds to human MerTK and cyno MerTK. In one embodiment, an anti-MerTK antibody as disclosed herein binds to human MerTK and mouse MerTK. In one embodiment, an anti-MerTK antibody as disclosed herein binds to human MerTK, cyno MerTK and mouse MerTK. In one embodiment, an anti-MerTK antibody as disclosed herein binds to human MerTK, cyno MerTK, mouse MerTK and rat MerTK. In one embodiment, an anti-MerTK antibody as disclosed herein binds specifically to mouse MerTK.

In certain embodiments, an anti-MerTK antibody as disclosed herein binds to an Ig-like domain of MerTK. In one embodiment, an anti-MerTK antibody that binds to an Ig-like domain of MerTK binds to one or more amino acid residues in the Ig-like domain corresponding to amino acid residues 76-195 of MerTK SEQ ID NO: 129, e.g., the anti-MerTK antibody binds to at least 1, 2, 3, 4, 5, 6, 7, 8, 9, or 10 amino acids or 1-2, 1-3, 1-4, 1-5, 1-6, 1-7, 1-8, 1-9, or 1-10 amino acid residues of residues 76-195 of MerTK SEQ ID NO: 129. In one embodiment, an anti-MerTK antibody that binds to an Ig-like domain of MerTK binds to one or more amino acid residues in the Ig-like domain corresponding to amino acid residues 199-283 of MerTK SEQ ID NO: 129, e.g., the anti-MerTK antibody binds to at least 1, 2, 3, 4, 5, 6, 7, 8, 9, or 10 amino acids or 1-2, 1-3, 1-4, 1-5, 1-6, 1-7, 1-8, 1-9, or 1-10 amino acid residues of residues 199-283 of MerTK SEQ ID NO: 129.

In certain embodiments, an anti-MerTK antibody as disclosed herein binds to a fibronectin-like domain of MerTK. In one embodiment, an anti-MerTK antibody that binds to an fibronectin-like domain of MerTK binds to one or more amino acid residues in the fibronectin-like domain corresponding to amino acid residues 286-384 of MerTK SEQ ID NO: 129, e.g., the anti-MerTK antibody binds to at least 1, 2, 3, 4, 5, 6, 7, 8, 9, or 10 amino acids or 1-2, 1-3, 1-4, 1-5, 1-6, 1-7, 1-8, 1-9, or 1-10 amino acid residues of residues 286-384 of MerTK SEQ ID NO: 129. In one embodiment, an anti-MerTK antibody that binds to a fibronectin-like domain of MerTK binds to one or more amino acid residues in the fibronectin-like domain corresponding to amino acid residues 388-480 of MerTK SEQ ID NO: 129, e.g., the anti-MerTK antibody binds to at least 1, 2, 3, 4, 5, 6, 7, 8, 9, or 10 amino acids or 1-2, 1-3, 1-4, 1-5, 1-6, 1-7, 1-8, 1-9, or 1-10 amino acid residues of residues 388-480 of MerTK SEQ ID NO: 129.

In an exemplary embodiment, an anti-MerTK antibody as disclosed herein binds to an Ig-like domain of human and cyno MerTK. In one embodiment, such an antibody binds to human and cyno MerTK with a Kd at 37° C. that is approximately the same, e.g., the antibody binds to cyno MerTK at 37° C. with a Kd that is not more than 10%, 15% or 20% different than the Kd of the antibody at 37° C. for human MerTK. In certain embodiments, such an antibody binds to human and cyno MerTK with a Kd at 37° C. that is at least 20 fold, 25 fold or 50 fold better than the Kd of the antibody at 37° C. for mouse and rat MerTK.

### 3. Antibody Fragments

In certain embodiments, an anti-MerTK antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

### 4. Chimeric and Humanized Antibodies

In certain embodiments, an anti-MerTK antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 5. Human Antibodies

In certain embodiments, an anti-MerTK antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al, Proc. Natl. Acad. Sci. US, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91(2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### 6. Library-Derived Antibodies

Anti-MerTK antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, NJ, 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, NJ, 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

### 7. Multispecific Antibodies

In certain embodiments, an anti-MerTK antibody provided herein is a multispecific antibody, e.g. a bispecific antibody. Multispecific antibodies are monoclonal antibodies that have binding specificities for at least two different sites. In certain embodiments, one of the binding specificities is for MerTK and the other is for any other antigen. In certain embodiments, bispecific antibodies may bind to two different epitopes of MerTK. Bispecific antibodies may also be used to localize cytotoxic agents to cells which express MerTK. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds to MerTK as well as another, different antigen (see, US 2008/0069820, for example).

### 8. Antibody Variants

In certain embodiments, amino acid sequence variants of the anti-MerTK antibody provided herein are contemplated. For example, it may be desirable to improve the binding affinity and/or other biological properties of the anti-MerTK antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### a) Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

### b) Glycosylation Variants

In certain embodiments, an anti-MerTK antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

### c) Fc Region Variants

In certain embodiments, one or more amino acid modifications may be introduced into the Fc region of an anti-MerTK antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

In an exemplary embodiment, an anti-MerTK antibody disclosed herein comprises a LALPG mutation in the Fc region.

### d) Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

### e) Antibody Derivatives

In certain embodiments, an anti-MerTK antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### B. Recombinant Methods and Compositions

Antibodies may be produced using recombinant methods and compositions, e.g., as described in U.S. Pat. No. 4,816,567. In one embodiment, isolated nucleic acid encoding an anti-MerTK antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti-MerTK antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-MerTK antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, NJ, 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

### C. Assays

Anti-MerTK antibodies provided herein may be identified, screened for, or characterized for their physical/chemical properties and/or biological activities by various assays known in the art.

In one aspect, an antibody of the invention is tested for its antigen binding activity, e.g., by known methods such as ELISA, Western blot, etc.

In another aspect, competition assays may be used to identify an antibody that competes with one or more of the anti-MerTK antibodies disclosed herein for binding to MerTK. In certain embodiments, such a competing antibody binds to the same epitope (e.g., a linear or a conformational epitope) that is bound by one or more of the anti-MerTK antibodies disclosed herein. Detailed exemplary methods for mapping an epitope to which an antibody binds are provided in Morris (1996) “Epitope Mapping Protocols,” in Methods in Molecular Biology vol. 66 (Humana Press, Totowa, NJ).

In an exemplary competition assay, immobilized MerTK is incubated in a solution comprising a first labeled antibody that binds to MerTK and a second unlabeled antibody that is being tested for its ability to compete with the first antibody for binding to MerTK. The second antibody may be present in a hybridoma supernatant. As a control, immobilized MerTK is incubated in a solution comprising the first labeled antibody but not the second unlabeled antibody. After incubation under conditions permissive for binding of the first antibody to MerTK, excess unbound antibody is removed, and the amount of label associated with immobilized MerTK is measured. If the amount of label associated with immobilized MerTK is substantially reduced in the test sample relative to the control sample, then that indicates that the second antibody is competing with the first antibody for binding to MerTK. See Harlow and Lane (1988) Antibodies: A Laboratory Manual ch. 14 (Cold Spring Harbor Laboratory, Cold Spring Harbor, NY).

In another aspect, assays are provided for identifying anti-MerTK antibodies thereof having biological activity. Biological activity may include, e.g., reducing MerTK-mediated phagocytic activity, reducing MerTK-mediated clearance of apoptotic cells, and/or enhancing tumor immunogenicity of a checkpoint inhibitor. Antibodies having such biological activity in vivo and/or in vitro are also provided.

In certain embodiments, an antibody of the invention is tested for such biological activity. Examples of assays suitable for measuring such biological activity are described further herein, including the Exemplification section below.

### D. Immunoconjugates

The invention also provides immunoconjugates comprising an anti-MerTK antibody herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In another embodiment, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A.).

### E. Methods and Compositions for Diagnostics and Detection

In certain embodiments, any of the anti-MerTK antibodies provided herein is useful for detecting the presence of MerTK in a biological sample. The term “detecting” as used herein encompasses quantitative or qualitative detection.

In one embodiment, an anti-MerTK antibody for use in a method of diagnosis or detection is provided. In a further aspect, a method of detecting the presence of MerTK in a biological sample is provided. In certain embodiments, the method comprises contacting the biological sample with an anti-MerTK antibody as described herein under conditions permissive for binding of the anti-MerTK antibody to MerTK, and detecting whether a complex is formed between the anti-MerTK antibody and MerTK. Such method may be an in vitro or in vivo method. In one embodiment, an anti-MerTK antibody is used to select subjects eligible for therapy with an anti-MerTK antibody, e.g. where MerTK is a biomarker for selection of patients.

In certain embodiments, labeled anti-MerTK antibodies are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luciferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### F. Pharmaceutical Compositions and Formulations

Also provided herein are pharmaceutical compositions and formulations comprising an anti-MerTK antibody, and a pharmaceutically acceptable carrier.

In some embodiments, an anti-MerTK antibody described herein is in a formulation comprising the antibody at an amount of about 60 mg/mL, histidine acetate in a concentration of about 20 mM, sucrose in a concentration of about 120 mM, and polysorbate (e.g., polysorbate 20) in a concentration of 0.04% (w/v), and the formulation has a pH of about 5.8. In some embodiments, the anti-PDL1 antibody described herein is in a formulation comprising the antibody in an amount of about 125 mg/mL, histidine acetate in a concentration of about 20 mM, sucrose is in a concentration of about 240 mM, and polysorbate (e.g., polysorbate 20) in a concentration of 0.02% (w/v), and the formulation has a pH of about 5.5.

After preparation of the anti-MerTK antibody of interest (e.g., techniques for producing antibodies which can be formulated as disclosed herein are elaborated herein and are known in the art), the pharmaceutical formulation comprising it is prepared. In certain embodiments, the anti-MerTK antibody to be formulated has not been subjected to prior lyophilization and the formulation of interest herein is an aqueous formulation. In certain embodiments, the anti-MerTK antibody is a full length antibody. In one embodiment, the anti-MerTK antibody in the formulation is an antibody fragment, such as an F(ab′)₂, in which case problems that may not occur for the full length antibody (such as clipping of the antibody to Fab) may need to be addressed. The therapeutically effective amount of anti-MerTK antibody present in the formulation is determined by taking into account the desired dose volumes and mode(s) of administration, for example. From about 25 mg/mL to about 150 mg/mL, or from about 30 mg/mL to about 140 mg/mL, or from about 35 mg/mL to about 130 mg/mL, or from about 40 mg/mL to about 120 mg/mL, or from about 50 mg/mL to about 130 mg/mL, or from about 50 mg/mL to about 125 mg/mL, or from about 50 mg/mL to about 120 mg/mL, or from about 50 mg/mL to about 110 mg/mL, or from about 50 mg/mL to about 100 mg/mL, or from about 50 mg/mL to about 90 mg/mL, or from about 50 mg/mL to about 80 mg/mL, or from about 54 mg/mL to about 66 mg/mL is an exemplary antibody concentration in the formulation.

An aqueous formulation is prepared comprising the antibody in a pH-buffered solution. In some embodiments, the buffer of the present disclosure has a pH in the range from about 5.0 to about 7.0. In certain embodiments the pH is in the range from about 5.0 to about 6.5, the pH is in the range from about 5.0 to about 6.4, in the range from about 5.0 to about 6.3, the pH is in the range from about 5.0 to about 6.2, the pH is in the range from about 5.0 to about 6.1, the pH is in the range from about 5.5 to about 6.1, the pH is in the range from about 5.0 to about 6.0, the pH is in the range from about 5.0 to about 5.9, the pH is in the range from about 5.0 to about 5.8, the pH is in the range from about 5.1 to about 6.0, the pH is in the range from about 5.2 to about 6.0, the pH is in the range from about 5.3 to about 6.0, the pH is in the range from about 5.4 to about 6.0, the pH is in the range from about 5.5 to about 6.0, the pH is in the range from about 5.6 to about 6.0, the pH is in the range from about 5.7 to about 6.0, or the pH is in the range from about 5.8 to about 6.0. In some embodiments, the formulation has a pH of 6.0 or about 6.0. In some embodiments, the formulation has a pH of 5.9 or about 5.9. In some embodiments, the formulation has a pH of 5.8 or about 5.8. In some embodiments, the formulation has a pH of 5.7 or about 5.7. In some embodiments, the formulation has a pH of 5.6 or about 5.6. In some embodiments, the formulation has a pH of 5.5 or about 5.5. In some embodiments, the formulation has a pH of 5.4 or about 5.4. In some embodiments, the formulation has a pH of 5.3 or about 5.3. In some embodiments, the formulation has a pH of 5.2 or about 5.2. Examples of buffers that will control the pH within this range include histidine (such as L-histidine) or sodium acetate. In certain embodiments, the buffer contains histidine acetate or sodium acetate in the concentration of about 15 mM to about 25 mM. In some embodiments, the buffer contains histidine acetate or sodium acetate in the concentration of about 15 mM to about 25 mM, about 16 mM to about 25 mM, about 17 mM to about 25 mM, about 18 mM to about 25 mM, about 19 mM to about 25 mM, about 20 mM to about 25 mM, about 21 mM to about 25 mM, about 22 mM to about 25 mM, about 15 mM, about 16 mM, about 17 mM, about 18 mM, about 19 mM, about 20 mM, about 21 mM, about 22 mM, about 23 mM, about 24 mM, or about 25 mM. In one embodiment, the buffer is histidine acetate or sodium acetate in an amount of about 20 mM, pH 5.0. In one embodiment, the buffer is histidine acetate or sodium acetate in an amount of about 20 mM, pH 5.1. In one embodiment, the buffer is histidine acetate or sodium acetate in an amount of about 20 mM, pH 5.2. In one embodiment, the buffer is histidine acetate or sodium acetate in an amount of about 20 mM, pH 5.3. In one embodiment, the buffer is histidine acetate or sodium acetate in an amount of about 20 mM, pH 5.4. In one embodiment, the buffer is histidine acetate or sodium acetate in an amount of about 20 mM, pH 5.5. In one embodiment, the buffer is histidine acetate or sodium acetate in an amount of about 20 mM, pH 5.6. In one embodiment, the buffer is histidine acetate or sodium acetate in an amount of about 20 mM, pH 5.7. In one embodiment, the buffer is histidine acetate or sodium acetate in an amount of about 20 mM, pH 5.8. In one embodiment, the buffer is histidine acetate or sodium acetate in an amount of about 20 mM, pH 5.9. In one embodiment, the buffer is histidine acetate or sodium acetate in an amount of about 20 mM, pH 6.0. In one embodiment, the buffer is histidine acetate or sodium acetate in an amount of about 20 mM, pH 6.1. In one embodiment, the buffer is histidine acetate or sodium acetate in an amount of about 20 mM, pH 6.2. In one embodiment, the buffer is histidine acetate or sodium acetate in an amount of about 20 mM, pH 6.3. In one embodiment, the buffer is histidine acetate or sodium acetate in an amount of about 25 mM, pH 5.2. In one embodiment, the buffer is histidine acetate or sodium acetate in an amount of about 25 mM, pH 5.3. In one embodiment, the buffer is histidine acetate or sodium acetate in an amount of about 25 mM, pH 5.4. In one embodiment, the buffer is histidine acetate or sodium acetate in an amount of about 25 mM, pH 5.5. In one embodiment, the buffer is histidine acetate or sodium acetate in an amount of about 25 mM, pH 5.6. In one embodiment, the buffer is histidine acetate or sodium acetate in an amount of about 25 mM, pH 5.7. In one embodiment, the buffer is histidine acetate or sodium acetate in an amount of about 25 mM, pH 5.8. In one embodiment, the buffer is histidine acetate or sodium acetate in an amount of about 25 mM, pH 5.9. In one embodiment, the buffer is histidine acetate or sodium acetate in an amount of about 25 mM, pH 6.0. In one embodiment, the buffer is histidine acetate or sodium acetate in an amount of about 25 mM, pH 6.1. In one embodiment, the buffer is histidine acetate or sodium acetate in an amount of about 25 mM, pH 6.2. In one embodiment, the buffer is histidine acetate or sodium acetate in an amount of about 25 mM, pH 6.3.

In some embodiments, the formulation further comprises sucrose in an amount of about 60 mM to about 240 mM. In some embodiments, sucrose in the formulation is about 60 mM to about 230 mM, about 60 mM to about 220 mM, about 60 mM to about 210 mM, about 60 mM to about 200 mM, about 60 mM to about 190 mM, about 60 mM to about 180 mM, about 60 mM to about 170 mM, about 60 mM to about 160 mM, about 60 mM to about 150 mM, about 60 mM to about 140 mM, about 80 mM to about 240 mM, about 90 mM to about 240 mM, about 100 mM to about 240 mM, about 110 mM to about 240 mM, about 120 mM to about 240 mM, about 130 mM to about 240 mM, about 140 mM to about 240 mM, about 150 mM to about 240 mM, about 160 mM to about 240 mM, about 170 mM to about 240 mM, about 180 mM to about 240 mM, about 190 mM to about 240 mM, about 200 mM to about 240 mM, about 80 mM to about 160 mM, about 100 mM to about 140 mM, or about 110 mM to about 130 mM. In some embodiments, sucrose in the formulation is about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, or about 240 mM.

In some embodiments, the anti-MerTK antibody concentration in the formulation is about 40 mg/ml to about 125 mg/ml. In some embodiments, the antibody concentration in the formulation is about 40 mg/ml to about 120 mg/ml, about 40 mg/ml to about 110 mg/ml, about 40 mg/ml to about 100 mg/ml, about 40 mg/ml to about 90 mg/ml, about 40 mg/ml to about 80 mg/ml, about 40 mg/ml to about 70 mg/ml, about 50 mg/ml to about 120 mg/ml, about 60 mg/ml to about 120 mg/ml, about 70 mg/ml to about 120 mg/ml, about 80 mg/ml to about 120 mg/ml, about 90 mg/ml to about 120 mg/ml, or about 100 mg/ml to about 120 mg/ml. In some embodiments, the anti-MerTK antibody concentration in the formulation is about 60 mg/ml. In some embodiments, the anti-MerTK antibody concentration in the formulation is about 65 mg/ml. In some embodiments, the anti-MerTK antibody concentration in the formulation is about 70 mg/ml. In some embodiments, the anti-MerTK antibody concentration in the formulation is about 75 mg/ml. In some embodiments, the anti-MerTK antibody concentration in the formulation is about 80 mg/ml. In some embodiments, the anti-MerTK antibody concentration in the formulation is about 85 mg/ml. In some embodiments, the anti-MerTK antibody concentration in the formulation is about 90 mg/ml. In some embodiments, the anti-MerTK antibody concentration in the formulation is about 95 mg/ml. In some embodiments, the anti-MerTK antibody concentration in the formulation is about 100 mg/ml. In some embodiments, the anti-MerTK antibody concentration in the formulation is about 110 mg/ml. In some embodiments, the anti-MerTK antibody concentration in the formulation is about 125 mg/ml.

In some embodiments, a surfactant is added to the anti-MerTK antibody formulation. Exemplary surfactants include nonionic surfactants such as polysorbates (e.g. polysorbates 20, 80 etc) or poloxamers (e.g. poloxamer 188, etc.). The amount of surfactant added is such that it reduces aggregation of the formulated antibody and/or minimizes the formation of particulates in the formulation and/or reduces adsorption. For example, the surfactant may be present in the formulation in an amount from about 0.001% to about 0.5% (w/v). In some embodiments, the surfactant (e.g., polysorbate 20) is from about 0.005% to about 0.2%, from about 0.005% to about 0.1%, from about 0.005% to about 0.09%, from about 0.005% to about 0.08%, from about 0.005% to about 0.07%, from about 0.005% to about 0.06%, from about 0.005% to about 0.05%, from about 0.005% to about 0.04%, from about 0.008% to about 0.06%, from about 0.01% to about 0.06%, from about 0.02% to about 0.06%, from about 0.01% to about 0.05%, or from about 0.02% to about 0.04%. In certain embodiments, the surfactant (e.g., polysorbate 20) is present in the formulation in an amount of 0.005% or about 0.005%. In certain embodiments, the surfactant (e.g., polysorbate 20) is present in the formulation in an amount of 0.006% or about 0.006%. In certain embodiments, the surfactant (e.g., polysorbate 20) is present in the formulation in an amount of 0.007% or about 0.007%. In certain embodiments, the surfactant (e.g., polysorbate 20) is present in the formulation in an amount of 0.008% or about 0.008%. In certain embodiments, the surfactant (e.g., polysorbate 20) is present in the formulation in an amount of 0.009% or about 0.009%. In certain embodiments, the surfactant (e.g., polysorbate 20) is present in the formulation in an amount of 0.01% or about 0.01%. In certain embodiments, the surfactant (e.g., polysorbate 20) is present in the formulation in an amount of 0.02% or about 0.02%. In certain embodiments, the surfactant (e.g., polysorbate 20) is present in the formulation in an amount of 0.03% or about 0.03%. In certain embodiments, the surfactant (e.g., polysorbate 20) is present in the formulation in an amount of 0.04% or about 0.04%. In certain embodiments, the surfactant (e.g., polysorbate 20) is present in the formulation in an amount of 0.05% or about 0.05%. In certain embodiments, the surfactant (e.g., polysorbate 20) is present in the formulation in an amount of 0.06% or about 0.06%. In certain embodiments, the surfactant (e.g., polysorbate 20) is present in the formulation in an amount of 0.07% or about 0.07%. In certain embodiments, the surfactant (e.g., polysorbate 20) is present in the formulation in an amount of 0.08% or about 0.08%. In certain embodiments, the surfactant (e.g., polysorbate 20) is present in the formulation in an amount of 0.1% or about 0.1%. In certain embodiments, the surfactant (e.g., polysorbate 20) is present in the formulation in an amount of 0.2% or about 0.2%. In certain embodiments, the surfactant (e.g., polysorbate 20) is present in the formulation in an amount of 0.3% or about 0.3%. In certain embodiments, the surfactant (e.g., polysorbate 20) is present in the formulation in an amount of 0.4% or about 0.4%. In certain embodiments, the surfactant (e.g., polysorbate 20) is present in the formulation in an amount of 0.5% or about 0.5%.

In one embodiment, the formulation contains the above-identified agents (e.g., antibody, buffer, sucrose, and/or surfactant) and is essentially free of one or more preservatives, such as benzyl alcohol, phenol, m-cresol, chlorobutanol and benzethonium Cl. In another embodiment, a preservative may be included in the formulation, particularly where the formulation is a multidose formulation. The concentration of preservative may be in the range from about 0.1% to about 2%, preferably from about 0.5% to about 1%. One or more other pharmaceutically acceptable carriers, excipients or stabilizers such as those described in Remington's Pharmaceutical Sciences 16th edition, Osol, A. Ed. (1980) may be included in the formulation provided that they do not adversely affect the desired characteristics of the formulation. Acceptable carriers, excipients or stabilizers are nontoxic to recipients at the dosages and concentrations employed and include; additional buffering agents; co-solvents; anti-oxidants including ascorbic acid and methionine; chelating agents such as EDTA; metal complexes (e.g. Zn-protein complexes); biodegradable polymers such as polyesters; and/or salt-forming counterions. Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

The formulation herein may also contain more than one protein as necessary for the particular indication being treated, preferably those with complementary activities that do not adversely affect the other protein. For example, where the antibody is anti-MerTK, it may be combined with another agent (e.g., a chemotherapeutic agent and/or an anti-neoplastic agent).

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The composition and formulation herein may also contain more than one active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the anti-MerTK antibody, which matrices are in the form of shaped articles, e.g. films, or microcapsules. The formulations to be used for in vivo administration are generally sterile. Sterility may be readily accomplished, e.g., by filtration through sterile filtration membranes.

### III. Methods of Treatment and Uses

In one aspect, the present disclosure provides a method of treating an individual having cancer including administering to the individual an effective amount of an anti-MerTK antibody as described above.

### (i) Monotherapy

In some embodiments, an anti-MerTK antibody of the present disclosure is administered as a monotherapy to treat an individual having cancer. As used herein, “cancer” refers to or describes the physiological condition in mammals that is typically characterized by unregulated cell growth. In certain embodiments, the cancer may be a solid cancer or a hematologic cancer. Solid cancers are generally characterized by tumor mass formation in specific tissues. “Tumor,” as used herein, refers to all neoplastic cell growth and proliferation, whether malignant or benign, and all pre-cancerous and cancerous cells and tissues. Non-limiting examples of solid cancers to be treated with an anti-MerTK antibody of the present disclosure include carcinoma, lymphoma, blastoma, and sarcoma. More particular examples of such cancers include, but not limited to, squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer and gastrointestinal stromal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, superficial spreading melanoma, lentigo maligna melanoma, acral lentiginous melanomas, nodular melanomas, as well as abnormal vascular proliferation associated with phakomatoses, edema (such as that associated with brain tumors), Meigs' syndrome, brain, head and neck cancer, and associated metastases. In certain embodiments, cancers that are amenable to treatment by anti-MerTK antibodies of the present disclosure include breast cancer, colorectal cancer, rectal cancer, non-small cell lung cancer, glioblastoma, renal cell cancer, prostate cancer, liver cancer, pancreatic cancer, soft-tissue sarcoma, kaposi's sarcoma, carcinoid carcinoma, head and neck cancer, ovarian cancer, and mesothelioma. In some embodiments, the cancer is selected from: small cell lung cancer, glioblastoma, neuroblastomas, melanoma, breast carcinoma, gastric cancer, colorectal cancer (CRC), and hepatocellular carcinoma. Yet, in some embodiments, the cancer is selected from: non-small cell lung cancer, colorectal cancer, glioblastoma and breast carcinoma, including metastatic forms of those cancers. In some embodiments, the cancer is colorectal cancer, including colon cancer and rectal cancer.

In contrast, hematologic cancers originate in the blood or bone marrow. In some embodiments, the hematologic cancer to be treated with an anti-MerTK antibody of the present disclosure is leukemia. Examples of leukemias include, without limitation, chronic lymphocytic leukemia (CLL); acute lymphoblastic leukemia (ALL); hairy cell leukemia; chronic myeloblastic leukemia; and acute myeloblastic leukemia. In some embodiments, the hematologic cancer to be treated with an anti-MerTK antibody of the present disclosure is lymphoma. Non-limiting examples of lymphoma include T-cell lymphoma (such as adult T-cell leukemia/lymphoma; hepatosplenic T-cell lymphoma; peripheral T-cell lymphoma, anaplastic large cell lymphoma; and angioimmunoblastic T cell lymphoma), B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; diffuse large B-cell lymphoma; mantle cell lymphoma; Burkitt lymphoma; AIDS-related lymphoma; and Waldenstrom's Macroglobulinemia), Hodgkin's lymphoma, and post-transplant lymphoproliferative disorder (PTLD). In some embodiments, the hematologic cancer to be treated with an anti-MerTK antibody of the present disclosure is myeloma. In a specific embodiment, the myeloma is plasmacytoma or multiple myeloma. In certain embodiments, cancers that are amenable to treatment by anti-MerTK antibodies of the present disclosure include non-Hodgkin's lymphoma and multiple myeloma.

In another aspect, provided herein are methods for treating or delaying progression of cancer in an individual comprising administering to the individual an effective amount of an anti-MerTK antibody as described in the present disclosure. In some embodiments, the treatment results in a sustained response in the individual after cessation of the treatment. The methods described herein may find use in treating conditions where enhanced immunogenicity is desired such as increasing tumor immunogenicity for the treatment of cancer. Also provided herein are methods of enhancing immune function in an individual having cancer comprising administering to the individual an effective amount of an anti-MerTK antibody as described in the present disclosure. In some embodiments, the cancer expresses functional STING, functional Cx43, and functional cGAS polypeptides. Functional proteins are proteins that are able to carry out their regular functions in a cell. Examples of functional proteins may include wild-type proteins, tagged proteins, and mutated proteins that retain or improve protein function as compared to a wild-type protein. Protein function can be measured by any method known to those of skill in the art, including assaying for protein or mRNA expression and sequencing genomic DNA or mRNA. In some embodiments, the cancer comprises tumor-associated macrophages that express functional STING polypeptides. In some embodiments, the cancer comprises tumor cells that express functional cGAS polypeptides. In some embodiments, the cancer comprises tumor cells that express functional Cx43 polypeptides. In some embodiments, the cancer is colorectal cancer, including colon cancer and rectal cancer.

Also provided herein are methods of reducing MerTK-mediated clearance of apoptotic cells in an individual comprising administering to the individual an effective amount of an anti-MerTK antibody as described in the present disclosure to reduce MerTK-mediated clearance of apoptotic cells. In some embodiments, the clearance of apoptotic cells is reduced by 1-10 fold, 1-8 fold, 1-5 fold, 1-4 fold, 1-3 fold, 1-2 fold, 2-10 fold, 2-8 fold, 2-5 fold, 2-4 fold, 2-3 fold, 3-10 fold, 3-8 fold, 3-5 fold, 3-4 fold, or by at least about 1.1 fold, 1.2 fold, 1.3 fold, 1.4 fold, 1.5 fold, 1.6 fold, 1.7 fold, 1.8 fold, 1.9 fold, 2.0 fold, 2.1 fold, 2.2 fold, 2.3 fold, 2.4 fold, 2.5 fold, 2.6 fold, 2.7 fold, 2.8 fold, 2.9 fold, 3.0 fold, 3.1 fold, 3.2 fold, 3.3 fold, 3.4 fold, 3.5 fold, 3.6 fold, 3.7 fold, 3.8 fold, 3.9 fold, 4.0 fold, 4.1 fold, 4.2 fold, 4.3 fold, 4.4 fold, 4.5 fold, 4.6 fold, 4.7 fold, 4.8 fold, 4.9 fold, 5.0 fold, 5.1 fold, 5.2 fold, 5.3 fold, 5.4 fold, 5.5 fold, 5.6 fold, 5.7 fold, 5.8 fold, 5.9 fold, 6.0 fold, 6.1 fold, 6.2 fold, 6.3 fold, 6.4 fold, 6.5 fold, 6.6 fold, 6.7 fold, 6.8 fold, 6.9 fold, 7.0 fold, 7.1 fold, 7.2 fold, 7.3 fold, 7.4 fold, 7.5 fold, 7.6 fold, 7.7 fold, 7.8 fold, 7.9 fold, or 8.0 fold. Reduction of MerTK-mediated clearance of apoptotic cells may be determined by comparing the level of MerTK-mediated clearance of apoptotic cells in a sample from an individual after administration of an effective amount of an anti-MerTK antibody or an immunoconjugate thereof to a reference level of MerTK-mediated clearance of apoptotic cells. In some embodiments, the reference level is the level of MerTK-mediated clearance of apoptotic cells a reference sample. In some embodiments, the reference sample is taken from the subject taken prior to administration of an effective amount of an anti-MerTK antibody or an immunoconjugate thereof. In some embodiments, the sample comprises tumor tissue or tumor cells.

In some embodiments, an anti-MerTK antibody of the present disclosure reduces phagocytic activity of apoptotic cells by about 10-100%, 20-100%, 30-100%, 40-100%, 50-100%, 60-100%, 70-100%, 75-100%, 80-100%, 85-100%, 90-100%, 95-100%, 10-95%, 20-95%, 30-95%, 40-95%, 50-95%, 60-95%, 70-95%, 75-95%, 80-95%, 85-95%, 90-95%, 10-90%, 20-90%, 30-90%, 40-90%, 50-90%, 60- 90%, 70-90%, 75-90%, 80-90%, 85-90%, 10-85%, 20-85%, 30-85%, 40-85%, 50-85%, 60-85%, 70-85%, 75-85%, 80-85%, 10-80%, 20-80%, 30-80%, 40-80%, 50-80%, 60-80%, 70-80%, 75-80%, 10-75%, 20- 75%, 30-75%, 40-75%, 50-75%, 60-75%, 70-75%, 10-70%, 20-70%, 30-70%, 40-70%, 50-70%, 60-70%, 10-65%, 20-65%, 30-65%, 40-65%, 50-65%, 60-65%, 10-60%, 20-60%, 30-60%, 40-60%, 50-60%, 10- 55%, 20-55%, 30-55%, 40-55%, 50-55%, 10-40%, 20-40%, or 30-40%, or by at least about 10%, 20%, 30%, 40%, 50%, 60%, 65%, 70%, 75%, 80%, 85%, 90%, 95%, 96%, 97%, 98% or 99%. In some embodiments, the anti-MerTK antibody has a half maximal inhibitory concentration (IC50) for reducing phagocytic activity of apoptotic cells of about 1 pM-50 pM, 1 pM-100 pM, 1 pM-500 pM, 1 pM-1 nM, 1 pM-1.5 nM, 5 pM-50 pM, 5 pM-100 pM, 5 pM-500 pM, 5 pM-1 nM, 5 pM-1.5 nM, 10 pM-50 pM, 10 pM-100 pM, 10 pM-500 pM, 10 pM-1 nM, 10 pM-1.5 nM, 50 pM-100 pM, 50 pM-500 pM, 50 pM-1 nM, 50 pM-1.5 nM, 100 pM-500 pM, 100 pM-1 nM, or 100 pM-1.5 nM.

In some embodiments, the individual is a human.

The anti-MerTK antibody may be administered intravenously, intramuscularly, subcutaneously, topically, orally, transdermally, intraperitoneally, intraorbitally, by implantation, by inhalation, intrathecally, intraventricularly, or intranasally. The appropriate dosage of the anti-MerTK antibody may be determined based on the type of disease to be treated, the severity and course of the disease, the clinical condition of the individual, the individual's clinical history and response to the treatment, and the discretion of the attending physician.

(ii) Combinations with an Additional Therapy

In some embodiments, the uses and methods may further comprise an additional therapy or administration of an effective amount of an additional therapeutic agent. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy. In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PI3K/AKT/mTOR pathway, HSP90 inhibitor, tubulin inhibitor, apoptosis inhibitor, and/or chemopreventative agent.

In some embodiments, the additional therapy is an antagonist directed against B7-H3 (also known as CD276), e.g., a blocking antibody, MGA271, an antagonist directed against a TGF beta, e.g., metelimumab (also known as CAT-192), fresolimumab (also known as GC1008), or LY2157299, a treatment comprising adoptive transfer of a T cell (e.g., a cytotoxic T cell or CTL) expressing a chimeric antigen receptor (CAR), a treatment comprising adoptive transfer of a T cell comprising a dominant-negative TGF beta receptor, e.g, a dominant-negative TGF beta type II receptor, a treatment comprising a HERCREEM protocol (see, e.g., ClinicalTrials.gov Identifier NCT00889954), an agonist directed against CD137 (also known as TNFRSF9, 4-1BB, or ILA), e.g., an activating antibody, urelumab (also known as BMS-663513), an agonist directed against CD40, e.g., an activating antibody, CP-870893, an agonist directed against OX40 (also known as CD134), e.g., an activating antibody, administered in conjunction with a different anti-OX40 antibody (e.g., AgonOX)., an agonist directed against CD27, e.g., an activating antibody, CDX-1127, indoleamine-2,3-dioxygenase (IDO), 1-methyl-D-tryptophan (also known as 1-D-MT), an antibody-drug conjugate (in some embodiments, comprising mertansine or monomethyl auristatin E (MMAE)), an anti-NaPi2b antibody-MMAE conjugate (also known as DNIB0600A or RG7599), trastuzumab emtansine (also known as T-DM1, ado-trastuzumab emtansine, or KADCYLA®, Genentech), DMUC5754A, an antibody-drug conjugate targeting the endothelin B receptor (EDNBR), e.g., an antibody directed against EDNBR conjugated with MMAE, an angiogenesis inhibitor, an antibody directed against a VEGF, e.g., VEGF-A, bevacizumab (also known as AVASTIN®, Genentech), an antibody directed against angiopoietin 2 (also known as Ang2), MEDI3617, an antineoplastic agent, an agent targeting CSF-1R (also known as M-CSFR or CD115), anti-CSF-1R (also known as IMC-CS4), an interferon, for example interferon alpha or interferon gamma, Roferon-A, GM-CSF (also known as recombinant human granulocyte macrophage colony stimulating factor, rhu GM-CSF, sargramostim, or Leukine®), IL-2 (also known as aldesleukin or Proleukin®), IL-12, an antibody targeting CD20 (in some embodiments, the antibody targeting CD20 is obinutuzumab (also known as GA101 or Gazyva®) or rituximab), an antibody targeting GITR (in some embodiments, the antibody targeting GITR is TRX518), in conjunction with a cancer vaccine (in some embodiments, the cancer vaccine is a peptide cancer vaccine, which in some embodiments is a personalized peptide vaccine; in some embodiments the peptide cancer vaccine is a multivalent long peptide, a multi-peptide, a peptide cocktail, a hybrid peptide, or a peptide-pulsed dendritic cell vaccine (see, e.g., Yamada et al., Cancer Sci, 104:14-21, 2013)), in conjunction with an adjuvant, a TLR agonist, e.g., Poly-ICLC (also known as Hiltonol®), LPS, MPL, or CpG ODN, tumor necrosis factor (TNF) alpha, IL-1, HMGB1, an IL-10 antagonist, an IL-4 antagonist, an IL-13 antagonist, an HVEM antagonist, an ICOS agonist, e.g., by administration of ICOS-L, or an agonistic antibody directed against ICOS, a treatment targeting CX3CL1, a treatment targeting CXCL10, a treatment targeting CCL5, an LFA-1 or ICAM1 agonist, a Selectin agonist, a targeted therapy, an inhibitor of B-Raf, vemurafenib (also known as Zelboraf®, dabrafenib (also known as Tafinlar®), erlotinib (also known as Tarceva®), an inhibitor of a MEK, such as MEK1 (also known as MAP2K1) or MEK2 (also known as MAP2K2). cobimetinib (also known as GDC-0973 or XL-518), trametinib (also known as Mekinist®), an inhibitor of K-Ras, an inhibitor of c-Met, onartuzumab (also known as MetMAb), an inhibitor of Alk, AF802 (also known as CH5424802 or alectinib), an inhibitor of a phosphatidylinositol 3-kinase (PI3K), BKM120, idelalisib (also known as GS-1101 or CAL-101), perifosine (also known as KRX-0401), an Akt, MK2206, GSK690693, GDC-0941, an inhibitor of mTOR, sirolimus (also known as rapamycin), temsirolimus (also known as CCI-779 or Torisel®), everolimus (also known as RAD001), ridaforolimus (also known as AP-23573, MK-8669, or deforolimus), OSI-027, AZD8055, INK128, a dual PI3K/mTOR inhibitor, XL765, GDC-0980, BEZ235 (also known as NVP-BEZ235), BGT226, GSK2126458, PF-04691502, or PF-05212384 (also known as PKI-587). In some embodiments, the additional therapeutic agent is CT-011 (also known as Pidilizumab or MDV9300; CAS Registry No. 1036730-42-3; CureTech/Medivation). CT-011, also known as hBAT or hBAT-1, is an antibody described in WO2009/101611.

(iii) Combinations with Immune Checkpoint Inhibitors

In some embodiments, the additional therapeutic agent is an immune checkpoint inhibitor. In certain aspects, the application provides methods for enhancing immune function in an individual having cancer comprising administering an effective amount of a combination of an anti-MerTK antibody and an immune checkpoint inhibitor. In certain embodiments, the anti-MERTK antibody increases the immune effect of an immune checkpoint inhibitor by about 2 fold, 3 fold, 5 fold, 8 fold, 10 fold, 15 fold or 20 fold. In certain embodiments, the anti-MERTK antibody increases the immune effect of an immune checkpoint inhibitor by about 1-2 fold, 1-5 fold, 1-10 fold, 1-15 fold, 1-20 fold, 1-25 fold, 1-30 fold, 1-50 fold, 1-75 fold, 1-100 fold, 1-150 fold, 1-200 fold, 1-250 fold, 1.5-2 fold, 1.5-5 fold, 1.5-10 fold, 1.5-15 fold, 1.5-20 fold, 1.5-25 fold, 1.5-30 fold, 1.5-50 fold, 1.5-75 fold, 1.5-100 fold, 1.5-150 fold, 1.5-200 fold, 1.5-250 fold, 2-5 fold, 2-10 fold, 2-15 fold, 2-20 fold, 2-25 fold, 2-30 fold, 2-50 fold, 2-75 fold, 2-100 fold, 2-150 fold, 2-200 fold, 2-250 fold, 2.5-5 fold, 2.5-10 fold, 2.5-15 fold, 2.5-20 fold, 2.5-25 fold, 2.5-30 fold, 2.5-50 fold, 2.5-75 fold, 2.5-100 fold, 2.5-150 fold, 2.5-200 fold, 2.5-250 fold, 5-10 fold, 5-15 fold, 5-20 fold, 5-25 fold, 5-30 fold, 5-50 fold, 5-75 fold, 5-100 fold, 5-150 fold, 5-200 fold, 5-250 fold, 10-15 fold, 10-20 fold, 10-25 fold, 10-30 fold, 10-50 fold, 10-75 fold, 10-100 fold, 10-150 fold, 10-200 fold, 10-250 fold, 20-25 fold, 20-30 fold, 20-50 fold, 20-75 fold, 20-100 fold, 20-150 fold, 20-200 fold, 20-250 fold, 25-30 fold, 25-50 fold, 25-75 fold, 25-100 fold, 25-150 fold, 25-200 fold, or 25-250 fold or by at least about 1 fold, 2 fold, 5 fold, 10 fold, 15 fold 20 fold 25 fold, 30 fold, 40 fold, 50 fold 60 fold, 70 fold, 75 fold, 80 fold, 90 fold, 100 fold, 125 fold, 150 fold, 200 fold, 225 fold or 250 fold.

In some embodiments, the individual has cancer that is resistant (has been demonstrated to be resistant) to one or more immune checkpoint inhibitors. In some embodiments, resistance to immune checkpoint inhibitors includes recurrence of cancer or refractory cancer. Recurrence may refer to the reappearance of cancer, in the original site or a new site, after treatment. In some embodiments, resistance to immune checkpoint inhibitors includes progression of the cancer during treatment with the immune checkpoint inhibitors. In some embodiments, resistance to immune checkpoint inhibitors includes cancer that does not respond to treatment. The cancer may be resistant at the beginning of treatment or it may become resistant during treatment. In some embodiments, the cancer is at early stage or at late stage.

Further details regarding therapeutic immune checkpoint inhibitors are provided below and in, e.g., Byun et al. (2017) Nat Rev Endocrinol. 13: 195-207; La-Beck et al. (2015) Pharmacotherapy. 35(10): 963-976; Buchbinder et al. (2016) Am J Clin Oncol. 39(1): 98-106; Michot et al. (2016) Eur J Cancer. 54: 139-148, and Topalian et al. (2016) Nat Rev Cancer. 16: 275-287.

### CTLA4 Inhibitors

In some embodiments, the immune checkpoint inhibitor is a cytotoxic T-lymphocyte-associated protein 4 (CTLA4) (also known as CD152) inhibitor. In some embodiments, the CTLA-4 inhibitor is a blocking antibody, ipilimumab (also known as MDX-010, MDX-101, or Yervoy®), tremelimumab (also known as ticilimumab or CP-675,206).

### PD-1 Axis Binding Antagonists

In some embodiments, the immune checkpoint inhibitor is a PD-1 axis binding antagonist.

Provided herein are methods for treating cancer in an individual comprising administering to the individual an effective amount of a PD-1 axis binding antagonist and an anti-MerTK antibody of the present disclosure. Also provided herein are methods of enhancing immune function or response in an individual (e.g., an individual having cancer) comprising administering to the individual an effective amount of a PD-1 axis binding antagonist and an anti-MerTK antibody of the present disclosure.

In such methods, the PD-1 axis binding antagonist includes a PD-1 binding antagonist, a PDL1 binding antagonist, and/or a PDL2 binding antagonist. Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 binding antagonist is a molecule that inhibits the binding of PD-1 to its ligand binding partner(s). In a specific aspect the PD-1 ligand binding partners are PDL1 and/or PDL2. In another embodiment, a PDL1 binding antagonist is a molecule that inhibits the binding of PDL1 to its binding partner(s). In a specific aspect, PDL1 binding partner(s) are PD-1 and/or B7-1. In another embodiment, the PDL2 binding antagonist is a molecule that inhibits the binding of PDL2 to its binding partner(s). In a specific aspect, a PDL2 binding partner is PD-1. The antagonist may be an antibody, an antigen binding fragment thereof, an immunoadhesin, a fusion protein, an oligopeptide or a small molecule. If the antagonist is an antibody, in some embodiments the antibody comprises a human constant region selected from the group consisting of IgG1, IgG2, IgG3 and IgG4.

### A. Anti-PD-1 Antibodies

In some embodiments, the PD-1 binding antagonist is an anti-PD-1 antibody (e.g., a human antibody, a humanized antibody, or a chimeric antibody). A variety of anti-PD-1 antibodies can be utilized in the methods disclosed herein. In any of the embodiments herein, the PD-1 antibody can bind to a human PD-1 or a variant thereof. In some embodiments the anti-PD-1 antibody is a monoclonal antibody. In some embodiments, the anti-PD-1 antibody is an antibody fragment selected from the group consisting of Fab, Fab′, Fab′-SH, Fv, scFv, and (Fab′)₂ fragments. In some embodiments, the anti-PD-1 antibody is a chimeric or humanized antibody. In other embodiments, the anti-PD-1 antibody is a human antibody.

In some embodiments, the anti-PD-1 antibody is nivolumab (CAS Registry Number: 946414-94-4). Nivolumab (Bristol-Myers Squibb/Ono), also known as MDX-1106-04, MDX-1106, ONO-4538, BMS-936558, and OPDIVO®, is an anti-PD-1 antibody described in WO2006/121168. In some embodiments, the anti-PD-1 antibody comprises a heavy chain and a light chain sequence, wherein:

(a) the heavy chain comprises the
amino acid sequence:
(SEQ ID NO: 118)
QVQLVESGGGVVQPGRSLRLDCKASGITFSNSGMHWVRQAPGKGLEWVAV
IWYDGSKRYYADSVKGRFTISRDNSKNTLFLQMNSLRAEDTAVYYCATND
DYWGQGTLVTVSSASTKGPSVFPLAPCSRSTSESTAALGCLVKDYFPEPV
TVSWNSGALTSGVHTFPAVLQSSGLYSLSSVVTVPSSSLGTKTYTCNVDH
KPSNTKVDKRVESKYGPPCPPCPAPEFLGGPSVFLFPPKPKDTLMISRTP
EVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLY
SRLTVDKSRWQEGNVFSCSVMHEALHNHYTQKSLSLSLGK,
and
(b) the light chain comprises the
amino acid sequence:
(SEQ ID NO: 119)
EIVLTQSPATLSLSPGERATLSCRASQSVSSYLAWYQQKPGQAPRLLIYD
ASNRATGIPARFSGSGSGTDFTLTISSLEPEDFAVYYCQQSSNWPRTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

In some embodiments, the anti-PD-1 antibody comprises the six HVR sequences from SEQ ID NO: 118 and SEQ ID NO: 119 (e.g., the three heavy chain HVRs from SEQ ID NO:118 and the three light chain HVRs from SEQ ID NO: 119). In some embodiments, the anti-PD-1 antibody comprises the heavy chain variable domain from SEQ ID NO: 118 and the light chain variable domain from SEQ ID NO: 119.

In some embodiments, the anti-PD-1 antibody is pembrolizumab (CAS Registry Number: 1374853-91-4). Pembrolizumab (Merck), also known as MK-3475, Merck 3475, lambrolizumab, SCH-900475, and KEYTRUDA®, is an anti-PD-1 antibody described in WO2009/114335. In some embodiments, the anti-PD-1 antibody comprises a heavy chain and a light chain sequence, wherein:

(a) the heavy chain comprises the
amino acid sequence:
(SEQ ID NO: 120)
QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRD
YRFDMGFDYWGQGTTVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK,
and
(b) the light chain comprises the
amino acid sequence:
(SEQ ID NO: 121)
EIVLTQSPATLSLSPGERATLSCRASKGVSTSGYSYLHWYQQKPGQAPRL
LIYLASYLESGVPARFSGSGSGTDFTLTISSLEPEDFAVYYCQHSRDLPL
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC.

In some embodiments, the anti-PD-1 antibody comprises the six HVR sequences from SEQ ID NO: 120 and SEQ ID NO: 121 (e.g., the three heavy chain HVRs from SEQ ID NO: 120 and the three light chain HVRs from SEQ ID NO:121). In some embodiments, the anti-PD-1 antibody comprises the heavy chain variable domain from SEQ ID NO: 120 and the light chain variable domain from SEQ ID NO: 121.

In some embodiments, the anti-PD-1 antibody is MEDI-0680 (AMP-514; AstraZeneca). MEDI-0680 is a humanized IgG4 anti-PD-1 antibody.

In some embodiments, the anti-PD-1 antibody is PDR001 (CAS Registry No. 1859072-53-9; Novartis). PDR001 is a humanized IgG4 anti-PD-1 antibody that blocks the binding of PDL1 and PDL2 to PD-1.

In some embodiments, the anti-PD-1 antibody is REGN2810 (Regeneron). REGN2810 is a human anti-PD-1 antibody.

In some embodiments, the anti-PD-1 antibody is BGB-108 (BeiGene). In some embodiments, the anti-PD-1 antibody is BGB-A317 (BeiGene).

In some embodiments, the anti-PD-1 antibody is JS-001 (Shanghai Junshi). JS-001 is a humanized anti-PD-1 antibody.

In some embodiments, the anti-PD-1 antibody is STI-A1110 (Sorrento). STI-A1110 is a human anti-PD-1 antibody.

In some embodiments, the anti-PD-1 antibody is INCSHR-1210 (Incyte). INCSHR-1210 is a human IgG4 anti-PD-1 antibody.

In some embodiments, the anti-PD-1 antibody is PF-06801591 (Pfizer).

In some embodiments, the anti-PD-1 antibody is TSR-042 (also known as ANB011; Tesaro/AnaptysBio).

In some embodiments, the anti-PD-1 antibody is AM0001 (ARMO Biosciences).

In some embodiments, the anti-PD-1 antibody is ENUM 244C8 (Enumeral Biomedical Holdings). ENUM 244C8 is an anti-PD-1 antibody that inhibits PD-1 function without blocking binding of PDL1 to PD-1.

In some embodiments, the anti-PD-1 antibody is ENUM 388D4 (Enumeral Biomedical Holdings). ENUM 388D4 is an anti-PD-1 antibody that competitively inhibits binding of PDL1 to PD-1.

In some embodiments, the PD-1 antibody comprises the six HVR sequences (e.g., the three heavy chain HVRs and the three light chain HVRs) and/or the heavy chain variable domain and light chain variable domain from a PD-1 antibody described in WO2015/112800 (Applicant: Regeneron), WO2015/112805 (Applicant: Regeneron), WO2015/112900 (Applicant: Novartis), US20150210769 (Assigned to Novartis), WO2016/089873 (Applicant: Celgene), WO2015/035606 (Applicant: Beigene), WO2015/085847 (Applicants: Shanghai Hengrui Pharmaceutical/Jiangsu Hengrui Medicine), WO2014/206107 (Applicants: Shanghai Junshi Biosciences/Junmeng Biosciences), WO2012/145493 (Applicant: Amplimmune), U.S. Pat. No. 9,205,148 (Assigned to MedImmune), WO2015/119930 (Applicants: Pfizer/Merck), WO2015/119923 (Applicants: Pfizer/Merck), WO2016/032927 (Applicants: Pfizer/Merck), WO2014/179664 (Applicant: AnaptysBio), WO2016/106160 (Applicant: Enumeral), and WO2014/194302 (Applicant: Sorrento).

### B. Anti-PDL1 Antibodies

In some embodiments, the PD-1 axis binding antagonist is an anti-PDL1 antibody. A variety of anti-PDL1 antibodies are contemplated and described herein. In any of the embodiments herein, the isolated anti-PDL1 antibody can bind to a human PDL1, for example a human PDL1 as shown in UniProtKB/Swiss-Prot Accession No. Q9NZQ7.1, or a variant thereof. In some embodiments, the anti-PDL1 antibody is capable of inhibiting binding between PDL1 and PD-1 and/or between PDL1 and B7-1. In some embodiments, the anti-PDL1 antibody is a monoclonal antibody. In some embodiments, the anti-PDL1 antibody is an antibody fragment selected from the group consisting of Fab, Fab′-SH, Fv, scFv, and (Fab′)₂ fragments. In some embodiments, the anti-PDL1 antibody is a humanized antibody. In some embodiments, the anti-PDL1 antibody is a human antibody. Examples of anti-PDL1 antibodies useful for the methods of the present disclosure, and methods for making thereof are described in PCT patent application WO 2010/077634 A1 and U.S. Pat. No. 8,217,149, which are incorporated herein by reference.

In some embodiments, the anti-PDL1 antibody is atezolizumab (CAS Registry Number: 1422185-06-5). Atezolizumab (Genentech), also known as MPDL3280A, is an anti-PDL1 antibody.

In some embodiments, the anti-PDL1 antibody comprises a heavy chain variable region and a light chain variable region, wherein:

(a) the heavy chain variable region comprises an
HVR-H1, HVR-H2, and HVR-H3 sequence of
(SEQ ID NO: 122)
GFTFSDSWIH,
(SEQ ID NO: 123)
AWISPYGGSTYYADSVKG
and
(SEQ ID NO: 124)
RHWPGGFDY,
respectively, and
(b) the light chain variable region comprises an
HVR-L1, HVR-L2, and HVR-L3 sequence of
(SEQ ID NO: 125)
RASQDVSTAVA,
(SEQ ID NO: 126)
SASFLYS
and
(SEQ ID NO: 127)
QQYLYHPAT,
respectively.

In some embodiments, the anti-PDL1 antibody is MPDL3280A, also known as atezolizumab and TECENTRIQ® (CAS Registry Number: 1422185-06-5). In some embodiments, the anti-PDL1 antibody comprises a heavy chain and a light chain sequence, wherein:

(a) the heavy chain variable region sequence
comprises the amino acid sequence:
(SEQ ID NO: 128)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSS,
and
(b) the light chain variable region sequence
comprises the amino acid sequence:
(SEQ ID NO: 129)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKR.

In some embodiments, the anti-PDL1 antibody comprises a heavy chain and a light chain sequence, wherein:

(a) the heavy chain comprises the
amino acid sequence:
(SEQ ID NO: 130)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYAST
YRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPG,
and
(b) the light chain comprises the
amino acid sequence:
(SEQ ID NO: 131)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

In some embodiments, the anti-PDL1 antibody is avelumab (CAS Registry Number: 1537032-82-8). Avelumab, also known as MSB0010718C, is a human monoclonal IgG1 anti-PDL1 antibody (Merck KGaA, Pfizer). In some embodiments, the anti-PDL1 antibody comprises a heavy chain and a light chain sequence, wherein:

(a) the heavy chain comprises the
amino acid sequence:
(SEQ ID NO: 132)
EVQLLESGGGLVQPGGSLRLSCAASGFTFSSYIMMWVRQAPGKGLEWVSS
IYPSGGITFYADTVKGRFTISRDNSKNTLYLQMNSLRAEDTAVYYCARIK
LGTVTTVDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPG,
and
(b) the light chain comprises the
amino acid sequence:
(SEQ ID NO: 133)
QSALTQPASVSGSPGQSITISCTGTSSDVGGYNYVSWYQQHPGKAPKLMI
YDVSNRPSGVSNRFSGSKSGNTASLTISGLQAEDEADYYCSSYTSSSTRV
FGTGTKVTVLGQPKANPTVTLFPPSSEELQANKATLVCLISDFYPGAVTV
AWKADGSPVKAGVETTKPSKQSNNKYAASSYLSLTPEQWKSHRSYSCQVT
HEGSTVEKTVAPTECS.

In some embodiments, the anti-PDL1 antibody comprises the six HVR sequences from SEQ ID NO: 132 and SEQ ID NO: 133 (e.g., the three heavy chain HVRs from SEQ ID NO:132 and the three light chain HVRs from SEQ ID NO: 133). In some embodiments, the anti-PDL1 antibody comprises the heavy chain variable domain from SEQ ID NO: 132 and the light chain variable domain from SEQ ID NO: 133.

In some embodiments, the anti-PDL1 antibody is durvalumab (CAS Registry Number: 1428935-60-7). Durvalumab, also known as MEDI4736, is an Fc optimized human monoclonal IgG1 kappa anti-PDL1 antibody (MedImmune, AstraZeneca) described in WO2011/066389 and US2013/034559. In some embodiments, the anti-PDL1 antibody comprises a heavy chain and a light chain sequence, wherein:

(a) the heavy chain comprises the
amino acid sequence:
(SEQ ID NO: 134)
EVQLVESGGGLVQPGGSLRLSCAASGFTFSRYWMSWVRQAPGKGLEWVAN
IKQDGSEKYYVDSVKGRFTISRDNAKNSLYLQMNSLRAEDTAVYYCAREG
GWFGELAFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKRVEPKSCDKTHTCPPCPAPEFEGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPASIEKTISKAKGQPREP
QVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPG,
and
(b) the light chain comprises the
amino acid sequence:
(SEQ ID NO: 135)
EIVLTQSPGTLSLSPGERATLSCRASQRVSSSYLAWYQQKPGQAPRLLIY
DASSRATGIPDRFSGSGSGTDFTLTISRLEPEDFAVYYCQQYGSLPWTFG
QGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC.

In some embodiments, the anti-PDL1 antibody comprises the six HVR sequences from SEQ ID NO:134 and SEQ ID NO:135 (e.g., the three heavy chain HVRs from SEQ ID NO:134 and the three light chain HVRs from SEQ ID NO:135). In some embodiments, the anti-PDL1 antibody comprises the heavy chain variable domain from SEQ ID NO: 134 and the light chain variable domain from SEQ ID NO: 135.

In some embodiments, the anti-PDL1 antibody is MDX-1105 (Bristol Myers Squibb). MDX-1105, also known as BMS-936559, is an anti-PDL1 antibody described in WO2007/005874.

In some embodiments, the anti-PDL1 antibody is LY3300054 (Eli Lilly).

In some embodiments, the anti-PDL1 antibody is STI-A1014 (Sorrento). STI-A1014 is a human anti-PDL1 antibody.

In some embodiments, the anti-PDL1 antibody is KN035 (Suzhou Alphamab). KN035 is single-domain antibody (dAB) generated from a camel phage display library.

In some embodiments, the anti-PDL1 antibody comprises a cleavable moiety or linker that, when cleaved (e.g., by a protease in the tumor microenvironment), activates an antibody antigen binding domain to allow it to bind its antigen, e.g., by removing a non-binding steric moiety. In some embodiments, the anti-PDL1 antibody is CX-072 (CytomX Therapeutics).

In some embodiments, the PDL1 antibody comprises the six HVR sequences (e.g., the three heavy chain HVRs and the three light chain HVRs) and/or the heavy chain variable domain and light chain variable domain from a PDL1 antibody described in US20160108123 (Assigned to Novartis), WO2016/000619 (Applicant: Beigene), WO2012/145493 (Applicant: Amplimmune), U.S. Pat. No. 9,205,148 (Assigned to MedImmune), WO2013/181634 (Applicant: Sorrento), and WO2016/061142 (Applicant: Novartis).

In a still further specific aspect, the PD-1 or PDL1 antibody has reduced or minimal effector function. In a still further specific aspect the minimal effector function results from an “effector-less Fc mutation” or a glycosylation mutation. In still a further embodiment, the effector-less Fc mutation is an N297A or D265A/N297A substitution in the constant region. In some embodiments, the isolated anti-PDL1 antibody is aglycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. Removal of glycosylation sites form an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site another amino acid residue (e.g., glycine, alanine or a conservative substitution).

In some embodiments, the anti-MERTK antibody increases the immune effect of the anti-PDL1 antibody about 3 fold after 20 days of combination treatment. In some embodiments, the anti-MERTK antibody increases the immune effect of the anti-PDL1 antibody about 10 fold after 30 days of treatment.

### C. Other PD-1 Inhibitors

In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. AMP-224 (CAS Registry No. 1422184-00-6; GlaxoSmithKline/MedImmune), also known as B7-DCIg, is a PDL2-Fc fusion soluble receptor described in WO2010/027827 and WO2011/066342.

In some embodiments, the PD-1 binding antagonist is a peptide or small molecule compound. In some embodiments, the PD-1 binding antagonist is AUNP-12 (PierreFabre/Aurigene). See, e.g., WO2012/168944, WO2015/036927, WO2015/044900, WO2015/033303, WO2013/144704, WO2013/132317, and WO2011/161699.

In some embodiments, the PDL1 binding antagonist is a small molecule that inhibits PD-1. In some embodiments, the PDL1 binding antagonist is a small molecule that inhibits PDL1. In some embodiments, the PDL1 binding antagonist is a small molecule that inhibits PDL1 and VISTA. In some embodiments, the PDL1 binding antagonist is CA-170 (also known as AUPM-170). In some embodiments, the PDL1 binding antagonist is a small molecule that inhibits PDL1 and TIM3. In some embodiments, the small molecule is a compound described in WO2015/033301 and WO2015/033299.

### Enhancement of Immune Function

In another aspect, provided herein are methods for enhancing immune function in an individual having cancer comprising administering an effective amount of a combination of an anti-MerTK antibody and an immune checkpoint inhibitor. Various aspects of immune function that may be enhanced by the anti-MerTK antibodies described herein and methods for measuring such enhancement are described below.

In some embodiments of the methods of the present disclosure, the cancer (in some embodiments, a sample of the patient's cancer as examined using a diagnostic test) has elevated levels of T cell infiltration. As used herein, T cell infiltration of a cancer may refer to the presence of T cells, such as tumor-infiltrating lymphocytes (TILs), within or otherwise associated with the cancer tissue. It is known in the art that T cell infiltration may be associated with improved clinical outcome in certain cancers (see, e.g., Zhang et al., N. Engl. J. Med. 348(3):203-213 (2003)).

However, T cell exhaustion is also a major immunological feature of cancer, with many tumor-infiltrating lymphocytes (TILs) expressing high levels of inhibitory co-receptors and lacking the capacity to produce effector cytokines (Wherry, E. J. Nature immunology 12: 492-499 (2011); Rabinovich, G. A., et al., Annual review of immunology 25:267-296 (2007)). In some embodiments of the methods of the present disclosure, the individual has a T cell dysfunctional disorder. In some embodiments of the methods of the present disclosure, the T cell dysfunctional disorder is characterized by T cell anergy or decreased ability to secrete cytokines, proliferate or execute cytolytic activity. In some embodiments of the methods of the present disclosure, the T cell dysfunctional disorder is characterized by T cell exhaustion. In some embodiments of the methods of the present disclosure, the T cells are CD4+ and CD8+ T cells. In some embodiments, the T cells are CD4+ and/or CD8+ T cells.

In some embodiments, CD8+ T cells are characterized, e.g., by presence of CD8b expression (e.g., by rtPCR using e.g., Fluidigm) (Cd8b is also known as T-cell surface glycoprotein CD8 beta chain; CD8 antigen, alpha polypeptide p37; Accession No. is NM_172213). In some embodiments, CD8+ T cells are from peripheral blood. In some embodiments, CD8+ T cells are from tumor.

In some embodiments, Treg cells are characterized, e.g., by presence of Fox3p expression (e.g., by rtPCR e.g., using Fluidigm) (Foxp3 is also known as forkhead box protein P3; scurfin; FOXP3delta7; immunodeficiency, polyendocrinopathy, enteropathy, X-linked; the accession no. is NM_014009). In some embodiments, Treg are from peripheral blood. In some embodiments, Treg cells are from tumor.

In some embodiments, inflammatory T cells are characterized, e.g., by presence of Tbet and/or CXCR3 expression (e.g., by rtPCR using, e.g., Fluidigm). In some embodiments, inflammatory T cells are from peripheral blood. In some embodiments, inflammatory T cells are from tumor.

In some embodiments of the methods of the present disclosure, CD4 and/or CD8 T cells exhibit increased release of cytokines selected from the group consisting of IFN-γ, TNF-α and interleukins. Cytokine release may be measured by any means known in the art, e.g., using Western blot, ELISA, or immunohistochemical assays to detect the presence of released cytokines in a sample containing CD4 and/or CD8 T cells.

In some embodiments of the methods of the present disclosure, the CD4 and/or CD8 T cells are effector memory T cells. In some embodiments of the methods of the present disclosure, the CD4 and/or CD8 effector memory T cells are characterized by having the expression of CD44^{high} CD62^{low}. Expression of CD44^{high} CD62^{low} may be detected by any means known in the art, e.g., by preparing single cell suspensions of tissue (e.g., a cancer tissue) and performing surface staining and flow cytometry using commercial antibodies against CD44 and CD62L. In some embodiments of the methods of the present disclosure, the CD4 and/or CD8 effector memory T cells are characterized by having expression of CXCR3 (also known as C-X-C chemokine receptor type 3; Mig receptor; IP10 receptor; G protein-coupled receptor 9; interferon-inducible protein 10 receptor; Accession No. NM_001504). In some embodiments, the CD4 and/or CD8 effector memory T cells are from peripheral blood. In some embodiments, the CD4 and/or CD8 effector memory T cells are from tumor.

In some embodiments of the methods of the present disclosure, Treg function is suppressed relative to prior to the administration of the combination. In some embodiments, T cell exhaustion is decreased relative to prior to the administration of the combination.

In some embodiments, number of Treg is decreased relative to prior to the administration of the combination. In some embodiments, plasma interferon gamma is increased relative to prior to the administration of the combination. Treg number may be assessed, e.g., by determining percentage of CD4+Fox3p+ CD45+ cells (e.g., by FACS analysis). In some embodiments, absolute number of Treg, e.g., in a sample, is determined. In some embodiments, Treg are from peripheral blood. In some embodiments, Treg are from tumor.

In some embodiments, T cell priming, activation and/or proliferation is increased relative to prior to the administration of the combination. In some embodiments, the T cells are CD4+ and/or CD8+ T cells. In some embodiments, T cell proliferation is detected by determining percentage of Ki67+CD8+ T cells (e.g., by FACS analysis). In some embodiments, T cell proliferation is detected by determining percentage of Ki67+CD4+ T cells (e.g., by FACS analysis). In some embodiments, the T cells are from peripheral blood. In some embodiments, the T cells are from tumor.

### Dosage and Administration

Any of the anti-MerTK antibodies described herein and any immune checkpoint inhibitors known in the art or described herein may be used in the methods of the present disclosure.

In some embodiments, the combination therapy of the present disclosure comprises administration of an anti-MerTK antibody and an immune checkpoint inhibitor. The anti-MerTK antibody and the immune checkpoint inhibitor may be administered in any suitable manner known in the art. For example, the anti-MerTK antibody and the immune checkpoint inhibitor may be administered sequentially (at different times) or concurrently (at the same time). In some embodiments, the immune checkpoint inhibitor is in a separate composition as the anti-MerTK antibody. In some embodiments, the immune checkpoint inhibitor is in the same composition as the anti-MerTK antibody.

The anti-MerTK antibody and the immune checkpoint inhibitor may be administered by the same route of administration or by different routes of administration. In some embodiments, the immune checkpoint inhibitor is administered intravenously, intramuscularly, subcutaneously, topically, orally, transdermally, intraperitoneally, intraorbitally, by implantation, by inhalation, intrathecally, intraventricularly, or intranasally. In some embodiments, the anti-MerTK antibody is administered intravenously, intramuscularly, subcutaneously, topically, orally, transdermally, intraperitoneally, intraorbitally, by implantation, by inhalation, intrathecally, intraventricularly, or intranasally. An effective amount of the immune checkpoint inhibitor and the anti-MerTK antibody may be administered for prevention or treatment of disease. The appropriate dosage of the anti-MerTK antibody and/or the immune checkpoint inhibitor may be determined based on the type of disease to be treated, the type of the immune checkpoint inhibitor and the anti-MerTK antibody, the severity and course of the disease, the clinical condition of the individual, the individual's clinical history and response to the treatment, and the discretion of the attending physician. In some embodiments, combination treatment with anti-MerTK antibody and an immune checkpoint inhibitor (e.g., anti-PD-1 or anti-PDL1 antibody) are synergistic, whereby an efficacious dose of an anti-MerTK antibody in the combination is reduced relative to efficacious dose of the anti-MerTK antibody as a single agent.

As a general proposition, the therapeutically effective amount of the antibody administered to human will be in the range of about 0.01 to about 50 mg/kg of patient body weight whether by one or more administrations. In some embodiments, the antibody used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In some embodiments, the antibody is administered at 15 mg/kg. However, other dosage regimens may be useful. In one embodiment, an anti-MerTK antibody described herein or an anti-PDL1 antibody described herein is administered to a human at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The dose of the antibody administered in a combination treatment may be reduced as compared to a single treatment. The progress of this therapy is easily monitored by conventional techniques.

### (iv) Uses

In one aspect, the present disclosure provides the anti-MerTK antibodies as described above for use as a medicament. In some embodiments, the use is in treating cancer. In some embodiments, the use is in reducing MerTK-mediated clearance of apoptotic cells. Further provided herein are uses of the anti-MerTK antibodies as described above in the manufacture of a medicament. In some embodiments, the medicament is for treatment of cancer. In some embodiments, the cancer expresses functional cGAS-STING cytosolic DNA sensing pathway proteins. These proteins are part of the cGAS-STING signaling pathway and function in innate immunity to detect the presence of cytosolic DNA in order to trigger the expression of inflammatory genes. Examples of cGAS-STING cytosolic DNA sensing pathway proteins include but are not limited to cGAS, STING, TBK-1, IRF3, p50, p60, p65, NF-κB, ISRE, IKK, and STAT6. In some embodiments, the cancer expresses functional STING, functional Cx43, and functional cGAS polypeptides. Functional proteins are proteins that are able to carry out their regular functions in a cell. Examples of functional proteins may include wild-type proteins, tagged proteins, and mutated proteins that retain or improve protein function as compared to a wild-type protein. Protein function can be measured by any methods known to those of skill in the art, including assaying for protein or mRNA expression and sequencing genomic DNA or mRNA. In some embodiments, the cancer comprises tumor-associated macrophages that express functional STING polypeptides. In some embodiments, the cancer comprises tumor cells that express functional cGAS polypeptides. In some embodiments, the cancer comprises tumor cells that express functional Cx43 polypeptides. In certain embodiments, the cancer is colon cancer. In some embodiments, the medicament is for reducing MerTK-mediated clearance of apoptotic cells.

In another aspect, the individual has cancer that expresses (has been shown to express e.g., in a diagnostic test) PDL1 biomarker. In some embodiments, the patient's cancer expresses low PDL1 biomarker. In some embodiments, the patient's cancer expresses high PDL1 biomarker. In some embodiments of any of the methods, assays and/or kits, the PDL1 biomarker is absent from the sample when it comprises 0% of the sample.

In some embodiments of any of the methods, assays and/or kits, the PDL1 biomarker is present in the sample when it comprises more than 0% of the sample. In some embodiments, the PDL1 biomarker is present in at least 1% of the sample. In some embodiments, the PDL1 biomarker is present in at least 5% of the sample. In some embodiments, the PDL1 biomarker is present in at least 10% of the sample.

In some embodiments of any of the methods, assays and/or kits, the PDL1 biomarker is detected in the sample using a method selected from the group consisting of FACS, Western blot, ELISA, immunoprecipitation, immunohistochemistry, immunofluorescence, radioimmunoassay, dot blotting, immunodetection methods, HPLC, surface plasmon resonance, optical spectroscopy, mass spectrometry, HPLC, qPCR, RT-qPCR, multiplex qPCR or RT-qPCR, RNA-seq, microarray analysis, SAGE, MassARRAY technique, and FISH, and combinations thereof.

In some embodiments of any of the methods, assays and/or kits, the PDL1 biomarker is detected in the sample by protein expression. In some embodiments, protein expression is determined by immunohistochemistry (IHC). In some embodiments, the PDL1 biomarker is detected using an anti-PDL1 antibody. In some embodiments, the PDL1 biomarker is detected as a weak staining intensity by IHC. In some embodiments, the PDL1 biomarker is detected as a moderate staining intensity by IHC. In some embodiments, the PDL1 biomarker is detected as a strong staining intensity by IHC. In some embodiments, the PDL1 biomarker is detected on tumor cells, tumor infiltrating immune cells, stromal cells and any combinations thereof. In some embodiments, the staining is membrane staining, cytoplasmic staining or combinations thereof.

In some embodiments of any of the methods, assays and/or kits, the absence of the PDL1 biomarker is detected as absent or no staining in the sample. In some embodiments of any of the methods, assays and/or kits, the presence of the PDL1 biomarker is detected as any staining in the sample.

### IV. Methods of Detection

In some aspects, the present disclosure provides anti-MerTK antibodies or immunoconjugates thereof for use in detection of MerTK protein and cells expression MerTK protein.

In certain embodiments, the presence and/or expression level/amount of protein in a sample is examined using IHC and staining protocols. IHC staining of tissue sections has been shown to be a reliable method of determining or detecting presence of proteins in a sample. In some embodiments, MerTK is detected by immunohistochemistry. In some embodiments, elevated protein expression is determined using IHC. In one embodiment, expression level of MerTK is determined using a method comprising: (a) performing IHC analysis of a sample (such as a subject cancer sample) with an antibody; and b) determining expression level of the protein in the sample. In some embodiments, IHC staining intensity is determined relative to a reference. In some embodiments, the reference is a reference value. In some embodiments, the reference is a reference sample (e.g., control cell line staining sample or tissue sample from non-cancerous patient).

IHC may be performed in combination with additional techniques such as morphological staining and/or fluorescence in-situ hybridization. Two general methods of IHC are available; direct and indirect assays. According to the first assay, binding of antibody to the target antigen is determined directly. This direct assay uses a labeled reagent, such as a fluorescent tag or an enzyme-labeled primary antibody, which can be visualized without further antibody interaction. In a typical indirect assay, unconjugated primary antibody binds to the antigen and then a labeled secondary antibody binds to the primary antibody. Where the secondary antibody is conjugated to an enzymatic label, a chromogenic or fluorogenic substrate is added to provide visualization of the antigen. Signal amplification occurs because several secondary antibodies may react with different epitopes on the primary antibody.

The primary and/or secondary antibody used for IHC typically will be labeled with a detectable moiety. Numerous labels are available which can be generally grouped into the following categories: (a) Radioisotopes, such as ³⁵S, ¹⁴C, ¹²⁵I, ³H, and ¹³¹I; (b) colloidal gold particles; (c) fluorescent labels including, but are not limited to, rare earth chelates (europium chelates), Texas Red, rhodamine, fluorescein, dansyl, Lissamine, umbelliferone, phycocrytherin, phycocyanin, or commercially available fluorophores such SPECTRUM ORANGE7 and SPECTRUM GREEN7 and/or derivatives of any one or more of the above; (d) various enzyme-substrate labels are available and U.S. Pat. No. 4,275,149 provides a review of some of these. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like.

Examples of enzyme-substrate combinations include, for example, horseradish peroxidase (HRPO) with hydrogen peroxidase as a substrate; alkaline phosphatase (AP) with para-Nitrophenyl phosphate as chromogenic substrate; and β-D-galactosidase (0-D-Gal) with a chromogenic substrate (e.g., p-nitrophenyl-β-D-galactosidase) or fluorogenic substrate (e.g., 4-methylumbelliferyl-β-D-galactosidase). For a general review of these, see U.S. Pat. Nos. 4,275,149 and 4,318,980.

Specimens thus prepared may be mounted and coverslipped. Slide evaluation is then determined, e.g., using a microscope, and staining intensity criteria, routinely used in the art, may be employed. In one embodiment, it is understood that when cells and/or tissue from a tumor is examined using IHC, staining is generally determined or assessed in tumor cell and/or tissue (as opposed to stromal or surrounding tissue that may be present in the sample). In some embodiments, it is understood that when cells and/or tissue from a tumor is examined using IHC, staining includes determining or assessing in tumor infiltrating immune cells, including intratumoral or peritumoral immune cells.

### V. Articles of Manufacture or Kits

In another embodiment of the present disclosure, an article of manufacture or a kit is provided comprising an anti-MerTK antibody. In some embodiments, the article of manufacture or kit further comprises a package insert comprising instructions for using the anti-MerTK antibody to treat or delay progression of cancer in an individual or to enhance immune function of an individual having cancer. Any of the anti-MerTK antibodies described herein may be included in the article of manufacture or kits. The article of manufacture or kit may further comprise an immune checkpoint inhibitor. In some embodiments, the immune checkpoint inhibitor is an anti-PDL1 antibody.

In some embodiments, the immune checkpoint inhibitor and the anti-MerTK antibody are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

The specification is considered to be sufficient to enable one skilled in the art to practice the compositions and methods of the present disclosure. Various modifications in addition to those shown and described herein will become apparent to those skilled in the art from the foregoing description and fall within the scope of the appended claims. All publications, patents, and patent applications cited herein are hereby incorporated by reference in their entirety for all purposes.

### EXAMPLES

The present disclosure will be more fully understood by reference to the following examples. They should not, however, be construed as limiting the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

### Example 1: Generating Rabbit Anti-MerTK Monoclonal Antibodies and Humanization

Monoclonal antibodies against MerTK were generated in rabbits. Then, the antibodies were humanized, and residues that were important for stability and affinity were identified.

### Generating Rabbit Anti-MerTK Monoclonal Antibodies

New Zealand White rabbits were immunized with human and mouse MerTK. Individual B-cells were isolated using a modified protocol derived from published literature (Offner et al. PLoS ONE 9(2), 2014). Human and mouse MerTK⁺ B cells were sorted into single wells using direct FACS sorting of IgG⁺. B-cell culture supernatants were analyzed via primary ELISA screening for human and mouse MerTK binding, and B-cells were lysed and stored at −80° C.

The light chain and heavy chain variable regions of MerTK specific B cells were amplified by PCR and cloned into expression vectors as described in the published literature (Offner et al. PLoS ONE 9(2), 2014). Each recombinant rabbit monoclonal antibody was expressed in Expi293 cells and purified with protein A. Purified anti-MerTK antibodies were then subjected to functional characterization, affinity determination, and epitope binning.

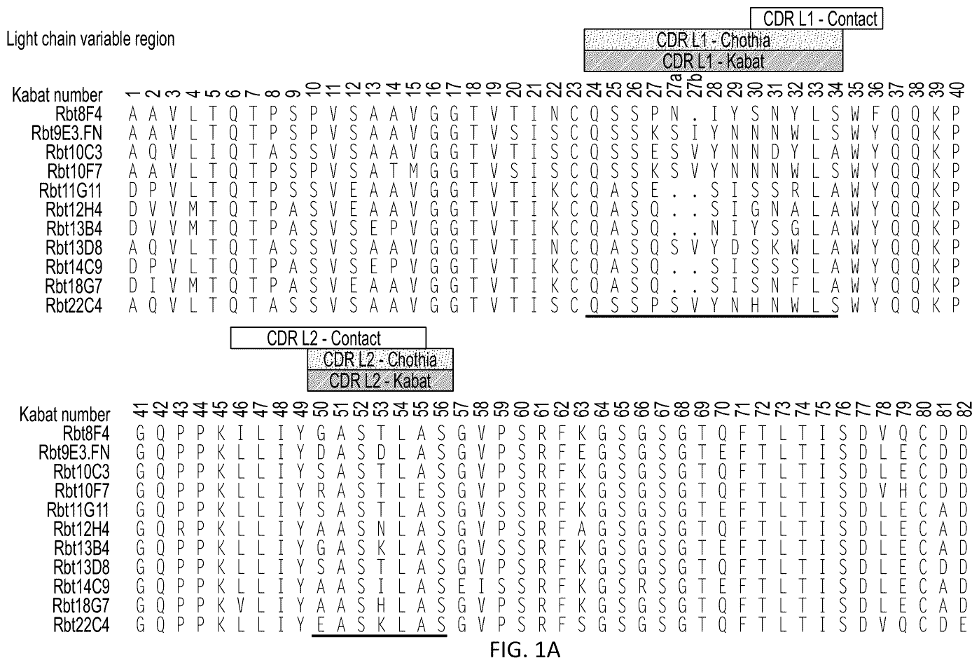

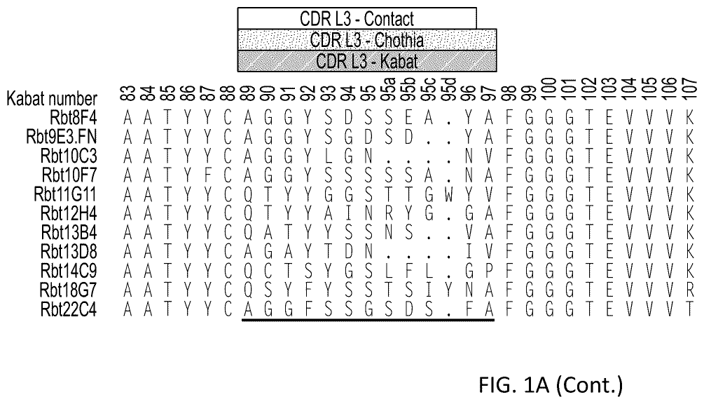

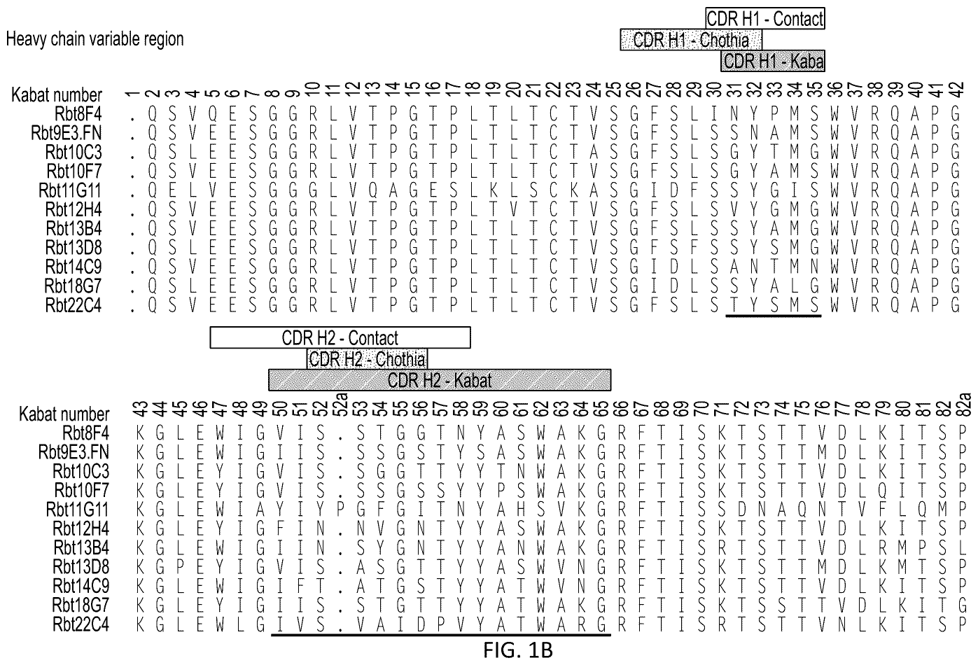

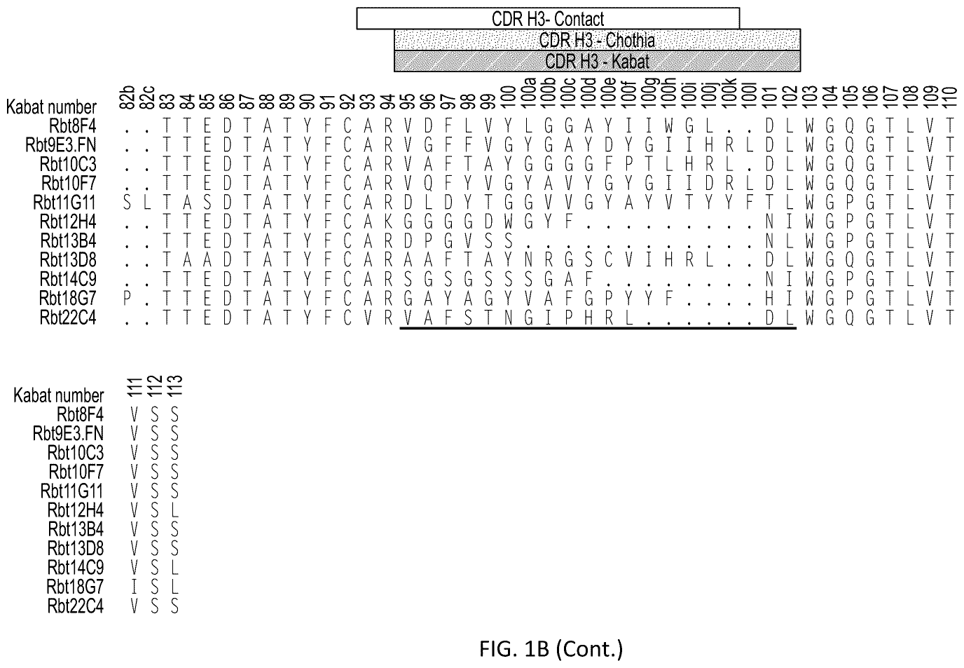

Residue numbers referenced for each antibody are matched to Kabat et al., Sequences of proteins of immunological interest, 5th Ed., Public Health Service, National Institutes of Health, Bethesda, MD (1991). FIGS. 1A and 1B, respectively, show the aligned sequences of the light chain and heavy chain variable regions for each anti-MerTK rabbit antibody. The CDR sequences, as defined by Kabat et al., are underlined in FIGS. 1A and 1B.

### MerTK Antibody Humanization

### Step 1: Generating Primary Humanized Antibodies

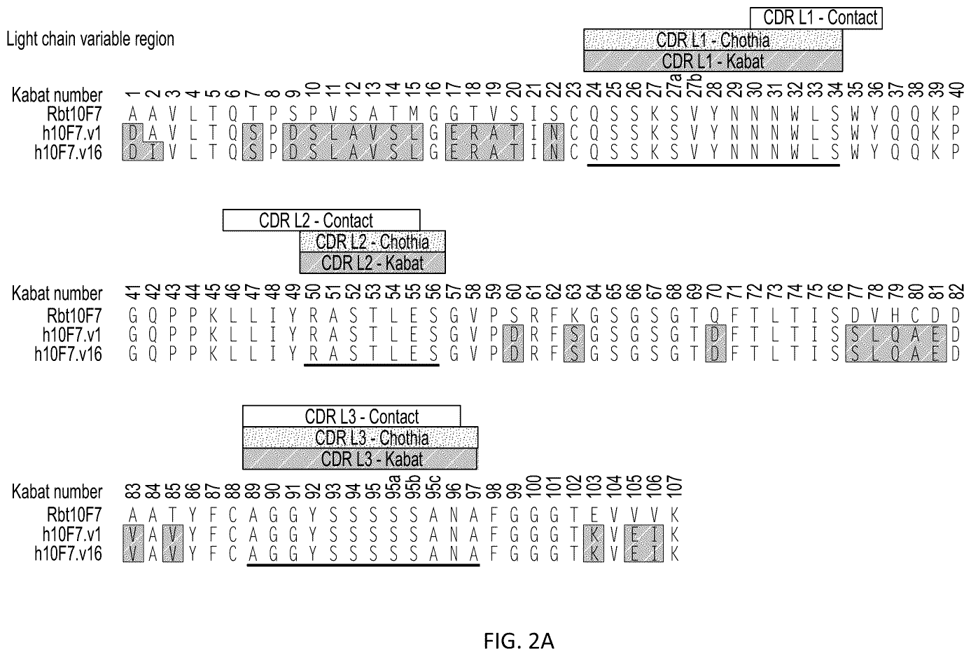

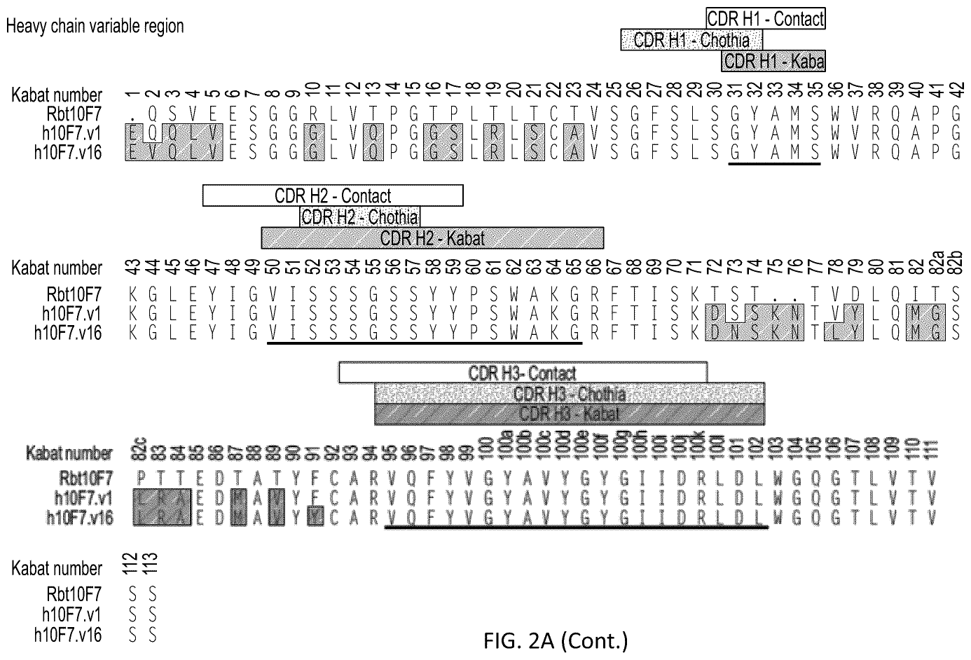

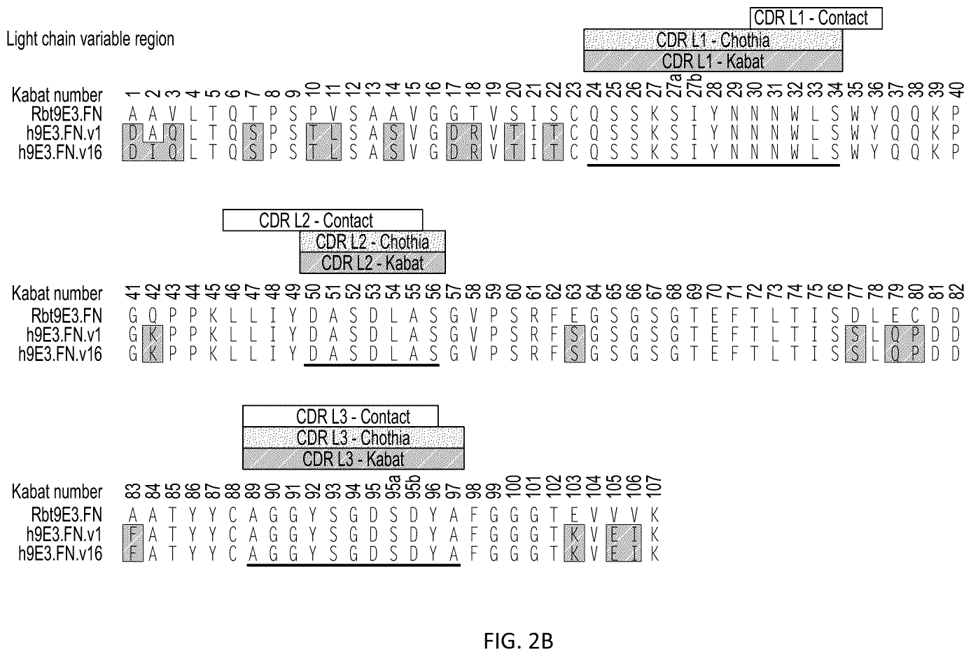

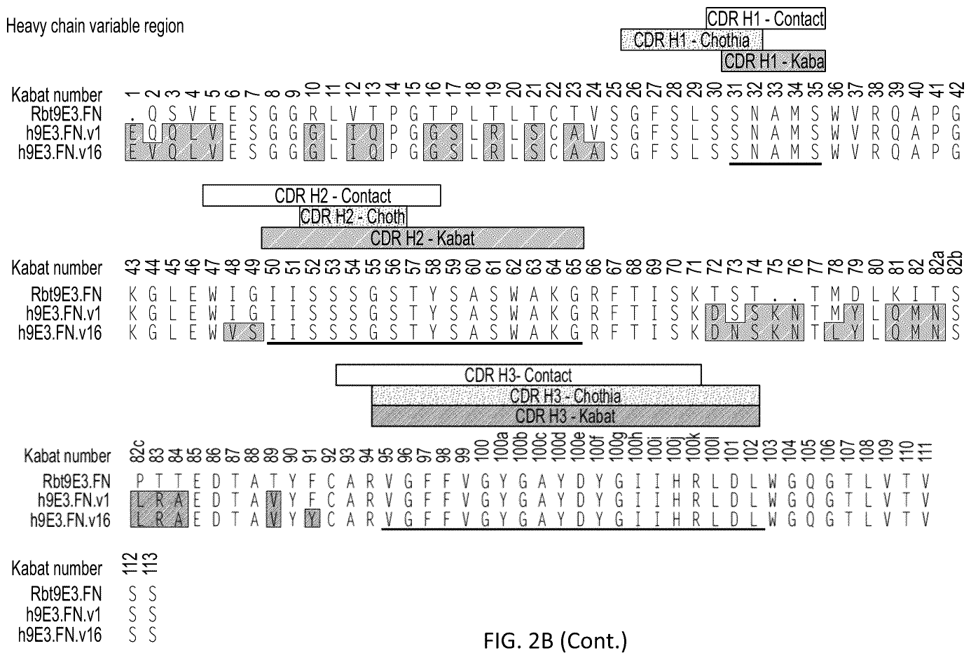

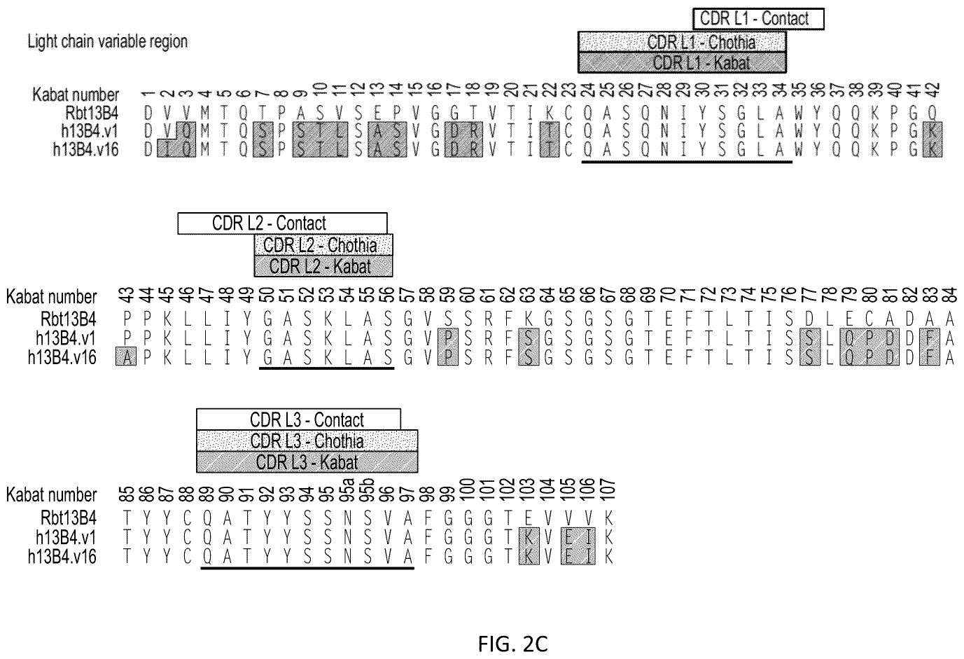

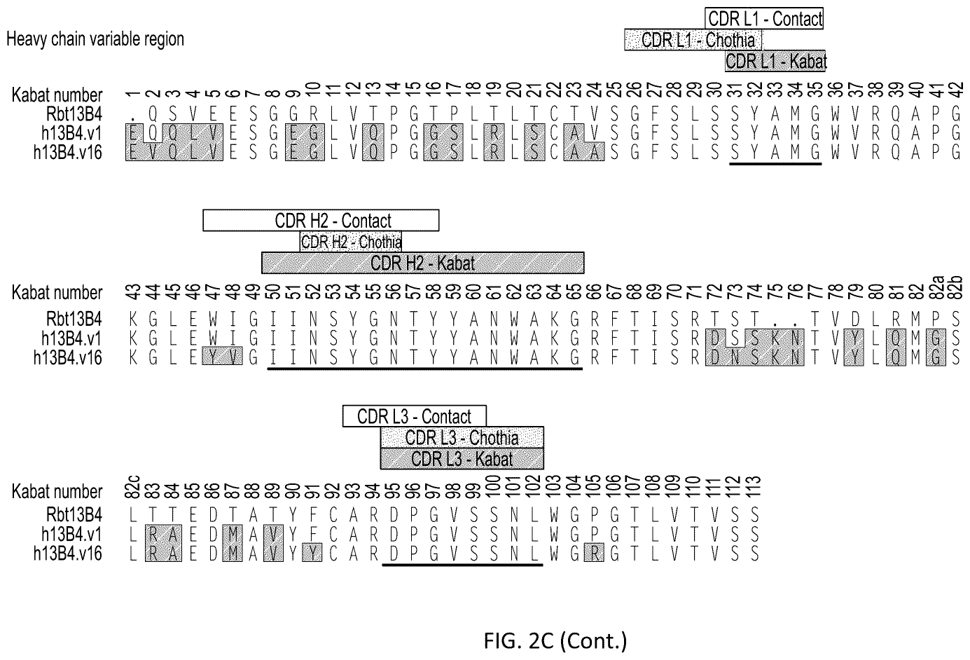

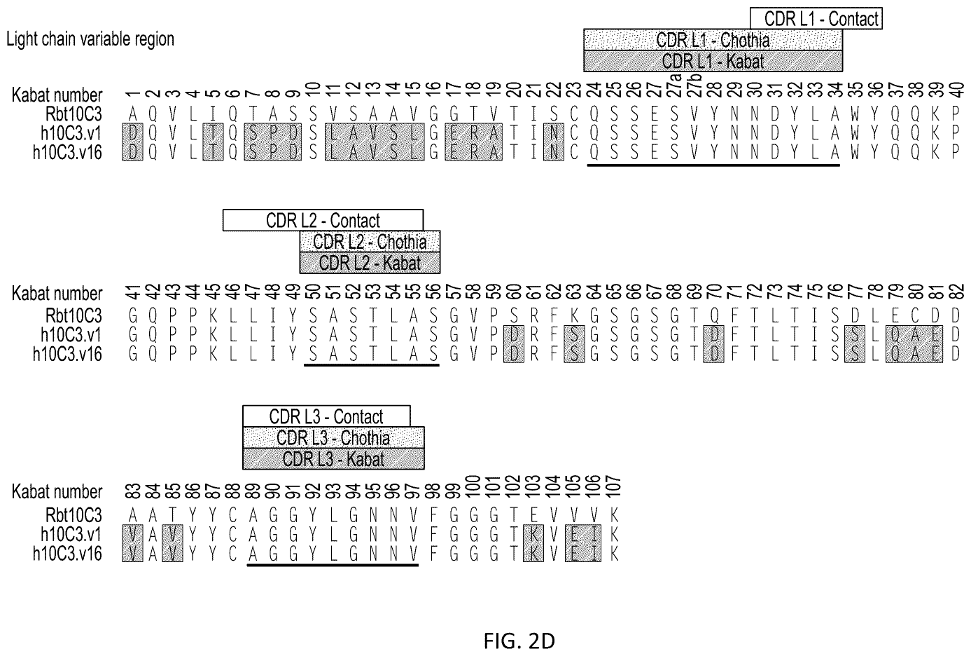

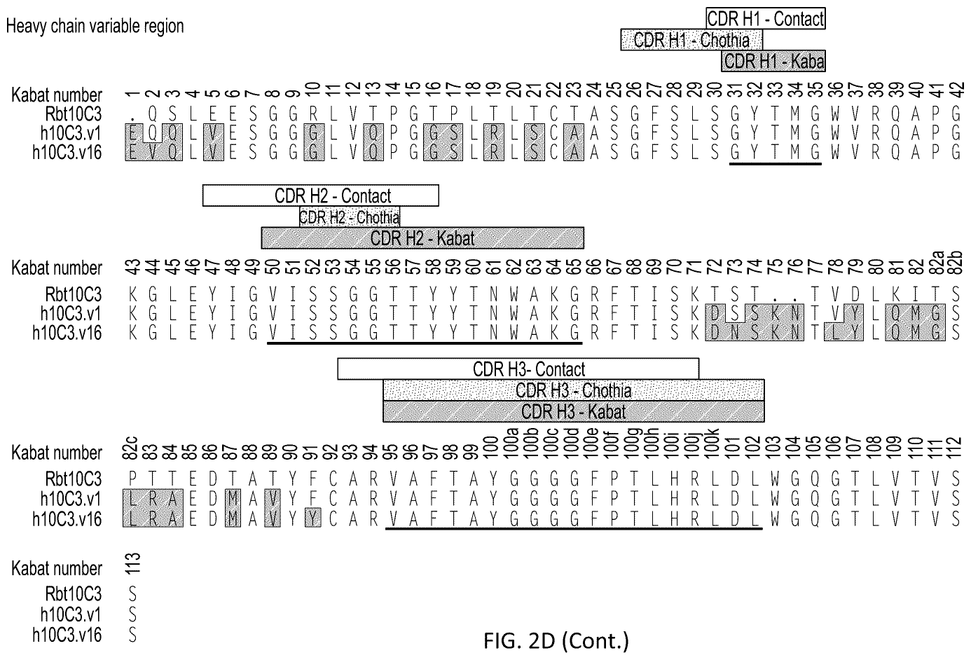

Residue numbers for each antibody referenced are matched to Kabat et al. First, hypervariable regions of each rabbit antibody were engineered into their closest human germline acceptor framework to generate primary humanized antibodies, Version 1 (labeled “v1”) (human IgG1) (FIGS. 2A-2D). Specifically, the rabbit antibody light chain variable domain (VL) positions 24-34 (L1), 50-56 (L2) and 89-97 (L3) and the heavy chain variable domain (VH) positions 26-35 (H1), 50-65 (H2) and 95-102 (H3) were retained for the CDRs from each rabbit antibody (FIGS. 2A-2D). In the framework, rabbit residues at “Vernier” zones, which may adjust CDR structure and fine-tune the antigen fit (See, e.g., Foote and Winter, J. Mol. Biol. 224: 487-499 (1992)), were also included. FIGS. 2A-2D show the aligned sequences of each antibody after the first step of humanization.

### Step 2: Framework Polishing Humanized Antibodies

Each rabbit residue at the framework “Vernier” zone of the primary humanized antibody, Version 1, was mutated to a human residue according to its corresponding closest human acceptor framework.

Each humanized mutation variant was subject to BIAcore analysis to determine the important rabbit residues for binding and stability. Binding affinity determinations were obtained using Surface Plasmon Resonance (SRP) measurements from a BIAcore™-T200 instrument. Briefly, each humanized mutation variant antibody was captured to achieve approximately 100 RU (Response Units). Then, 3-fold serial dilutions of human MerTK (0.4 nM to 100 nM) diluted in HBS-EP buffer (0.01M HEPES pH 7.4, 0.15M NaCl, 3 mM EDTA and 0.05% v/v surfactant P20) was injected into the BIAcore™-T200 instrument at 37° C. with a flow rate of 30 μl/min. Association rates (kₒₙ) and dissociation rates (k_{off}) were calculated using a simple one-to-one Langmuir binding model (BIAcore T200 evaluation software version 2.0). The equilibrium dissociation constant (K_{D}) was calculated as the ratio k_{off}/kₒₙ.

TABLES 2-5 identify the important residues for binding and stability in gray shading. The important residues of clone h10C3.V1 were Q2 and L4 in the light chain variable region and 148, G49S, and K71 in the heavy chain variable region (TABLE 2). The important residues of clone h10F7.V1 were L4 and F87 in the light chain variable region and V24, 148, G49, K71, and S73 in the heavy chain variable region (TABLE 3). The important residues of clone h9E3.FN.V1 were L4 and P43 in the light chain variable region and K71 in the heavy chain variable region (TABLE 4). The important residues of clone h13B4.V1 were G49 and V78 in the heavy chain variable region (TABLE 5).

TABLE 2

| | | KD (nM) |
|---|---|---|
| h10C3.V1 (Parent) | | 13.8 |
| LC | Q2I | 17.1 |
| LC | L4M | 12.3 |
| HC | Q2V | 12.5 |
| HC | I48V | 17.1 |
| HC | G49S | 30.2 |
| HC | K71R | 20.0 |
| HC | S73N | 15.0 |
| HC | V78L | 7.4 |
| HC | F91Y | 14.9 |

TABLE 3

| | | KD (nM) |
|---|---|---|
| h10F7.V1 (Parent) | | 7.3 |
| LC | A2I | 5.9 |
| LC | L4M | 6.2 |
| LC | F87Y | 8.8 |
| HC | Q2V | 5.9 |
| HC | V24A | 9.4 |
| HC | I48V | 9.3 |
| HC | G49S | 9.5 |
| HC | K71R | 15.3 |
| HC | S73N | 7.6 |
| HC | V78L | 5.1 |
| HC | F91Y | 6.4 |

TABLE 4

| | | KD (nM) |
|---|---|---|
| h9E3.FN.V1 (Parent) | | 12.2 |
| LC | A2I | 10.7 |
| LC | L4M | 7.5 |
| LC | P43A | 15.0 |
| HC | Q2V | 10.2 |
| HC | V24A | 8.7 |
| HC | I48V | 10.2 |
| HC | G49S | 5.6 |
| HC | K71R | 41.3 |
| HC | S73N | 12.3 |
| HC | M78L | 12.5 |
| HC | F91Y | 11.5 |

TABLE 5

| | | KD (nM) |
|---|---|---|
| h13B4.V1 (Parent) | | 4.8 |
| LC | V2I | 5.6 |
| LC | P43A | 4.6 |
| HC | Q2V | 4.5 |
| HC | V24A | 6.0 |
| HC | W47Y | 3.1 |
| HC | I48V | 5.8 |
| HC | G49S | 51.5 |
| HC | S73N | 4.7 |
| HC | V78L | 9.2 |
| HC | F91Y | 4.5 |
| HC | P105R | 4.2 |

To generate the final humanized framework polished antibodies, the important binding and stability rabbit framework residues were maintained while the other residues were changed to the closest human germline framework residues. FIGS. 2A-2D show the aligned sequences of each antibody including the sequences of the final humanized framework polished antibody versions (v.14 or v.16).

A summary of the rabbit and humanized antibody sequences is provided in TABLES 6-8.

TABLE 6

| | | | | | | |
|---|---|---|---|---|---|---|
| Name | CDR Ll | CDR L2 | CDR L3 | CDR H1 | CDR H2 | CDR H3 |

Antibodies binding fibronectin-like domains

| | | | | | | |
|---|---|---|---|---|---|---|
| Rbt8F4 | QSSPNI | GASTLAS | AGGYSDSS | NYPMS | VISSTGGT | VDFLVYLGG |
| | YSNYLS | (SEQ ID NO: 2) | EAYA | (SEQ ID NO: 4) | NYASWAKG | AYIIWGLDL |
| | (SEQ ID | | (SEQ ID | | (SEQ ID | (SEQ ID NO: 6) |
| | NO: 1) | | NO: 3) | | NO: 5) | |
| Rbt9E3.FN | QSSKSIYN | DASDLAS | AGGYSGDS | SNAMS | IISSSGST | VGFFVGYGAY |
| | NNWLS | (SEQ ID NO: 8) | DYA | (SEQ ID NO: 10) | YSASWAKG | DYGIIHRLDL |
| | (SEQ ID | | (SEQ ID | | (SEQ ID | (SEQ ID NO: 12) |
| | NO: 7) | | NO: 9) | | NO: 11) | |
| h9E3.FN.v1 | QSSKSIYN | DASDLAS | AGGYSGDS | SNAMS | IISSSGST | VGFFVGYGAY |
| | NNWLS | (SEQ ID NO: 8) | DYA | (SEQ ID NO: 10) | YSASWAKG | DYGIIHRLDL |
| | (SEQ ID | | (SEQ ID | | (SEQ ID | (SEQ ID NO: 12) |
| | NO: 7) | | NO: 9) | | NO: 11) | |
| h9E3.FN.v16 | QSSKSIYN | DASDLAS | AGGYSGDS | SNAMS | IISSSGST | VGFFVGYGAY |
| | NNWLS | (SEQ ID NO: 8) | DYA | (SEQ ID NO: 10) | YSASWAKG | DYGIIHRLDL |
| | (SEQ ID | | (SEQ ID | | (SEQ ID | (SEQ ID NO: 12) |
| | NO: 7) | | NO: 9) | | NO: 11) | |
| Rbt10C3 | QSSESVYN | SASTLAS | AGGYLGNNV | GYTMG | VISSGGTT | VAFTAYGGGG |
| | NDYLA | (SEQ ID NO: 14) | (SEQ ID | (SEQ ID NO: 16) | YYTNWAKG | FPTLHRLDL |
| | (SEQ ID | | NO: 15) | | (SEQ ID | (SEQ ID NO: 18) |
| | NO: 13) | | | | NO: 17) | |
| h10C3.v1 | QSSESVYN | SASTLAS | AGGYLGNNV | GYTMG | VISSGGTT | VAFTAYGGGG |
| | NDYLA | (SEQ ID NO: 14) | (SEQ ID | (SEQ ID NO: 16) | YYTNWAKG | FPTLHRLDL |
| | (SEQ ID | | NO: 15) | | (SEQ ID | (SEQ ID NO: 18) |
| | NO: 13) | | | | NO: 17) | |
| h10C3.v14 | QSSESVYN | SASTLAS | AGGYLGNNV | GYTMG | VISSGGTT | VAFTAYGGGG |
| | NDYLA | (SEQ ID NO: 14) | (SEQ ID | (SEQ ID NO: 16) | YYTNWAKG | FPTLHRLDL |
| | (SEQ ID | | NO: 15) | | (SEQ ID | (SEQ ID NO: 18) |
| | NO: 13) | | | | NO: 17) | |
| Rbt10F7 | QSSKSVY | RASTLES | AGGYSSSS | GYAMS | VISSSGSS | VQFYVGYAVY |
| | NNNWLS | (SEQ ID NO: 20) | SANA | (SEQ ID NO: 22) | YYPSWAKG | GYGIIDRLDL |
| | (SEQ ID | | (SEQ ID | | (SEQ ID | (SEQ ID NO: 24) |
| | NO: 19) | | NO: 21) | | NO: 23) | |
| h10F7.v1 | QSSKSVY | RASTLES | AGGYSSSS | GYAMS | VISSSGSS | VQFYVGYAVY |
| | NNNWLS | (SEQ ID NO: 20) | SANA | (SEQ ID NO: 22) | YYPSWAKG | GYGIIDRLDL |
| | (SEQ ID | | (SEQ ID | | (SEQ ID | (SEQ ID NO: 24) |
| | NO: 19) | | NO: 21) | | NO: 23) | |
| h10F7.v16 | QSSKSVY | RASTLES | AGGYSSSS | GYAMS | VISSSGSS | VQFYVGYAVY |
| | NNNWLS | (SEQ ID NO: 20) | SANA | (SEQ ID NO: 22) | YYPSWAKG | GYGIIDRLDL |
| | (SEQ ID | | (SEQ ID | | (SEQ ID | (SEQ ID NO: 24) |
| | NO: 19) | | NO: 21) | | NO: 23) | |
| Rbt13D8 | QASQSVY | SASTLAS | AGAYTDNIV | SYSMG | VISASGTT | AAFTAYNRG |
| | DSKWLA | (SEQ ID NO: 14) | (SEQ ID | (SEQ ID NO: 27) | YYASWVNG | SCVIHRLDL |
| | (SEQ ID | | NO: 26) | | (SEQ ID | (SEQ ID NO: 29) |
| | NO: 25) | | | | NO: 28) | |
| Rbt22C4 | QSSPSVYN | EASKLAS | AGGFSSGS | TYSMS | IVSVAIDP | VAFSTNGIPHRLDL |
| | HNWLS | (SEQ ID NO: 31) | DSFA | (SEQ ID NO: 33) | VYATWARG | (SEQ ID NO: 35) |
| | (SEQ ID | | (SEQ ID | | (SEQ ID | |
| | NO: 30) | | NO: 32) | | NO: 34) | |

Antibodies binding Ig-like domains

| | | | | | | |
|---|---|---|---|---|---|---|
| Rbt11G11 | QASESISS | SASTLAS | QTYYGGST | SYGIS | YIYPGFGIT | DLDYTGGVVG |
| | RLA | (SEQ ID NO: 14) | TGWYV | (SEQ ID NO: 38) | NYAHSVKG | YAYVTYYFTL |
| | (SEQ ID | | (SEQ ID | | (SEQ ID | (SEQ ID NO: 40) |
| | NO: 36) | | NO: 37) | | NO: 39) | |
| Rbt12H4 | QASQSIGN | AASNLAS | QTYYAINR | VYGMG | FINNVGNT | GGGGDWGYFNI |
| | ALA | (SEQ ID NO: 42) | YGGA | (SEQ ID NO: 44) | YYASWAKG | (SEQ ID NO: 46) |
| | (SEQ ID | | (SEQ ID | | (SEQ ID | |
| | NO: 41) | | NO: 43) | | NO: 45) | |
| Rbt13B4 | QASQNIYS | GASKLAS | QATYYSSN | SYAMG | IINSYGNT | DPGVSSNL |
| | GLA | (SEQ ID NO: 48) | SVA | (SEQ ID NO: 50) | YYANWAKG | (SEQ ID NO: 52) |
| | (SEQ ID | | (SEQ ID | | (SEQ ID | |
| | NO: 47) | | NO: 49) | | NO: 51) | |
| h13B4.v1 | QASQNIYS | GASKLAS | QATYYSSN | SYAMG | IINSYGNT | DPGVSSNL |
| | GLA | (SEQ ID NO: 48) | SVA | (SEQ ID NO: 50) | YYANWAKG | (SEQ ID NO: 52) |
| | (SEQ ID | | (SEQ ID | | (SEQ ID | |
| | NO: 47) | | NO: 49) | | NO: 51) | |
| h13B4.v16 | QASQNIYS | GASKLAS | QATYYSSN | SYAMG | IINSYGNT | DPGVSSNL |
| | GLA | (SEQ ID NO: 48) | SVA | (SEQ ID NO: 50) | YYANWAKG | (SEQ ID NO: 52) |
| | (SEQ ID | | (SEQ ID | | (SEQ ID | |
| | NO: 47) | | NO: 49) | | NO: 51) | |
| Rbt14C9 | QASQSISS | AASILAS | QCTSYGSL | ANTMN | IFTATGST | SGSGSSSGAFNI |
| | SLA | (SEQ ID NO: 54) | FLGP | (SEQ ID NO: 56) | YYATWVNG | (SEQ ID NO: 58) |
| | (SEQ ID | | (SEQ ID | | (SEQ ID | |
| | NO: 53) | | NO: 55) | | NO: 57) | |
| Rbt18G7 | QASQSISN | AASHLAS | QSYFYSST | SYALG | IISSTGTT | GAYAGYVA |
| | FLA | (SEQ ID NO: 60) | SIYNA | (SEQ ID NO: 62) | YYATWAKG | FGPYYFHI |
| | (SEQ ID | | (SEQ ID | | (SEQ ID | (SEQ ID NO: 64) |
| | NO: 59) | | NO: 61) | | NO: 63) | |

TABLE 7

| | | |
|---|---|---|
| Name | Light Chain Variable Region | Heavy Chain Variable Region |

Antibodies binding fibronectin-like domains

| | | |
|---|---|---|
| Rbt8F4 | AAVLTQTPSPVSAAVGGTVTINCQSSPNIY | QSVQESGGRLVTPGTPLTLTCTVSGFSLINY |
| | SNYLSWFQQKPGQPPKILIYGASTLASGVP | PMSWVRQAPGKGLEWIGVISSTGGTNYASWA |
| | SRFKGSGSGTQFTLTISDVQCDDAATYYCA | KGRFTISKTSTTVDLKITSPTTEDTATYFCA |
| | GGYSDSSEAYAFGGGTEVVVK | RVDFLVYLGGAYIIWGLDLWGQGTLVTVSS |
| | (SEQ ID NO: 65) | (SEQ ID NO: 83) |
| Rbt9E3.FN | AAVLTQTPSPVSAAVGGTVSISCQSSKSIY | QSVEESGGRLVTPGTPLTLTCTVSGFSLSSNA |
| | NNNWLSWYQQKPGQPPKLLIYDASDLASGV | MSWVRQAPGKGLEWIGIISSSGSTYSASWAK |
| | PSRFEGSGSGTEFTLTISDLECDDAATYYC | GRFTISKTSTTMDLKITSPTTEDTATYFCAR |
| | AGGYSGDSDYAFGGGTEVVVK | VGFFVGYGAYDYGIIHRLDLWGQGTLVTVSS |
| | (SEQ ID NO: 66) | (SEQ ID NO: 84) |
| h9E3.FN.v1 | DAQLTQSPSTLSASVGDRVTITCQSSKSIY | EQQLVESGGGLIQPGGSLRLSCAVSGFSLSSN |
| | NNNWLSWYQQKPGKPPKLLIYDASDLASGV | AMSWVRQAPGKGLEWIGIISSSGSTYSASWAK |
| | PSRFSGSGSGTEFTLTISSLQPDDFATYYC | GRFTISKDSSKNTMYLQMNSLRAEDTAVYFCA |
| | AGGYSGDSDYAFGGGTKVEIK | RVGFFVGYGAYDYGIIHRLDLWGQGTLVTVSS |
| | (SEQ ID NO: 67) | (SEQ ID NO: 85) |
| h9E3.FN.v16 | DIQLTQSPSTLSASVGDRVTITCQSSKSIY | EVQLVESGGGLIQPGGSLRLSCAASGFSLSSN |
| | NNNWLSWYQQKPGKPPKLLIYDASDLASGV | AMSWVRQAPGKGLEWVSIISSSGSTYSASWAK |
| | PSRFSGSGSGTEFTLTISSLQPDDFATYYC | GRFTISKDNSKNTLYLQMNSLRAEDTAVYYCA |
| | AGGYSGDSDYAFGGGTKVEIK | RVGFFVGYGAYDYGIIHRLDLWGQGTLVTVSS |
| | (SEQ ID NO: 68) | (SEQ ID NO: 86) |
| Rbt10C3 | AQVLIQTASSVSAAVGGTVTISCQSSESVY | QSLEESGGRLVTPGTPLTLTCTASGFSLSGYT |
| | NNDYLAWYQQKPGQPPKLLIYSASTLASGV | MGWVRQAPGKGLEYIGVISSGGTTYYTNWAKG |
| | PSRFKGSGSGTQFTLTISDLECDDAATYYC | RFTISKTSTTVDLKITSPTTEDTATYFCARVA |
| | AGGYLGNNVFGGGTEVVVK | FTAYGGGGFPTLHRLDLWGQGTLVTVSS |
| | (SEQ ID NO: 69) | (SEQ ID NO: 87) |
| h10C3.v1 | DQVLTQSPDSLAVSLGERATINCQSSESVY | EQQLVESGGGLVQPGGSLRLSCAASGFSLSGY |
| | NNDYLAWYQQKPGQPPKLLIYSASTLASGV | TMGWVRQAPGKGLEYIGVISSGGTTYYTNWAK |
| | PDRFSGSGSGTDFTLTISSLQAEDVAVYYC | GRFTISKDSSKNTVYLQMGSLRAEDMAVYFCA |
| | AGGYLGNNVFGGGTKVEIK | RVAFTAYGGGGFPTLHRLDLWGQGTLVTVSS |
| | (SEQ ID NO: 70) | (SEQ ID NO: 88) |
| h10C3.v14 | DQVLTQSPDSLAVSLGERATINCQSSESVY | EVQLVESGGGLVQPGGSLRLSCAASGFSLSGY |
| | NNDYLAWYQQKPGQPPKLLIYSASTLASGV | TMGWVRQAPGKGLEYIGVISSGGTTYYTNWAK |
| | PDRFSGSGSGTDFTLTISSLQAEDVAVYYC | GRFTISKDNSKNTLYLQMGSLRAEDMAVYYCA |
| | AGGYLGNNVFGGGTKVEIK | RVAFTAYGGGGFPTLHRLDLWGQGTLVTVSS |
| | (SEQ ID NO: 70) | (SEQ ID NO: 89) |
| Rbt10F7 | AAVLTQTPSPVSATMGGTVSISCQSSKSVY | QSVEESGGRLVTPGTPLTLTCTVSGFSLSGYA |
| | NNNWLSWYQQKPGQPPKLLIYRASTLESGV | MSWVRQAPGKGLEYIGVISSSGSSYYPSWAKG |
| | PSRFKGSGSGTQFTLTISDVHCDDAATYFC | RFTISKTSTTVDLQITSPTTEDTATYFCARVQ |
| | AGGYSSSSSANAFGGGTEVVVK | FYVGYAVYGYGIIDRLDLWGQGTLVTVSS |
| | (SEQ ID NO: 71) | (SEQ ID NO: 90) |
| h10F7.v1 | DAVLTQSPDSLAVSLGERATINCQSSKSVY | EQQLVESGGGLVQPGGSLRLSCAVSGFSLSGY |
| | NNNWLSWYQQKPGQPPKLLIYRASTLESGV | AMSWVRQAPGKGLEYIGVISSSGSSYYPSWAK |
| | PDRFSGSGSGTDFTLTISSLQAEDVAVYFC | GRFTISKDSSKNTVYLQMGSLRAEDMAVYFCA |
| | AGGYSSSSSANAFGGGTKVEIK | RVQFYVGYAVYGYGIIDRLDLWGQGTLVTVSS |
| | (SEQ ID NO: 72) | (SEQ ID NO: 91) |
| h10F7.v16 | DIVLTQSPDSLAVSLGERATINCQSSKSVY | EVQLVESGGGLVQPGGSLRLSCAVSGFSLSGY |
| | NNNWLSWYQQKPGQPPKLLIYRASTLESGV | AMSWVRQAPGKGLEYIGVISSSGSSYYPSWAK |
| | PDRFSGSGSGTDFTLTISSLQAEDVAVYFC | GRFTISKDNSKNTLYLQMGSLRAEDMAVYYCA |
| | AGGYSSSSSANAFGGGTKVEIK | RVQFYVGYAVYGYGIIDRLDLWGQGTLVTVSS |
| | (SEQ ID NO: 73) | (SEQ ID NO: 92) |
| Rbt13D8 | AQVLTQTASSVSAAVGGTVTINCQASQSVY | QSLEESGGRLVTPGTPLTLTCTVSGFSFSSYS |
| | DSKWLAWYQQKPGQPPKLLIYSASTLASGV | MGWVRQAPGKGPEYIGVISASGTTYYASWVNG |
| | PSRFKGSGSGTQFTLTISDLECDDAATYYC | RFTISKTSTTMDLKMTSPTAADTATYFCARAA |
| | AGAYTDNIVFGGGTEVVVK | FTAYNRGSCVIHRLDLWGQGTLVTVSS |
| | (SEQ ID NO: 74) | (SEQ ID NO: 93) |
| Rbt22C4 | AQVLTQTASSVSAAVGGTVTISCQSSPSVY | QSVEESGGRLVTPGTPLTLTCTVSGFSLSTYS |
| | NHNWLSWYQQKPGQPPKLLIYEASKLASGV | MSWVRQAPGKGLEWLGIVSVAIDPVYATWARG |
| | PSRFSGSGSGTQFTLTISDVQCDEAATYYC | RFTISRTSTTVNLKITSPTTEDTATYFCVRVA |
| | AGGFSSGSDSFAFGGGTEVVVT | FSTNGIPHRLDLWGQGTLVTVSS |
| | (SEQ ID NO: 75) | (SEQ ID NO: 94) |

Antibodies binding Ig-like domains

| | | |
|---|---|---|
| Rbt11G11 | DPVLTQTPSSVEAAVGGTVTIKCQASESIS | QELVESGGGLVQAGESLKLSCKASGIDFSSYG |
| | SRLAWYQQKPGQPPKLLIYSASTLASGVSS | ISWVRQAPGKGLEWIAYIYPGFGITNYAHSVK |
| | RFKGSGSGTEFTLTISDLECADAATYYCQT | GRFTISSDNAQNTVFLQMPSLTASDTATYFCA |
| | YYGGSTTGWYVFGGGTEVVVK | RDLDYTGGVVGYAYVTYYFTLWGPGTLVTVSS |
| | (SEQ ID NO: 76) | (SEQ ID NO: 95) |
| Rbt12H4 | DVVMTQTPASVEAAVGGTVTIKCQASQSIG | QSVEESGGRLVTPGTPLTVTCTVSGFSLSVYG |
| | NALAWYQQKPGQRPKLLIYAASNLASGVPS | MGWVRQAPGKGLEYIGFINNVGNTYYASWAKG |
| | RFAGSGSGTQFTLTISDLECADAATYYCQT | RFTISKTSTTVDLKITSPTTEDTATYFCAKGG |
| | YYAINRYGGAFGGGTEVVVK | GGDWGYFNIWGPGTLVTVSL |
| | (SEQ ID NO: 77) | (SEQ ID NO: 96) |
| Rbt13B4 | DVVMTQTPASVSEPVGGTVTIKCQASQNIY | QSVEESGGRLVTPGTPLTLTCTVSGFSLSSYA |
| | SGLAWYQQKPGQPPKLLIYGASKLASGVSS | MGWVRQAPGKGLEWIGIINSYGNTYYANWAKG |
| | RFKGSGSGTEFTLTISDLECADAATYYCQA | RFTISRTSTTVDLRMPSLTTEDTATYFCARDP |
| | TYYSSNSVAFGGGTEVVVK | GVSSNLWGPGTLVTVSS |
| | (SEQ ID NO: 78) | (SEQ ID NO: 97) |
| h13B4.v1 | DVQMTQSPSTLSASVGDRVTITCQASQNIY | EQQLVESGEGLVQPGGSLRLSCAVSGFSLSSY |
| | SGLAWYQQKPGKPPKLLIYGASKLASGVPS | AMGWVRQAPGKGLEWIGIINSYGNTYYANWAK |
| | RFSGSGSGTEFTLTISSLQPDDFATYYCQA | GRFTISRDSSKNTVYLQMGSLRAEDMAVYFCA |
| | TYYSSNSVAFGGGTKVEIK | RDPGVSSNLWGPGTLVTVSS |
| | (SEQ ID NO: 79) | (SEQ ID NO: 98) |
| h13B4.v16 | DIQMTQSPSTLSASVGDRVTITCQASQNIY | EVQLVESGEGLVQPGGSLRLSCAASGFSLSSY |
| | SGLAWYQQKPGKAPKLLIYGASKLASGVPS | AMGWVRQAPGKGLEYVGIINSYGNTYYANWAK |
| | RFSGSGSGTEFTLTISSLQPDDFATYYCQA | GRFTISRDNSKNTVYLQMGSLRAEDMAVYYCA |
| | TYYSSNSVAFGGGTKVEIK | RDPGVSSNLWGRGTLVTVSS |
| | (SEQ ID NO: 80) | (SEQ ID NO: 99) |
| Rbt14C9 | DPVLTQTPASVSEPVGGTVTIKCQASQSIS | QSVEESGGRLVTPGTPLTLTCTVSGIDLSANT |
| | SSLAWYQQKPGQPPKLLIYAASILASEISS | MNWVRQAPGKGLEWIGIFTATGSTYYATWVNG |
| | RFKGSRSGTEFTLTISDLECADAATYYCQC | RFTISKTSTTVDLKITSPTTEDTATYFCARSG |
| | TSYGSLFLGPFGGGTEVVVK | SGSSSGAFNIWGPGTLVTVSL |
| | (SEQ ID NO: 81) | (SEQ ID NO: 100) |
| Rbt18G7 | DIVMTQTPASVEAAVGGTVTIKCQASQSIS | QSLEESGGRLVTPGTPLTLTCTVSGIDLSSYA |
| | NFLAWYQQKPGQPPKVLIYAASHLASGVPS | LGWVRQAPGKGLEYIGIISSTGTTYYATWAKG |
| | RFKGSGSGTQFTLTISDLECADAATYYCQS | RFTISKTSSTTVDLKITGPTTEDTATYFCARG |
| | YFYSSTSIYNAFGGGTEVVVR | AYAGYVAFGPYYFHIWGPGTLVTISL |
| | (SEQ ID NO: 82) | (SEQ ID NO: 101) |

TABLE 8

| Name | Heavy Chain Sequence | Light Chain Sequence |
|---|---|---|
| h9E3.FN.v1 | EQQLVESGGGLIQPGGSLRLSCAVSGFSLSS | DAQLTQSPSTLSASVGDRVTITCQSSKSIYNNNWL |
| Human | NAMSWVRQAPGKGLEWIGIISSSGSTYSAS | SWYQQKPGKPPKLLIYDASDLASGVPSRFSGSGS |
| LALAPG | WAKGRFTISKDSSKNTMYLQMNSLRAEDT | GTEFTLTISSLQPDDFATYYCAGGYSGDSDYAFG |
| | AVYFCARVGFFVGYGAYDYGIIHRLDLWG | GGTKVEIKRTVAAPSVFIFPPSDEQLKSGTASVVC |
| | QGTLVTVSSASTKGPSVFPLAPSSKSTSGGT | LLNNFYPREAKVQWKVDNALQSGNSQESVTEQD |
| | AALGCLVKDYFPEPVTVSWNSGALTSGVH | SKDSTYSLSSTLTLSKADYEKHKVYACEVTHQGL |
| | TFPAVLQSSGLYSLSSVVTVPSSSLGTQTYI | SSPVTKSFNRGEC (SEQ ID NO: 110) |
| | CNVNHKPSNTKVDKKVEPKSCDKTHTCPP | |
| | CPAPEAAGGPSVFLFPPKPKDTLMISRTPEV | |
| | TCVVVDVSHEDPEVKFNWYVDGVEVHNA | |
| | KTKPREEQYNSTYRVVSVLTVLHQDWLNG | |
| | KEYKCKVSNKALGAPIEKTISKAKGQPREP | |
| | QVYTLPPSREEMTKNQVSLTCLVKGFYPSD | |
| | IAVEWESNGQPENNYKTTPPVLDSDGSFFL | |
| | YSKLTVDKSRWQQGNVFSCSVMHEALHN | |
| | HYTQKSLSLSPG (SEQ ID NO: 102) | |
| h9E3.FN.v16 | EVQLVESGGGLIQPGGSLRLSCAASGFSLSS | DIQLTQSPSTLSASVGDRVTITCQSSKSIYNNNWL |
| Human | NAMSWVRQAPGKGLEWVSIISSSGSTYSAS | SWYQQKPGKPPKLLIYDASDLASGVPSRFSGSGS |
| LALAPG | WAKGRFTISKDNSKNTLYLQMNSLRAEDT | GTEFTLTISSLQPDDFATYYCAGGYSGDSDYAFG |
| | AVYYCARVGFFVGYGAYDYGIIHRLDLWG | GGTKVEIKRTVAAPSVFIFPPSDEQLKSGTASVVC |
| | QGTLVTVSSASTKGPSVFPLAPSSKSTSGGT | LLNNFYPREAKVQWKVDNALQSGNSQESVTEQD |
| | AALGCLVKDYFPEPVTVSWNSGALTSGVH | SKDSTYSLSSTLTLSKADYEKHKVYACEVTHQGL |
| | TFPAVLQSSGLYSLSSVVTVPSSSLGTQTYI | SSPVTKSFNRGEC (SEQ ID NO: 111) |
| | CNVNHKPSNTKVDKKVEPKSCDKTHTCPP | |
| | CPAPEAAGGPSVFLFPPKPKDTLMISRTPEV | |
| | TCVVVDVSHEDPEVKFNWYVDGVEVHNA | |
| | KTKPREEQYNSTYRVVSVLTVLHQDWLNG | |
| | KEYKCKVSNKALGAPIEKTISKAKGQPREP | |
| | QVYTLPPSREEMTKNQVSLTCLVKGFYPSD | |
| | IAVEWESNGQPENNYKTTPPVLDSDGSFFL | |
| | YSKLTVDKSRWQQGNVFSCSVMHEALHN | |
| | HYTQKSLSLSPG (SEQ ID NO: 103) | |
| h10C3.v1 | EQQLVESGGGLVQPGGSLRLSCAASGFSLS | DQVLTQSPDSLAVSLGERATINCQSSESVYNNDY |
| Human | GYTMGWVRQAPGKGLEYIGVISSGGTTYY | LAWYQQKPGQPPKLLIYSASTLASGVPDRFSGSG |
| LALAPG | TNWAKGRFTISKDSSKNTVYLQMGSLRAE | SGTDFTLTISSLQAEDVAVYYCAGGYLGNNVFGG |
| | DMAVYFCARVAFTAYGGGGFPTLHRLDL | GTKVEIKRTVAAPSVFIFPPSDEQLKSGTASVVCL |
| | WGQGTLVTVSSASTKGPSVFPLAPSSKSTS | LNNFYPREAKVQWKVDNALQSGNSQESVTEQDS |
| | GGTAALGCLVKDYFPEPVTVSWNSGALTS | KDSTYSLSSTLTLSKADYEKHKVYACEVTHQGLS |
| | GVHTFPAVLQSSGLYSLSSVVTVPSSSLGTQ | SPVTKSFNRGEC (SEQ ID NO: 112) |
| | TYICNVNHKPSNTKVDKKVEPKSCDKTHT | |
| | CPPCPAPEAAGGPSVFLFPPKPKDTLMISRT | |
| | PEVTCVVVDVSHEDPEVKFNWYVDGVEV | |
| | HNAKTKPREEQYNSTYRVVSVLTVLHQDW | |
| | LNGKEYKCKVSNKALGAPIEKTISKAKGQP | |
| | REPQVYTLPPSREEMTKNQVSLTCLVKGFY | |
| | PSDIAVEWESNGQPENNYKTTPPVLDSDGS | |
| | FFLYSKLTVDKSRWQQGNVFSCSVMHEAL | |
| | HNHYTQKSLSLSPG (SEQ ID NO: 104) | |
| h10C3.v14 | EVQLVESGGGLVQPGGSLRLSCAASGFSLS | DQVLTQSPDSLAVSLGERATINCQSSESVYNNDY |
| Human | GYTMGWVRQAPGKGLEYIGVISSGGTTYY | LAWYQQKPGQPPKLLIYSASTLASGVPDRFSGSG |
| LALAPG | TNWAKGRFTISKDNSKNTLYLQMGSLRAE | SGTDFTLTISSLQAEDVAVYYCAGGYLGNNVFGG |
| | DMAVYYCARVAFTAYGGGGFPTLHRLDL | GTKVEIKRTVAAPSVFIFPPSDEQLKSGTASVVCL |
| | WGQGTLVTVSSASTKGPSVFPLAPSSKSTS | LNNFYPREAKVQWKVDNALQSGNSQESVTEQDS |
| | GGTAALGCLVKDYFPEPVTVSWNSGALTS | KDSTYSLSSTLTLSKADYEKHKVYACEVTHQGLS |
| | GVHTFPAVLQSSGLYSLSSVVTVPSSSLGTQ | SPVTKSFNRGEC (SEQ ID NO: 113) |
| | TYICNVNHKPSNTKVDKKVEPKSCDKTHT | |
| | CPPCPAPEAAGGPSVFLFPPKPKDTLMISRT | |
| | PEVTCVVVDVSHEDPEVKFNWYVDGVEV | |
| | HNAKTKPREEQYNSTYRVVSVLTVLHQDW | |
| | LNGKEYKCKVSNKALGAPIEKTISKAKGQP | |
| | REPQVYTLPPSREEMTKNQVSLTCLVKGFY | |
| | PSDIAVEWESNGQPENNYKTTPPVLDSDGS | |
| | FFLYSKLTVDKSRWQQGNVFSCSVMHEAL | |
| | HNHYTQKSLSLSPG (SEQ ID NO: 105) | |
| h10F7.v1 | EQQLVESGGGLVQPGGSLRLSCAVSGFSLS | DAVLTQSPDSLAVSLGERATINCQSSKSVYNNNW |
| Human | GYAMSWVRQAPGKGLEYIGVISSSGSSYYP | LSWYQQKPGQPPKLLIYRASTLESGVPDRFSGSGS |
| LALAPG | SWAKGRFTISKDSSKNTVYLQMGSLRAED | GTDFTLTISSLQAEDVAVYFCAGGYSSSSSANAFG |
| | MAVYFCARVQFYVGYAVYGYGIIDRLDL | GGTKVEIKRTVAAPSVFIFPPSDEQLKSGTASVVC |
| | WGQGTLVTVSSASTKGPSVFPLAPSSKSTS | LLNNFYPREAKVQWKVDNALQSGNSQESVTEQD |
| | GGTAALGCLVKDYFPEPVTVSWNSGALTS | SKDSTYSLSSTLTLSKADYEKHKVYACEVTHQGL |
| | GVIITFPAVLQSSGLYSLSSVVTVPSSSLGTQ | SSPVTKSFNRGEC (SEQ ID NO: 114) |
| | TYICNVNHKPSNTKVDKKVEPKSCDKTHT | |
| | CPPCPAPEAAGGPSVFLFPPKPKDTLMISRT | |
| | PEVTCVVVDVSHEDPEVKFNWYVDGVEV | |
| | HNAKTKPREEQYNSTYRVVSVLTVLHQDW | |
| | LNGKEYKCKVSNKALGAPIEKTISKAKGQP | |
| | REPQVYTLPPSREEMTKNQVSLTCLVKGFY | |
| | PSDIAVEWESNGQPENNYKTTPPVLDSDGS | |
| | FFLYSKLTVDKSRWQQGNVFSCSVMHEAL | |
| | HNHYTQKSLSLSPG (SEQ ID NO: 106) | |
| h10F7.v16 | EVQLVESGGGLVQPGGSLRLSCAVSGFSLS | DIVLTQSPDSLAVSLGERATINCQSSKSVYNNNW |
| Human | GYAMSWVRQAPGKGUEYIGVISSSGSSYYP | LSWYQQKPGQPPKLLIYRASTLESGVPDRFSGSGS |
| LALAPG | SWAKGRFTISKDNSKNTLYLQMGSLRAED | GTDFTLTISSLQAEDVAVYFCAGGYSSSSSANAFG |
| | MAVYYCARVQFYVGYAVYGYGIIDRLDL | GGTKVEIKRTVAAPSVFIFPPSDEQLKSGTASVVC |
| | WGQGTLVTVSSASTKGPSVFPLAPSSKSTS | LLNNFYPREAKVQWKVDNALQSGNSQESVTEQD |
| | GGTAALGCLVKDYFPEPVTVSWNSGALTS | SKDSTYSLSSTLTLSKADYEKHKVYACEVTHQGL |
| | GVHTFPAVLQSSGLYSLSSVVTVPSSSLGTQ | SSPVTKSFNRGEC (SEQ ID NO: 115) |
| | TYICNVNHKPSNTKVDKKVEPKSCDKTHT | |
| | CPPCPAPEAAGGPSVFLFPPKPKDTLMISRT | |
| | PEVTCVVVDVSHEDPEVKFNWYVDGVEV | |
| | HNAKTKPREEQYNSTYRVVSVLTVLHQDW | |
| | LNGKEYKCKVSNKALGAPIEKTISKAKGQP | |
| | REPQVYTLPPSREEMTKNQVSLTCLVKGFY | |
| | PSDIAVEWESNGQPENNYKTTPPVLDSDGS | |
| | FFLYSKLTVDKSRWQQGNVFSCSVMHEAL | |
| | HNHYTQKSLSLSPG (SEQ ID NO: 107) | |
| h13B4.v1 | EQQLVESGEGLVQPGGSLRLSCAVSGFSLS | DVQMTQSPSTLSASVGDRVTITCQASQNIYSGLA |
| Human | SYAMGWVRQAPGKGLEWIGIINSYGNTYY | WYQQKPGKPPKLLIYGASKLASGVPSRFSGSGSG |
| LALAPG | ANWAKGRFTISRDSSKNTVYLQMGSLRAE | TEFTLTISSLQPDDFATYYCQATYYSSNSVAFGGG |
| | DMAVYFCARDPGVSSNLWGPGTLVTVSSA | TKVEIKRTVAAPSVFEFPPSDEQLKSGTASVVCLL |
| | STKGPSVFPLAPSSKSTSGGTAALGCLVKD | NNFYPREAKVQWKVDNALQSGNSQESVTEQDSK |
| | YFPEPVTVSWNSGALTSGVHTFPAVLQSSG | DSTYSLSSTLTLSKADYEKHKVYACEVTHQGLSS |
| | LYSLSSVVTVPSSSLGTQTYICNVNHKPSNT | PVTKSFNRGEC (SEQ ID NO: 116) |
| | KVDKKVEPKSCDKTHTCPPCPAPEAAGGPS | |
| | VFLFPPKPKDTLMISRTPEVTCVVVDVSHE | |
| | DPEVKFNWYVDGVEVHNAKTKPREEQYN | |
| | STYRVVSVLTVLHQDWLNGKEYKCKVSN | |
| | KALGAPIEKTISKAKGQPREPQVYTLPPSRE | |
| | EMTKNQVSLTCLVKGFYPSDIAVEWESNG | |
| | QPENNYKTTPPVLDSDGSFFLYSKLTVDKS | |
| | RWQQGNVFSCSVMHEALHNHYTQKSLSLS | |
| | PG (SEQ ID NO: 108) | |
| h13B4.v16 | EVQLVESGEGLVQPGGSLRLSCAASGFSLS | DIQMTQSPSTLSASVGDRVTITCQASQNIYSGLA |
| Human | SYAMGWVRQAPGKGLEYVGIINSYGNTYY | WYQQKPGKAPKLLIYGASKLASGVPSRFSGSGSG |
| LALAPG | ANWAKGRFTISRDNSKNTVYLQMGSLRAE | TEFTLTISSLQPDDFATYYCQATYYSSNSVAFGGG |
| | DMAVYYCARDPGVSSNLWGRGTLVTVSS | TKVEIKRTVAAPSVFIFPPSDEQLKSGTASVVCLL |
| | ASTKGPSVFPLAPSSKSTSGGTAALGCLVK | NNFYPREAKVQWKVDNALQSGNSQESVTEQDSK |
| | DYFPEPVTVSWNSGALTSGVHTFPAVLQSS | DSTYSLSSTLTLSKADYEKHKVYACEVTHQGLSS |
| | GLYSLSSVVTVPSSSLGTQTYICNVNHKPSN | PVTKSFNRGEC (SEQ ID NO: 117) |
| | TKVDKKVEPKSCDKTHTCPPCPAPEAAGGP | |
| | SVFLFPPKPKDTLMISRTPEVTCVVVDVSHE | |
| | DPEVKFNWYVDGVEVHNAKTKPREEQYN | |
| | STYRVVSVLTVLHQDWLNGKEYKCKVSN | |
| | KALGAPIEKTISKAKGQPREPQVYTLPPSRE | |
| | EMTKNQVSLTCLVKGFYPSDIAVEWESNG | |
| | QPENNYKTTPPVLDSDGSFFLYSKLTVDKS | |
| | RWQQGNVFSCSVMHEALHNHYTQKSLSLS | |
| | PG (SEQ ID NO: 109) | |

In certain embodiments, each of SEQ ID NOs: 102-109 may optionally comprise a lysine (K) at the C-terminal end of the amino acid sequence, e.g., each sequence may end in PGK rather than in PG.

### Example 2: Antibody Binding Affinity

Each rabbit and humanized antibody was subjected to a binding assay to determine its affinity to MerTK derived from various species.

All binding affinity determinations were obtained using Surface Plasmon Resonance (SPR) measurements from a BIAcore™-T200 instrument. Briefly, each rabbit or humanized antibody was captured to achieve approximately 100 RU (Response Units). Then, 3-fold serial dilutions of MerTK from various species (0.4 nM to 100 nM) diluted in HBS-EP buffer (0.01M HEPES pH 7.4, 0.15M NaCl, 3 mM EDTA and 0.05% v/v surfactant P20) was injected into the BIAcore™-T200 instrument at 25° C. or 37° C. with a flow rate of 30 μl/min. Association rates (kₒₙ) and dissociation rates (k_{off}) were calculated using a simple one-to-one Langmuir binding model (BIAcore T200 evaluation software version 2.0). The equilibrium dissociation constant (K_{D}) was calculated as the ratio k_{off}/kₒₙ.

TABLE 9 shows the equilibrium dissociation constant, K_{D}, measured via BIAcore analysis for each rabbit anti-MerTK antibody binding to human, cynomolgus monkey, and mouse MerTK protein. TABLES 10-13 compare the K_{D} measured for rabbit anti-MerTK monoclonal antibodies to their matched antibodies after the first step of humanization (V1). TABLES 14-17 compare the K_{D} for each antibody binding to human, cynomolgus monkey, rat, and mouse MerTK protein, after the final step of humanization (humanized polished mAb) to the K_{D} of the same antibody after the first step of humanization (V1). The polished humanized mAb are h10C3.v14, h9E3.FN.v1, h10F7.v16, and h13B4.v16 respectively.

TABLE 9

| | BIAcore (KD:nM) at 25° C. | | |
|---|---|---|---|
| MerTK | Human | Cyno | Mouse |
| Antibody | MerTK | MerTK | MerTK |
| Rbt8F4 | 44 | 14.7 | 2.3 |
| Rbt9E3.FN | 2.6 | 2.5 | 0.6 |
| Rbt10C3 | 3.4 | 3.0 | 0.7 |
| Rbt10F7 | 7.0 | 4.7 | 4.1 |
| Rbt11G11 | 31.3 | 14.3 | 4.1 |
| Rbt12H4 | 18 | 13.9 | 8.5 |
| Rbt13B4 | 2.9 | 1.8 | >1000 |
| Rbt13D8 | 4.3 | 3.8 | 1.2 |
| Rbt14C9 | >1000 | NA | 0.6 |
| Rbt18G7 | 2.3 | 4.3 | 1.7 |
| Rbt22C4 | 94 | 82.2 | 2.2 |

TABLE 10

| | Clone | | | |
|---|---|---|---|---|
| | 10C3 (FN Binder) at 37° C. | | | |
| | Human | Cyno | Rat | Mouse |
| | K_{D}: | K_{D}: | K_{D}: | K_{D}: |
| MerTK | nM | nM | nM | nM |
| Rabbit mAb | 9.9 | 4.1 | 2.4 | 0.8 |
| Humanized | 12.9 | 5.3 | 3.3 | 1.1 |
| mAb V1 | | | | |

TABLE 11

| | Clone | | | |
|---|---|---|---|---|
| | 9E3.FN (FN Binder) at 37° C. | | | |
| | Human | Cyno | Rat | Mouse |
| | K_{D}: | K_{D}: | K_{D}: | K_{D}: |
| MerTK | nM | nM | nM | nM |
| Rabbit mAb | 5.1 | 2.5 | 1.7 | 0.6 |
| Humanized | 10.6 | 4.5 | 3.7 | 1.5 |
| mAb V1 | | | | |

TABLE 12

| | Clone | | | |
|---|---|---|---|---|
| | 10F7 (FN Binder) at 37° C. | | | |
| | Human | Cyno | Rat | Mouse |
| | K_{D}: | K_{D}: | K_{D}: | K_{D}: |
| MerTK | nM | nM | nM | nM |
| Rabbit mAb | 10.9 | 5.1 | 5.8 | 2.4 |
| Humanized | 7.1 | 3.1 | 3.6 | 1.5 |
| mAb V1 | | | | |

TABLE 13

| | Clone | | | |
|---|---|---|---|---|
| | 13B4 (Ig Binder) at 37° C. | | | |
| | Human | Cyno | Rat | Mouse |
| | K_{D}: | K_{D}: | K_{D}: | K_{D}: |
| MerTK | nM | nM | nM | nM |
| Rabbit mAb | 4.9 | 5.9 | >100 | >100 |
| Humanized | 4.8 | 5.6 | >100 | >100 |
| mAb V1 | | | | |

TABLE 14

| | Clone | | | |
|---|---|---|---|---|
| | 10C3 (FN Binder) at 37° C. | | | |
| | Human | Cyno | Rat | Mouse |
| | K_{D}: | K_{D}: | K_{D}: | K_{D}: |
| MerTK | nM | nM | nM | nM |
| Humanized | 11.6 | 5.3 | 3.3 | 1 |
| V1 mAb | | | | |
| *Humanized | 6.9 | 3.2 | 2.4 | 0.9 |
| polished mAb | | | | |

TABLE 15

| | Clone | | | |
|---|---|---|---|---|
| | 9E3.FN (FN Binder) at 37° C. | | | |
| | Human | Cyno | Rat | Mouse |
| | K_{D}: | K_{D}: | K_{D}: | K_{D}: |
| MerTK | nM | nM | nM | nM |
| Humanized | 11.2 | 4.8 | 3.8 | 1.4 |
| V1 mAb | | | | |
| *Humanized | 5.8 | 2.5 | 3 | 1.1 |
| polished mAb | | | | |

TABLE 16

| | Clone | | | |
|---|---|---|---|---|
| | 10F7 (FN Binder) at 37° C. | | | |
| | Human | Cyno | Rat | Mouse |
| | K_{D}: | K_{D}: | K_{D}: | K_{D}: |
| MerTK | nM | nM | nM | nM |
| Humanized | 6.7 | 3 | 4.1 | 1.5 |
| V1 mAb | | | | |
| *Humanized | 4.9 | 2.1 | 3.2 | 1.4 |
| polished mAb | | | | |

TABLE 17

| | Clone | | | |
|---|---|---|---|---|
| | 13B4 (Ig Binder) at 37° C. | | | |
| | Human | Cyno | Rat | Mouse |
| | K_{D}: | K_{D}: | K_{D}: | K_{D}: |
| MerTK | nM | nM | nM | nM |
| Humanized | 4.3 | 5 | >100 | >100 |
| V1 mAb | | | | |
| *Humanized | 5.1 | 5.7 | >100 | >100 |
| polished mAb | | | | |

The results confirmed that most of the rabbit antibodies are cross species MerTK binders, except for 14C9, which is a mouse specific MerTK binder and 13B4, which is a human specific MerTK binder. The results further indicated that after step 1 humanization, there is a slight affinity improvement against all four species of MerTK for antibody 10F7, but not for 10C3 and 9E3.FN, which show slight affinity drop. For antibody 13B4, it is comparable before and after humanization. After step 2 of humanization, affinity improved against all four species of MerTK for 10C3, 9E3.FN, and 10F7, but not for 13B4.

### Example 3: Antibody Epitope Characterization

The isolated anti-MerTK antibodies were characterized by epitope binning and binding analysis to determine epitope domain specificity.

### Epitope Binning

A 96×96 array-based SPR imaging system (Carterra USA) was used to epitope bin a panel of MerTK monoclonal antibodies. First, each anti-MerTK rabbit antibody, diluted at 10 ug/ml in 10 mM sodium acetate buffer pH4.5, was directly immobilized onto a SPR sensorprism CMD 200M sensor chip (XanTec Bioanalytics, Germany) using amine-coupling chemistry in a Continuous Flow Microspotter (Carterra, USA). Then, MerTK, at 100 nM, was injected over the sensor chip for 4 minutes to allow binding, followed by another 4 minute binding of each binning rabbit antibody at 10 ug/ml.

The surface was regenerated between each cycle using 10 mM Glycine pH1.5, and the experiment was conducted at 25° C. using HBS-EP buffer (0.01M HEPES pH 7.4, 0.15M NaCl, 3 mM EDTA and 0.05% surfactant P20). The IBIS MX96 SPRi instrument (Carterra USA) was used to record the binding response to the immobilized antibodies. The binding data was analyzed using Wasatch binning software tool to generate an epitope network plot.

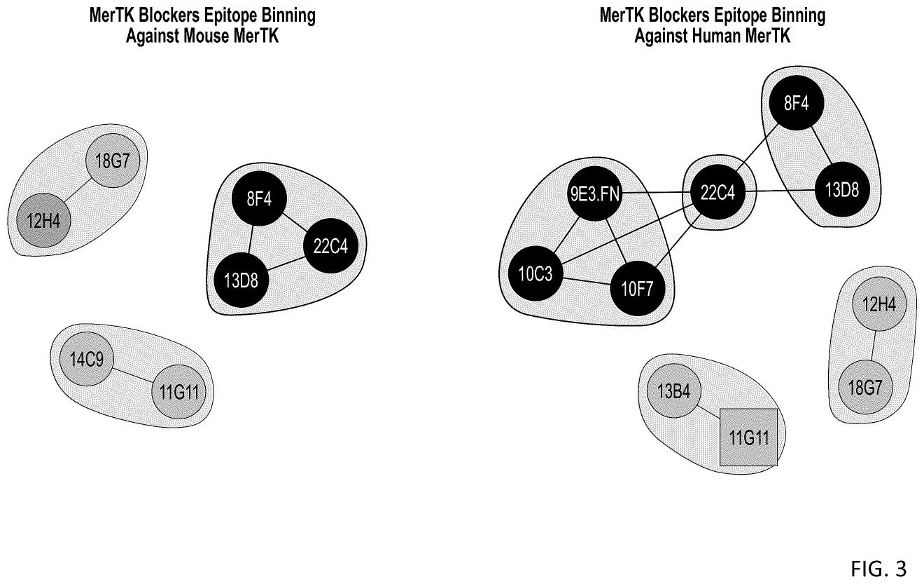

The results of the binning experiment in FIG. 3 indicate which antibodies compete for binding with each other on certain MerTK epitopes. Antibodies 8F4, 22C4, and 13D8, raised against mouse MerTK, and antibodies 10C3, 9E3.FN, 10F7, 22C4, 8F4, and 13D8, raised against human MerTK, competed for binding with each other (FIG. 3). Antibodies 12H4, 18G7, 14C9, and 11G11, raised against mouse MerTK, and antibodies 13B4, 12H4, 18G7, and 1 IG11, raised against human MerTK, competed with each other (FIG. 3). As described below, antibodies 10C3, 9E3.FN, 10F7, 22C4, 8F4, and 13D8 bind to MerTK's fibronectin-like domain, and antibodies 13B4, 12H4, 18G7, and 11G11 bind to MerTK's Ig-like domain.

### Epitope Binding Analysis

Epitope specificity of the rabbit antibodies was also determined by binding experiments. Each rabbit antibody was tested for binding to four domains from human MerTK or mouse MerTK: the extracellular domain (HuMER R26-A499 or MuMER E23-S496), which includes both Ig-like domains and both fibronectin-like domains, the Ig-like 1&2 domains (HuMER G76-P284 or MuMER A70-P279), the Ig-like 1 domain (HuMER G76-G195 or MuMER A70-G190), and the Ig-like 2 domain (HuMER G195-P284 or MuMER G190-P279).

Binding affinity determinations were obtained using Surface Plasmon Resonance (SRP) measurements from a BIAcore™-T200 instrument. Briefly, each rabbit antibody was captured to achieve approximately 100 RU (Response Units). Then, 3-fold serial dilutions of the various MerTK domains (0.4 nM to 100 nM) diluted in HBS-EP buffer (0.01M HEPES pH 7.4, 0.15M NaCl, 3 mM EDTA and 0.05% v/v surfactant P20) was injected into the BIAcore™-T200 instrument at 25° C. or 37° C. with a flow rate of 30 μl/min. Association rates (kₒₙ) and dissociation rates (k_{off}) were calculated using a simple one-to-one Langmuir binding model (BIAcore T200 evaluation software version 2.0). The equilibrium dissociation constant (K_{D}) was calculated as the ratio k_{off}/kₒₙ.

Antibody epitope determination was assessed by BIAcore analysis for rabbit antibodies binding against both the human and mouse MerTK extracellular domain (HuMER R26-A499 and MuMER E23-S496), Ig1&2 domain, Ig1-only domain, and Ig2-only domain (TABLE 18). Human MerTK and its domains are shown in light gray, while mouse MerTK and its domains are shown in dark gray.

The results of the epitope binding analysis demonstrate that cross-reactive FN domain antibodies, Rbt8F4, Rbt22C4 and Rbt13D8, do not bind human or mouse Ig1 and Ig2 domains at 1 uM (TABLE 18). Epitope binding data was not collected for antibodies Rbt9E3.FN, Rbt10C3 and Rbt10F7. However, Wasatch binning demonstrated that the epitope-specificity of Rbt9E3.FN, Rbt10C3 and Rbt10F7 overlaps with FN domain antibodies, Rbt8F4, Rbt22C4 and Rbt13D8 (FIG. 3). Therefore, the results suggested that Rbt9E3.FN, Rbt10C3 and Rbt10F7 are FN binding domain antibodies that do not bind the isolated Ig1 and Ig2 domains.

The results of the epitope binding analysis further demonstrate that antibodies Rbt11G11, Rbt12H4, Rbt18G7, Rbt13B4, and Rbt14C9, are Ig domain binding antibodies (TABLE 18). Antibodies Rbt11G11, Rbt12H4, and Rbt18G7 are cross-reactive Ig domain antibodies that bind both human and mouse MerTK Ig (TABLE 18). In contrast, Rbt13B4 and Rbt14C9 are species-specific Ig domain antibodies which bind human and mouse Ig, respectively (TABLE 18).

TABLE 18

| | | BIAcore Analysis (K_{D}:nM) at 25° C. | | | | | | | |
|---|---|---|---|---|---|---|---|---|---|
| Epitope | | HuMER | HuMER · | HuMER · | HuMER · | MuMER | MuMER · | MuMER · | MuMER · |
| on | MerTK | (R26- | Ig1&2 | Ig1 | Ig2 | (E23- | Ig1&2 | Ig1 | Ig2 |
| HuMER | Antibody | A499) | (G76-P284) | (G76-G195) | (G195-P284) | S496) | (A70-P279) | (A70-G190) | (G190-P279) |
| FN | Rbt8F4 | 44 | >1000 | >1000 | >1000 | 2.3 | >1000 | na | na |
| FN | Rbt22C4 | 94 | >1000 | >1000 | >1000 | 2.2 | >1000 | na | na |
| FN | Rbt13D8 | 4.3 | >1000 | >1000 | >1000 | 1.2 | >1000 | na | na |
| *FN | *Rbt9E3.FN | 2.6 | na | na | na | 0.6 | na | na | na |
| *FN | *Rbt10C3 | 3.4 | na | na | na | 0.7 | na | na | na |
| *FN | *Rbt10F7 | 7.0 | na | na | na | 4.1 | na | na | na |
| Ig1 | Rbt11G11 | 31.3 | >1000 | 39.8 | >1000 | 4.1 | 3.4 | na | na |
| Ig1 | Rbt12H4 | 18 | 92 | 16.7 | >1000 | 8.5 | 11 | 2.2 | >1000 |
| Ig1 | Rbt18G7 | 2.3 | 21 | 0.5 | >1000 | 1.7 | 1.9 | 0.2 | >1000 |
| Ig1 | Rb13B4 | 2.9 | 8.8 | 0.3 | >1000 | >1000 | >1000 | >1000 | >1000 |
| — | Rbt14C9 | >1000 | na | na | na | 0.6 | 0.5 | 0.1 | >1000 |

### Example 4: Anti-MerTK Inhibits Human and Mouse Macrophage Phagocytosis In Vitro

Efferocytosis assays were carried out to evaluate the in vitro macrophage phagocytosis inhibiting activity of anti-MerTK antibodies.

Briefly, efferocytosis, the phagocytosis of apoptotic cells, was quantified using the IncuCyte real time imaging platform. Apoptotic cells were labeled with pH-sensitive probes (pHrodo). pHrodo will only fluoresce in the acidic environment of the phagolysosome once it has been phagocytosed by a macrophage. Phagocytosis events were quantified as total fluorescence intensity (TFI) and normalized by the number of macrophages per well. The maximum normalized TFI observed was designated 100% Phagocytic Activity. The maximum phagocytosis inhibition (0% Phagocytic Activity) was designated as the autofluorescence generated by the pHrodo-labeled apoptotic cells alone in control wells without macrophages.

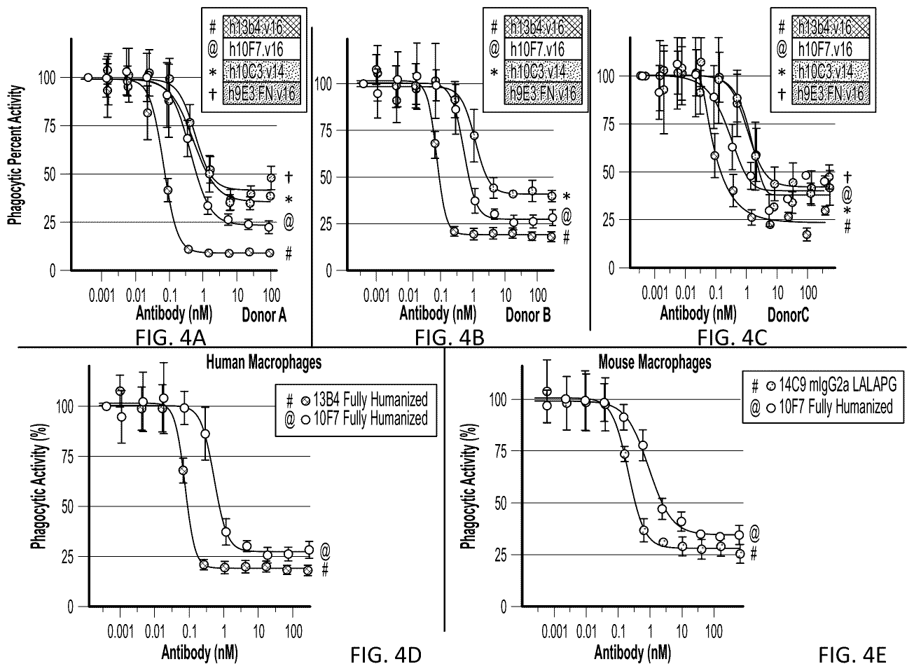

The efferocytosis assays demonstrated that humanized anti-MerTK antibodies can inhibit human macrophage phagocytosis of apoptotic cells (FIGS. 4A, 4B & 4C). The results suggested that humanized antibody h13B4.v16 was the most potent inhibitor of phagocytosis (TABLE 19). Further, anti-MerTK antibody h13B4.v16 (13B4 Fully Humanized), an Ig-domain binding antibody, was found to be 5.2× more potent at inhibiting human macrophage phagocytosis compared to anti-MerTK antibody h10F7.v16 (10F7 Fully Humanized), a fibronectin-domain binding antibody (TABLE 20; FIG. 4D).

In addition, FIG. 4E shows the results of an efferocytosis assay assessing the ability of anti-MerTK antibodies to inhibit mouse macrophage phagocytosis. The results demonstrated that the anti-MerTK antibodies have the ability to block mouse macrophage phagocytosis (FIG. 4E). Further, the results showed that anti-MerTK antibody 14C9 mIgG2a LALAPG, an Ig-domain binding antibody, is 4.8× more potent at inhibiting mouse macrophage phagocytosis compared to anti-MerTK antibody h10F7.v16 (10F7 Fully Humanized), a fibronectin-domain binding antibody (TABLE 21).

TABLE 19

| | Donor A | | Donor B | | Donor C | | Average (3 Donors) | |
|---|---|---|---|---|---|---|---|---|
| Fully | | Max | | Max | | Max | | Max |
| Humanized | IC50 | Inhibition | IC50 | Inhibition | IC50 | Inhibition | IC50 | Inhibition |
| Antibodies | (nM) | (%) | (nM) | (%) | (nM) | (%) | (nM) | (%) |
| h13B4.v16 | 0.07 | 91 | 0.08 | 82 | 0.09 | 77 | 0.08 | 83 |
| h10F7.v16 | 0.42 | 76 | 0.53 | 73 | 0.27 | 60 | 0.41 | 69 |
| h10C3.v14 | 0.52 | 64 | 1.2 | 58 | 1.2 | 62 | 0.95 | 61 |
| h9E3.FN.v16 | 0.59 | 58 | NA | NA | 1.5 | 55 | 1.02 | 57 |

TABLE 20

| | Average (3 Donors) | |
|---|---|---|
| Anti-MerTK Antibodies | IC50 (nM) | Max Inhibition (%) |
| 13B4 Fully Humanized | 0.08 | 83 |
| 10F7 Fully Humanized | 0.41 | 69 |

TABLE 21

| | Average | |
|---|---|---|
| Anti-MerTK Antibodies | IC50 (nM) | Max Inhibition (%) |
| 14C9 mIgG2a LALAPG | 0.19 | 73 |
| 10F7 Fully Humanized | 0.91 | 64 |

### Example 5: Anti-MerTK Inhibits the Clearance of Apoptotic Cells In Vivo

An apoptotic cell clearance assay was carried out to evaluate the in vivo activity of anti-MerTK antibodies (Seitz, H. M. et. al., Macrophages and dendritic cells use different Axl/Mertk/Tyro3 receptors in clearance of apoptotic cells, J Immunol. 178(9) 5635-5642 (2007)).

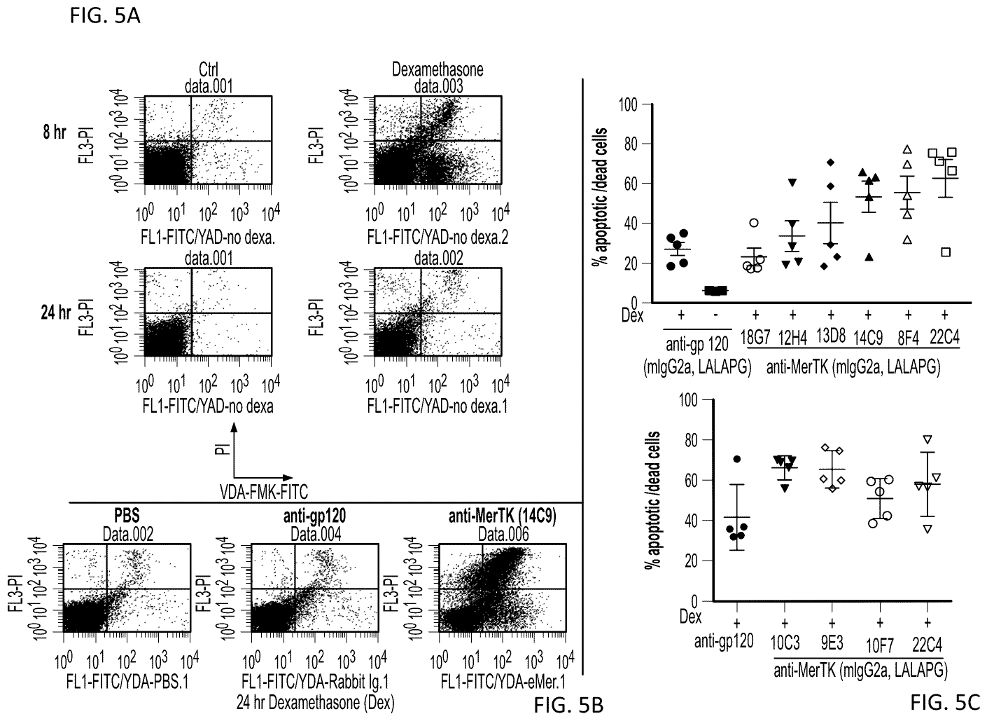

Briefly, 5-7 week-old C57BL/6 mice were injected intraperitoneally with 0.2 mg/25 g dexamethasone (Dex). Eight or twenty-four hours later, the thymus was isolated and dissociated into a single-cell suspension. Cells were stained with VAD-FMK-FITC (1:500 in PBS, Promega, Cat #G7461) to detect active caspase 3-positive apoptotic cells. Propidium iodide was used to stain dead cells (1:1000, Biochemika, Cat #: 70335). The cells were analyzed on a BD FACSCalibur flow cytometer. Accumulation of apoptotic cells were measured by VAD-FMK-FITC single positive cells (early apoptotic cells), and PI/VAD-FMK-FITC double positive cells (late apoptotic cells). FIG. 5A demonstrates that apoptotic cells accumulated 8 hours after Dex treatment and were mostly cleared by 24 hours.

The clearance of apoptotic cells from the thymus is dependent on MerTK expressed on macrophages. Therefore, a panel of function blocking anti-MerTK antibodies was tested for the ability of each antibody to inhibit the clearance of apoptotic/dead cells. Anti-MerTK (clone 14C9, mIgG2a, LALAPG) but not the control antibody anti-gp120 (mIgG2a, LALAPG) blocked the clearance of apoptotic cells in the thymus 24 hours after Dex treatment (FIG. 5B). Quantification of apoptotic/dead cell accumulation in the thymus 24 hours after Dex injection in mice treated with anti-gp120 or anti-MerTK demonstrated that anti-MerTK antibodies blocked the clearance of apoptotic cells relative to the anti-gp120 control (FIG. 5C).

### Example 6: Therapeutic Effect of Anti-MerTK Antibodies in MC-38 Syngeneic Tumor Model

Tumor efficacy studies were carried out in the MC-38 syngeneic tumor model to determine whether anti-MerTK antibodies affect tumor growth.

Age-matched 6-8 week old female C57BL/6 mice were inoculated subcutaneously in the right unilateral flank with 1×10⁵ MC-38 tumor cells suspended in Hanks's Buffered Saline Solution (HBSS) and phenol red-free Matrigel (BD Bioscience). When tumors reached volumes of 150-250 mm³ (day 0), mice were sorted into different treatment groups of n=10. Anti-MerTK antibodies (mIgG2a, LALAPG) or control anti-gp120 (mIgG2a, LALAPG) antibodies were administered at 30 mg kg⁻¹ via intravenous (IV) injection on days 1 and 5, followed by intra-peritoneal (IP) injection on days 9 and 13. Anti-PDL1 antibody was administered at 30 mg kg⁻¹ via IV injection on day 1, followed by IP injection on days 5, 9 and 13 at 5 mg kg⁻¹. Tumor volumes were measured and calculated twice per week using the modified ellipsoid formula ½ (length×width²). Tumors >2,000 mm³ were considered progressed.

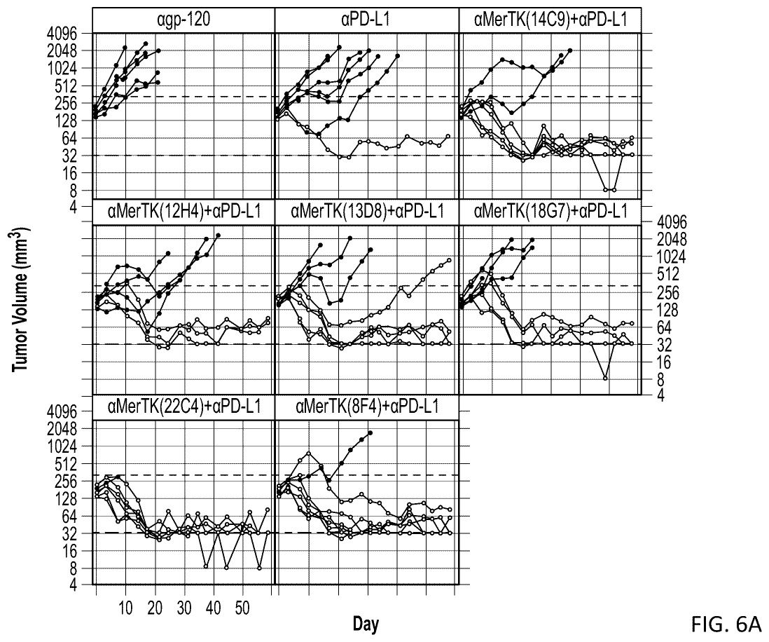

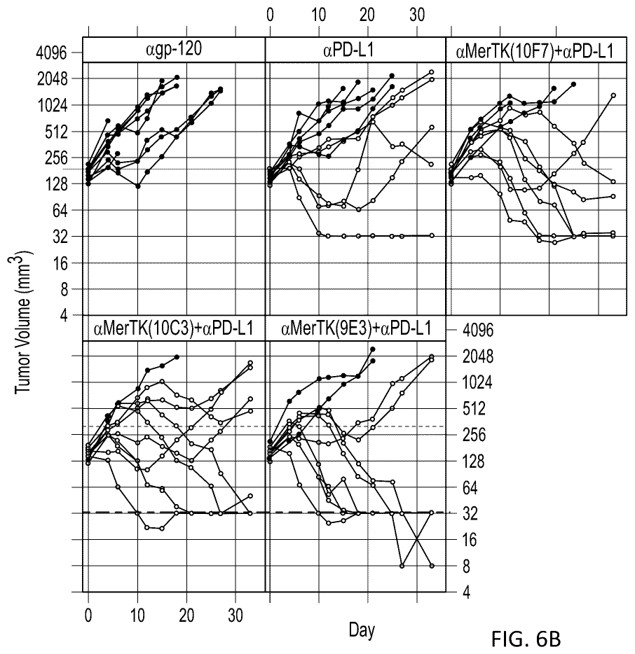

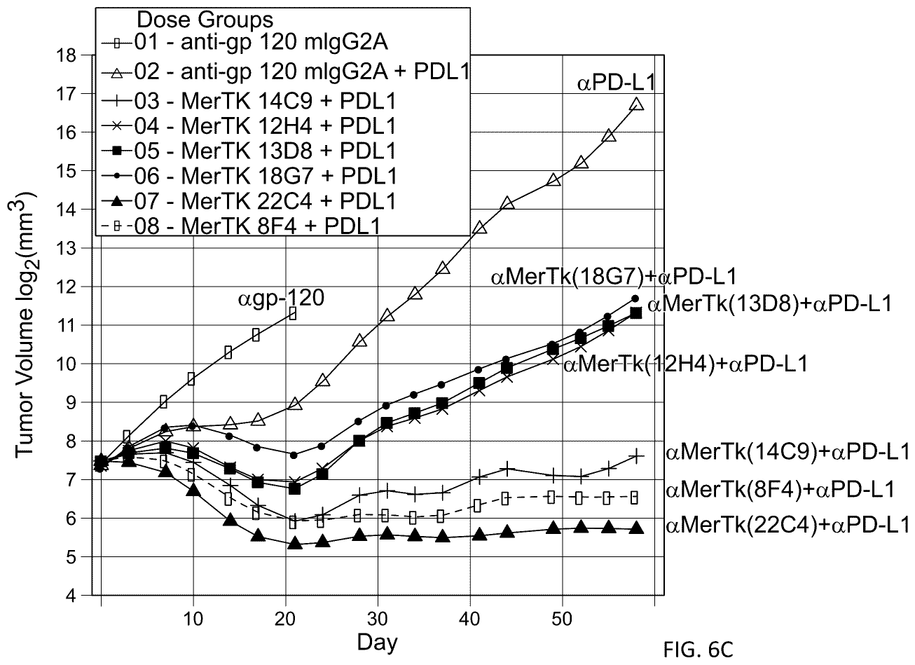

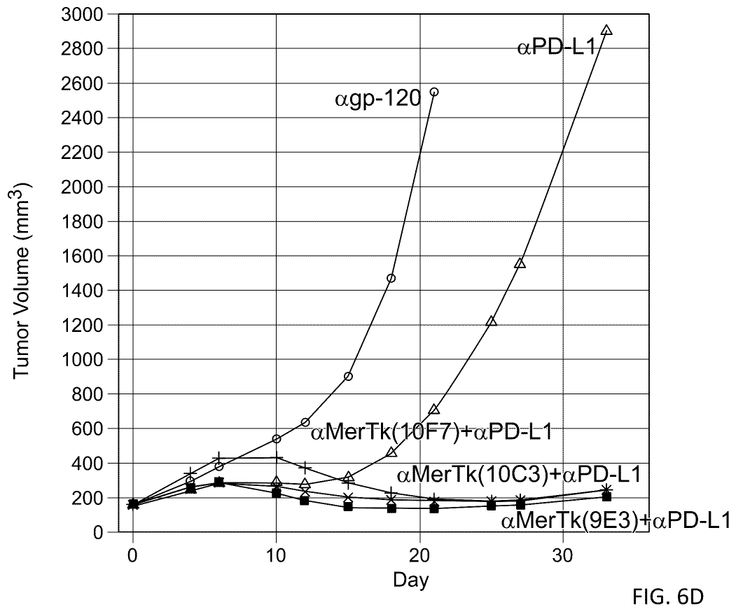

In the tumor volume tracking plots, gray lines represent the tumor size of animals that were still in the study as of the date of data collection (FIGS. 6A & 6B). Red lines represent animals with ulcerated or progressed tumors that were euthanized and removed from study (FIGS. 6A & 6B). Red horizontal dashed lines indicate a doubling in tumor volume from the start of treatment while green horizontal dashed lines represent the smallest measureable tumor volume (FIGS. 6A & 6B). Animals with tumors in the area below the green dashed line were considered to have had a complete response.

As a monotherapy, anti-PDL1, an immune checkpoint inhibitor, exhibited moderate anti-tumor activity (FIGS. 6A-6D). Changes in individual tumor size (FIGS. 6A & 6B) and mean tumor size (FIGS. 6C & 6D), were measured over time for each treatment group. Combination treatment with anti-MerTK antibodies greatly enhanced the anti-tumor efficacy of the anti-PDL1 antibodies (FIGS. 6A-6D).

In the normal physiological context of solid tumors, the rapid removal of dying tumor cells by MerTK-expressing tumor associated macrophages (TAMs) is immunologically silent. Without being bound by theory, it is believed that blockade of MerTK, accomplished in the above-described experiments with anti-MerTK antibodies, could activate the innate proinflammatory response, which in turn could further enhance the adaptive T cell response unleashed by anti-PD-1 therapy.

### Example 7: Anti-MerTK Antibody Reduces Clearance of Apoptotic Thymocytes In Vitro and In Vivo

Efferocytosis assays were carried out to evaluate the in vitro macrophage phagocytosis inhibiting activity of an anti-MerTK antibody (clone 14C9, reformatted into a mIgG2a, LALAPG framework).

For in vitro efferocytosis assays, thymus tissue was harvested from 4-6 week old C57BL/6N mice and minced to yield a single-cell suspension. Apoptosis of thymocytes was induced by 2 M dexamethasone at 37° C. for 5 hours. Membrane integrity and exposure of phosphatidylserine on cell surfaces were assessed using APC Annexin V Apoptosis Detection Kit with PI (Biolegend). Apoptotic thymocytes were labeled with 1 μg/ml pHrodo Red succinimidyl ester. Macrophages were pre-incubated with 30 μg/ml control antibody or anti-MerTK 14C9 (mIgG2a LALAPG) one hour prior to adding pHrodo Red-labeled apoptotic cells. pHrodo will only fluoresce in the acidic environment of the phagolysosome once it has been phagocytosed by a macrophage. After 45 minutes incubation, the remaining apoptotic cells were washed away, and macrophages were labeled with FITC-conjugated anti-CD11b antibody (eBioscience, clone M1/70). After fluorescence images were taken, the cells were detached from the cell culture plate for quantification by FACS analysis.

For in vivo efferocytosis assays, 5-7 week-old C57BL/6N mice were dosed with 20 mg/kg anti-MerTK 14C9 (mIgG2a LALAPG) antibody and then injected intraperitoneally with 0.2 mg/25 g dexamethasone (Dex) one hour later. Eight or twenty-four hours later, the thymus was isolated and dissociated into a single-cell suspension. Cells were stained with VAD-FMK-FITC (1:500 in PBS, Promega, Cat #G7461) to detect active caspase 3-positive apoptotic cells. Propidium iodide was used to stain dead cells (1:1000, Biochemika, Cat #: 70335). The cells were analyzed on a BD FACSCalibur flow cytometer. Accumulation of apoptotic cells were measured by VAD-FMK-FITC single positive cells (early apoptotic cells) and PI/VAD-FMK-FITC double positive cells (late apoptotic cells).

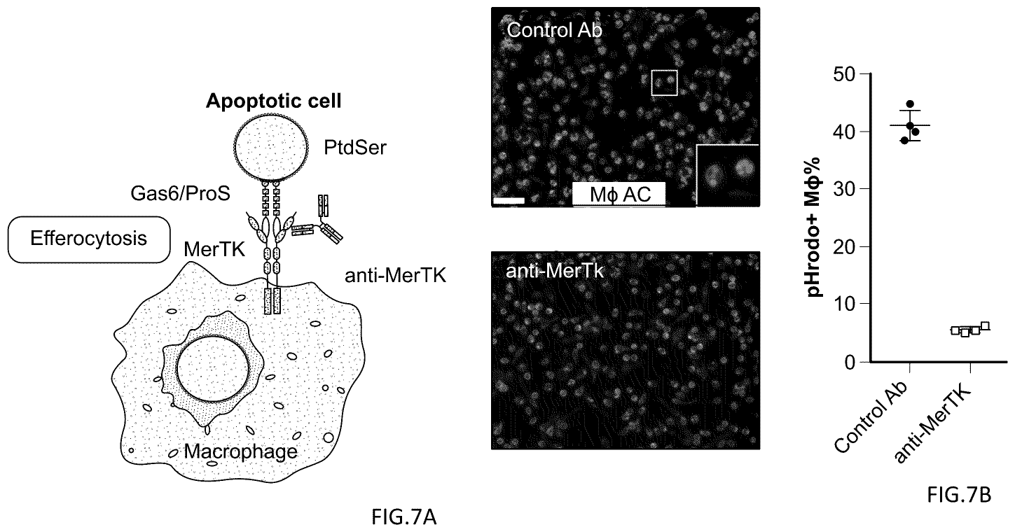

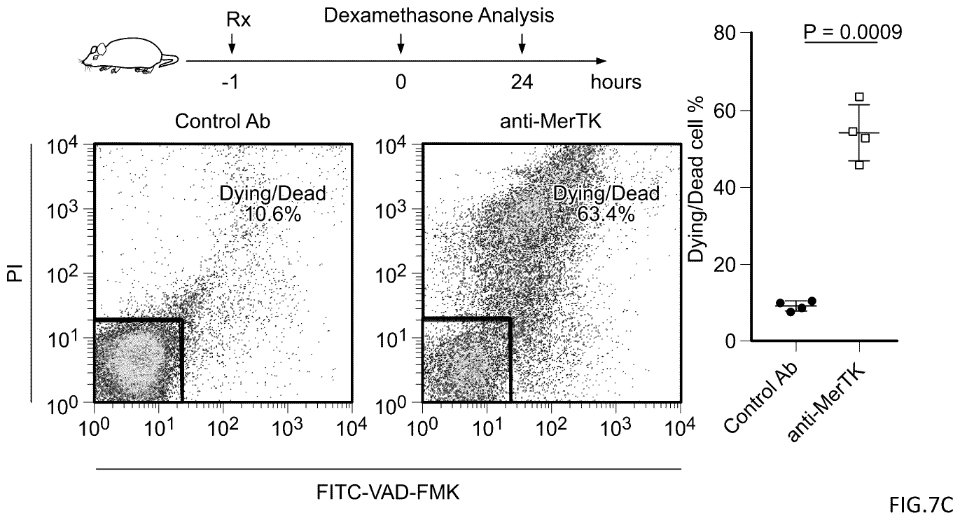

In the in vitro efferocytosis assay, anti-MerTK 14C9 (mIgG2a LALAPG) substantially reduced the uptake of apoptotic thymocytes by peritoneal macrophages (FIG. 7B). Moreover, in the in vivo assays, anti-MerTK 14C9 (mIgG2a LALAPG) effectively inhibited the clearance of apoptotic thymocytes in mice treated with dexamethasone (FIGS. 7C & 8B). This in vivo result was consistent with the defective efferocytosis observed in MerTK deficient mice (Scott, R. S. et al. Phagocytosis and clearance of apoptotic cells is mediated by MER. Nature 411, 207-211 (2001)), demonstrating the functional effectiveness of the anti-MerTK antibody.

### Example 8: Anti-MerTK Antibody Inhibits Ligand-Mediated MerTK Signaling

MerTK ligand-dependent AKT phosphorylation was measured to evaluate the effect of anti-MerTK antibody on ligand-mediated MerTK signaling.

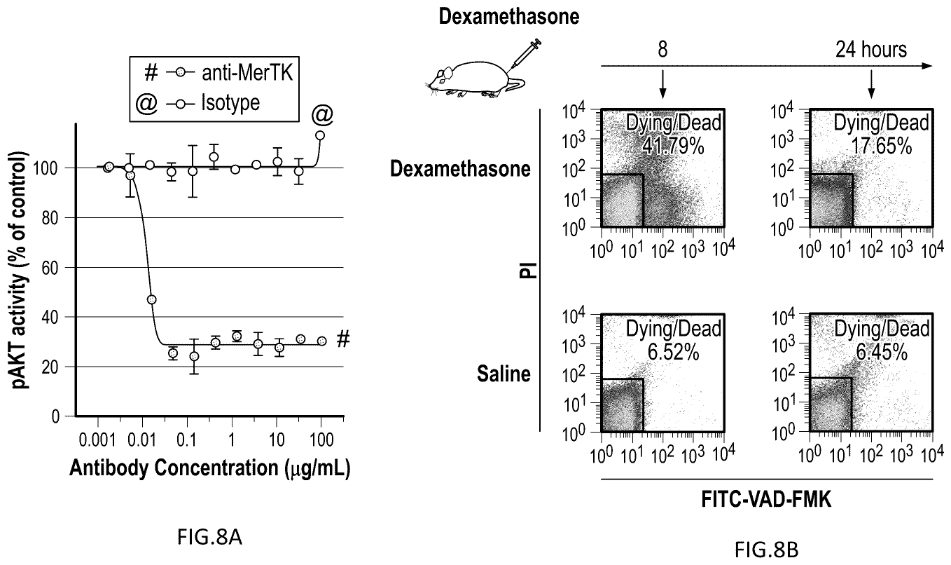

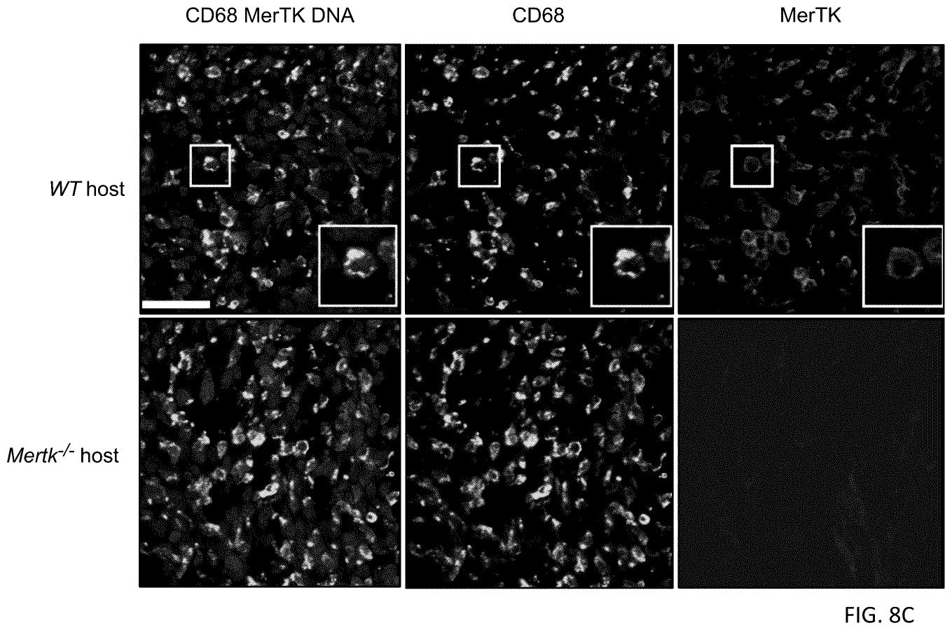

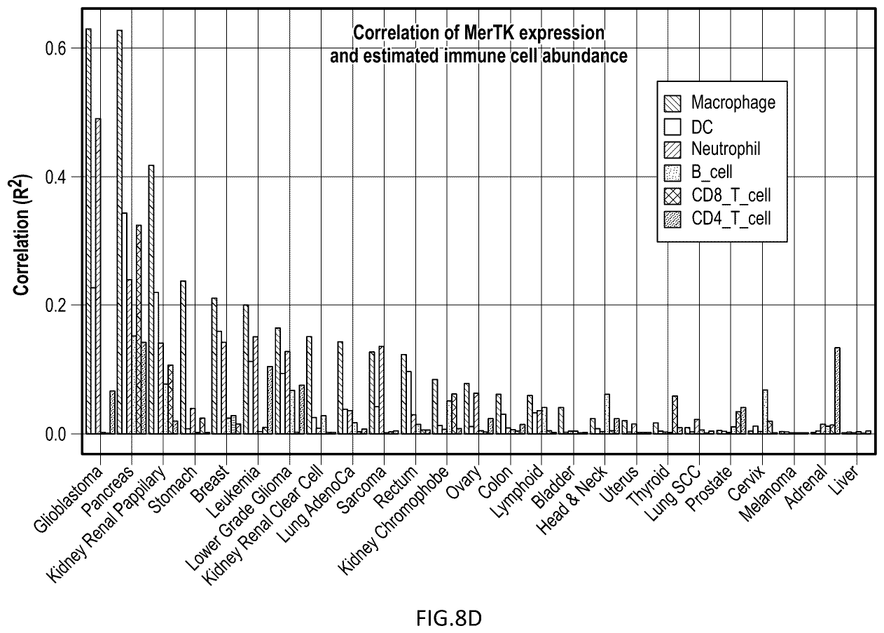

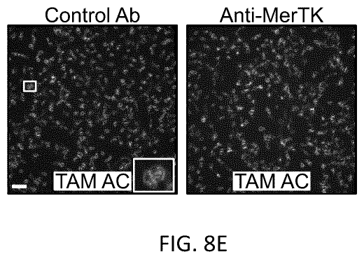

Briefly, J774A.1 mouse macrophages from an exponentially growing culture were seeded at a density of 2.0×105 cells/well on a 96-well plate in RPMI medium+10% FBS. The following day, cells were washed with 200 μL of serum free RPMI twice and incubated in 200 μL of serum free RPMI for 4 hours. After serum starvation, 10 μg/mL recombinant human GAS6-Fc protein, which is a ligand for MerTK, was added and incubated for 20 minutes. Phospho-AKT (pAKT) measurements were taken from treated cell lysates using the Phospho-AKT-1 (Ser473) HTRF Kit (Cisbio, #63ADK078PEG) following the manufacturer's instructions (standard protocol for two-plate assay protocol in 20 μL final volume). The AKT phosphorylation assay demonstrated that anti-MerTK antibody potently inhibits ligand-mediated MerTK signaling compared to an isotype control, as measured by pAKT activity in macrophages (FIG. 8A).

### Example 9: Effect of Anti-MerTK Antibody on Tumor-Associated Macrophages

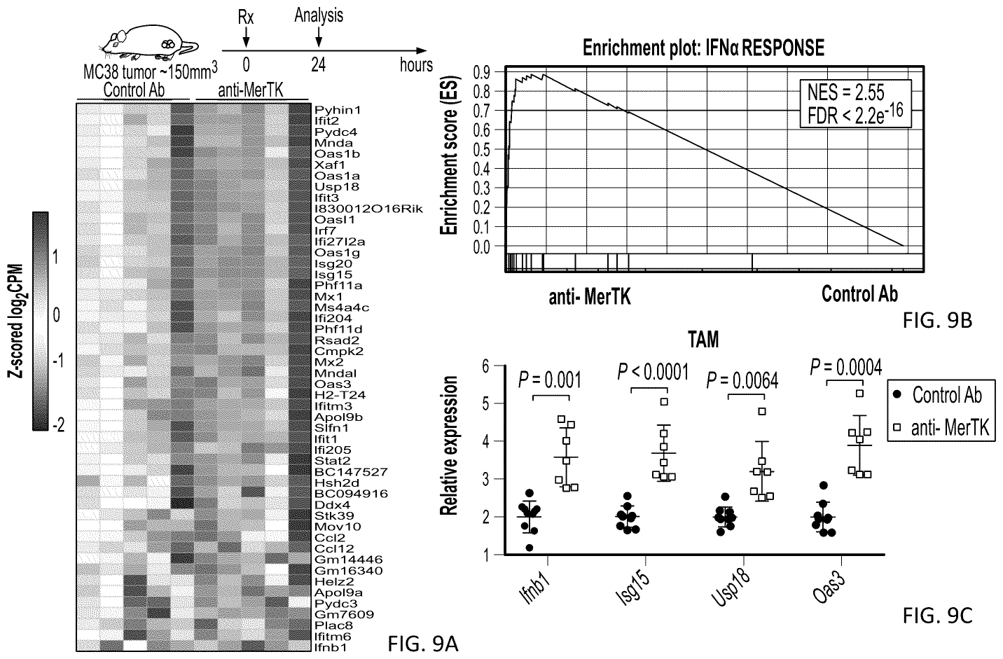

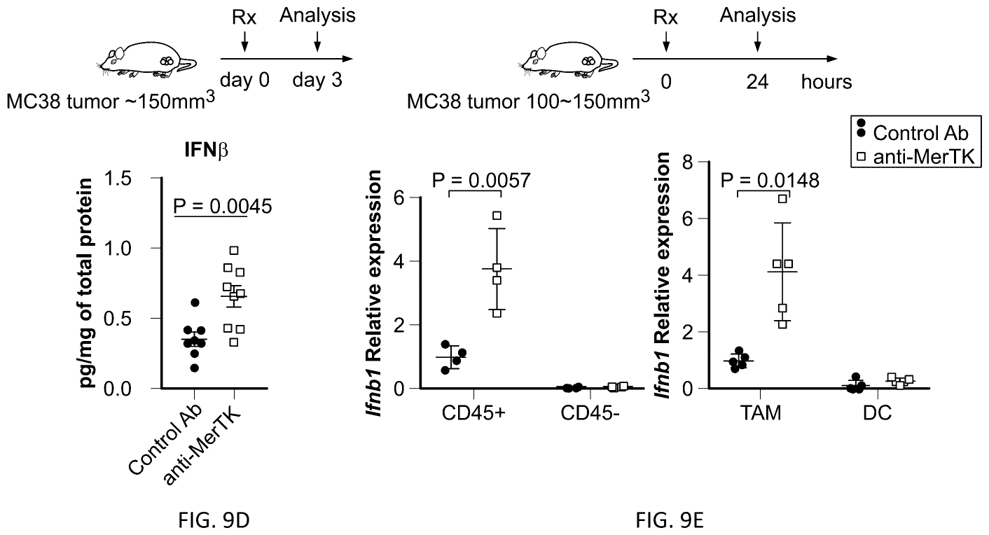

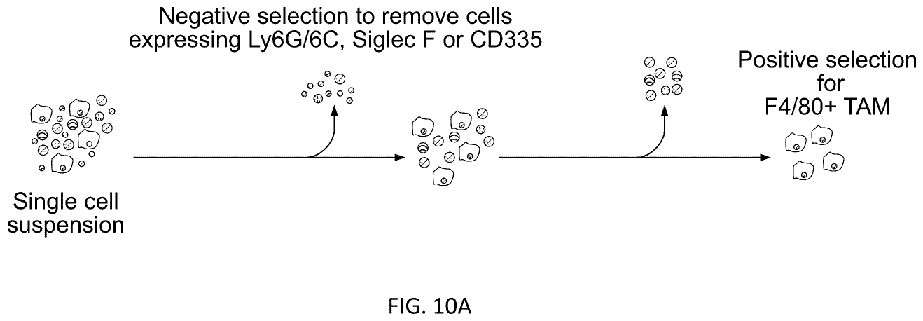

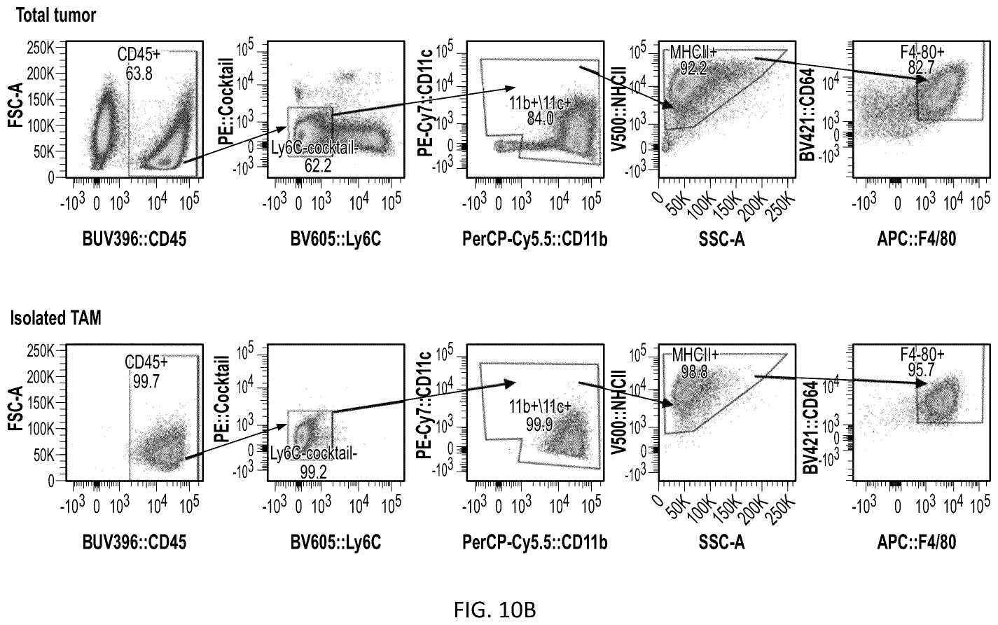

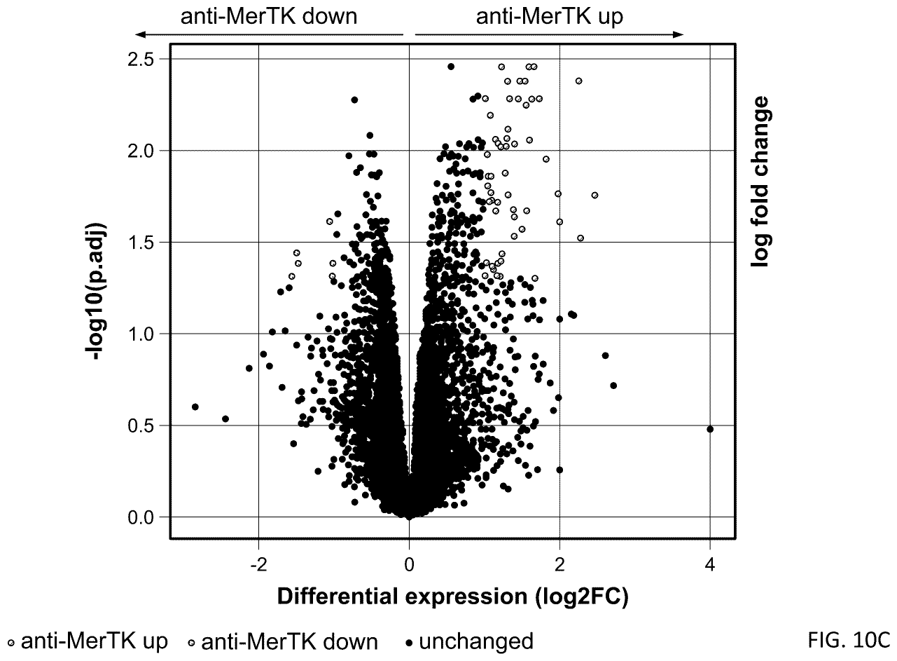

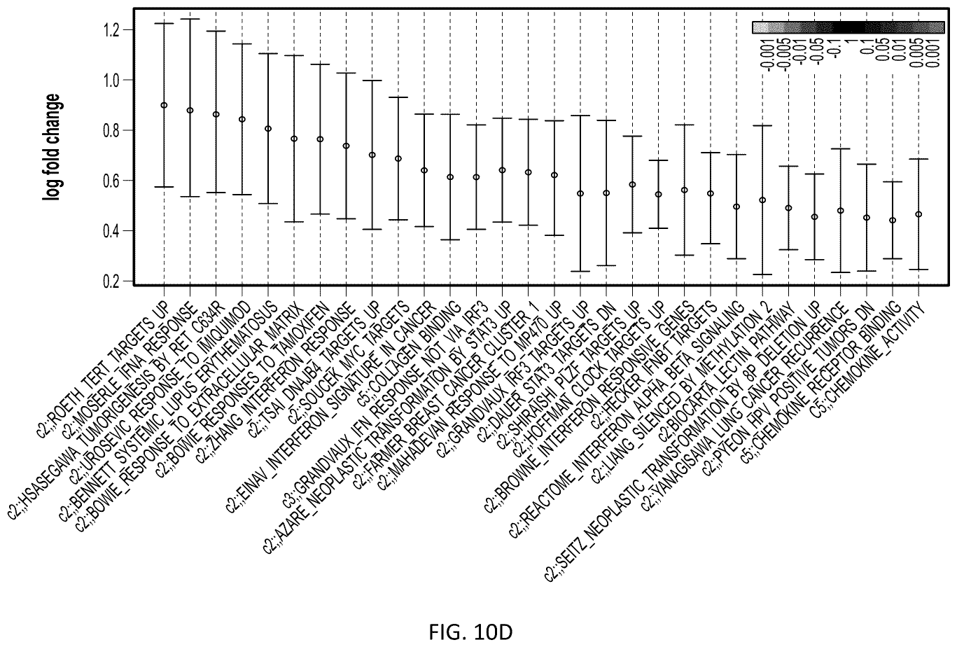

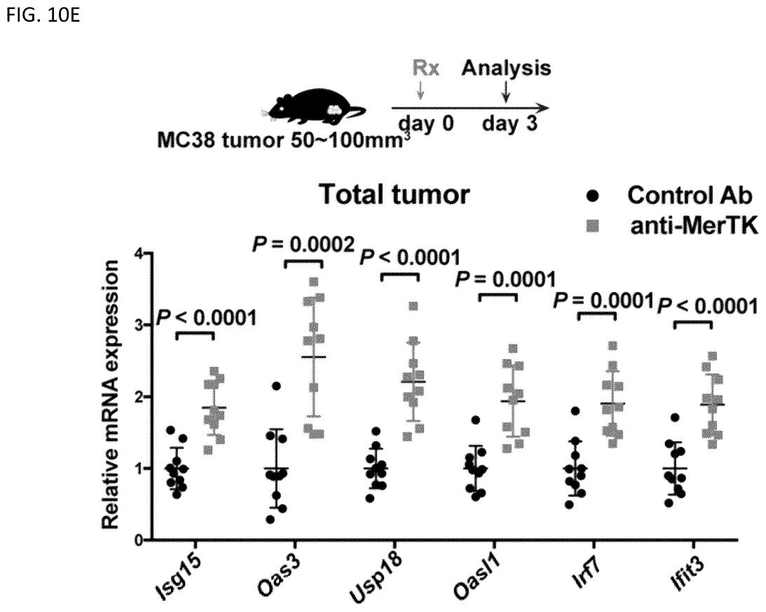

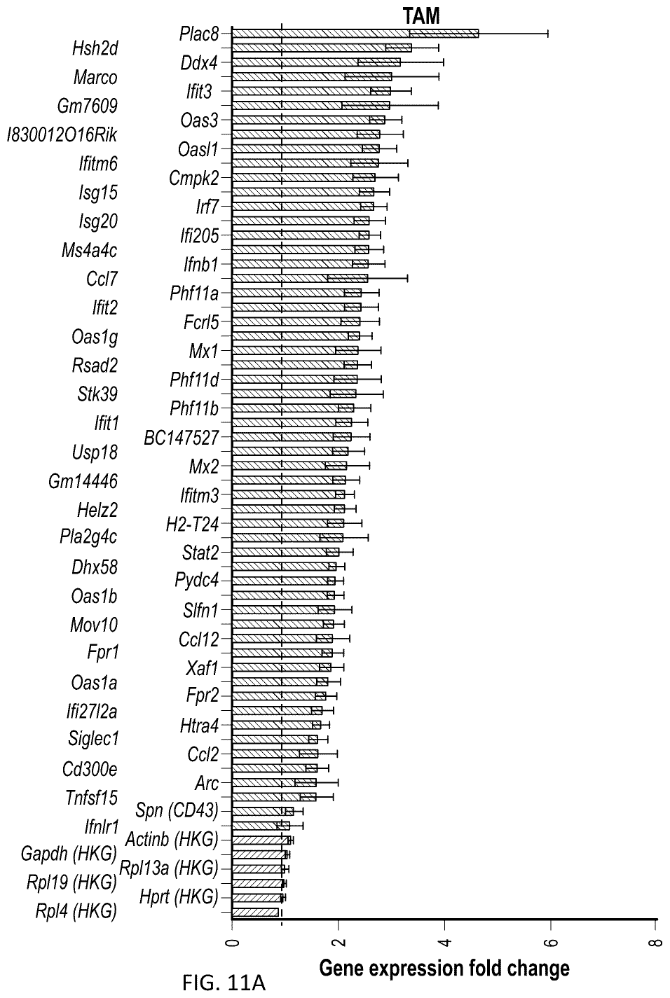

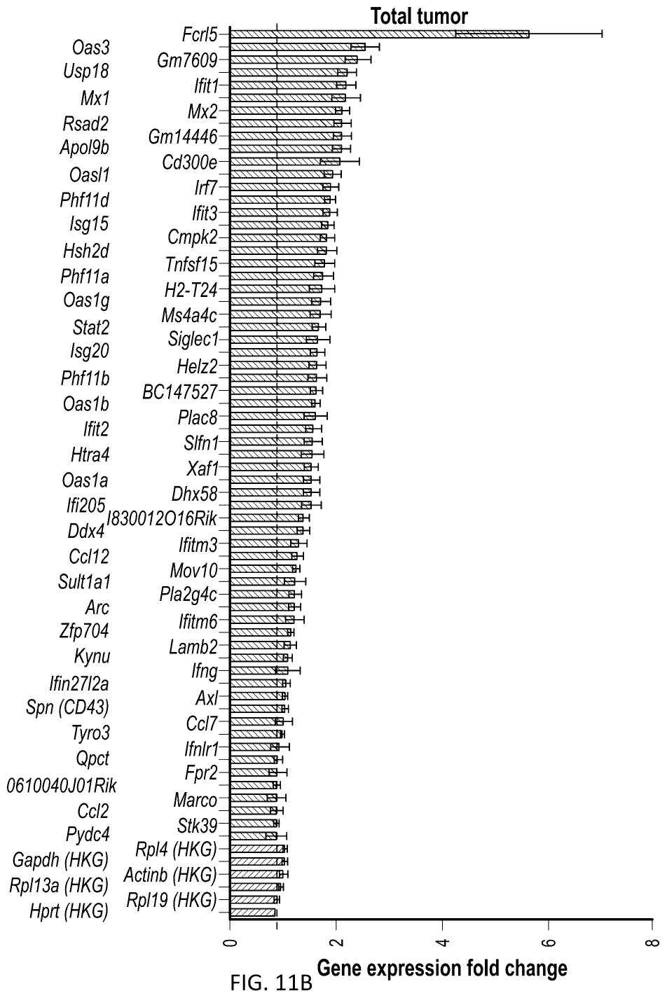

MerTK expression and distribution studies were carried out in tumor associated macrophages (TAMs), one of the most abundant tumor infiltrating immune cells. To isolate TAMs, tumors were harvested and dissociated into single cell suspensions. Live cells were enriched using Lymphocyte M media (Cedarlane Labs). CD335+, Siglec F+, and anti-Ly6G/6C+ cells were labeled with biotin-conjugated antibodies and depleted with anti-biotin MACSiBead™ Particles (Miltenyi Biotec). TAMs were then purified with anti-F4/80 Microbeads (Miltenyi Biotec) (FIG. 10A). The purity of isolated TAMs was confirmed to be >90% as assessed by FACS (FIG. 10B). Fluorescence microscopy was used to determine MerTK distribution in TAMs and the ability of TAMs to clear apoptotic cells (FIGS. 8C & 8E). qPCR and transcriptome analyses were performed to identify genes that are differentially expressed in cells treated with anti-MerTK 14C9 (mIgG2a LALAPG) or a control antibody (FIGS. 9, 10, 11, & 13).

Analysis of MC38 syngeneic murine colon adenocarcinoma tumors growing in wild-type (WT) or Mertk^{-/-} mice showed specific expression of MerTK in TAMs (FIG. 8C). In addition, TAMs from MC38 tumors were able to engulf apoptotic cells and, importantly, anti-MerTK 14C9 (mIgG2a LALAPG) inhibited this uptake (FIG. 8E). These results demonstrate that MerTK plays an important role in the clearance of apoptotic cells by TAMs in the tumor microenvironment and that treatment of TAMs with anti-MerTK antibody inhibits this uptake.

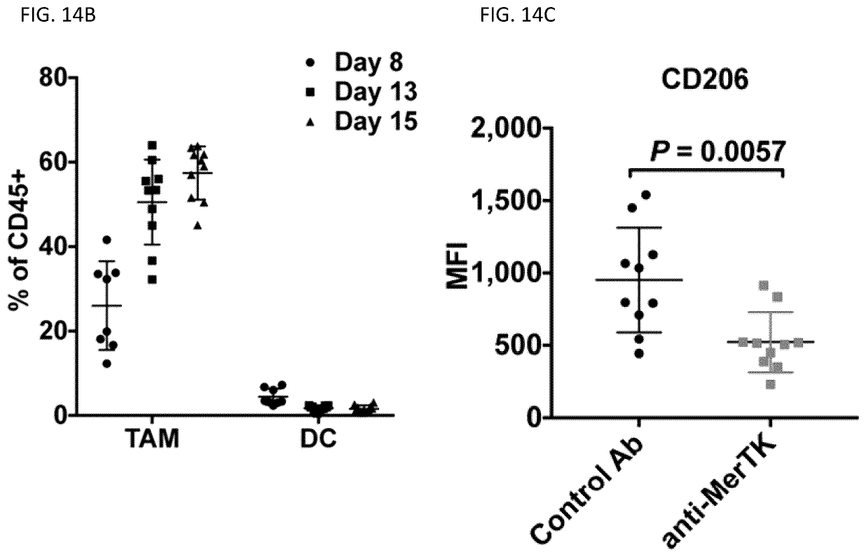

Transcriptome analysis of TAMs from established MC38 tumors treated with anti-MerTK antibody was performed to determine the impact of MerTK inhibition on TAMs. The transcriptome analysis revealed that TAMs from mice treated with anti-MerTK 14C9 (mIgG2a LALAPG) displayed significant changes in gene expression as compared to TAMs treated with the control antibody (FIGS. 9A & 10C). Gene set enrichment analysis revealed Type I IFN response as the most prominently up-regulated gene signature (FIGS. 9B & 10D). qPCR analysis confirmed the upregulation of Ifnb1 and multiple interferon stimulated genes (ISGs) in TAMs from anti-MerTK 14C9 (mIgG2a LALAPG) treated tumors (FIGS. 9C & 11A). A significant increase of IFNβ protein (FIG. 9D) and concomitant induction of ISGs was also observed in in tumor samples (FIGS. 10E & 11B). The upregulation of Ifnb1 expression was restricted to CD45+ immune cells and the basal level expression of IFNβ was much higher in CD45+ immune cells than in CD45− cells. In addition, IFNβ was significantly upregulated in TAMs but not in DCs (FIG. 9E), and TAMs were considerably more abundant than DCs in the MC38 tumors (FIG. 14B).

### Example 10: Distribution of MerTK in Human Cancers

The distribution of MerTK expression in human cancers was determined using expression data from The Cancer Genome Atlas (TCGA). Expression data in TCGA samples were obtained as described by Daemen et al. (Daemen, A. et al. Pan-Cancer Metabolic Signature Predicts Co-Dependency on Glutaminase and De Novo Glutathione Synthesis Linked to a High-Mesenchymal Cell State. Cell Metab 28, 383-399 e389 (2018)). Gene expression in form of RPKMs served as input for TIMER software (Li, T. et al. TIMER: A Web Server for Comprehensive Analysis of Tumor-Infiltrating Immune Cells. Cancer Res 77, e108-e110 (2017)) to calculate relative levels of six tumor-infiltrating immune subsets. It was confirmed that MerTK was not part of the signatures used to estimate immune set abundance. Pearson correlation coefficients between gene expression level and immune cell type estimates were computed for each cell type and indication. In human cancers, MerTK expression exhibited greater correlation with the abundances of TAMs compared to other immune cell types (FIG. 8D), consistent with MerTK being expressed by TAMs.

### Example 11: Anti-MerTK Antibody Induces the Local Type I IFN Response in the Tumor Microenvironment

The relationship between anti-MerTK antibody treatment and the Type I IFN response was investigated. Briefly, female C57BL/6 mice were inoculated subcutaneously in the right unilateral flank with 1×10⁵ MC38 tumor cells suspended in Hanks's Buffered Saline Solution (HBSS) and phenol red-free Matrigel (1:1 v/v) (BD Bioscience) and then treated with 20 mg/kg anti-MerTK 14C9 (mIgG2a LALAPG) antibody or control antibody. Three days after treatment, tumors were homogenized in PBS supplemented with Halt™ Protease and Phosphatase Inhibitor Cocktail (ThermoFisher Scientific) in gentleMACS M Tubes (Miltenyi Biotec) using gentleMACS Dissociator (Miltenyi Biotec) following the manufacturer's protocol. For every 100 mg of tumor tissue, 500 μL of buffer was used. Tumor homogenates were clarified by centrifugation at 12,000×g for 20 minutes at 4° C. Homogenates were normalized based on total protein concentrations determined by BCA Protein Assay Kit (Piece). IFN-β and CCL7 (MCP-3) were assayed using High Sensitivity Mouse IFN Beta ELISA Kit (PBL Assay Science) and Mouse MCP-3 Instant ELISA Kit (Invitrogen), respectively. Other cytokines/chemokines were assayed using MILLIPLEX MAP Mouse Cytokine/chemokine Magnetic Beads Penal-Premixed 15-Plex and 32-Plex (Millipore). Cytokine/chemokine results were expressed as pg/mg of total protein in tumor homogenate.

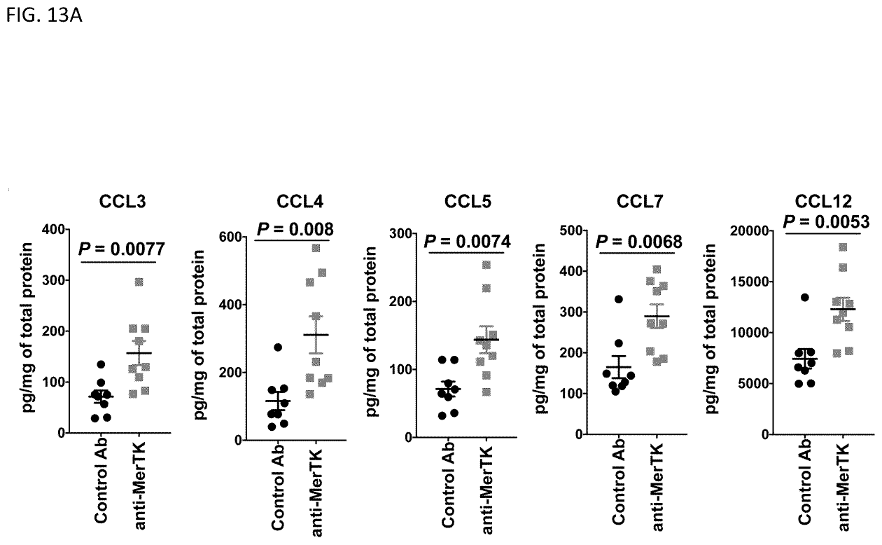

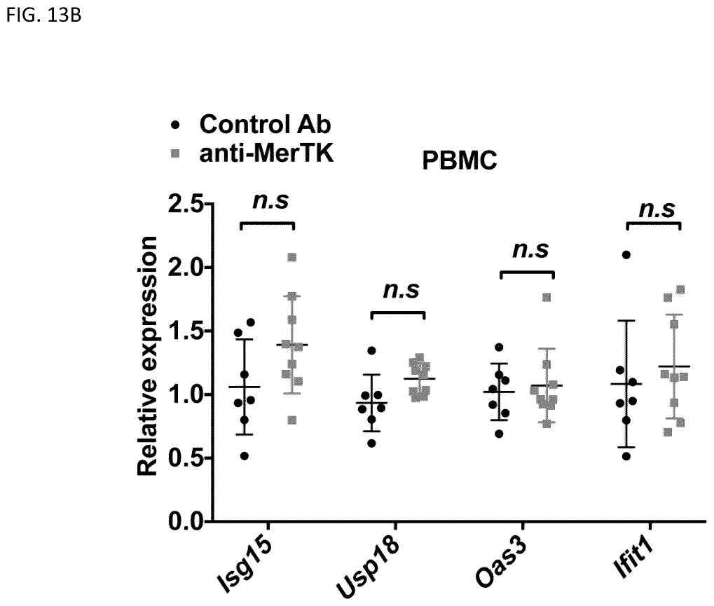

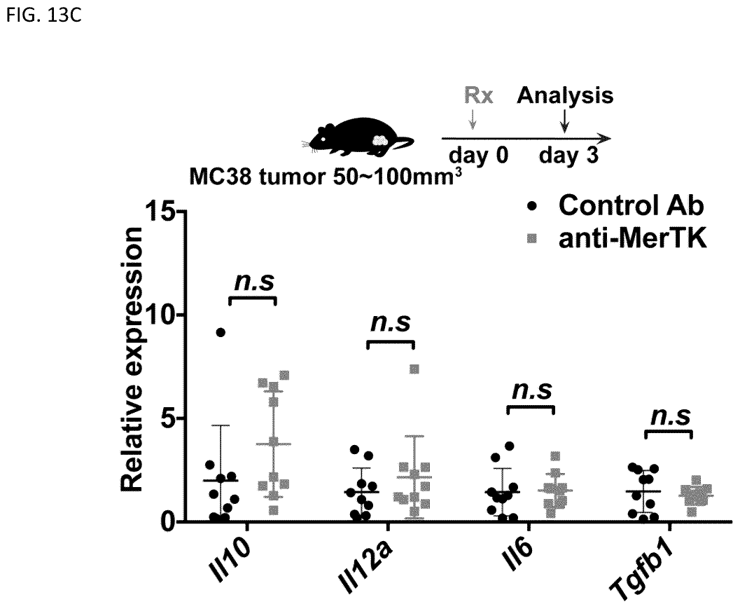

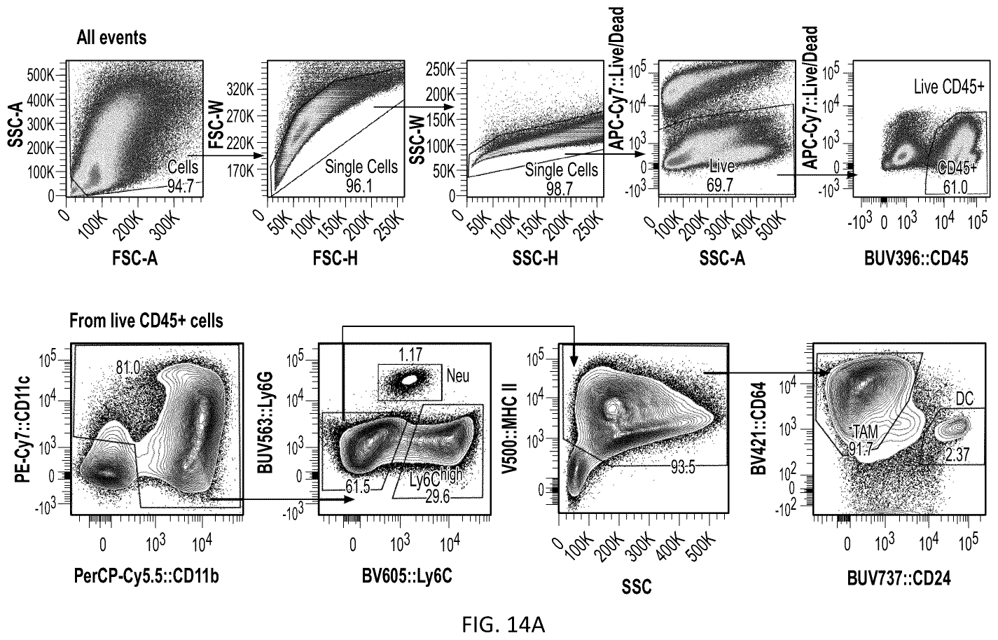

Type I IFNs activate autocrine and/or paracrine production of cytokines and chemokines that modulate innate and adaptive immune responses. In line with this, protein levels of the cytokines or chemokines CCL3, CCL4, CCL5, CCL7, and CCL12 in anti-MerTK antibody treated tumor homogenates were observed (FIG. 13A) The type I IFN response appeared to be restricted to the tumor site, as no significant changes in ISG expression were found in peripheral blood mononuclear cells (PBMCs) collected from tumor bearing mice treated with anti-MerTK antibody (FIG. 13B). Significant changes in the expression of cytokines that were previously reported to be linked to MerTK activation, including IL10, TGFβ1, IL6 and IL12a (FIG. 13C) were not observed. In summary, these data demonstrate that anti-MerTK antibodies can induce the local Type I IFN response in the tumor microenvironment.

### Example 12: Anti-MerTK Antibody Enhances Antitumor Immunity

Given that anti-MerTK antibody induced a type I IFN response and that Type I IFNs positively regulate various aspects of antigen-presenting cells (APCs), an antigen presentation assay was performed to determine whether antigen presentation by TAMs and tumor-associated DCs is enhanced by anti-MerTK antibody. Briefly, female C57BL/6 mice were inoculated subcutaneously in the right unilateral flank with 5×106 MC38.OVA tumor cells suspended in Hanks's Buffered Saline Solution (HBSS) and phenol red-free Matrigel (1:1 v/v) (BD Bioscience). When tumors reached volumes of 100-150 mm³ (day 0), mice were administered anti-MerTK 14C9 (mIgG2a LALAPG) antibody or control antibody anti-gp120 via intraperitoneal (IP) injection at a dose of 20 mg/kg. Tumors were later analyzed for antigen presentation enhancement. In the MC38.OVA tumor model, H-2K^{b} bound OVA-derived SIINFEKL peptide can be readily detected for monitoring antigen presentation. The anti-H-2K^{b}-SIINFEKL (Biolegend, clone 25-D1.16) was used to specifically detect OVA-derived peptide SIINFEKL bound to MHC class I H-2K^{b}, but not unbound H-2K^{b} or H-2K^{b} bound to other peptides.

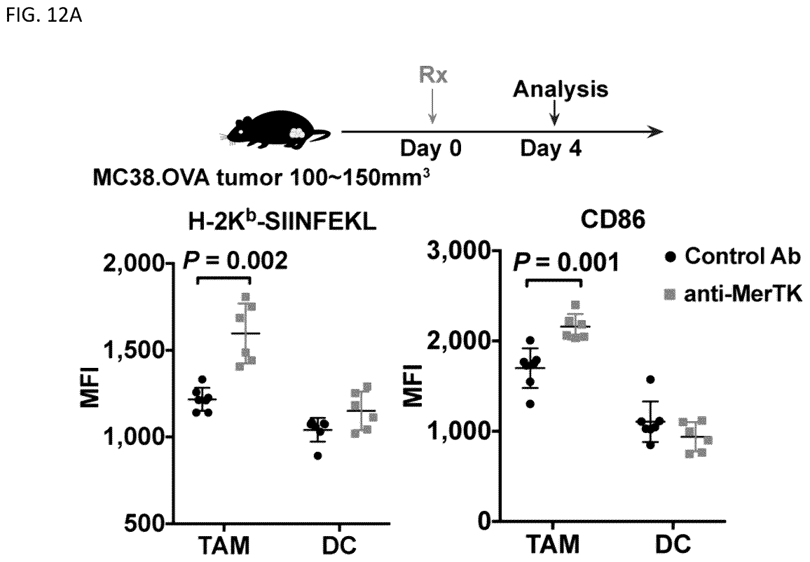

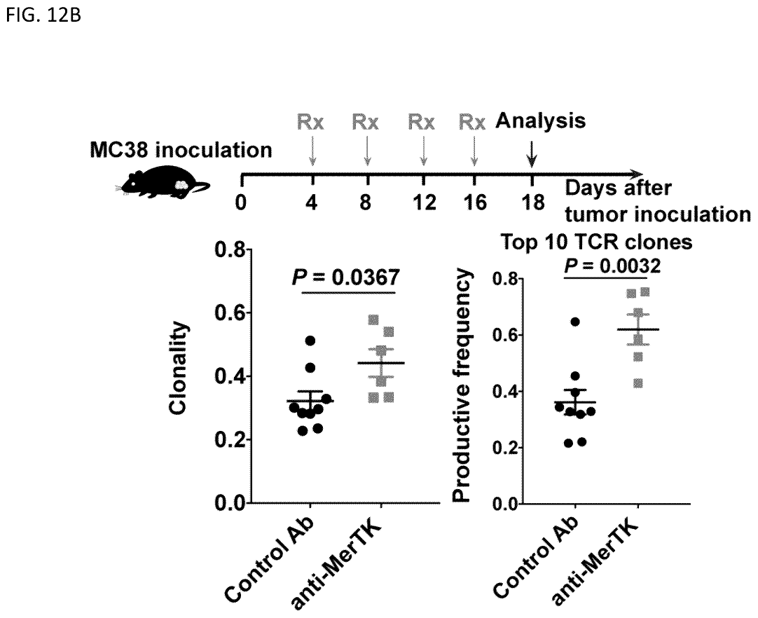

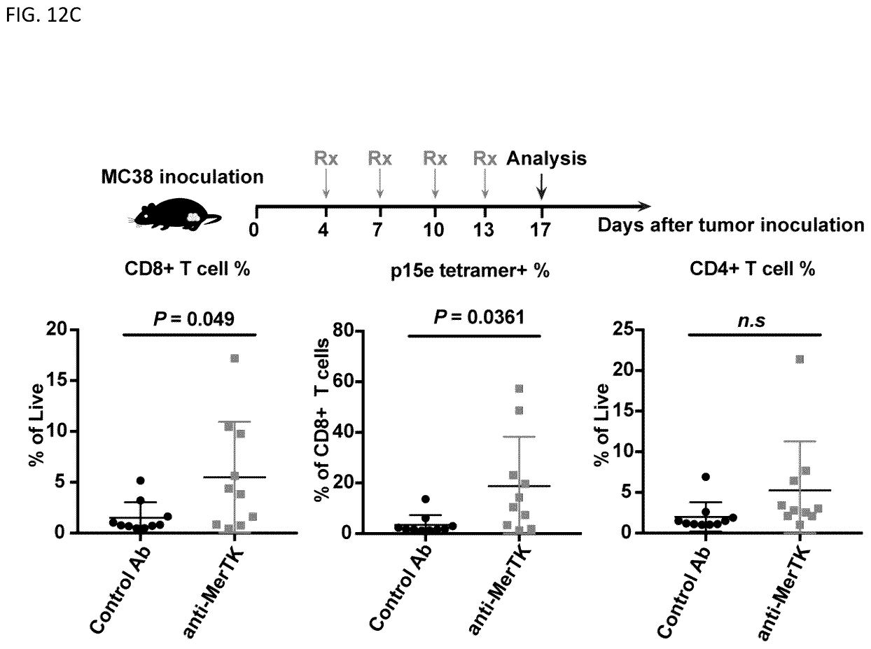

Anti-MerTK antibody significantly increased the level of H-2K^{b}-SIINFEKL complex on TAMs (FIG. 12A). CD86, a costimulatory molecule for T cell activation, was also elevated in TAMs but not DCs (FIG. 12A). A downregulation of CD206, an “M2-like” macrophage marker, on TAMs after anti-MerTK antibody treatment was also observed (FIG. 14C). These findings suggest that anti-MerTK antibody induces an immunogenic reprogramming of tumor microenvironment, which in turn could enhance the adaptive T cell response.

Tumor-infiltrating lymphocyte (TIL) clonality reflects the frequency of T cells with a specific TCR chain usage at the tumor site. To determine whether anti-MerTK antibody treatment affects clonal expansion of antigen-specific TILs, tumor-infiltrating T cells were enriched using Dynabeads Mouse Pan T Kit (ThermoFisher Scientific). Genomic DNA from enriched T cells was extracted using AllPrep DNA/RNA/Protein Mini Kit (Qiagen) and subjected to TCRβ CDR3 sequencing using the Immunoseq platform at survey level (Adaptive Biotechnologies). Sequencing results were analyzed using ImmunoSEQ Analyzer (Adaptive Biotechnologies). Clonality scores were calculated as 1-(entropy)/log 2(number of productive unique sequences), where the entropy takes into account the varying clone frequency.

Anti-MerTK 14C9 (mIgG2a LALAPG) treatment led to a significant increase in TIL clonality (FIG. 12B), indicating clonal expansion of antigen-specific TILs. In addition, anti-MerTK 14C9 (mIgG2a LALAPG) treatment increased the frequency of total CD8+ T cells, as well as antigen specific CD8+ T cells, e.g., T cells that recognize pi5e, an endogenous antigen presented by MC38 tumor cells (FIG. 12C). Therefore, MerTK blockade enhances the immune recognition of tumor cells and tumor-specific CD8+ T response.

### Example 13: Anti-MerTK Antibody is Effective in Combination with Anti-PD-1, Anti-PD-L1, and Gemcitabine

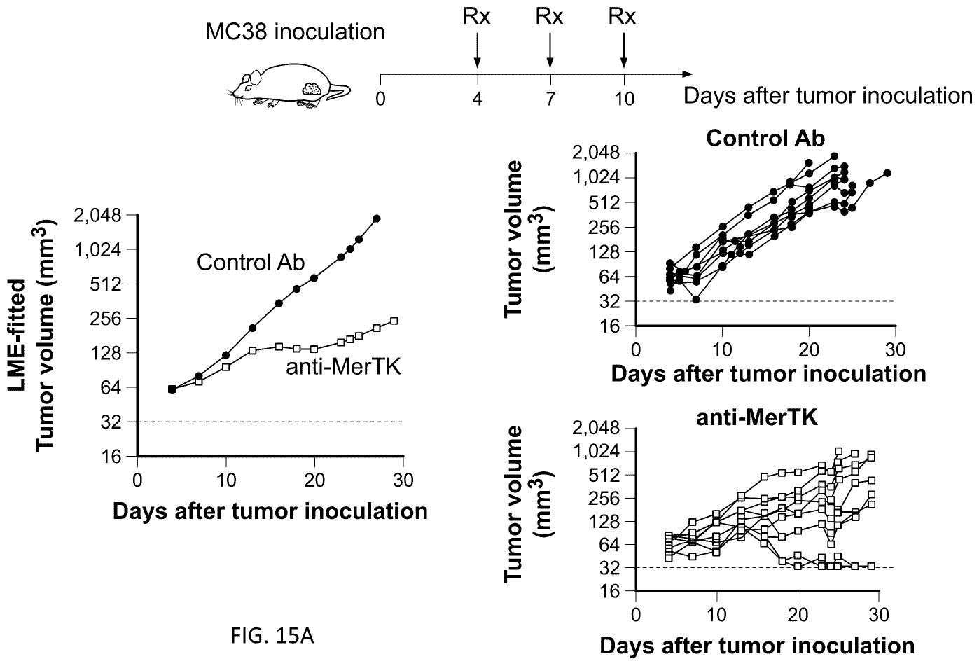

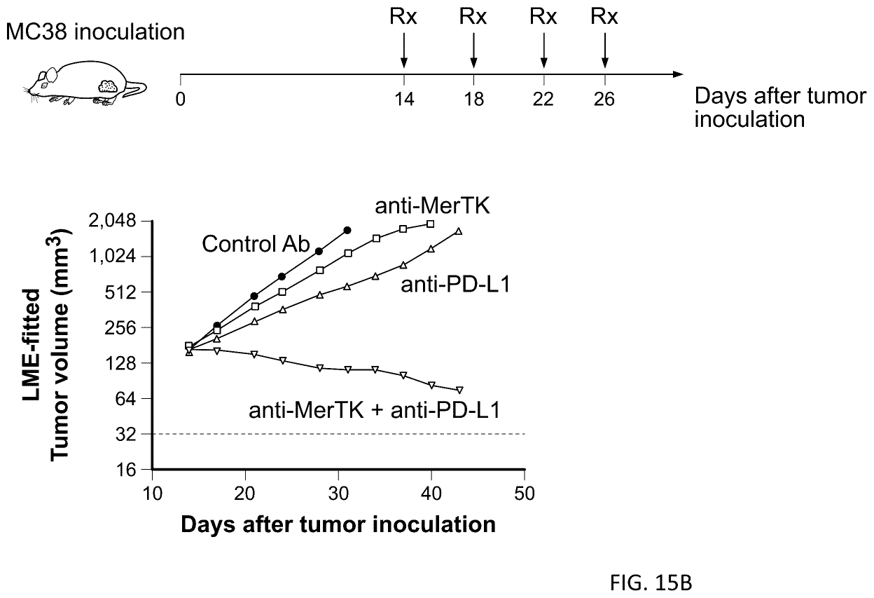

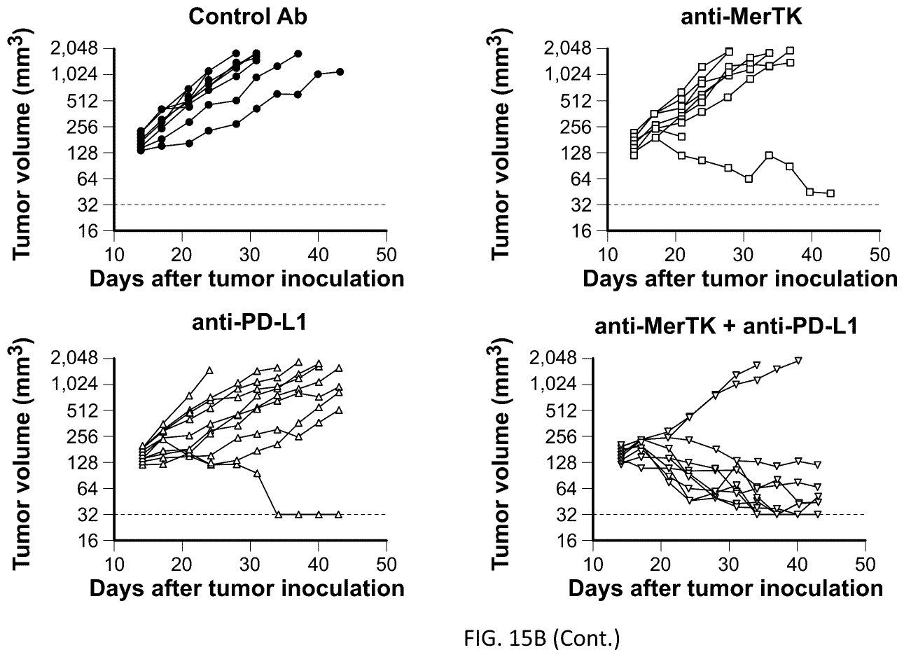

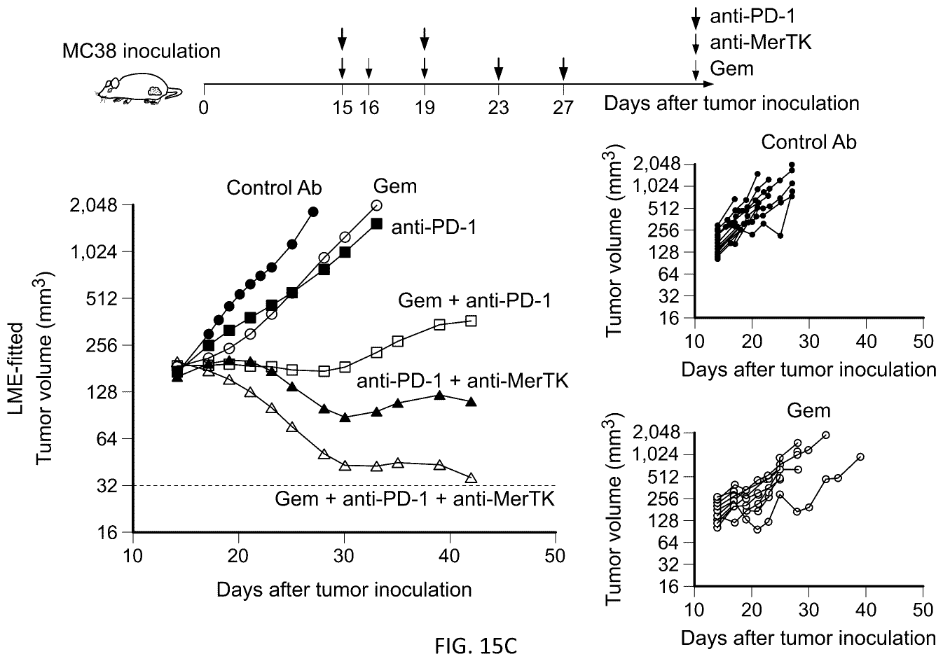

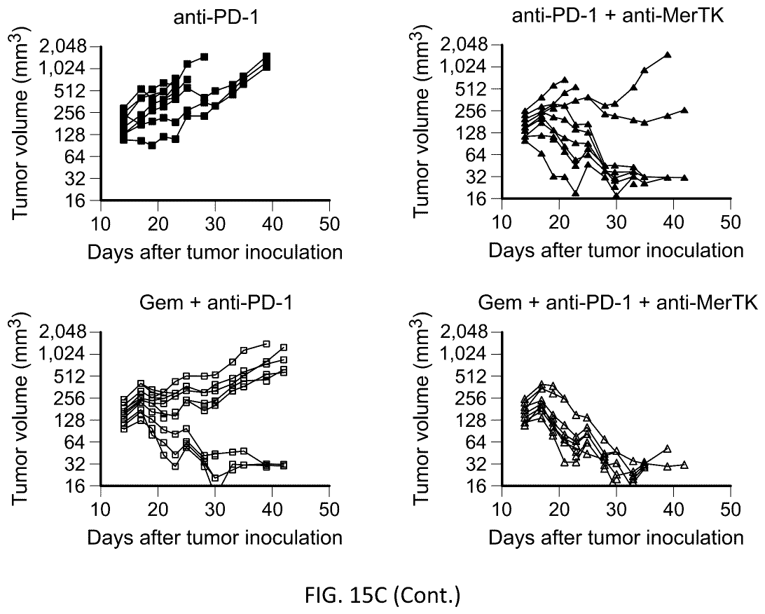

To further characterize the effectiveness of anti-MerTK antibody as a combination therapy, tumor growth assays were performed as previously described. Briefly, female C57BL/6 mice were inoculated subcutaneously in the right unilateral flank with 1×10⁵ MC38 tumor cells suspended in Hanks's Buffered Saline Solution (HBSS) and phenol red-free Matrigel (1:1 v/v) (BD Bioscience). On predetermined days post inoculation, mice were administered (1) the anti-MerTK antibody as a monotherapy (FIG. 15A); (2) anti-MerTK antibody and anti-PD-L1 antibody as a combination therapy (FIG. 15B); or (3) anti-MerTK antibody, anti-PD-1 antibody, and the chemotherapeutic gemcitabine as a combination therapy (FIG. 15C). Anti-MerTK 14C9 (mIgG2a LALAPG) was administered at 20 mg/kg, anti-PD-L1 was administered at 10 mg/kg, anti-PD1 was administered at 8 mg/kg, and gemcitabine was administered at 120 mg/kg. Treatments were administered either at an early stage of tumor progression (FIG. 15A) or when tumors were fully established (FIGS. 15B & 15C).

When treatment started at the early stage of tumor progression, single-agent anti-MerTK antibody was able to significantly reduce the tumor growth (FIG. 15A). In comparison, in the intervention setting of treating fully established tumors, anti-MerTK antibody or anti-PD-L1 antibody alone had a marginal effect (FIG. 15B). In contrast, simultaneous treatment with anti-MerTK antibody and anti-PD-L1 antibody exhibited a robust antitumor effect (FIG. 15B). Similarly, treatment with anti-MerTK antibody significantly improved the efficacy of an antibody targeting PD-1 (the receptor for PD-L1) (FIG. 15C). The chemotherapy drug gemcitabine moderately improved anti-PD-1 antibody therapy. However, addition of anti-MerTK antibody to the combination therapy of gemcitabine plus anti-PD-1 antibody resulted in complete regression of all treated tumors (FIG. 15C).

### Example 14: Anti-MerTK Antibody Antitumor Effect Depends on the Presence of Functional STING in the Tumor Host

To explore the role of Type I IFN signaling in anti-MerTK-induced antitumor immune responses, a functional neutralizing antibody against IFNAR1 (anti-IFNAR1 clone MAR1-5A3 BioXCell) was used to interfere with Type I IFN signaling and tumor growth assays were performed as previously described. Briefly, female C57BL/6 mice were inoculated subcutaneously in the right unilateral flank with 1×10⁵ MC38 tumor cells suspended in Hanks's Buffered Saline Solution (HBSS) and phenol red-free Matrigel (1:1 v/v) (BD Bioscience). On predetermined days post inoculation, mice were administered (1) the anti-MerTK 14C9 (mIgG2a LALAPG) antibody as a monotherapy; (2) the anti-IFNAR1 antibody as a monotherapy; (3) anti-MerTK 14C9 (mIgG2a LALAPG) and anti-PD-L1 antibody as a combination therapy; or (4) anti-MerTK 14C9 (mIgG2a LALAPG), anti-PD-L1 antibody, and anti-INFAR1 antibody as a combination therapy.

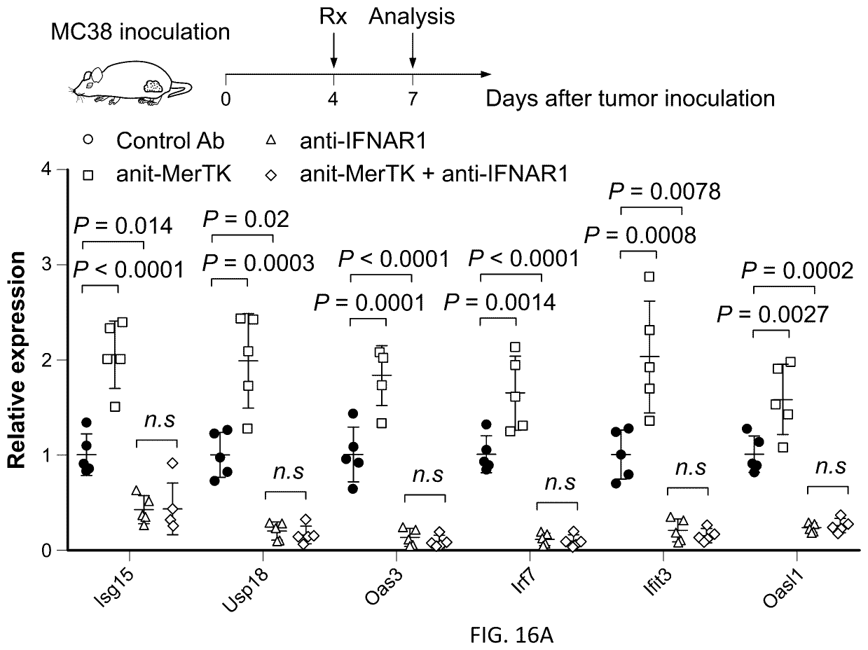

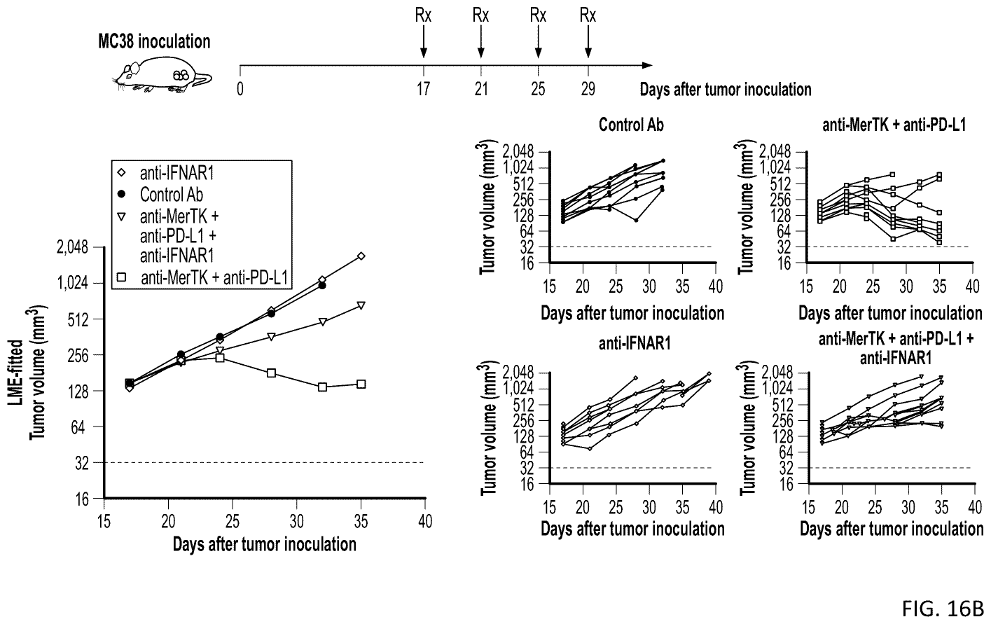

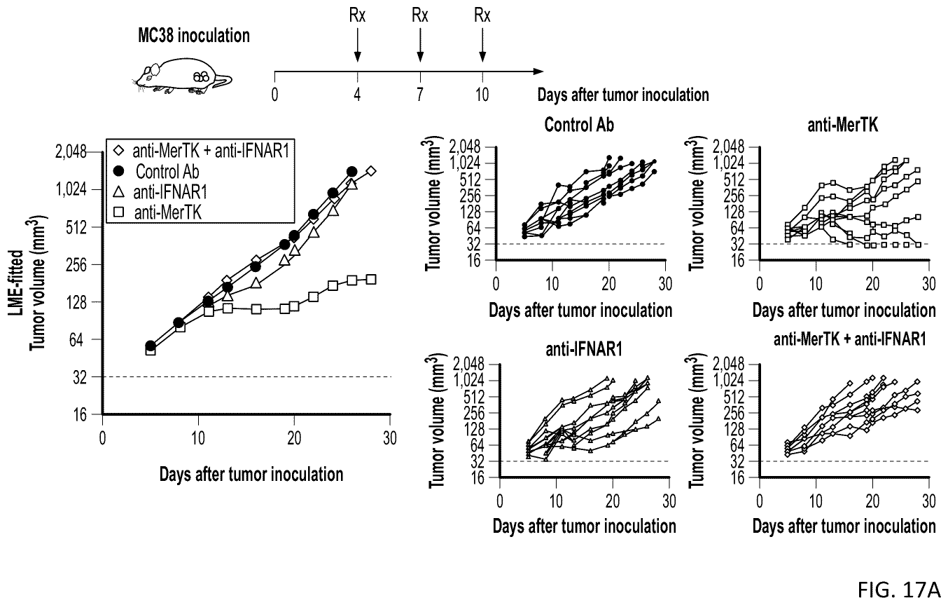

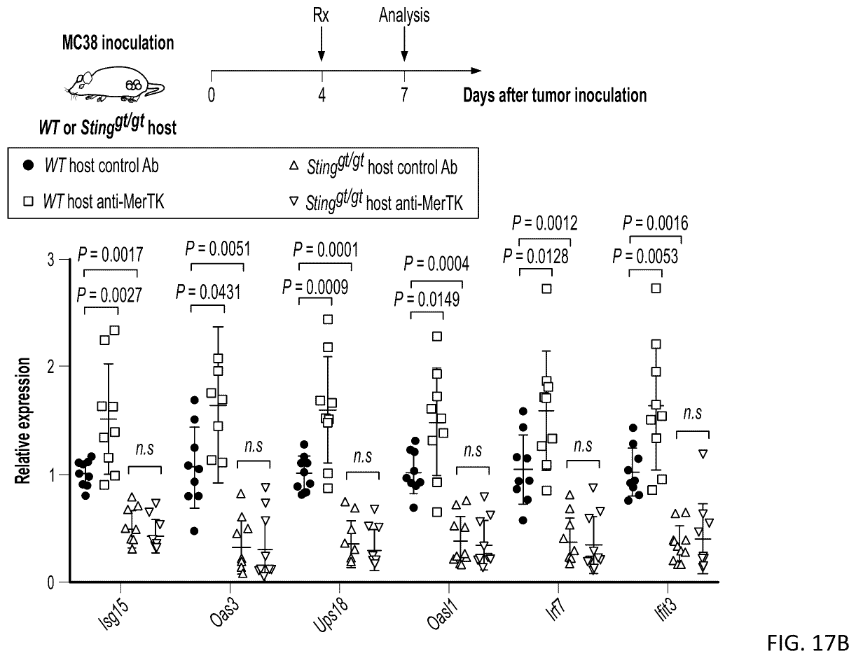

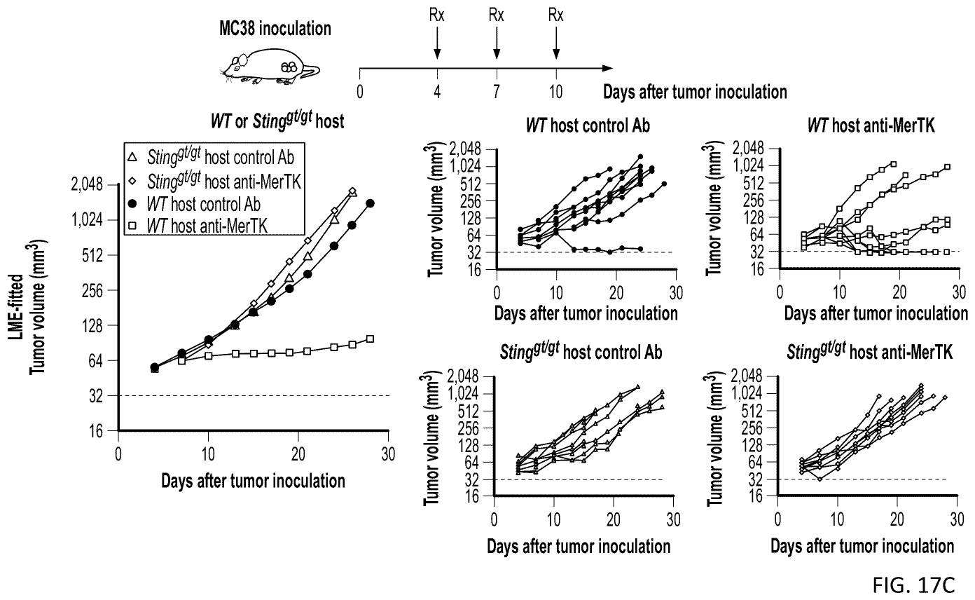

Anti-IFNAR1 antibody treatment completely abolished the modulation of ISGs by MerTK blockade (FIG. 16A). Blocking type I IFN signaling also negated the antitumor activity of anti-MerTK 14C9 (mIgG2a LALAPG) either as a single agent (FIG. 17A) or in combination with anti-PD-L1 (FIG. 16B). These results demonstrate that the antitumor effect of anti-MerTK antibody depends on intact type I IFN signaling.

The STING pathway has emerged as a key signaling mechanism that drives the antitumor type I IFN response (Woo, S. R. et al. STING-dependent cytosolic DNA sensing mediates innate immune recognition of immunogenic tumors. Immunity 41, 830-842 (2014); Deng, L. et al. STING-Dependent Cytosolic DNA Sensing Promotes Radiation-Induced Type I Interferon-Dependent Antitumor Immunity in Immunogenic Tumors. Immunity 41, 843-852 (2014)). To determine the role of STING signaling for the antitumor effect of MerTK blockade, tumor studies with WT and STING-defective (Sting^{gt/gt}) mice were carried out. In contrast to the WT mice, no upregulation of ISGs was detected in Sting^{gt/gt} mice after anti-MerTK antibody treatment (FIG. 17B). Furthermore, the antitumor effect of MerTK inhibition was lost in the absence of functional STING in mice (FIG. 17C). These data demonstrate that the antitumor effect of anti-MerTK antibody depends on the presence of functional STING in the host.

### Example 15: Anti-MerTK Antibody Antitumor Effect Depends on the Presence of Functional cGAS in Tumor Cells

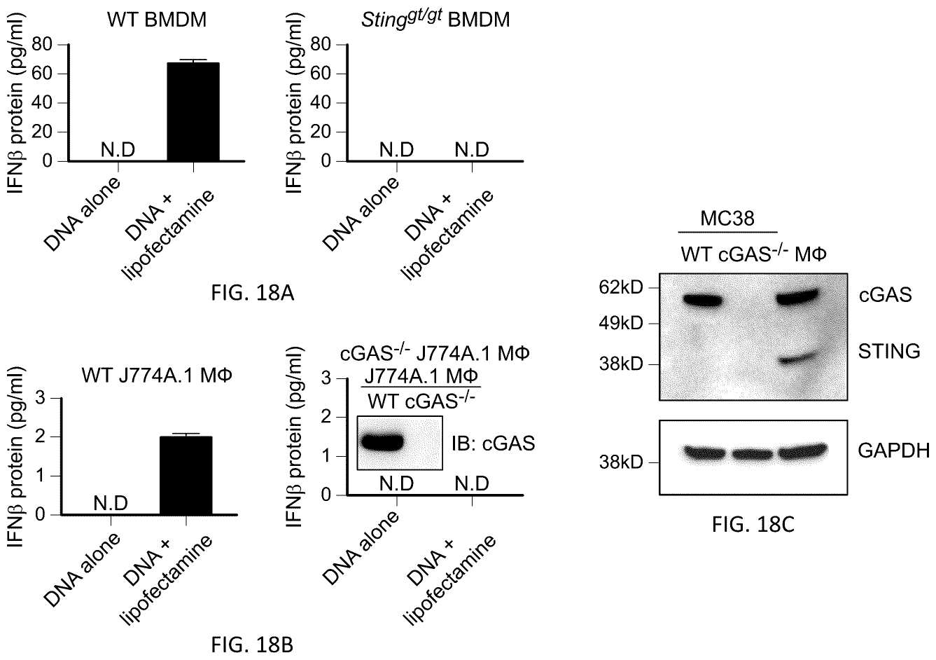

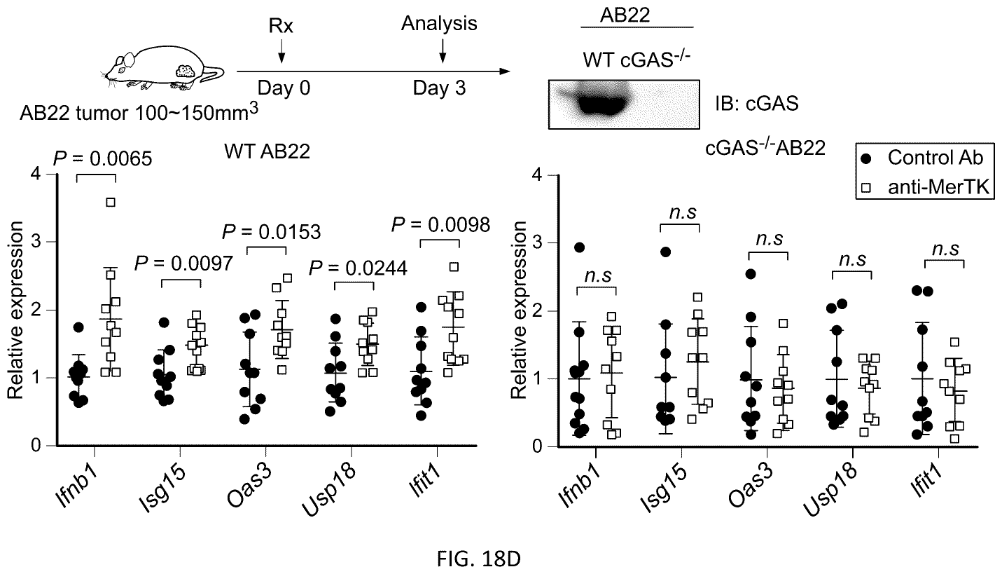

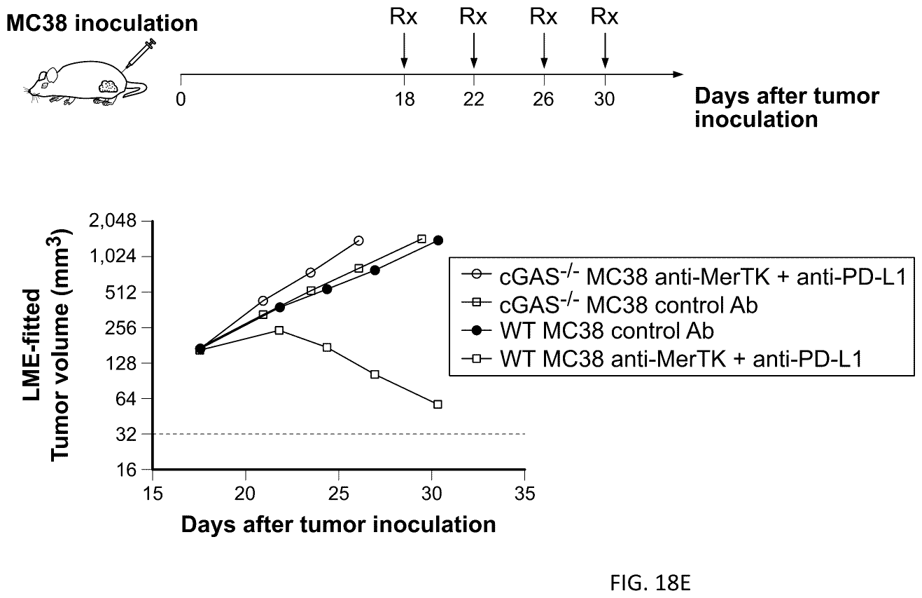

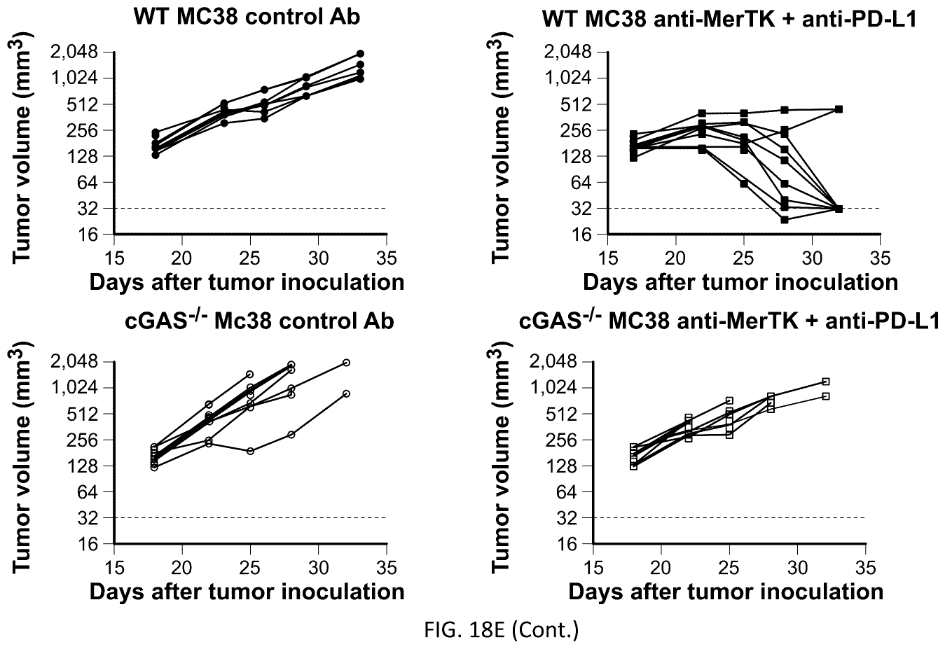

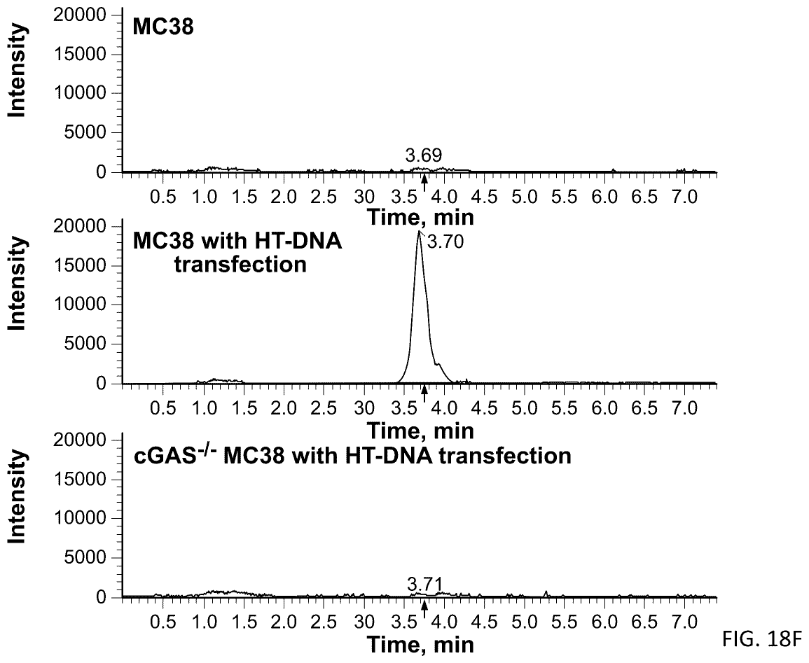

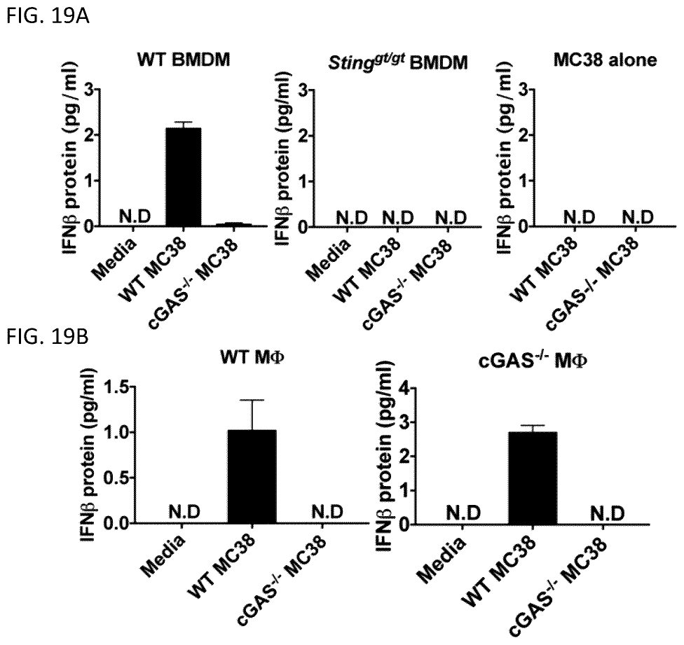

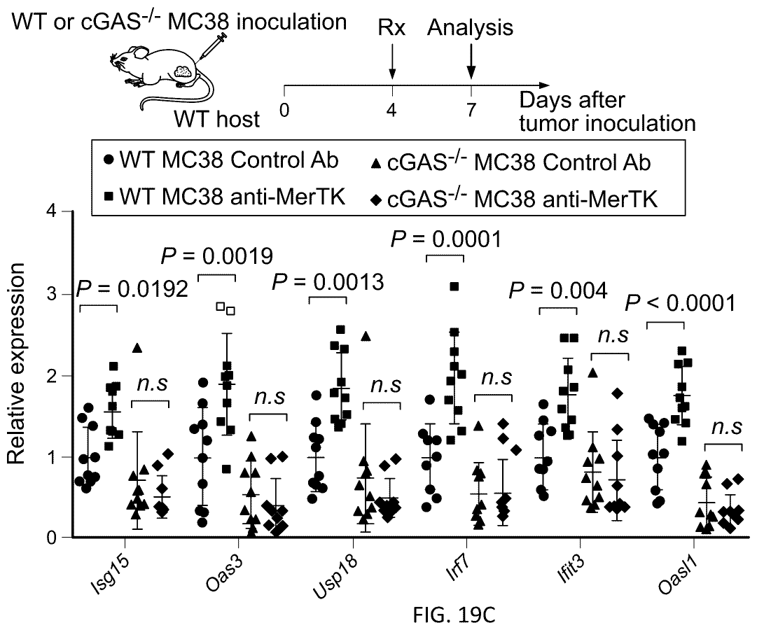

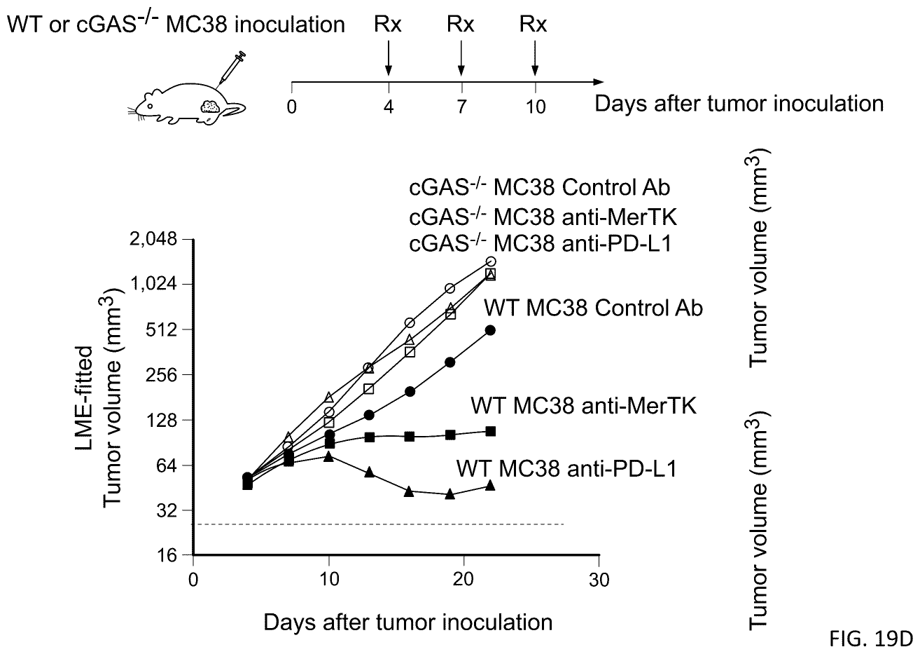

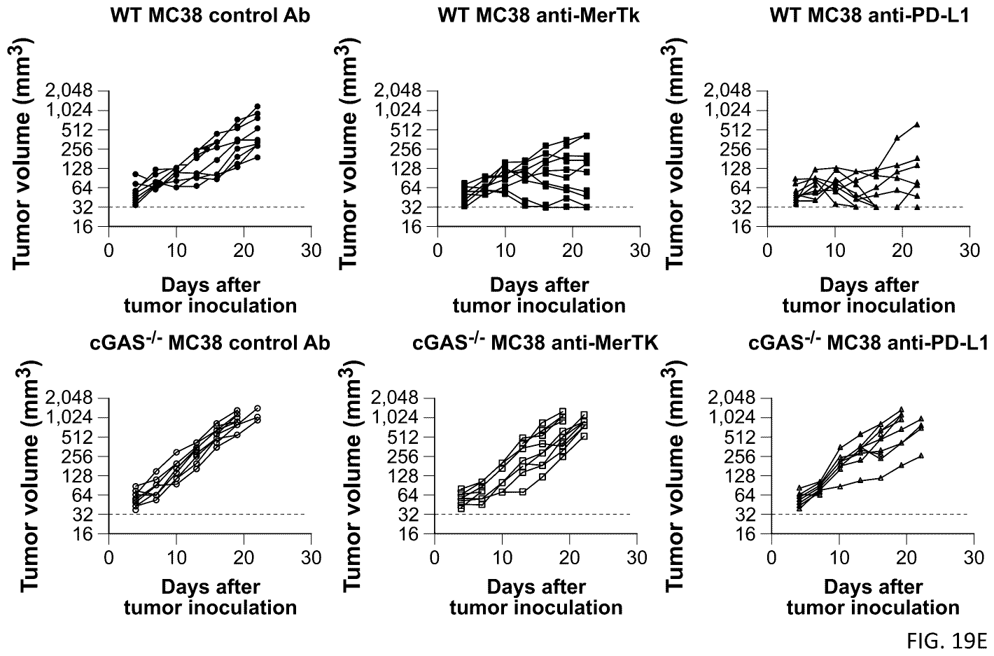

Cytoplasmic DNA transfection experiments were carried out to evaluate the effect of STING and cGAS on the anti-MerTK antibody antitumor effect. Briefly, WT bone marrow-derived macrophages (BMDMs), Sting^{gt/gt} BMDMs, WT J774A.1 macrophages, and cGAS^{-/-} J774A.1 macrophages were transfected with Herring testes-DNA (HT-DNA) using lipofectamine 3000 (Invitrogen) and then irradiated by 250 mJ/cm² UV-C using a UV crosslinker (Stratagene) to induce apoptosis and the resulting amount of IFN-beta protein was measured using the High Sensitivity Mouse IFN Beta ELISA Kit (PBL Assay Science). Functional cGAS and STING were required in macrophages for IFNβ induction in response to exogenously delivered cytosolic DNA through liposome-mediated transfection (FIGS. 18A & 18B). Western blot analysis of cGAS and STING expression in MC38 tumor cells and J774A.1 macrophages determined that J774A.1 macrophages express cGAS and STING, while MC38 tumor cells only express cGAS (FIG. 18C). Consistent with a lack of STING expression, MC38 cells themselves did not produce any detectable IFNβ after UV radiation (FIGS. 18C & 19A). IFNβ was induced when UV-irradiated tumors cells were co-cultured with WT but not Sting^{gt/gt} macrophages (FIG. 19A).

In another experiment, dying tumor cells were transfected with DNA as described above, cocultured with macrophages for 24 hours, and IFNβ protein levels in culture supernatant were determined using the High Sensitivity Mouse IFN Beta ELISA Kit (PBL Assay Science). Macrophages deficient in cGAS were still able to produce IFNβ when co-cultured with dying tumor cells (FIG. 19B). To investigate whether the tumor cells were providing functional cGAS to the macrophages, resulting in IFN-beta expression, cGAS^{-/-} MC38 cells were tested for the ability to induce IFN-beta expression. This showed that cGAS^{-/-} MC38 cells were unable to stimulate IFNβ production, regardless of the genotype of macrophages (FIGS. 19A & 19B). These results support a model wherein STING in macrophages is trans-activated by tumor-derived cGAS.

To investigate the significance of tumor-derived cGAS in trans-activating STING in vivo, we carried out tumor studies with cGAS^{-/-} MC38 or AB22 tumor cells. Briefly, C57BL/6N mice were inoculated with 1×10⁵ WT or cGAS MC38 cells or BALB/c mice were inoculated with 1×10⁷ WT or cGAS^{-/-} AB22 cells then treated with anti-MerTK 14C9 (mIgG2a LALAPG) or control antibody as described in Example 11. For early stage tumor investigation, mice were administered anti-MerTK 14C9 (mIgG2a LALAPG) or control antibody 4 days after inoculation (FIG. 19C) or anti-MerTK 14C9 (mIgG2a LALAPG), anti-PD-L1, or control antibody 4, 7, and 10 days after inoculation with tumor cells (FIGS. 19D & 19E). For established tumor investigation, mice were administered anti-MerTK 14C9 (mIgG2a LALAPG) in combination with anti-PD-L1 or control antibody 18, 22, 26, and 30 days after inoculation (FIG. 18E), or tumors were grown to volumes of 100-150 mm³, and then mice were administered anti-MerTK 14C9 (mIgG2a LALAPG) or control antibody that day (FIG. 18D).

After anti-MerTK antibody treatment the type I IFN response observed in MC38 tumors was completely lost in cGAS^{-/-} MC38 tumors (FIG. 19C). Similar results were obtained in mesothelioma AB22 tumors (FIG. 18D). Importantly, cGAS deficiency rendered tumors resistant to single-agent treatment of anti-MerTK antibody or anti-PD-L1 antibody in early tumor progression setting (FIGS. 19D & 19E) or to combination therapy when treating fully established tumors (FIG. 18E). Therefore, the anti-MerTK antibody antitumor effect depends on the presence of functional cGAS in tumor cells.

### Example 16: Anti-MerTK Antibody Antitumor Effect Potentially Depends on the Presence of Gap Junctions Between Tumor Cells and Macrophages

It is known that the activation of cGAS leads to production of cGAMP. To determine if tumor cell-derived cGAMP is responsible for the activation of STING in immune cells, protein quantification by LC-MS/MS was used to measure cGAMP production in WT and cGAS^{-/-} MC38 tumor cells transfected with DNA. cGAMP increased following transfection with HT-DNA in WT tumor cells, but cGAS^{-/-} tumor cells lost the capacity to generate cGAMP in response to cytosolic DNA (FIG. 18F). To determine if tight junctions facilitate tumor cell-derived cGAMP transmission into macrophages, dye transfer assays and IFN-beta transfer assays between tumor and macrophage cells were performed. For dye transfer assays, donor cells (WTMC38 tumor cells, Cx43/MC38 tumor cells, or J774A.1 macrophages) were stained with 0.5 μg/ml Calcein-AM dye (ThermoFisher) in PBS at 37° C. for 30 minutes and washed extensively with culture medium to remove free dye. Calcein-loaded donor cells were co-cultured with recipient cells (WT or Cx43′ MC38 tumor cells) at a ratio of 3:1 for 4-5 hours. Cells were analyzed by FACS to assess dye transfer. To increase Cx43 expression, J774A.1 macrophages were stimulated with 0.5 μg/ml LPS (Invivogen) overnight before their use for dye transfer experiment. PE-Texas Red conjugated anti-CD11b (ThermoFisher) was used to distinguish macrophages from tumor cells.

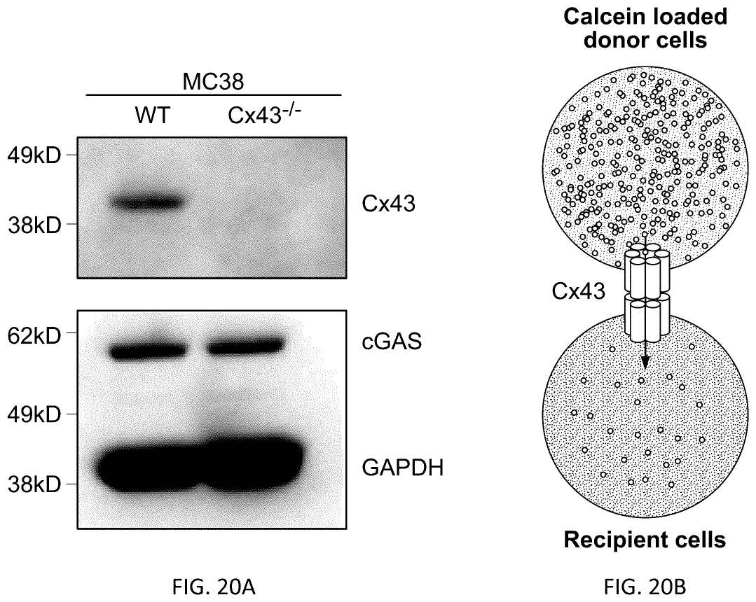

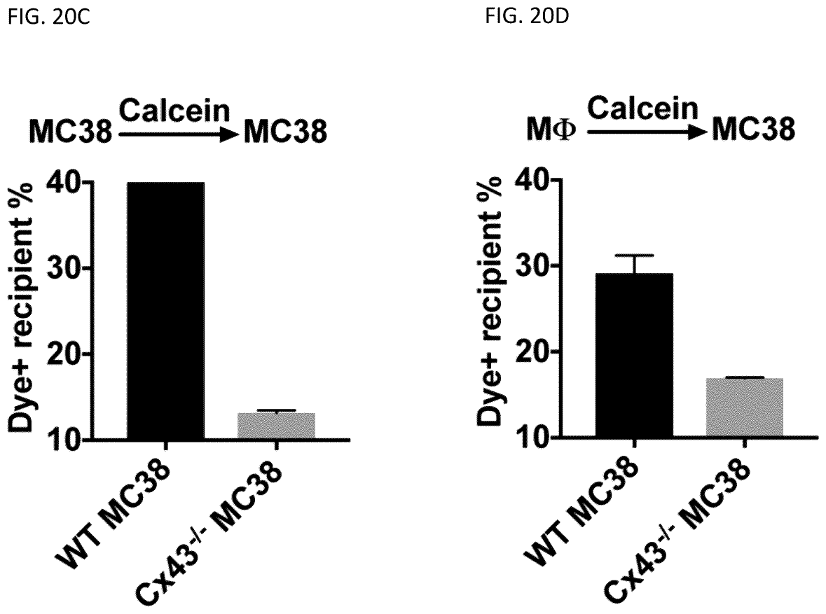

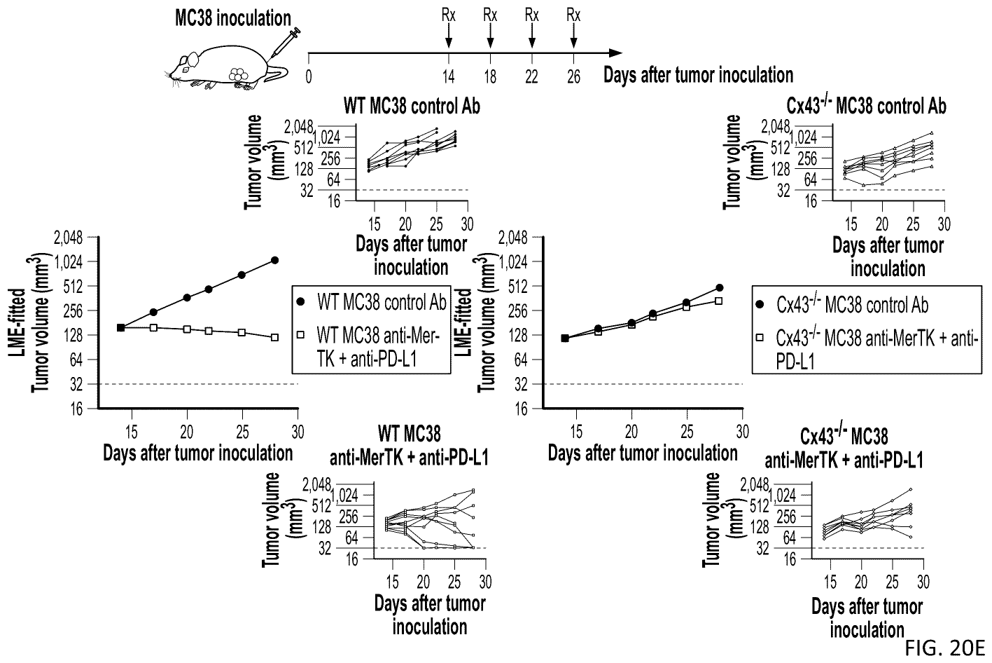

Cx43 is the most ubiquitously expressed connexin family protein (Cx), which assemble to form gap junctions between neighboring cells. Loss of Cx43 abolished the dye transfer between MC38 cells (FIGS. 20B & 20C), confirming Cx43 is the key connexin for forming functional gap junctions. The dye transfer experiment also showed Cx43-dependent intercellular communication between macrophages and MC38 tumor cells (FIG. 20D).

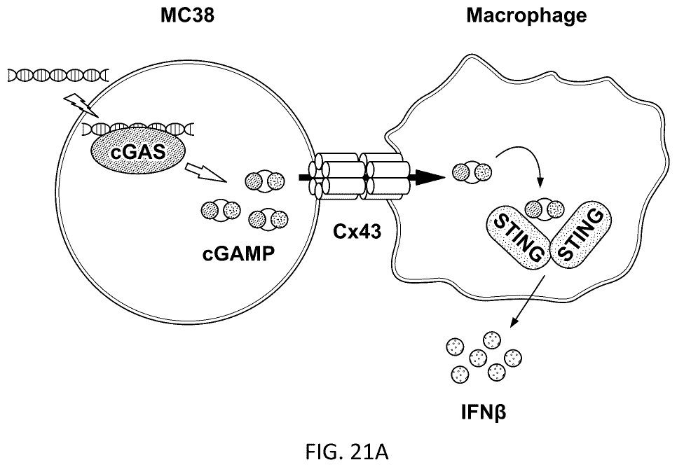

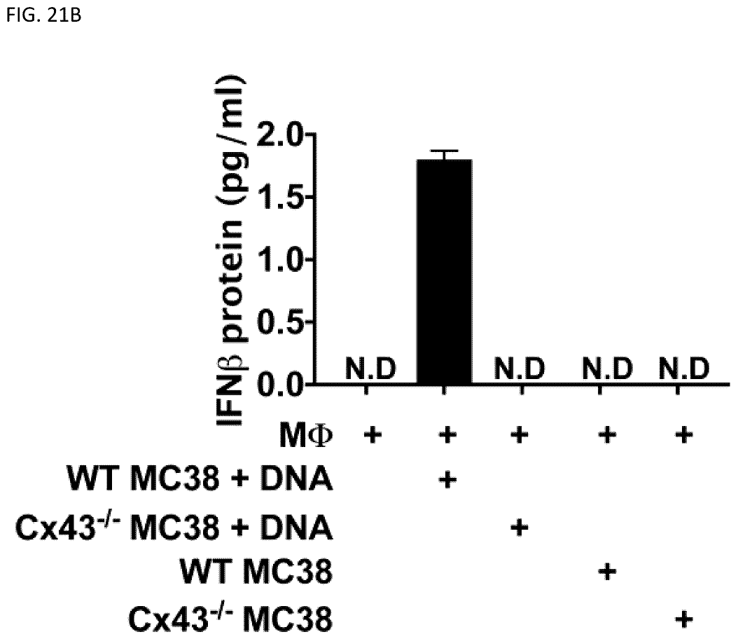

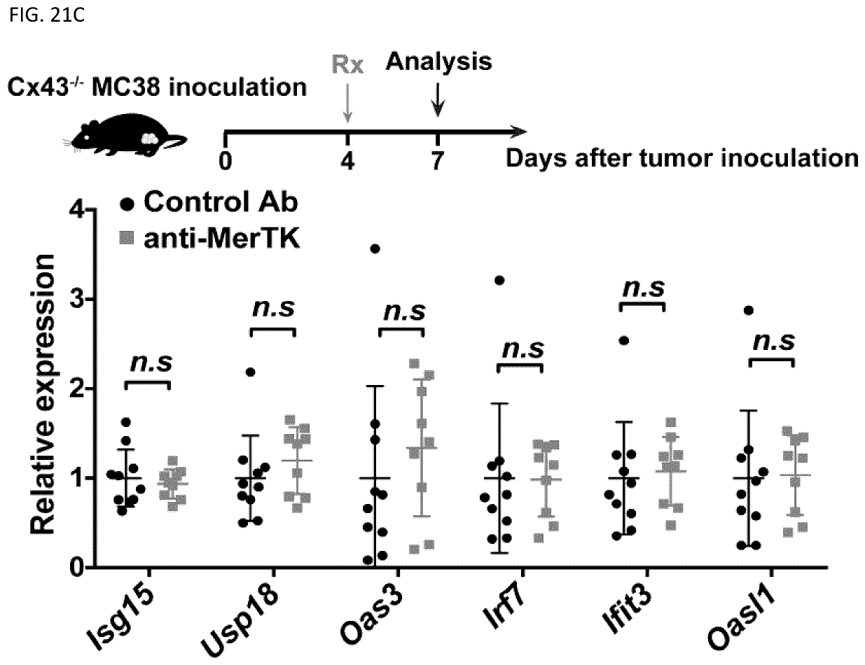

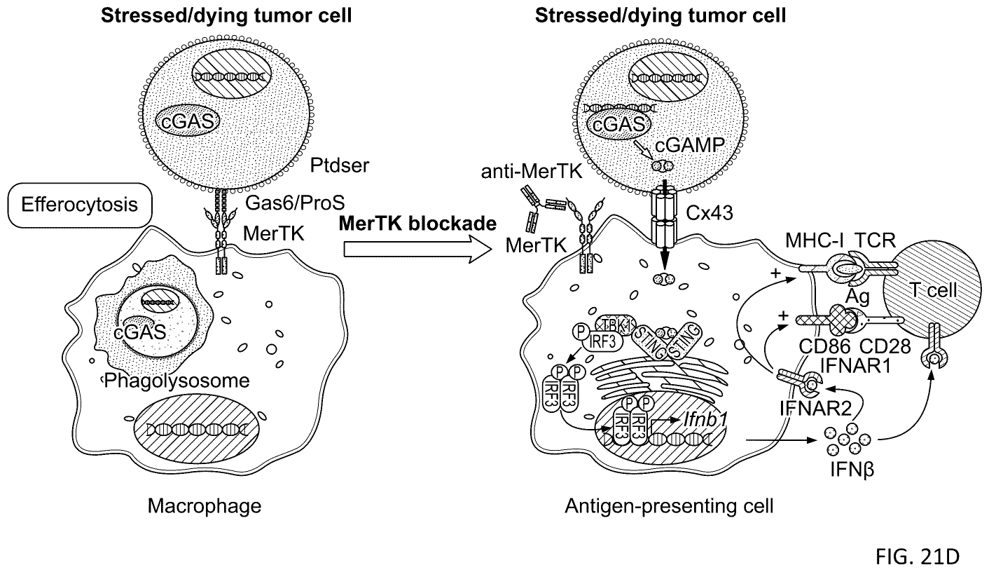

In another experiment, DNA was transfected into WT or Cx43′ MC38 tumor cells to induce the production of cGAMP. After DNA transfection, 5×10⁵ tumor cells were co-cultured with 5×10⁵ LPS-treated J774A.1 macrophages for 24 hours to allow cGAMP transfer. IFNβ protein in culture supernatant was measured with the High Sensitivity Mouse IFN Beta ELISA Kit (PBL Assay Science). Since MC38 tumor cells are not able to produce IFNβ due to lack of STING expression, the production of IFNβ reflects a productive transfer of cGAMP from tumor cells to macrophages. DNA-transfected WT MC38 cells, but not Cx43^{-/-} MC38 cells, induced IFNβ production (FIG. 21B). Given that WT and Cx43 MC38 cells expressed similar level of cGAS (FIG. 20A), the failed induction of IFNβ by Cx43^{-/-} MC38 cells is likely due to the defective gap junctions. Collectively, these data support the possibility of a gap junction-dependent transfer of cGAMP from tumor cells to macrophages.

WT and Cx43′ MC38 tumor cells were further investigated to determine whether defective gap junctions abolish the antitumor effect of anti-MerTK 14C9 (mIgG2a LALAPG) in this model. Briefly, C57BL/6N mice were inoculated with Cx43−/− MC38 cells as described in Example 11 and treated with anti-MerTK 14C9 (mIgG2a LALAPG) 4 days later. After anti-MerTK antibody treatment, unlike WT MC38 tumors, no significant changes in the expression of ISGs in Cx43′ MC38 tumors were observed (FIG. 21C).

The effect of Cx43 loss on the anti-MerTK antibody antitumor effect was also investigated. Briefly, C57BL/6N mice were inoculated with 1×10⁵ WT or cGAS^{-/-} MC38 cells or BALB/c mice were inoculated with 1×10⁷ WT or Cx43/MC38 cells then treated with anti-MerTK 14C9 (mIgG2a LALAPG) or control antibody as described in Example 11. Mice were administered anti-MerTK 14C9 (mIgG2a LALAPG) and anti-PD-L1 as a combination therapy or control antibody at 14, 18, 22, and 26 days after inoculation with tumor cells. Cx43^{-/-} MC38 tumors became resistant to the combination therapy of anti-MerTK 14C9 (mIgG2a LALAPG) and anti-PD-L1 (FIG. 20E). Collectively, these results demonstrate that anti-MerTK antibody is effective at treating tumors and that the effectiveness of anti-MerTK antibody is dependent on the presence of host STING, tumor-derived cGAS, and tight junctions between tumor and macrophage cells.

### Example 17: Anti-MerTK Antibody Blocks Ongoing Clearance of Apoptotic Cells by Tumor Associated Macrophages (TAMs)

Cell free DNA (cfDNA) in blood circulation is released by damaged or dead cells (Wan, J. C. M. et al. (2017) Nat. Rev. Cancer 17:223-238). In cancer patients or tumor bearing mice, a subpopulation of cfDNA is tumor-derived, called circulating tumor DNA (ctDNA). In this Example, a SNP was utilized to distinguish host-derived cfDNA from tumor-derived ctDNA in an MC38 tumor model to investigate the effect of anti-MerTK antibody treatment.

MC38 tumor cells were inoculated into C57BL/6J mice and tumors were allowed to establish. Anti-MerTK or control antibody was administered after tumors were established. Three days post treatment, whole blood was collected by cardiac puncture into Cell-free DNA BCT tubes (Streck). Plasma was obtained by a double spin procedure (1,600 g for 10 minutes, separation, followed by 16,000 g for 10 minutes). cfDNA (12.5 μL for 200 μL of plasma) was obtained using MagMAX™ Cell-Free DNA Isolation Kit (ThermoFisher Scientific) following the manufacturer's protocol.

To assay the levels of host-derived cfDNA and MC38-derived ctDNA, multiplexing droplet digital PCR (Bio-Rad Laboratories) was performed using an assay containing primers and probes targeting SNPs of gene Jmjd1c (rs13480628, ThermoFisher Scientific). C57BL/6J mice and MC38 cells express a “T” and a “C” allele at this locus, respectively. For droplet digital PCR, 4 μL of isolated cfDNA was used in each 20 L-reaction, and each sample was analyzed in duplicates. Sample analysis was performed using QuantaSoft software (Bio-Rad Laboratories), and target DNA (copies/μL of plasma) was calculated as the quantitative outcome. Size of isolated cfDNA was also confirmed to be predominantly ˜170 bp using Agilent Bioanalyzer 2100.

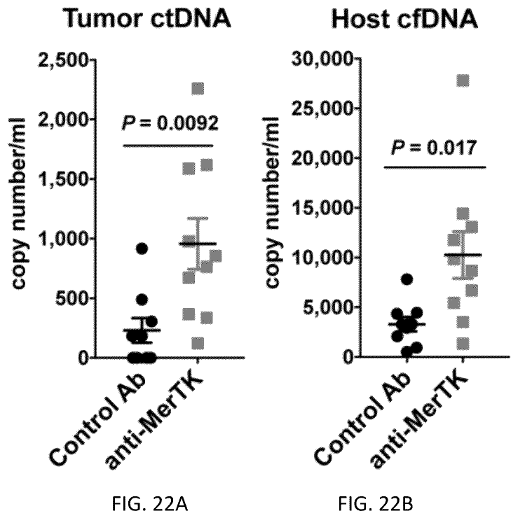

MC38 tumor cells were inoculated into C57BL/6J mice as described above, and anti-MerTK or control antibody was administered after tumors were established. Three days after anti-MerTK treatment, a significant increase of ctDNA in the plasma of tumor-bearing mice was detected (FIG. 22A). Anti-MerTK also increased the level of host-derived cfDNA in blood circulation (FIG. 22B). These results clearly demonstrate that in tumor microenvironment anti-MerTK was able to block the ongoing clearance of apoptotic cells by TAMs.

### Example 18: Analysis of Anti-MerTK Antibody Binding Affinity and Epitope Mapping

For binding affinity determinations of anti-MerTK antibodies of the present disclosure as a control along with commercial MerTK antibodies, Surface Plasmon Resonance (SPR) measurement with a BIAcore™-T200 instrument was used. First, two rabbit antibodies (Y323 and 10g86 D21F11) and the anti-MerTK antibody h13B4.v16 were captured by protein A sensor chip, and eight mouse antibodies (A3KCAT, 2D2, 7E5G1, 7N-20, 590H11G1E3, MAB891, MAB8911 and MAB8912-100) were captured by goat anti-mouse IgGs sensor chip respectively on each flow cell to achieve approximately 100 RU. Three-fold serial dilutions of human MerTK (0.4 nM to 100 nM) were injected in HBS-EP buffer (0.01M HEPES pH 7.4, 0.15M NaCl, 3 mM EDTA and 0.05% v/v surfactant P20) at 25° C. with a flow rate of 50 μl/min to record the binding response as a function of time. The sensorgram was fitted with one-to-one Langmuir binding model to calculate association rates (kₒₙ) and dissociation rates (k_{off}) (BIAcore T200 evaluation software version 2.0). The binding affinity (equilibrium dissociation constant (KD)) was calculated as the ratio k_{off}/kₒₙ.

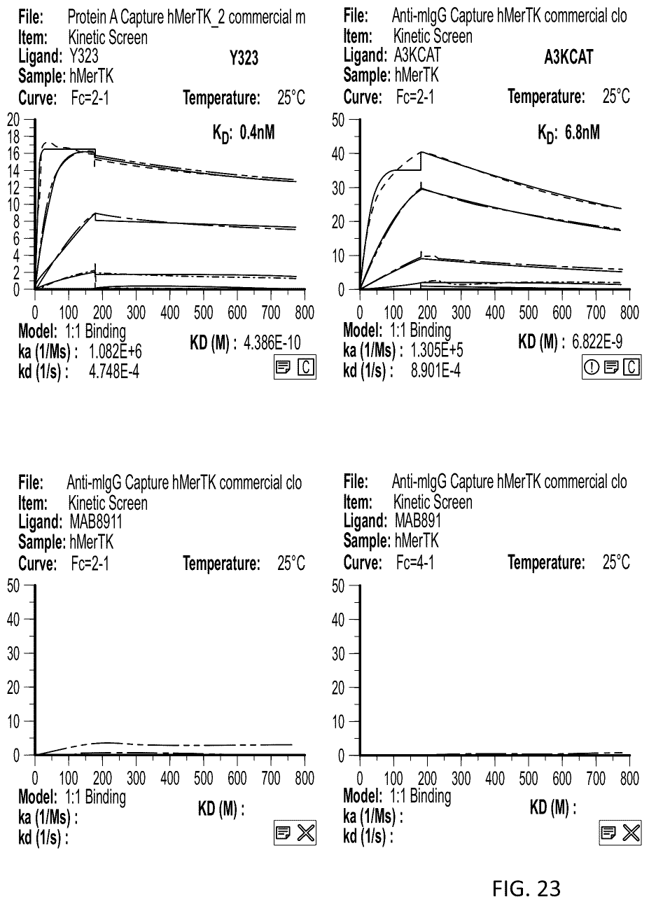

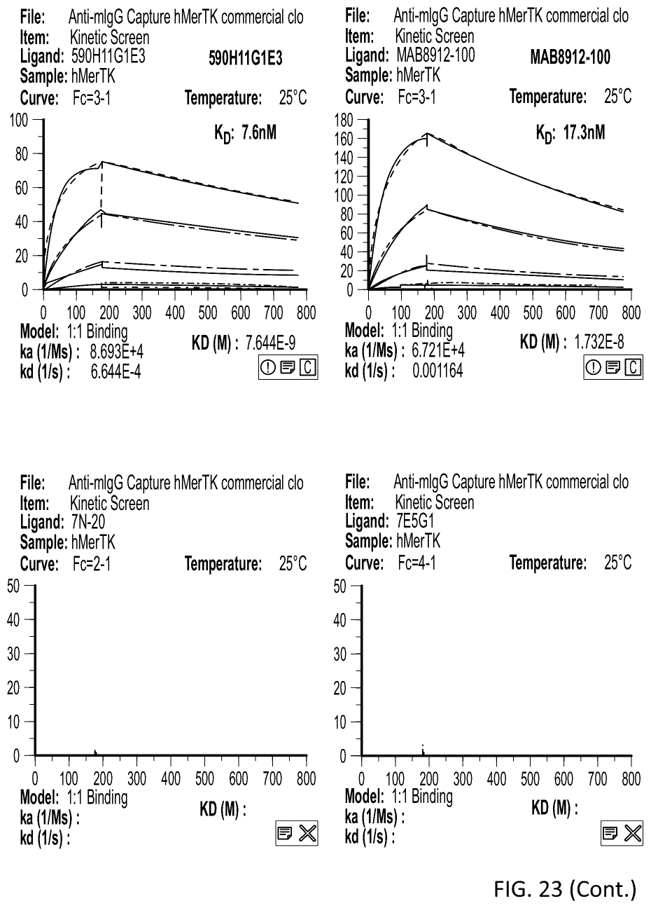

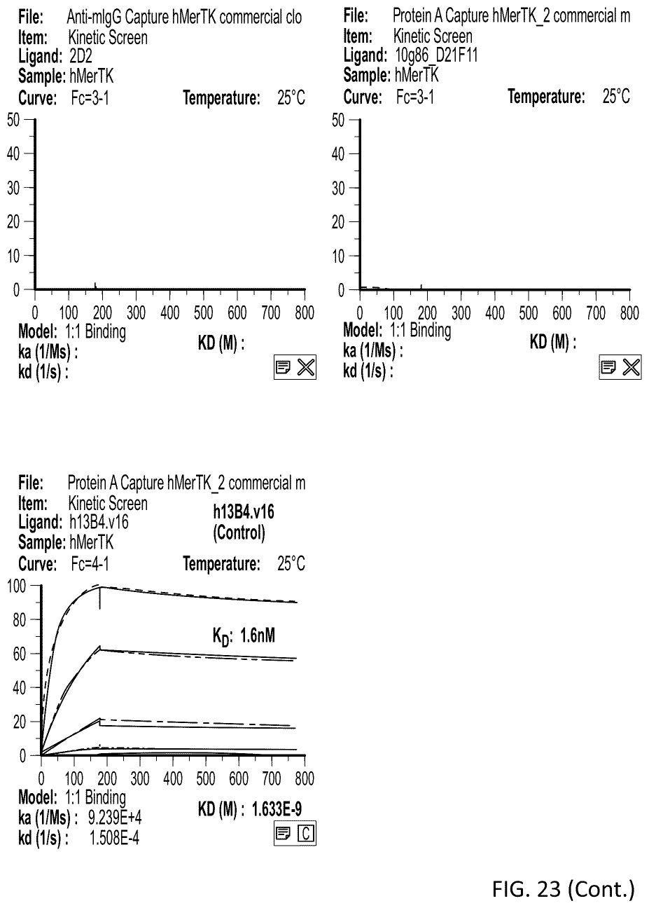

As shown in FIG. 23, only 4 out of 10 selected commercial antibodies showed binding to human MerTK. The results indicated a binding affinity to human MerTK of 0.4 nM for Y323, 6.8 nM for A3KCAT, 7.6 nM for 590H11G1E3, 17.3 nM for MAB8912-100 and 1.6 nM for h13B4.v16, while the remaining antibodies showed no binding. FIG. 23 shows that Y323 is a higher affinity antibody than h13B4.v16, including having about a 12-fold higher on-rate (ka) and 3-fold higher off-rate (kd) compared to h13B4.v16. In addition, as noted above, FIGS. 3, 4A-4C, & Table 19 demonstrate that h13B4.v16 possesses biological properties desired for an anti-MerTK antibody, such as more potent inhibition of efferocytosis. Accordingly, h13B4.v16 possesses unique binding characteristics including on and off rates, affinity, binding epitope, and the resulting desired biological effects, e.g., efferocytosis, which make this antibody a particularly useful therapeutic candidate.

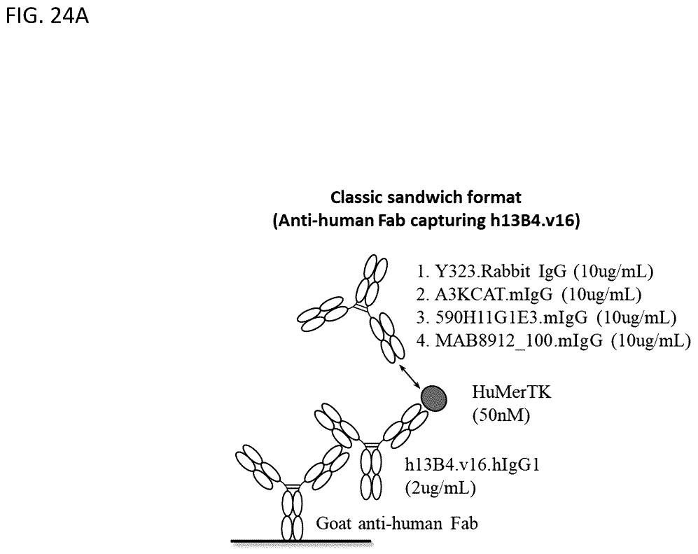

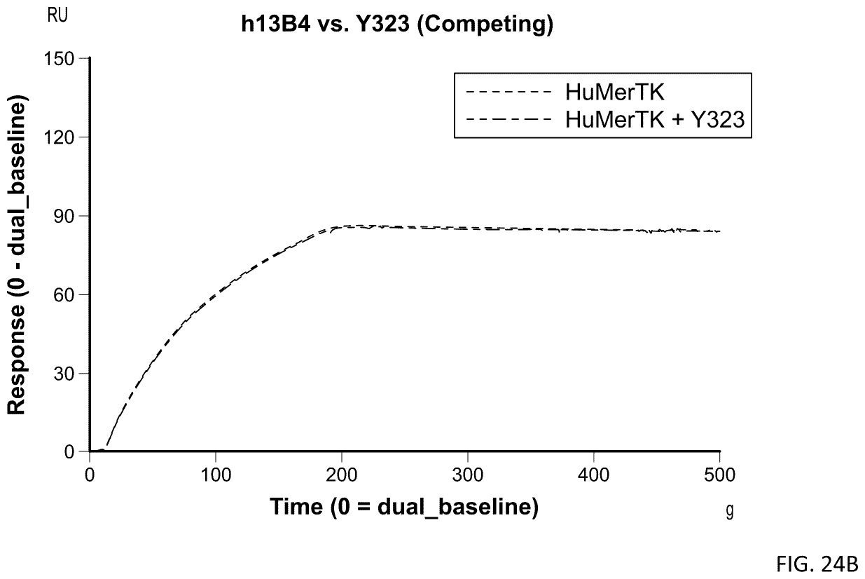

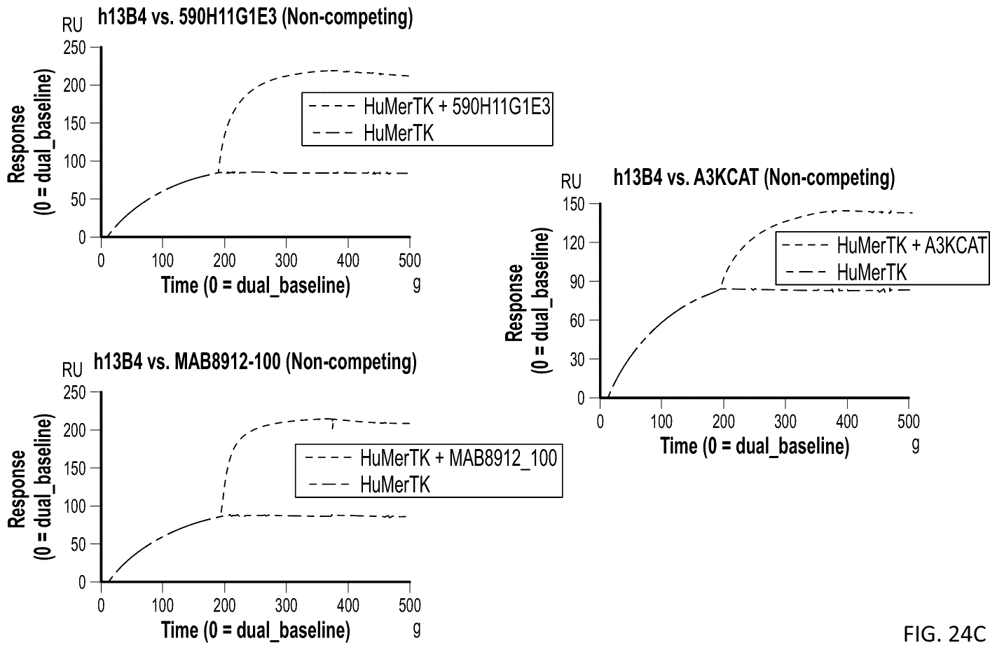

These 4 antibodies (Y323, A3KCAT, 590H11G1E3 and MAB8912-100) were further assessed to determine whether their binding epitope competes with h13B4.v16 for binding human MerTK. To conduct this experiment, the same BIAcore™-T200 instrument was used, and the classic sandwich format was applied (FIG. 24A). h13B4.v16 at 2 ug/mL was first captured by goat anti-human Fab sensor chip, and then 50 nM of human MerTK was injected at a flow rate of 50 μl/min in HBS-EP buffer to record 1^{st} binding, followed by the 2^{nd} binding with or without the tested antibody at 10 ug/mL of injection. If the 2^{nd} binding was observed, the tested antibody did not compete with the lead molecule, and vice versa, if the 2^{nd} binding was not observed, the tested antibody did compete with h13B4.v16.

The results indicated that only antibody Y323 competed with h13B4.v16 for binding to human MerTK (FIG. 24B). The remaining three antibodies did not compete with h13B4.v16 for binding to human MerTK (FIG. 24C).

Although the present disclosure has been described in some detail by way of illustration and example for purposes of clarity of understanding, the descriptions and examples should not be construed as limiting the scope of the present disclosure. The disclosures of all patent and scientific literature cited herein are expressly incorporated in their entirety by reference.

1. An isolated antibody that binds to MerTK, wherein the antibody reduces MerTK-mediated clearance of apoptotic cells.

2. The antibody of claim 1, wherein the antibody reduces MerTK-mediated clearance of apoptotic cells by phagocytes.

3. The antibody of claim 2, wherein the phagocytes are macrophages.

4. The antibody of claim 3, wherein the macrophages are tumor-associated macrophages.

5. The antibody of claim 1, wherein the clearance of apoptotic cells is reduced as measured in an apoptotic cell clearance assay at room temperature; or wherein the antibody reduces ligand-mediated MerTK signaling.

6. (canceled)

7. The antibody of claim 1, wherein the antibody induces a pro-inflammatory response or a type I IFN response.

8. The antibody of claim 1, wherein the antibody is a monoclonal antibody, and/or wherein the antibody is a human, humanized, or chimeric antibody.

9. (canceled)

10. The antibody of claim 1, wherein the antibody is an antibody fragment that binds MerTK.

11. The antibody of claim 1, wherein the antibody binds to a fibronectin-like domain or an immunoglobulin-like domain of MerTK.

12. (canceled)

13. An isolated antibody that binds to MerTK, wherein the antibody comprises:
(A) an HVR-H1 comprising the amino acid sequence of SEQ ID NO: 4, an HVR-H2 comprising the amino acid sequence of SEQ ID NO: 5, an HVR-H3 comprising the amino acid sequence of SEQ ID NO: 6, an HVR-L1 comprising the amino acid sequence of SEQ ID NO: 1, an HVR-L2 comprising the amino acid sequence of SEQ ID NO: 2, and an HVR-L3 comprising the amino acid sequence of SEQ ID NO: 3;
(B) an HVR-H1 comprising the amino acid sequence of SEQ ID NO: 10, an HVR-H2 comprising the amino acid sequence of SEQ ID NO: 11, an HVR-H3 comprising the amino acid sequence of SEQ ID NO: 12, an HVR-L1 comprising the amino acid sequence of SEQ ID NO: 7, an HVR-L2 comprising the amino acid sequence of SEQ ID NO: 8, and an HVR-L3 comprising the amino acid sequence of SEQ ID NO: 9;
(C) an HVR-H1 comprising the amino acid sequence of SEQ ID NO: 16, an HVR-H2 comprising the amino acid sequence of SEQ ID NO: 17, an HVR-H3 comprising the amino acid sequence of SEQ ID NO: 18, an HVR-L1 comprising the amino acid sequence of SEQ ID NO: 13, an HVR-L2 comprising the amino acid sequence of SEQ ID NO: 14, and an HVR-L3 comprising the amino acid sequence of SEQ ID NO: 15;
(D) an HVR-H1 comprising the amino acid sequence of SEQ ID NO: 22, an HVR-H2 comprising the amino acid sequence of SEQ ID NO: 23, an HVR-H3 comprising the amino acid sequence of SEQ ID NO: 24, an HVR-L1 comprising the amino acid sequence of SEQ ID NO: 19, an HVR-L2 comprising the amino acid sequence of SEQ ID NO: 20, and an HVR-L3 comprising the amino acid sequence of SEQ ID NO: 21;
(E) an HVR-H1 comprising the amino acid sequence of SEQ ID NO: 27, an HVR-H2 comprising the amino acid sequence of SEQ ID NO: 28, an HVR-H3 comprising the amino acid sequence of SEQ ID NO: 29, an HVR-L1 comprising the amino acid sequence of SEQ ID NO: 25, an HVR-L2 comprising the amino acid sequence of SEQ ID NO: 14, and an HVR-L3 comprising the amino acid sequence of SEQ ID NO: 26;
(F) an HVR-H1 comprising the amino acid sequence of SEQ ID NO: 33, an HVR-H2 comprising the amino acid sequence of SEQ ID NO: 34, an HVR-H3 comprising the amino acid sequence of SEQ ID NO: 35, an HVR-L1 comprising the amino acid sequence of SEQ ID NO: 30, an HVR-L2 comprising the amino acid sequence of SEQ ID NO: 31, and an HVR-L3 comprising the amino acid sequence of SEQ ID NO: 32;
(G) an HVR-H1 comprising the amino acid sequence of SEQ ID NO: 38, an HVR-H2 comprising the amino acid sequence of SEQ ID NO: 39, an HVR-H3 comprising the amino acid sequence of SEQ ID NO: 40, an HVR-L1 comprising the amino acid sequence of SEQ ID NO: 36, an HVR-L2 comprising the amino acid sequence of SEQ ID NO: 14, and an HVR-L3 comprising the amino acid sequence of SEQ ID NO: 37;
(H) an HVR-H1 comprising the amino acid sequence of SEQ ID NO: 44, an HVR-H2 comprising the amino acid sequence of SEQ ID NO: 45, an HVR-H3 comprising the amino acid sequence of SEQ ID NO: 46, an HVR-L1 comprising the amino acid sequence of SEQ ID NO: 41, an HVR-L2 comprising the amino acid sequence of SEQ ID NO: 42, and an HVR-L3 comprising the amino acid sequence of SEQ ID NO: 43;
(I) an HVR-H1 comprising the amino acid sequence of SEQ ID NO: 50, an HVR-H2 comprising the amino acid sequence of SEQ ID NO: 51, an HVR-H3 comprising the amino acid sequence of SEQ ID NO: 52, an HVR-L1 comprising the amino acid sequence of SEQ ID NO: 47, an HVR-L2 comprising the amino acid sequence of SEQ ID NO: 48, and an HVR-L3 comprising the amino acid sequence of SEQ ID NO: 49;
(J) an HVR-H1 comprising the amino acid sequence of SEQ ID NO: 56, an HVR-H2 comprising the amino acid sequence of SEQ ID NO: 57, an HVR-H3 comprising the amino acid sequence of SEQ ID NO: 58, an HVR-L1 comprising the amino acid sequence of SEQ ID NO: 53, an HVR-L2 comprising the amino acid sequence of SEQ ID NO: 54, and an HVR-L3 comprising the amino acid sequence of SEQ ID NO: 55; or
(K) an HVR-H1 comprising the amino acid sequence of SEQ ID NO: 62, an HVR-H2 comprising the amino acid sequence of SEQ ID NO: 63, an HVR-H3 comprising the amino acid sequence of SEQ ID NO: 64, an HVR-L1 comprising the amino acid sequence of SEQ ID NO: 59, an HVR-L2 comprising the amino acid sequence of SEQ ID NO: 60, and an HVR-L3 comprising the amino acid sequence of SEQ ID NO: 61.

14. (canceled)

15. (canceled)

16. The antibody of claim 13, comprising: (A) a VH comprising the amino acid sequence of SEQ ID NO: 83 and a VL comprising the amino acid sequence of SEQ ID NO: 65; (B) a VH comprising the amino acid sequence of SEQ ID NO: 84 and a VL comprising the amino acid sequence of SEQ ID NO: 66; (C) a VH comprising the amino acid sequence of SEQ ID NO: 85 and a VL comprising the amino acid sequence of SEQ ID NO: 67; (D) a VH comprising the amino acid sequence of SEQ ID NO: 86 and a VL comprising the amino acid sequence of SEQ ID NO: 68; (E) a VH comprising the amino acid sequence of SEQ ID NO: 87 and a VL comprising the amino acid sequence of SEQ ID NO: 69; (F) a VH comprising the amino acid sequence of SEQ ID NO: 88 and a VL comprising the amino acid sequence of SEQ ID NO: 70; (G) a VH comprising the amino acid sequence of SEQ ID NO: 89 and a VL comprising the amino acid sequence of SEQ ID NO: 70; (H) a VH comprising the amino acid sequence of SEQ ID NO: 90 and a VL comprising the amino acid sequence of SEQ ID NO: 71; (I) a VH comprising the amino acid sequence of SEQ ID NO: 91 and a VL comprising the amino acid sequence of SEQ ID NO: 72; (J) a VH comprising the amino acid sequence of SEQ ID NO: 92 and a VL comprising the amino acid sequence of SEQ ID NO: 73; (K) a VH comprising the amino acid sequence of SEQ ID NO: 94 and a VL comprising the amino acid sequence of SEQ ID NO: 75; (L) a VH comprising the amino acid sequence of SEQ ID NO: 95 and a VL comprising the amino acid sequence of SEQ ID NO: 76; (M) a VH comprising the amino acid sequence of SEQ ID NO: 96 and a VL comprising the amino acid sequence of SEQ ID NO: 77; (N) a VH comprising the amino acid sequence of SEQ ID NO: 97 and a VL comprising the amino acid sequence of SEQ ID NO: 78; (O) a VH comprising the amino acid sequence of SEQ ID NO: 98 and a VL comprising the amino acid sequence of SEQ ID NO: 79; (P) a VH comprising the amino acid sequence of SEQ ID NO: 99 and a VL comprising the amino acid sequence of SEQ ID NO: 80; (Q) a VH comprising the amino acid sequence of SEQ ID NO: 100 and a VL comprising the amino acid sequence of SEQ ID NO: 81; or (R) a VH comprising the amino acid sequence of SEQ ID NO: 101 and a VL comprising the amino acid sequence of SEQ ID NO: 82.

17-28. (canceled)

29. The antibody of claim 13, wherein the antibody comprises a heavy chain comprising the amino acid sequence of SEQ ID NO: 102, SEQ ID NO: 103, SEQ ID NO: 104, SEQ ID NO: 105, SEQ ID NO: 106, SEQ ID NO: 107, SEQ ID NO: 108, or SEQ ID NO: 109.

30. The antibody of claim 13, wherein the antibody comprises a light chain comprising the amino acid sequence of SEQ ID NO: 110, SEQ ID NO: 111, SEQ ID NO: 112, SEQ ID NO: 113, SEQ ID NO: 114, SEQ ID NO: 115, SEQ ID NO: 116, or SEQ ID NO: 117.

31.-128. (canceled)

129. An isolated antibody that binds to the same epitope on MerTK as a reference antibody selected from the group consisting of:
(a) an antibody that comprises a VH comprising the amino acid sequence of SEQ ID NO: 83 and a VL comprising the amino acid sequence of SEQ ID NO: 65;
(b) an antibody that comprises a VH comprising the amino acid sequence of SEQ ID NO: 84 and a VL comprising the amino acid sequence of SEQ ID NO: 66;
(c) an antibody that comprises a VH comprising the amino acid sequence of SEQ ID NO: 85 and a VL comprising the amino acid sequence of SEQ ID NO: 67;
(d) an antibody that comprises a VH comprising the amino acid sequence of SEQ ID NO: 86 and a VL comprising the amino acid sequence of SEQ ID NO: 68;
(e) an antibody that comprises a VH comprising the amino acid sequence of SEQ ID NO: 87 and a VL comprising the amino acid sequence of SEQ ID NO: 69;
(f) an antibody that comprises a VH comprising the amino acid sequence of SEQ ID NO: 88 and a VL comprising the amino acid sequence of SEQ ID NO: 70;
(g) an antibody that comprises a VH comprising the amino acid sequence of SEQ ID NO: 89 and a VL comprising the amino acid sequence of SEQ ID NO: 70;
(h) an antibody that comprises a VH comprising the amino acid sequence of SEQ ID NO: 90 and a VL comprising the amino acid sequence of SEQ ID NO: 71;
(i) an antibody that comprises a VH comprising the amino acid sequence of SEQ ID NO: 91 and a VL comprising the amino acid sequence of SEQ ID NO: 72;
(j) an antibody that comprises a VH comprising the amino acid sequence of SEQ ID NO: 92 and a VL comprising the amino acid sequence of SEQ ID NO: 73;
(k) an antibody that comprises a VH comprising the amino acid sequence of SEQ ID NO: 93 and a VL comprising the amino acid sequence of SEQ ID NO: 74;
(l) an antibody that comprises a VH comprising the amino acid sequence of SEQ ID NO: 94 and a VL comprising the amino acid sequence of SEQ ID NO: 75;
(m) an antibody that comprises a VH comprising the amino acid sequence of SEQ ID NO: 95 and a VL comprising the amino acid sequence of SEQ ID NO: 76;
(n) an antibody that comprises a VH comprising the amino acid sequence of SEQ ID NO: 96 and a VL comprising the amino acid sequence of SEQ ID NO: 77;
(o) an antibody that comprises a VH comprising the amino acid sequence of SEQ ID NO: 97 and a VL comprising the amino acid sequence of SEQ ID NO: 78;
(p) an antibody that comprises a VH comprising the amino acid sequence of SEQ ID NO: 98 and a VL comprising the amino acid sequence of SEQ ID NO: 79;
(q) an antibody that comprises a VH comprising the amino acid sequence of SEQ ID NO: 99 and a VL comprising the amino acid sequence of SEQ ID NO: 80;
(r) an antibody that comprises a VH comprising the amino acid sequence of SEQ ID NO: 100 and a VL comprising the amino acid sequence of SEQ ID NO: 81; and
(s) an antibody that comprises a VH comprising the amino acid sequence of SEQ ID NO: 101 and a VL comprising the amino acid sequence of SEQ ID NO: 82.

130-165. (canceled)

166. The isolated antibody of claim 128, wherein the antibody binds to human MerTK.

167. The antibody of claim 13, wherein the antibody is a full length IgG1, IgG2, IgG3, or IgG4 antibody.

168. The antibody of claim 167, wherein the antibody is a full length IgG1 antibody.

169. The antibody of claim 168, wherein the antibody comprises a LALAPG mutation.

170-173. (canceled)

174. The antibody of claim 1, wherein:
(a) the antibody binds to Human MerTK with a dissociation constant of ≤100 nM at 25° C.;
(b) the antibody binds to Cyno MerTK with a dissociation constant of ≤100 nM at 25° C.;
(c) the antibody binds to Mouse MerTK with a dissociation constant of ≤10 nM at 25° C.; or
(d) the antibody binds to Rat MerTK with a dissociation constant of ≤10 nM at 37° C.

175-177. (canceled)

178. A The antibody of claim 13, wherein the antibody comprises:
(a) a heavy chain comprising the amino acid sequence of SEQ ID NO: 102 and a light chain comprising the amino acid sequence of SEQ ID NO: 110;
(b) a heavy chain comprising the amino acid sequence of SEQ ID NO: 103 and a light chain comprising the amino acid sequence of SEQ ID NO: 111;
(c) a heavy chain comprising the amino acid sequence of SEQ ID NO: 104 and a light chain comprising the amino acid sequence of SEQ ID NO: 112;
(d) a heavy chain comprising the amino acid sequence of SEQ ID NO: 105 and a light chain comprising the amino acid sequence of SEQ ID NO: 113;
(e) a heavy chain comprising the amino acid sequence of SEQ ID NO: 106 and a light chain comprising the amino acid sequence of SEQ ID NO: 114;
(f) a heavy chain comprising the amino acid sequence of SEQ ID NO: 107 and a light chain comprising the amino acid sequence of SEQ ID NO: 115;
(g) a heavy chain comprising the amino acid sequence of SEQ ID NO: 108 and a light chain comprising the amino acid sequence of SEQ ID NO: 116; or
(h) a heavy chain comprising the amino acid sequence of SEQ ID NO: 109 and a light chain comprising the amino acid sequence of SEQ ID NO: 117.

179-185. (canceled)

186. The isolated antibody of a claim 178, wherein the antibody reduces MerTK-mediated clearance of apoptotic cells.

187-190. (canceled)

191. The antibody of claim 178, wherein the antibody is a monoclonal antibody.

192-197. (canceled)

198. An isolated nucleic acid encoding the antibody of claim 13.

199. A vector comprising the nucleic acid of claim 198.

200. A host cell comprising the vector of claim 199.

201. A method of producing an anti-MerTK antibody comprising culturing the host cell of claim 200 in a cell culture under conditions suitable for expression of the antibody.

202. The method of claim 201, further comprising recovering the anti-MerTK antibody from the cell culture.

203. (canceled)

204. A pharmaceutical formulation comprising the antibody of claim 13 and a pharmaceutically acceptable carrier.

205-220. (canceled)

221. A method of treating an individual having cancer comprising administering to the individual an effective amount of the antibody of claim 13.

222. The method of claim 221, wherein the cancer expresses functional STING, functional Cx43, and functional cGAS polypeptides.

223-230. (canceled)

231. The method of claim 221, wherein the cancer is colon cancer.

232. A method of reducing MerTK-mediated clearance of apoptotic cells in an individual comprising administering to the individual an effective amount of the antibody of claim 13 to reduce MerTK-mediated clearance of apoptotic cells.

233. (canceled)


SEQUENCE LISTING (137 sequences)

SEQ ID NO: 1 - protein, 12 aa, synthetic construct
  REGION 1..12: note = Synthetic Construct
QSSPNIYSNYLS

SEQ ID NO: 2 - protein, 7 aa, synthetic construct
  REGION 1..7: note = Synthetic Construct
GASTLAS

SEQ ID NO: 3 - protein, 12 aa, synthetic construct
  REGION 1..12: note = Synthetic Construct
AGGYSDSSEAYA

SEQ ID NO: 4 - protein, 5 aa, synthetic construct
  REGION 1..5: note = Synthetic Construct
NYPMS

SEQ ID NO: 5 - protein, 16 aa, synthetic construct
  REGION 1..16: note = Synthetic Construct
VISSTGGTNYASWAKG

SEQ ID NO: 6 - protein, 18 aa, synthetic construct
  REGION 1..18: note = Synthetic Construct
VDFLVYLGGAYIIWGLDL

SEQ ID NO: 7 - protein, 13 aa, synthetic construct
  REGION 1..13: note = Synthetic Construct
QSSKSIYNNNWLS

SEQ ID NO: 8 - protein, 7 aa, synthetic construct
  REGION 1..7: note = Synthetic Construct
DASDLAS

SEQ ID NO: 9 - protein, 11 aa, synthetic construct
  REGION 1..11: note = Synthetic Construct
AGGYSGDSDYA

SEQ ID NO: 10 - protein, 5 aa, synthetic construct
  REGION 1..5: note = Synthetic Construct
SNAMS

SEQ ID NO: 11 - protein, 16 aa, synthetic construct
  REGION 1..16: note = Synthetic Construct
IISSSGSTYSASWAKG

SEQ ID NO: 12 - protein, 20 aa, synthetic construct
  REGION 1..20: note = Synthetic Construct
VGFFVGYGAYDYGIIHRLDL

SEQ ID NO: 13 - protein, 13 aa, synthetic construct
  REGION 1..13: note = Synthetic Construct
QSSESVYNNDYLA

SEQ ID NO: 14 - protein, 7 aa, synthetic construct
  REGION 1..7: note = Synthetic Construct
SASTLAS

SEQ ID NO: 15 - protein, 9 aa, synthetic construct
  REGION 1..9: note = Synthetic Construct
AGGYLGNNV

SEQ ID NO: 16 - protein, 5 aa, synthetic construct
  REGION 1..5: note = Synthetic Construct
GYTMG

SEQ ID NO: 17 - protein, 16 aa, synthetic construct
  REGION 1..16: note = Synthetic Construct
VISSGGTTYYTNWAKG

SEQ ID NO: 18 - protein, 19 aa, synthetic construct
  REGION 1..19: note = Synthetic Construct
VAFTAYGGGGFPTLHRLDL

SEQ ID NO: 19 - protein, 13 aa, synthetic construct
  REGION 1..13: note = Synthetic Construct
QSSKSVYNNNWLS

SEQ ID NO: 20 - protein, 7 aa, synthetic construct
  REGION 1..7: note = Synthetic Construct
RASTLES

SEQ ID NO: 21 - protein, 12 aa, synthetic construct
  REGION 1..12: note = Synthetic Construct
AGGYSSSSSANA

SEQ ID NO: 22 - protein, 5 aa, synthetic construct
  REGION 1..5: note = Synthetic Construct
GYAMS

SEQ ID NO: 23 - protein, 16 aa, synthetic construct
  REGION 1..16: note = Synthetic Construct
VISSSGSSYYPSWAKG

SEQ ID NO: 24 - protein, 20 aa, synthetic construct
  REGION 1..20: note = Synthetic Construct
VQFYVGYAVYGYGIIDRLDL

SEQ ID NO: 25 - protein, 13 aa, synthetic construct
  REGION 1..13: note = Synthetic Construct
QASQSVYDSKWLA

SEQ ID NO: 26 - protein, 9 aa, synthetic construct
  REGION 1..9: note = Synthetic Construct
AGAYTDNIV

SEQ ID NO: 27 - protein, 5 aa, synthetic construct
  REGION 1..5: note = Synthetic Construct
SYSMG

SEQ ID NO: 28 - protein, 16 aa, synthetic construct
  REGION 1..16: note = Synthetic Construct
VISASGTTYYASWVNG

SEQ ID NO: 29 - protein, 18 aa, synthetic construct
  REGION 1..18: note = Synthetic Construct
AAFTAYNRGSCVIHRLDL

SEQ ID NO: 30 - protein, 13 aa, synthetic construct
  REGION 1..13: note = Synthetic Construct
QSSPSVYNHNWLS

SEQ ID NO: 31 - protein, 7 aa, synthetic construct
  REGION 1..7: note = Synthetic Construct
EASKLAS

SEQ ID NO: 32 - protein, 12 aa, synthetic construct
  REGION 1..12: note = Synthetic Construct
AGGFSSGSDSFA

SEQ ID NO: 33 - protein, 5 aa, synthetic construct
  REGION 1..5: note = Synthetic Construct
TYSMS

SEQ ID NO: 34 - protein, 16 aa, synthetic construct
  REGION 1..16: note = Synthetic Construct
IVSVAIDPVYATWARG

SEQ ID NO: 35 - protein, 14 aa, synthetic construct
  REGION 1..14: note = Synthetic Construct
VAFSTNGIPHRLDL

SEQ ID NO: 36 - protein, 11 aa, synthetic construct
  REGION 1..11: note = Synthetic Construct
QASESISSRLA

SEQ ID NO: 37 - protein, 13 aa, synthetic construct
  REGION 1..13: note = Synthetic Construct
QTYYGGSTTGWYV

SEQ ID NO: 38 - protein, 5 aa, synthetic construct
  REGION 1..5: note = Synthetic Construct
SYGIS

SEQ ID NO: 39 - protein, 17 aa, synthetic construct
  REGION 1..17: note = Synthetic Construct
YIYPGFGITNYAHSVKG

SEQ ID NO: 40 - protein, 20 aa, synthetic construct
  REGION 1..20: note = Synthetic Construct
DLDYTGGVVGYAYVTYYFTL

SEQ ID NO: 41 - protein, 11 aa, synthetic construct
  REGION 1..11: note = Synthetic Construct
QASQSIGNALA

SEQ ID NO: 42 - protein, 7 aa, synthetic construct
  REGION 1..7: note = Synthetic Construct
AASNLAS

SEQ ID NO: 43 - protein, 12 aa, synthetic construct
  REGION 1..12: note = Synthetic Construct
QTYYAINRYGGA

SEQ ID NO: 44 - protein, 5 aa, synthetic construct
  REGION 1..5: note = Synthetic Construct
VYGMG

SEQ ID NO: 45 - protein, 16 aa, synthetic construct
  REGION 1..16: note = Synthetic Construct
FINNVGNTYYASWAKG

SEQ ID NO: 46 - protein, 11 aa, synthetic construct
  REGION 1..11: note = Synthetic Construct
GGGGDWGYFNI

SEQ ID NO: 47 - protein, 11 aa, synthetic construct
  REGION 1..11: note = Synthetic Construct
QASQNIYSGLA

SEQ ID NO: 48 - protein, 7 aa, synthetic construct
  REGION 1..7: note = Synthetic Construct
GASKLAS

SEQ ID NO: 49 - protein, 11 aa, synthetic construct
  REGION 1..11: note = Synthetic Construct
QATYYSSNSVA

SEQ ID NO: 50 - protein, 5 aa, synthetic construct
  REGION 1..5: note = Synthetic Construct
SYAMG

SEQ ID NO: 51 - protein, 16 aa, synthetic construct
  REGION 1..16: note = Synthetic Construct
IINSYGNTYYANWAKG

SEQ ID NO: 52 - protein, 8 aa, synthetic construct
  REGION 1..8: note = Synthetic Construct
DPGVSSNL

SEQ ID NO: 53 - protein, 11 aa, synthetic construct
  REGION 1..11: note = Synthetic Construct
QASQSISSSLA

SEQ ID NO: 54 - protein, 7 aa, synthetic construct
  REGION 1..7: note = Synthetic Construct
AASILAS

SEQ ID NO: 55 - protein, 12 aa, synthetic construct
  REGION 1..12: note = Synthetic Construct
QCTSYGSLFLGP

SEQ ID NO: 56 - protein, 5 aa, synthetic construct
  REGION 1..5: note = Synthetic Construct
ANTMN

SEQ ID NO: 57 - protein, 16 aa, synthetic construct
  REGION 1..16: note = Synthetic Construct
IFTATGSTYYATWVNG

SEQ ID NO: 58 - protein, 12 aa, synthetic construct
  REGION 1..12: note = Synthetic Construct
SGSGSSSGAFNI

SEQ ID NO: 59 - protein, 11 aa, synthetic construct
  REGION 1..11: note = Synthetic Construct
QASQSISNFLA

SEQ ID NO: 60 - protein, 7 aa, synthetic construct
  REGION 1..7: note = Synthetic Construct
AASHLAS

SEQ ID NO: 61 - protein, 13 aa, synthetic construct
  REGION 1..13: note = Synthetic Construct
QSYFYSSTSIYNA

SEQ ID NO: 62 - protein, 5 aa, synthetic construct
  REGION 1..5: note = Synthetic Construct
SYALG

SEQ ID NO: 63 - protein, 16 aa, synthetic construct
  REGION 1..16: note = Synthetic Construct
IISSTGTTYYATWAKG

SEQ ID NO: 64 - protein, 16 aa, synthetic construct
  REGION 1..16: note = Synthetic Construct
GAYAGYVAFGPYYFHI

SEQ ID NO: 65 - protein, 111 aa, synthetic construct
  REGION 1..111: note = Synthetic Construct
AAVLTQTPSPVSAAVGGTVTINCQSSPNIYSNYLSWFQQKPGQPPKILIYGASTLASGVPSRFKGSGSGTQFTLTISDVQCDDAATYYCAGGYSDSSEAYAFGGGTEVVVK

SEQ ID NO: 66 - protein, 111 aa, synthetic construct
  REGION 1..111: note = Synthetic Construct
AAVLTQTPSPVSAAVGGTVSISCQSSKSIYNNNWLSWYQQKPGQPPKLLIYDASDLASGVPSRFEGSGSGTEFTLTISDLECDDAATYYCAGGYSGDSDYAFGGGTEVVVK

SEQ ID NO: 67 - protein, 111 aa, synthetic construct
  REGION 1..111: note = Synthetic Construct
DAQLTQSPSTLSASVGDRVTITCQSSKSIYNNNWLSWYQQKPGKPPKLLIYDASDLASGVPSRFSGSGSGTEFTLTISSLQPDDFATYYCAGGYSGDSDYAFGGGTKVEIK

SEQ ID NO: 68 - protein, 111 aa, synthetic construct
  REGION 1..111: note = Synthetic Construct
DIQLTQSPSTLSASVGDRVTITCQSSKSIYNNNWLSWYQQKPGKPPKLLIYDASDLASGVPSRFSGSGSGTEFTLTISSLQPDDFATYYCAGGYSGDSDYAFGGGTKVEIK

SEQ ID NO: 69 - protein, 109 aa, synthetic construct
  REGION 1..109: note = Synthetic Construct
AQVLIQTASSVSAAVGGTVTISCQSSESVYNNDYLAWYQQKPGQPPKLLIYSASTLASGVPSRFKGSGSGTQFTLTISDLECDDAATYYCAGGYLGNNVFGGGTEVVVK

SEQ ID NO: 70 - protein, 109 aa, synthetic construct
  REGION 1..109: note = Synthetic Construct
DQVLTQSPDSLAVSLGERATINCQSSESVYNNDYLAWYQQKPGQPPKLLIYSASTLASGVPDRFSGSGSGTDFTLTISSLQAEDVAVYYCAGGYLGNNVFGGGTKVEIK

SEQ ID NO: 71 - protein, 112 aa, synthetic construct
  REGION 1..112: note = Synthetic Construct
AAVLTQTPSPVSATMGGTVSISCQSSKSVYNNNWLSWYQQKPGQPPKLLIYRASTLESGVPSRFKGSGSGTQFTLTISDVHCDDAATYFCAGGYSSSSSANAFGGGTEVVVK

SEQ ID NO: 72 - protein, 112 aa, synthetic construct
  REGION 1..112: note = Synthetic Construct
DAVLTQSPDSLAVSLGERATINCQSSKSVYNNNWLSWYQQKPGQPPKLLIYRASTLESGVPDRFSGSGSGTDFTLTISSLQAEDVAVYFCAGGYSSSSSANAFGGGTKVEIK

SEQ ID NO: 73 - protein, 112 aa, synthetic construct
  REGION 1..112: note = Synthetic Construct
DIVLTQSPDSLAVSLGERATINCQSSKSVYNNNWLSWYQQKPGQPPKLLIYRASTLESGVPDRFSGSGSGTDFTLTISSLQAEDVAVYFCAGGYSSSSSANAFGGGTKVEIK

SEQ ID NO: 74 - protein, 109 aa, synthetic construct
  REGION 1..109: note = Synthetic Construct
AQVLTQTASSVSAAVGGTVTINCQASQSVYDSKWLAWYQQKPGQPPKLLIYSASTLASGVPSRFKGSGSGTQFTLTISDLECDDAATYYCAGAYTDNIVFGGGTEVVVK

SEQ ID NO: 75 - protein, 112 aa, synthetic construct
  REGION 1..112: note = Synthetic Construct
AQVLTQTASSVSAAVGGTVTISCQSSPSVYNHNWLSWYQQKPGQPPKLLIYEASKLASGVPSRFSGSGSGTQFTLTISDVQCDEAATYYCAGGFSSGSDSFAFGGGTEVVVT

SEQ ID NO: 76 - protein, 111 aa, synthetic construct
  REGION 1..111: note = Synthetic Construct
DPVLTQTPSSVEAAVGGTVTIKCQASESISSRLAWYQQKPGQPPKLLIYSASTLASGVSSRFKGSGSGTEFTLTISDLECADAATYYCQTYYGGSTTGWYVFGGGTEVVVK

SEQ ID NO: 77 - protein, 110 aa, synthetic construct
  REGION 1..110: note = Synthetic Construct
DVVMTQTPASVEAAVGGTVTIKCQASQSIGNALAWYQQKPGQRPKLLIYAASNLASGVPSRFAGSGSGTQFTLTISDLECADAATYYCQTYYAINRYGGAFGGGTEVVVK

SEQ ID NO: 78 - protein, 109 aa, synthetic construct
  REGION 1..109: note = Synthetic Construct
DVVMTQTPASVSEPVGGTVTIKCQASQNIYSGLAWYQQKPGQPPKLLIYGASKLASGVSSRFKGSGSGTEFTLTISDLECADAATYYCQATYYSSNSVAFGGGTEVVVK

SEQ ID NO: 79 - protein, 109 aa, synthetic construct
  REGION 1..109: note = Synthetic Construct
DVQMTQSPSTLSASVGDRVTITCQASQNIYSGLAWYQQKPGKPPKLLIYGASKLASGVPSRFSGSGSGTEFTLTISSLQPDDFATYYCQATYYSSNSVAFGGGTKVEIK

SEQ ID NO: 80 - protein, 109 aa, synthetic construct
  REGION 1..109: note = Synthetic Construct
DIQMTQSPSTLSASVGDRVTITCQASQNIYSGLAWYQQKPGKAPKLLIYGASKLASGVPSRFSGSGSGTEFTLTISSLQPDDFATYYCQATYYSSNSVAFGGGTKVEIK

SEQ ID NO: 81 - protein, 110 aa, synthetic construct
  REGION 1..110: note = Synthetic Construct
DPVLTQTPASVSEPVGGTVTIKCQASQSISSSLAWYQQKPGQPPKLLIYAASILASEISSRFKGSRSGTEFTLTISDLECADAATYYCQCTSYGSLFLGPFGGGTEVVVK

SEQ ID NO: 82 - protein, 111 aa, synthetic construct
  REGION 1..111: note = Synthetic Construct
DIVMTQTPASVEAAVGGTVTIKCQASQSISNFLAWYQQKPGQPPKVLIYAASHLASGVPSRFKGSGSGTQFTLTISDLECADAATYYCQSYFYSSTSIYNAFGGGTEVVVR

SEQ ID NO: 83 - protein, 123 aa, synthetic construct
  REGION 1..123: note = Synthetic Construct
QSVQESGGRLVTPGTPLTLTCTVSGFSLINYPMSWVRQAPGKGLEWIGVISSTGGTNYASWAKGRFTISKTSTTVDLKITSPTTEDTATYFCARVDFLVYLGGAYIIWGLDLWGQGTLVTVSS

SEQ ID NO: 84 - protein, 125 aa, synthetic construct
  REGION 1..125: note = Synthetic Construct
QSVEESGGRLVTPGTPLTLTCTVSGFSLSSNAMSWVRQAPGKGLEWIGIISSSGSTYSASWAKGRFTISKTSTTMDLKITSPTTEDTATYFCARVGFFVGYGAYDYGIIHRLDLWGQGTLVTVSS

SEQ ID NO: 85 - protein, 128 aa, synthetic construct
  REGION 1..128: note = Synthetic Construct
EQQLVESGGGLIQPGGSLRLSCAVSGFSLSSNAMSWVRQAPGKGLEWIGIISSSGSTYSASWAKGRFTISKDSSKNTMYLQMNSLRAEDTAVYFCARVGFFVGYGAYDYGIIHRLDLWGQGTLVTVSS

SEQ ID NO: 86 - protein, 128 aa, synthetic construct
  REGION 1..128: note = Synthetic Construct
EVQLVESGGGLIQPGGSLRLSCAASGFSLSSNAMSWVRQAPGKGLEWVSIISSSGSTYSASWAKGRFTISKDNSKNTLYLQMNSLRAEDTAVYYCARVGFFVGYGAYDYGIIHRLDLWGQGTLVTVSS

SEQ ID NO: 87 - protein, 124 aa, synthetic construct
  REGION 1..124: note = Synthetic Construct
QSLEESGGRLVTPGTPLTLTCTASGFSLSGYTMGWVRQAPGKGLEYIGVISSGGTTYYTNWAKGRFTISKTSTTVDLKITSPTTEDTATYFCARVAFTAYGGGGFPTLHRLDLWGQGTLVTVSS

SEQ ID NO: 88 - protein, 127 aa, synthetic construct
  REGION 1..127: note = Synthetic Construct
EQQLVESGGGLVQPGGSLRLSCAASGFSLSGYTMGWVRQAPGKGLEYIGVISSGGTTYYTNWAKGRFTISKDSSKNTVYLQMGSLRAEDMAVYFCARVAFTAYGGGGFPTLHRLDLWGQGTLVTVSS

SEQ ID NO: 89 - protein, 127 aa, synthetic construct
  REGION 1..127: note = Synthetic Construct
EVQLVESGGGLVQPGGSLRLSCAASGFSLSGYTMGWVRQAPGKGLEYIGVISSGGTTYYTNWAKGRFTISKDNSKNTLYLQMGSLRAEDMAVYYCARVAFTAYGGGGFPTLHRLDLWGQGTLVTVSS

SEQ ID NO: 90 - protein, 125 aa, synthetic construct
  REGION 1..125: note = Synthetic Construct
QSVEESGGRLVTPGTPLTLTCTVSGFSLSGYAMSWVRQAPGKGLEYIGVISSSGSSYYPSWAKGRFTISKTSTTVDLQITSPTTEDTATYFCARVQFYVGYAVYGYGIIDRLDLWGQGTLVTVSS

SEQ ID NO: 91 - protein, 128 aa, synthetic construct
  REGION 1..128: note = Synthetic Construct
EQQLVESGGGLVQPGGSLRLSCAVSGFSLSGYAMSWVRQAPGKGLEYIGVISSSGSSYYPSWAKGRFTISKDSSKNTVYLQMGSLRAEDMAVYFCARVQFYVGYAVYGYGIIDRLDLWGQGTLVTVSS

SEQ ID NO: 92 - protein, 128 aa, synthetic construct
  REGION 1..128: note = Synthetic Construct
EVQLVESGGGLVQPGGSLRLSCAVSGFSLSGYAMSWVRQAPGKGLEYIGVISSSGSSYYPSWAKGRFTISKDNSKNTLYLQMGSLRAEDMAVYYCARVQFYVGYAVYGYGIIDRLDLWGQGTLVTVSS

SEQ ID NO: 93 - protein, 123 aa, synthetic construct
  REGION 1..123: note = Synthetic Construct
QSLEESGGRLVTPGTPLTLTCTVSGFSFSSYSMGWVRQAPGKGPEYIGVISASGTTYYASWVNGRFTISKTSTTMDLKMTSPTAADTATYFCARAAFTAYNRGSCVIHRLDLWGQGTLVTVSS

SEQ ID NO: 94 - protein, 119 aa, synthetic construct
  REGION 1..119: note = Synthetic Construct
QSVEESGGRLVTPGTPLTLTCTVSGFSLSTYSMSWVRQAPGKGLEWLGIVSVAIDPVYATWARGRFTISRTSTTVNLKITSPTTEDTATYFCVRVAFSTNGIPHRLDLWGQGTLVTVSS

SEQ ID NO: 95 - protein, 128 aa, synthetic construct
  REGION 1..128: note = Synthetic Construct
QELVESGGGLVQAGESLKLSCKASGIDFSSYGISWVRQAPGKGLEWIAYIYPGFGITNYAHSVKGRFTISSDNAQNTVFLQMPSLTASDTATYFCARDLDYTGGVVGYAYVTYYFTLWGPGTLVTVSS

SEQ ID NO: 96 - protein, 116 aa, synthetic construct
  REGION 1..116: note = Synthetic Construct
QSVEESGGRLVTPGTPLTVTCTVSGFSLSVYGMGWVRQAPGKGLEYIGFINNVGNTYYASWAKGRFTISKTSTTVDLKITSPTTEDTATYFCAKGGGGDWGYFNIWGPGTLVTVSL

SEQ ID NO: 97 - protein, 113 aa, synthetic construct
  REGION 1..113: note = Synthetic Construct
QSVEESGGRLVTPGTPLTLTCTVSGFSLSSYAMGWVRQAPGKGLEWIGIINSYGNTYYANWAKGRFTISRTSTTVDLRMPSLTTEDTATYFCARDPGVSSNLWGPGTLVTVSS

SEQ ID NO: 98 - protein, 116 aa, synthetic construct
  REGION 1..116: note = Synthetic Construct
EQQLVESGEGLVQPGGSLRLSCAVSGFSLSSYAMGWVRQAPGKGLEWIGIINSYGNTYYANWAKGRFTISRDSSKNTVYLQMGSLRAEDMAVYFCARDPGVSSNLWGPGTLVTVSS

SEQ ID NO: 99 - protein, 116 aa, synthetic construct
  REGION 1..116: note = Synthetic Construct
EVQLVESGEGLVQPGGSLRLSCAASGFSLSSYAMGWVRQAPGKGLEYVGIINSYGNTYYANWAKGRFTISRDNSKNTVYLQMGSLRAEDMAVYYCARDPGVSSNLWGRGTLVTVSS

SEQ ID NO: 100 - protein, 117 aa, synthetic construct
  REGION 1..117: note = Synthetic Construct
QSVEESGGRLVTPGTPLTLTCTVSGIDLSANTMNWVRQAPGKGLEWIGIFTATGSTYYATWVNGRFTISKTSTTVDLKITSPTTEDTATYFCARSGSGSSSGAFNIWGPGTLVTVSL

SEQ ID NO: 101 - protein, 122 aa, synthetic construct
  REGION 1..122: note = Synthetic Construct
QSLEESGGRLVTPGTPLTLTCTVSGIDLSSYALGWVRQAPGKGLEYIGIISSTGTTYYATWAKGRFTISKTSSTTVDLKITGPTTEDTATYFCARGAYAGYVAFGPYYFHIWGPGTLVTISL

SEQ ID NO: 102 - protein, 457 aa, synthetic construct
  REGION 1..457: note = Synthetic Construct
EQQLVESGGGLIQPGGSLRLSCAVSGFSLSSNAMSWVRQAPGKGLEWIGIISSSGSTYSASWAKGRFTISKDSSKNTMYLQMNSLRAEDTAVYFCARVGFFVGYGAYDYGIIHRLDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 103 - protein, 457 aa, synthetic construct
  REGION 1..457: note = Synthetic Construct
EVQLVESGGGLIQPGGSLRLSCAASGFSLSSNAMSWVRQAPGKGLEWVSIISSSGSTYSASWAKGRFTISKDNSKNTLYLQMNSLRAEDTAVYYCARVGFFVGYGAYDYGIIHRLDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 104 - protein, 456 aa, synthetic construct
  REGION 1..456: note = Synthetic Construct
EQQLVESGGGLVQPGGSLRLSCAASGFSLSGYTMGWVRQAPGKGLEYIGVISSGGTTYYTNWAKGRFTISKDSSKNTVYLQMGSLRAEDMAVYFCARVAFTAYGGGGFPTLHRLDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 105 - protein, 456 aa, synthetic construct
  REGION 1..456: note = Synthetic Construct
EVQLVESGGGLVQPGGSLRLSCAASGFSLSGYTMGWVRQAPGKGLEYIGVISSGGTTYYTNWAKGRFTISKDNSKNTLYLQMGSLRAEDMAVYYCARVAFTAYGGGGFPTLHRLDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 106 - protein, 457 aa, synthetic construct
  REGION 1..457: note = Synthetic Construct
EQQLVESGGGLVQPGGSLRLSCAVSGFSLSGYAMSWVRQAPGKGLEYIGVISSSGSSYYPSWAKGRFTISKDSSKNTVYLQMGSLRAEDMAVYFCARVQFYVGYAVYGYGIIDRLDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 107 - protein, 457 aa, synthetic construct
  REGION 1..457: note = Synthetic Construct
EVQLVESGGGLVQPGGSLRLSCAVSGFSLSGYAMSWVRQAPGKGLEYIGVISSSGSSYYPSWAKGRFTISKDNSKNTLYLQMGSLRAEDMAVYYCARVQFYVGYAVYGYGIIDRLDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 108 - protein, 445 aa, synthetic construct
  REGION 1..445: note = Synthetic Construct
EQQLVESGEGLVQPGGSLRLSCAVSGFSLSSYAMGWVRQAPGKGLEWIGIINSYGNTYYANWAKGRFTISRDSSKNTVYLQMGSLRAEDMAVYFCARDPGVSSNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 109 - protein, 445 aa, synthetic construct
  REGION 1..445: note = Synthetic Construct
EVQLVESGEGLVQPGGSLRLSCAASGFSLSSYAMGWVRQAPGKGLEYVGIINSYGNTYYANWAKGRFTISRDNSKNTVYLQMGSLRAEDMAVYYCARDPGVSSNLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 110 - protein, 218 aa, synthetic construct
  REGION 1..218: note = Synthetic Construct
DAQLTQSPSTLSASVGDRVTITCQSSKSIYNNNWLSWYQQKPGKPPKLLIYDASDLASGVPSRFSGSGSGTEFTLTISSLQPDDFATYYCAGGYSGDSDYAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - protein, 218 aa, synthetic construct
  REGION 1..218: note = Synthetic Construct
DIQLTQSPSTLSASVGDRVTITCQSSKSIYNNNWLSWYQQKPGKPPKLLIYDASDLASGVPSRFSGSGSGTEFTLTISSLQPDDFATYYCAGGYSGDSDYAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 216 aa, synthetic construct
  REGION 1..216: note = Synthetic Construct
DQVLTQSPDSLAVSLGERATINCQSSESVYNNDYLAWYQQKPGQPPKLLIYSASTLASGVPDRFSGSGSGTDFTLTISSLQAEDVAVYYCAGGYLGNNVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 216 aa, synthetic construct; identical to SEQ ID NO: 112
  REGION 1..216: note = Synthetic Construct

SEQ ID NO: 114 - protein, 219 aa, synthetic construct
  REGION 1..219: note = Synthetic Construct
DAVLTQSPDSLAVSLGERATINCQSSKSVYNNNWLSWYQQKPGQPPKLLIYRASTLESGVPDRFSGSGSGTDFTLTISSLQAEDVAVYFCAGGYSSSSSANAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 219 aa, synthetic construct
  REGION 1..219: note = Synthetic Construct
DIVLTQSPDSLAVSLGERATINCQSSKSVYNNNWLSWYQQKPGQPPKLLIYRASTLESGVPDRFSGSGSGTDFTLTISSLQAEDVAVYFCAGGYSSSSSANAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 216 aa, synthetic construct
  REGION 1..216: note = Synthetic Construct
DVQMTQSPSTLSASVGDRVTITCQASQNIYSGLAWYQQKPGKPPKLLIYGASKLASGVPSRFSGSGSGTEFTLTISSLQPDDFATYYCQATYYSSNSVAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 216 aa, synthetic construct
  REGION 1..216: note = Synthetic Construct
DIQMTQSPSTLSASVGDRVTITCQASQNIYSGLAWYQQKPGKAPKLLIYGASKLASGVPSRFSGSGSGTEFTLTISSLQPDDFATYYCQATYYSSNSVAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 118 - protein, 387 aa, synthetic construct
  REGION 1..387: note = Synthetic Construct
DGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 119 - protein, 214 aa, synthetic construct
  REGION 1..214: note = Synthetic Construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 447 aa, synthetic construct
  REGION 1..447: note = Synthetic Construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 121 - protein, 218 aa, synthetic construct
  REGION 1..218: note = Synthetic Construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - protein, 10 aa, synthetic construct
  REGION 1..10: note = Synthetic Construct
GFTFSDSWIH

SEQ ID NO: 123 - protein, 18 aa, synthetic construct
  REGION 1..18: note = Synthetic Construct
AWISPYGGSTYYADSVKG

SEQ ID NO: 124 - protein, 9 aa, synthetic construct
  REGION 1..9: note = Synthetic Construct
RHWPGGFDY

SEQ ID NO: 125 - protein, 11 aa, synthetic construct
  REGION 1..11: note = Synthetic Construct
RASQDVSTAVA

SEQ ID NO: 126 - protein, 7 aa, synthetic construct
  REGION 1..7: note = Synthetic Construct
SASFLYS

SEQ ID NO: 127 - protein, 9 aa, synthetic construct
  REGION 1..9: note = Synthetic Construct
QQYLYHPAT

SEQ ID NO: 128 - protein, 118 aa, synthetic construct [sequence omitted]

SEQ ID NO: 129 - protein, 108 aa, synthetic construct
  REGION 1..108: note = Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 130 - protein, 447 aa, synthetic construct
  REGION 1..447: note = Synthetic Construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 131 - protein, 214 aa, synthetic construct
  REGION 1..214: note = Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - protein, 449 aa, synthetic construct
  REGION 1..449: note = Synthetic Construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 133 - protein, 216 aa, synthetic construct
  REGION 1..216: note = Synthetic Construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 134 - protein, 450 aa, synthetic construct
  REGION 1..450: note = Synthetic Construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 135 - protein, 215 aa, synthetic construct
  REGION 1..215: note = Synthetic Construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 8 aa, synthetic construct
  REGION 1..8: note = Synthetic Construct
SIINFEKL

SEQ ID NO: 137 - protein, 999 aa, Homo sapiens
MGPAPLPLLLGLFLPALWRRAITEAREEAKPYPLFPGPFPGSLQTDHTPLLSLPHASGYQPALMFSPTQPGRPHTGNVAIPQVTSVESKPLPPLAFKHTVGHIILSEHKGVKFNCSISVPNIYQDTTISWWKDGKELLGAHHAITQFYPDDEVTAIIASFSITSVQRSDNGSYICKMKINNEEIVSDPIYIEVQGLPHFTKQPESMNVTRNTAFNLTCQAVGPPEPVNIFWVQNSSRVNEQPEKSPSVLTVPGLTEMAVFSCEAHNDKGLTVSKGVQINIKAIPSPPTEVSIRNSTAHSILISWVPGFDGYSPFRNCSIQVKEADPLSNGSVMIFNTSALPHLYQIKQLQALANYSIGVSCMNEIGWSAVSPWILASTTEGAPSVAPLNVTVFLNESSDNVDIRWMKPPTKQQDGELVGYRISHVWQSAGISKELLEEVGQNGSRARISVQVHNATCTVRIAAVTRGGVGPFSDPVKIFIPAHGWVDYAPSSTPAPGNADPVLIIFGCFCGFILIGLILYISLAIRKRVQETKFGNAFTEEDSELVVNYIAKKSFCRRAIELTLHSLGVSEELQNKLEDVVIDRNLLILGKILGEGEFGSVMEGNLKQEDGTSLKVAVKTMKLDNSSQREIEEFLSEAACMKDFSHPNVIRLLGVCIEMSSQGIPKPMVILPFMKYGDLHTYLLYSRLETGPKHIPLQTLLKFMVDIALGMEYLSNRNFLHRDLAARNCMLRDDMTVCVADFGLSKKIYSGDYYRQGRIAKMPVKWIAIESLADRVYTSKSDVWAFGVTMWEIATRGMTPYPGVQNHEMYDYLLHGHRLKQPEDCLDELYEIMYSCWRTDPLDRPTFSVLRLQLEKLLESLPDVRNQADVIYVNTQLLESSEGLAQGSTLAPLDLNIDPDSIIASCTPRAAISVVTAEVHDSKPHEGRYILNGGSEEWEDLTSAPSAAVTAEKNSVLPGERLVRNGVSWSHSSMLPLGSSLPDELLFADDSSEGSEVLM